(19) United States
(12) Patent Application Publication
(10) Pub. No.: US 2023/0063457 A1
(43) Pub. Date: Mar. 2, 2023

(54) DNA-PK INHIBITING COMPOUNDS

(71) Applicants: Andrew Ivor MINCHINTON, Vancouver (CA); Alastair Hugh KYLE, Vancouver (CA); James EVANS, Santa Cruz, CA (US); Samuel Edward MANN, Amersham,Buckinghamshire (GB); George HYND, Epping, Essex (GB); Provincial Health Services Authority, Vancouver (CA)

(72) Inventors: Andrew Ivor MINCHINTON, Vancouver (CA); Alastair Hugh KYLE, Vancouver (CA); James EVANS, Santa Cruz, CA (US); Samuel Edward MANN, Amersham, Buckinghamshire (GB); George HYND, Epping, Essex (GB)

(21) Appl. No.: 17/641,813

(22) PCT Filed: Sep. 11, 2019
(86) PCT No.: PCT/US2019/050577

§ 371 Date: Mar. 9, 2022

(51) Int. Cl. A61K 31/5377; A61K 45/06; A61P 35/00; C07D 471/04; A61K 31/55; C07D 519/00

(52) U.S. Cl. CPC A61K 31/5377; A61K 45/06; A61P 35/00; C07D 471/04; A61K 31/55; C07D 519/00; C07B 2200/05

(57) ABSTRACT

The present disclosure relates to DNA-PK inhibiting compounds and prodrugs thereof that are useful in the treatment of diseases, including cancer. In particular, the compounds sensitise cancers to therapies such as chemotherapy and radiotherapy.

### CROSS-REFERENCES TO RELATED APPLICATIONS

This application is a U.S. National Phase Application of PCT International Application No. PCT/US2019/050577, filed Sep. 11, 2019, which is incorporated herein in its entirety for all purposes.

### TECHNICAL FIELD

This invention relates to compounds that are useful as DNA-dependent protein kinase (DNA-PK) inhibitors and the use of the compounds to treat diseases, including cancer. In particular, the compounds inhibit DNA-PK and thus sensitise cancers to therapies such as chemotherapy and radiotherapy. Certain compounds of the invention are in the form of prodrugs that release the DNA-PK inhibitor in hypoxic tissue such as is known to occur in cancers.

### BACKGROUND OF THE INVENTION

Radiation therapy involves the exposure of a cancer to ionizing radiation (IR) at a dose that kill cells. Radiation therapy is administered as a beam of ionizing radiation typically from a linear accelerator, an x-ray machine, a cyclotron or ⁶⁰Cobalt unit or by implantation or temporary application of radioactive isotopes. Radiation therapy can be very effective, affording cure in a proportion of cases. Since it is not technically possible to selectively irradiate only the cancer cells, the dose-limiting factor associated with radiation therapy is the damage done to non-cancerous tissue. As a consequence, doses of radiation are prescribed which deliver the maximum dose of radiation to the tumour tissue, while exposing normal tissue to doses that produce tolerable side effects.

IR causes a variety of cellular damage but it is the damage to the cell's DNA that is believed to the primary cause of cell killing. The amount of DNA damage and the repair of that damage by DNA repair enzymes determines the extent of cell kill.

Cells have evolved pathways for the repair of its genetic material caused either by endogenous metabolism or exogenous sources of ionizing radiation. The pathways that have evolved are often specific for the type of chemical lesions produced in DNA. IR produces a variety of lesions including base damage, single strand breaks, DNA-DNA and DNA-protein crosslinks and double strand breaks. However, the principle lethal event caused by IR used in radiotherapy is believed to be the induction of DNA double strand breaks (DSB). DSB's are repaired by several enzymatic pathways. One is non-homologous end-joining (NHEJ) that occurs in all phases of the cell cycle. DSB's can also be repaired by homologous recombination (HR) in cells where the repair machinery has access to a homologous strand of DNA from a sister chromatid. As a consequence, HR occurs primarily in late S and G2 phases of the cell cycle. Other mechanisms elucidated include alt-End joining.

Hypoxic cells (cells at lower than normal physiological oxygen tension) are commonly found in human tumours. They arise either because the cellular proliferation within tumours results in cells becoming located beyond the diffusion distance of oxygen from the nearest functioning blood vessel (Thomlinson & Gray 1955 Br. J. Cancer 9 539-549) or as a result of temporary interruptions of blood flow (Chalin et al. 1987 Cancer Res. 47 597-601).

Hypoxic cells are resistant to ionizing radiation (IR) because molecular oxygen can react with the sites of initial molecule ionization making the damage more difficult to repair and because in the absence of oxygen spontaneous reductive reactions occur to restitute the original molecule. Thus, hypoxia reduces the effectiveness of radiotherapy. Clinical studies measuring oxygen tension in tumours Nordsmark et al. 2005 Radiother. Oncol. 77 18-24) and clinical trials of treatments which increase tumour oxygenation or drugs which act as oxygen mimetics Overgaard 2007 J. Clin. Oncol. 25 4066-4074) have confirmed the role of hypoxic cells as an impediment to the effectiveness of radiation therapy.

Hypoxic cells are less likely to be proliferating because of oxygen deprivation so are predominantly in the G1 phase of the cell cycle and thus DNA DSB in hypoxic cells would primarily be repaired by NHEJ.

Early attempts to sensitize hypoxic cells to ionizing radiation used 2-nitroimidazole compounds to selectively increase the initial number of DNA lesions caused by a given dose of radiation (Adams 1991 Int. J. Radit. Oncol. Biol. Phys. 20 643-644). The 2-nitroimidazoles misonidazole and etanidazole, completed Phase III studies but dose limiting toxicities resulted in these drugs achieving only marginal efficacy (Overgaard 1994 Oncology Res. 6 509-518

Later strategies to selectively kill hypoxic cells were based on compounds that were activated only under hypoxic conditions to release an active cytotoxic compound. The first example was tirapazamine which entered clinical trials in combination with cisplatin, carboplatin, paclitaxel, etoposide, vinorelbine, cyclophosphamide and other chemotherapy agents with or without concomitant radiation therapy (Brown 1993 Br. J. Cancer 67 1163-1170). Normal tissue toxicities prevented its approval as an anti-cancer drug.

Other hypoxia activated cytotoxins have being developed. For example, PR-104 is a dinitrobenzamide mustard that entered clinical trials for the treatment of certain solid cancers (Guise et al. 2010 Cancer Res. 70 1573-1584). However, it was found that the compound was reduced under oxygenated conditions and is therefore unlikely to be suitable as an anti-cancer therapy. TH-302 is a nitromidazole phosphoramidate mustard in clinical trials in combination with doxorubicin, gemcitabine, docetaxel, pemetrexed for the treatment of sarcomas, non-small cell lung cancer and advanced solid cancers is currently under clinical evaluation (Boyle & Travers 2006 Anticancer Agents Med. Chem. 6 281-286).

Hypoxic cells are likely to limit the effectiveness of anticancer chemotherapy in part because hypoxic cells often reside distal to blood vessels. The distance from blood vessels to hypoxic cells is estimated to be 100-200 μm. There is a significant body of evidence that suggests cancer chemotherapy agents may not effectively reach cells distal to blood vessels (Minchinton & Tannock 2006 Nat. Rev. Cancer 6 583-592). Increasing the sensitivity of hypoxic cells to DNA damage caused by cancer chemotherapy agents would have the effect of improving anticancer drug efficacy.

Head & Neck (H&N) cancer is an example of a cancer commonly treated with radiotherapy. H&N cancers accounts for 6% of all cancers, an estimated 650,000 new cases each year worldwide. The majority of H&N cancers are squamous cell carcinomas presenting as locally advanced tumours that require surgery, radiotherapy, a combination of surgery and radiotherapy and, more recently, chemotherapy for treatment. More than 50% of patients suffer a recurrence and die from their disease. Treatment for H&N cancer is complicated by the proximity of cancerous tissue to e.g. the lip, oral cavity, nasopharynx, oropharynx, larynx or hypopharynx to that of normal organs. Improvements to radiotherapy delivery have reduced the damage to normal tissues; however, the spinal cord, brainstem, salivary glands, swallowing structures, optic nerves, chiasm and temporal lobes of the brain are all critical organs that require protection and, therefore, necessitate radiation dose limitations. It has been said that contemporary treatment for locally advanced H&N cancer is at the “upper limit of human tolerance of acute toxicities”, and that currently unrecognized damage as a result of treatments are so acute that they actually “contribute substantially to patient mortality” (Corry et al 2010 Lancet 11 287-291).

DNA-PK (DNA-dependent protein kinase) is an enzyme involved in the repair of DNA DSBs. DNA-PK is a member of the PI3 kinase-like kinase (PIKK) family of atypical protein kinases. The important role of DNA-PK in cell survival following radiation therapy is well established. Small molecule DNA-PK inhibitors have demonstrated between 2 to 7-fold radiosensitization of cells in vitro and have been shown to inhibit DSB repair. Examples of small molecule DNA-PK inhibitors are provided in WO 2013/163190.

Currently, no hypoxia activated anticancer cytotoxic agents have been approved for clinical use. Therefore, despite the advances made in radiation therapy and chemotherapy and in targeting the hypoxic areas of tumours, there remains a need for compounds, compositions, and methods that can improve radiation-mediated killing of hypoxic cancer cells.

### BRIEF SUMMARY OF THE DISCLOSURE

In accordance with the present invention, there is provided a compound of formula (I), or prodrug thereof or a pharmaceutically acceptable salt or N-oxide of the compound of formula (I) or the prodrug thereof:

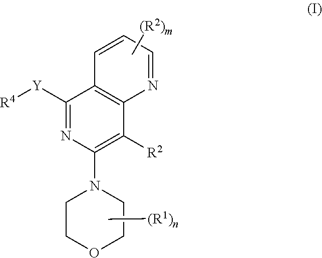

wherein
Y is independently selected from O and NR⁵;
R¹ is independently at each occurrence selected from C₁-C₆-alkyl and C₁-C₆-haloalkyl;
R² is independently selected from H, C₁-C₆-alkyl, C₁-C₆-haloalkyl, cyano and halo;
R³ is independently at each occurrence selected from C₁-C₆-alkyl, C₁-C₆-haloalkyl, cyano, halo, OR^{6a}, NR^{7a}R^{8a};
R⁴ is -L¹-L²-R^{9a};
R⁵ is independently selected from: H and C₁-C₆-alkyl;
or R⁴ and R⁵ together with the nitrogen to which they are attached form a 3- to 11-membered heterocycloalkyl group or a 5-membered heteroaryl group, said heterocycloalkyl group being optionally substituted with from 1 to 4 R^{10a} substituents and/or a single R¹¹ substituent and said heteroaryl group being optionally substituted with from 1 to 4 R^{12a} substituents and/or a single R¹¹ substituent; wherein said heterocycloalkyl group may be monocyclic, bicyclic or a spirocyclic bicycle;
-L¹- is independently either absent or is —C₁-C₆-alkylene, wherein said alkylene group is optionally substituted with from 1 to 4 R^{10b} substituents;
-L²- is independently either absent or is -L³-L⁴-;
-L³- is independently selected from: C₁-C₆-alkylene, C₃-C₈-cycloalkyl, 3- to 8-membered heterocycloalkyl, wherein said cycloalkyl or heterocycloalkyl group may be monocyclic, bicyclic or a spirocyclic bicycle and wherein said alkylene, cycloalkyl or heterocycloalkyl group may be optionally substituted with from 1 to 4 R^{10c} substituents;
-L⁴- is independently either absent or is selected from —NR^{13a}— and —O—;
R^{9a} and R^{9b} are each independently selected from: phenyl, naphthyl, 5, 6, 9 or 10 membered heteroaryl, 3- to 8-membered heterocycloalkyl, C₃-C₈-cycloalkyl and C₁-C₃-alkylene-R¹⁴; wherein R¹⁴ is independently selected from: phenyl, naphthyl, 5, 6, 9 or 10 membered heteroaryl, 3- to 8-membered heterocycloalkyl and C₃-C₈-cycloalkyl; wherein any phenyl, napthyl or heteroaryl group of which R^{9a} or R^{9b} is comprised is optionally substituted with from 1 to 4 R¹⁵ substituents and any alkylene, cycloalkyl or heterocycloalkyl group of which R^{9a} or R^{9b} is comprised is optionally substituted with from 1 to 4 R^{10d} substituents;
R¹¹ is -L⁵-L⁶-R^{9b};
-L⁵- is independently either absent or is selected from C₁-C₃-alkylene, C(O) and S(O)₂, wherein said alkylene group is optionally substituted with from 1 to 4 R^{10e} substituents;
-L⁶- is independently either absent or is independently selected from —NR^{13b}— and —O—;
R^{6a}, R^{6b}, R^{6c} and R^{6d} are each independently at each occurrence selected from: H, C₁-C₆-alkyl (which may be optionally substituted with from 1 to 3 O—C₁-C₄-alkyl groups) and C₁-C₆-haloalkyl;
R^{7a}, R^{7b}, R^{7c} and R^{7d} are each independently at each occurrence selected from H and C₁-C₆-alkyl (which may be optionally substituted with from 1 to 3 O—C₁-C₄-alkyl groups);
R^{8a}, R^{8b} and R^{8c} are each independently at each occurrence selected from H, C₁-C₆-alkyl (which may be optionally substituted with from 1 to 3 O—C₁-C₄-alkyl groups), C(O)—C₁-C₆-alkyl, S(O)₂—C₁-C₆-alkyl, C(O)—O—C₁-C₆-alkyl, C(O)-phenyl and S(O)₂-phenyl; wherein said phenyl groups are optionally substituted with from 1 to 4 R^{12b} groups;
R^{10a}, R^{10b}, R^{10c}, R^{10d} and R^{10e} are each independently at each occurrence selected from: ═O, ═S, C₁-C₆-alkyl, C₂-C₆-alkenyl, C₂-C₆-alkynyl, C₁-C₆-haloalkyl, cyano, halo, nitro, (CR^{7b}R^{7b})ₓOR^{6b}, (CR^{7b}R^{7b})ₓNR^{7b}R^{6b}, C(O)R^{7b}, C(O)NR^{7b}R^{7b}, C(O)OR^{7b}, S(O)₂R^{7b}, S(O)R^{7b}, S(O)₂NR^{7b}R^{7b} and phenyl; wherein said phenyl group is optionally substituted with from 1 to 4 R^{12c} groups;
R^{13a} and R^{13b} are each independently at each occurrence selected from H and C₁-C₆-alkyl;
R¹⁵ is independently at each occurrence selected from C₁-C₆-alkyl, C₂-C₆-alkenyl, C₂-C₆-alkynyl, C₁-C₆-haloalkyl, cyano, halo, nitro, (CR^{7c}R^{7c})ₓOR^{6c}, (CR^{7c}R^{7c})ₓNR^{7c}R^{6c}, C(O)R^{7c}, C(O)NR^{7c}R^{7c}, C(O)OR^{7c}, S(O)₂R^{7c}, S(O)R^{7c}, S(O)₂NR^{7c}R^{7c}, and phenyl; wherein said phenyl group is optionally substituted with from 1 to 4 R^{12d} groups;
R^{12a}, R^{12b}, R^{12c} and R^{12d} are each independently at each occurrence selected from: C₁-C₆-alkyl, C₂-C₆-alkenyl, C₂-C₆-alkynyl, C₁-C₆-haloalkyl, cyano, halo, nitro, OR^{6d}, NR^{7d}R¹⁷, C(O)R^{7d}, C(O)NR^{7d}R^{7d}, C(O)OR^{7d}, S(O)₂R^{7d}, S(O)R^{7d} and S(O)₂NR^{7d}R^{7d};
R¹⁷ is independently at each occurrence selected from H, C₁-C₆-alkyl, C(O)—C₁-C₆-alkyl, S(O)₂—C₁-C₆-alkyl and C(O)—O—C₁-C₆-alkyl;
n is an integer selected from 0, 1, 2 and 3;
m is an integer selected from 0, 1, 2, 3 and 4;
x is independently at each occurrence an integer selected from 0, 1, 2 and 3;
where the compound is optionally a prodrug of a compound of formula (I) or a salt or N-oxide of a prodrug of formula (I), the prodrug comprises a trigger moiety that releases the compound of formula (I) under reductive conditions.

The inventors have identified the compounds of formula (I) as potent DNA-PK inhibitors.

It may be that the compound is a prodrug of a compound of formula (I), or a salt or N-oxide of a prodrug of formula (I), and the prodrug comprises a trigger moiety that releases the compound of formula (I) under reductive conditions.

The inventors have identified that incorporating a trigger moiety that releases the compound of formula (I) under reductive conditions allows the selective release of the DNA-PK inhibitors of formula (I) in hypoxic tissue, such as occurs within solid tumours. Thus, said prodrugs are hypoxia-activated DNA-PK inhibitors that are expected to show reduced toxicity by employing two mechanisms for selectivity. Firstly, the compound have specificity for hypoxic cells and are therefore expected to exhibit reduced systemic DNA-PK inhibition in oxic cells in the body. Secondly, they would only impact cells sustaining DNA-damage resulting from e.g. radiotherapy. This double specificity has the potential to result in a wide safety margin.

The trigger moiety may have the structure:

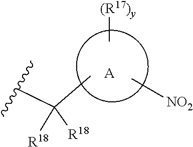

wherein ring A is a phenyl ring or a 5- or 6-membered heteroaryl ring;
R¹⁷ is independently at each occurrence selected from C₁-C₆-alkyl, C₁-C₆-haloalkyl, C₃-C₆-cycloalkyl, O—C₁-C₆-alkyl, cyano and halo;
R¹⁸ is independently at each occurrence selected from H, C₁-C₆-alkyl and C₁-C₆-haloalkyl; or the two R¹⁸ groups together form a C₃-C₆-cycloalkyl ring;
y is an integer from 0 to 3;
wherein the nitro group and the carbon attached to the two R¹⁸ groups are either attached to adjacent carbon atoms in Ring A or are attached to two carbon atoms in Ring A that are separated by two sp2 hybridised atoms selected from carbon and nitrogen.

The trigger moiety may be attached to that portion of the prodrug that will be released as the compound of formula (I) via a functional group derived from an attachment point on the compound of formula (I), said attachment point being selected from OH, NH, NH₂ and a quaternisable nitrogen.

In an embodiment, the compound of formula (I) is a compound of formula (II):

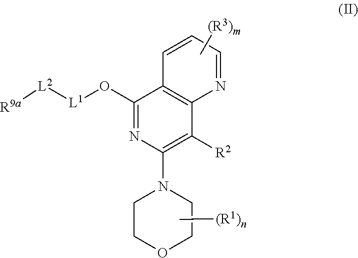

wherein R¹, R², R³, R^{9a}, -L¹-, -L²-, m and n are as described above for formula (I).

In an embodiment, the compound of formula (I) is a compound of formula (III):

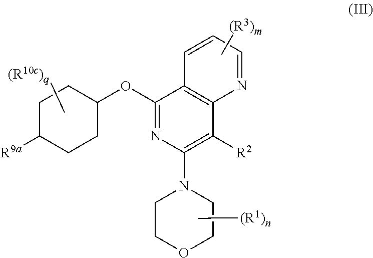

wherein R¹, R², R³, R^{9a}, R^{10c}, -L²-, m and n are as described above for formula (I); and
q is an integer selected from 0, 1, 2, 3 and 4.

In an embodiment, is provided a prodrug of a compound of formula (I), said prodrug having a structure according to formula (IV):

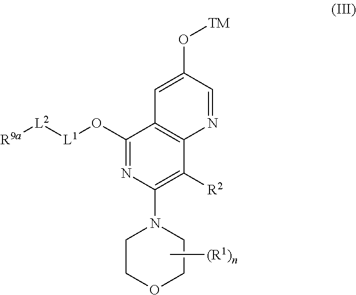

wherein R¹, R², R^{9a}, -L¹-, -L²-, m and n are as described above for formula (I); and wherein TM is the trigger moiety that releases a compound of formula (V) under reductive conditions:

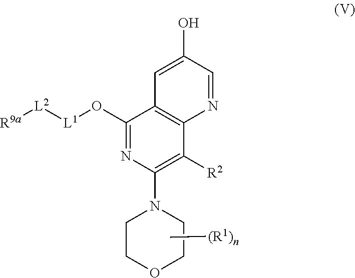

In an embodiment, the compound of formula (I) is a compound of formula (V).

In an embodiment, is provided a prodrug of a compound of formula (I), said prodrug having a structure according to formula (VI):

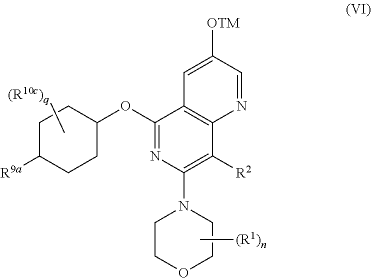

wherein R¹, R², R^{9a}, R^{10c}, -L¹-, -L²-, m and n are as described above for formula (I); q is an integer selected from 0, 1, 2, 3 and 4; and wherein TM is the trigger moiety that releases a compound of formula (VII) under reductive conditions:

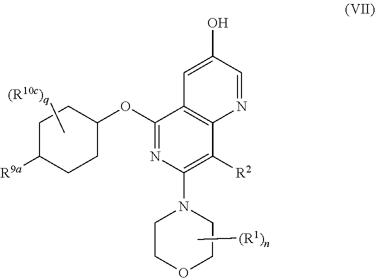

In an embodiment, the compound of formula (I) is a compound of formula (VII).

In an embodiment, the compound of formula (I) is a compound of formula (VIII):

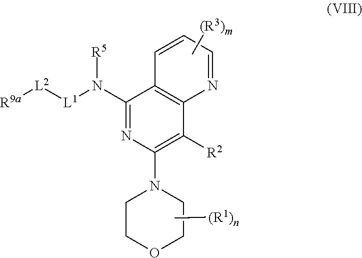

wherein R¹, R², R³, R⁵, R^{9a}, -L¹-, -L²-, m and n are as described above for formula (I).

In an embodiment, is provided a prodrug of a compound of formula (I), said prodrug having a structure according to formula (IX):

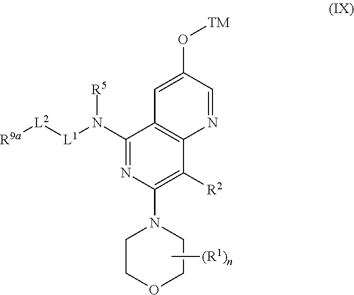

wherein R¹, R², R^{9a}, -L¹-, -L²-, m and n are as described above for formula (I); and wherein TM is the trigger moiety that releases a compound of formula (X) under reductive conditions:

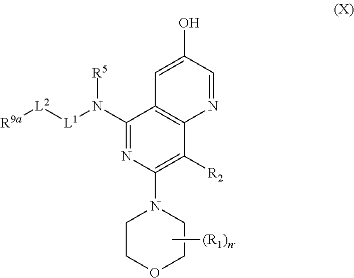

In an embodiment, the compound of formula (I) is a compound of formula (X).

In an embodiment, the compound of formula (I) is a compound of formula (XI):

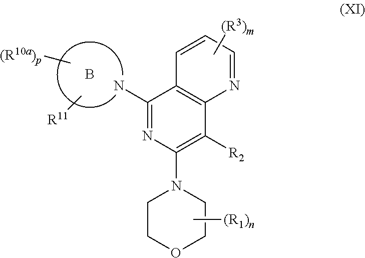

wherein R¹, R², R³, R^{10a}, R¹¹, m and n are as described above for formula (I); wherein ring B is a 3- to 11-membered heterocycloalkyl group that may be monocyclic, bicyclic or a spirocyclic bicycle; and p is an integer selected from 0, 1, 2, 3 and 4.

In an embodiment is provided a prodrug of a compound of formula (I), said prodrug having a structure according to formula (XII):

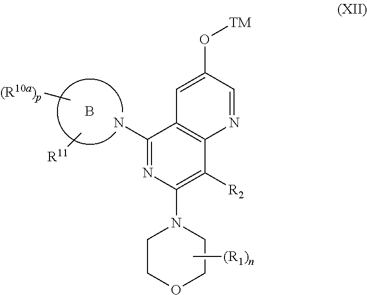

wherein R¹, R², R^{10a}, R¹¹, m and n are as described above for formula (I); and Ring B and p are as described above for formula (XI); wherein TM is the trigger moiety that releases a compound of formula (XIII) under reductive conditions:

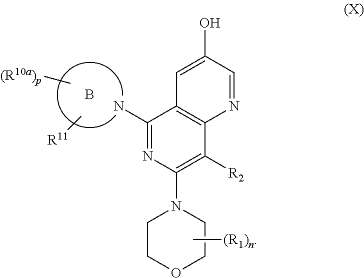

In an embodiment, the compound of formula (I) is a compound of formula (XIII).

The following statements apply to compounds or prodrugs of any of formulae (I) to (XIII). These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.

n may be 1 or 2. n may be 1. n is preferably 0.

R² may be H.

m may be 0. Alternatively, m may be 1. Where m is 1, it may be that R³ is selected from OH and NHR^{7a}. Where m is 1, it may be that the R³ group is positioned meta to the nitrogen in the pyridine ring to which (R³)ₘ is attached.

Where, R³ is OH or NHR^{7a}, these are convenient groups to which a trigger moiety may be attached to form a prodrug that releases a compound of formula (I) when subjected to reductive conditions. Thus, it may be that attached to the pyridine ring to which (R³)ₘ is attached (e.g. attached at the meta position relative to the pyridine nitrogen) is a OTM or NHTM group, wherein TM is the trigger moiety that releases a compound of formula (I) under reductive conditions.

Y may be O. In these embodiments, R⁴ will be -L¹-L²-R^{9a}.

Y may be NR⁵. In these embodiments, R⁴ may be -L¹-L²-R^{9a}.

-L¹- may be absent. -L¹- may be C₁-C₆-alkylene, e.g. C₁-C₃-alkylene. -L¹- may be CH₂.

-L²- may be absent. -L²- may be -L³-L⁴-.

-L³- may be C₃-C₆-cycloalkyl. -L³- may be cyclohexyl. Where -L³- is cyclohexyl, it may be that -L⁴-R⁹ is attached to the para position relative to the rest of the molecule. Thus, -L³- may have the structure:

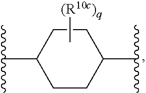

where q is an integer selected from 0, 1, 2, 3 and 4. -L³- may have the structure:

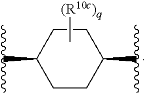

q may be 0.

-L³- may be a 3- to 8-membered heterocycloalkyl group wherein said heterocycloalkyl group may be monocyclic, bicyclic or a spirocyclic bicycle and wherein heterocycloalkyl group may be optionally substituted with from 1 to 4 R^{10c} substituents. -L³- may be a 3- to 8-membered heterocycloalkyl group comprising at least one nitrogen in the ring. The nitrogen (where there are is one nitrogen in the heterocycloalkyl ring) or a nitrogen (where there is more than one nitrogen in the heterocycloalkyl ring) may be the point of attachment of -L⁴-R^{9a} to -L³-. Thus, -L³- may be a piperidine ring, e.g. a piperidine ring in which the -L⁴-R^{9a} group is attached to the piperidine nitrogen. In these embodiments, the rest of the molecule may be attached to the piperidine ring para to the nitrogen. Where -L⁴-R^{9a} is attached to the nitrogen of a heterocycloalkyl ring, it may be that -L⁴- is absent. Where -L³- is a 3- to 8-membered heterocycloalkyl group it may be monocyclic. Where -L³- is a 3- to 8-membered heterocycloalkyl group (e.g. piperidine) it may be unsubstituted.

-L⁴- may be absent. -L⁴- may be selected from —NR^{13a}— (e.g. —NH—) and —O—. It may be that -L⁴- is —NR^{13a}—, e.g. —NH—.

Where -L⁴- is —NH—, this is a convenient group to which a trigger moiety may be attached to form a prodrug that releases a compound of formula (I) when subjected to reductive conditions. Thus, it may be that the compound is a prodrug in which a trigger moiety that releases a compound of formula (I) under reductive conditions is attached to the nitrogen of -L⁴-.

It may be that Y is NR⁵ and R⁴ and R⁵ together with the nitrogen to which they are attached form a 3- to 11-membered heterocycloalkyl group or a 5-membered heteroaryl group, said heterocycloalkyl group being optionally substituted with from 1 to 4 R^{10a} substituents and/or a single R¹¹ substituent and said heteroaryl group being optionally substituted with from 1 to 4 R^{12a} substituents and/or a single R¹¹ substituent; wherein said heterocycloalkyl group may be monocyclic, bicyclic or a spirocyclic bicycle. Preferably, said group is substituted with a single R¹¹ substituent. It may be that R⁴ and R⁵ together with the nitrogen to which they are attached form a 3- to 11-membered heterocycloalkyl group. Thus, it may be that R⁴ and R⁵ together with the nitrogen to which they are attached form a 3- to 11-membered heterocycloalkyl group said heterocycloalkyl group being optionally substituted with from 1 to 4 R^{10a} substituents; wherein said heterocycloalkyl group may be monocyclic, bicyclic or a spirocyclic bicycle and said heterocycloalkyl group is substituted with a single R¹¹ substituent.

Said 3- to 11-membered heterocycloalkyl group may comprise two nitrogen atoms in the ring system. Where the heterocycloalkyl groups comprise two nitrogen atoms in the ring system, it may be that R¹¹ is attached to the other nitrogen atom (i.e. the nitrogen atom that is not attached to R⁴, R⁵ and the rest of the molecule). Said group may be a piperazine. Said heterocycloalkyl group may be a bicyclic or a spirocyclic bicycle. Exemplary bicyclic groups formed of R⁴ and R⁵ and comprising two nitrogens include:

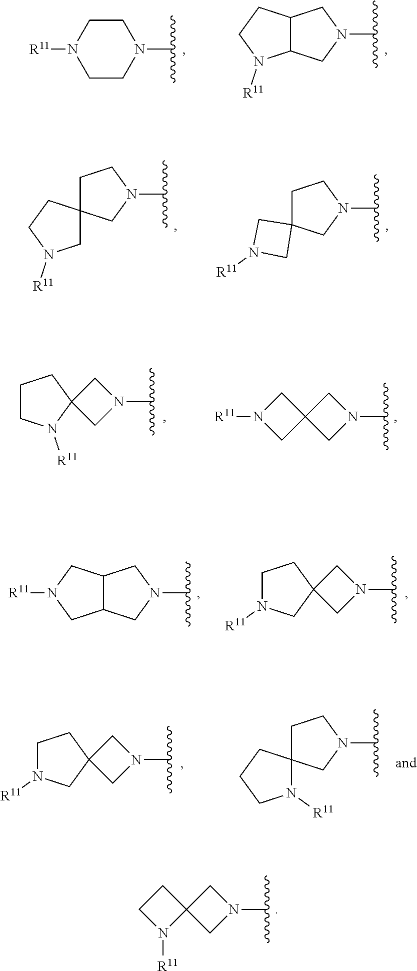

Where R¹¹ is attached to a nitrogen atom, it may be that -L⁵- and -L⁶- are absent.

Said 3- to 11-membered heterocycloalkyl group may comprise a single nitrogen atom in the ring system (i.e. the nitrogen atom that is not attached to R⁴, R⁵ and the rest of the molecule). Said heterocycloalkyl group may be monocylic. Said heterocycloalkyl group may be a fused or a spirocyclic bicycle. It may be that R⁴ and R⁵ together with the nitrogen to which they are attached form a 3- to 7-membered heterocycloalkyl group comprising a single nitrogen atom in the ring system. It may be that R⁴ and R⁵ together form a piperidine ring. Where R⁴ and R⁵ together form a piperidine ring, it may be that R¹¹ is attached to the ring para to the piperidine nitrogen. In the embodiments described in this paragraph, it may be that one of -L⁵- and -L⁶- is not absent. It may be that neither -L⁵- nor -L⁶- are absent.

The group formed by R⁴, R⁵ and the nitrogen to which they are attached may not be substituted with any R^{10a} groups.

-L⁵- may be absent. -L⁵- may be C₁-C₃-alkylene. Said alkylene group may be unsubstituted. -L⁵- may be selected from CH₂ and CH₂CH₂.

-L⁶- may be absent. -L⁶- may be selected from —NR^{13b}—, e.g. —NH— and —O—. -L⁶- may be NR^{13b}—, e.g. —NH—.

Where -L⁶- is —NH—, this is a convenient group to which a trigger moiety may be attached to form a prodrug that releases a compound of formula (I) when subjected to reductive conditions. Thus, it may be that the compound is a prodrug in which a trigger moiety that releases a compound of formula (I) under reductive conditions is attached to the nitrogen of -L⁶-.

R^{9a} and R^{9b} may each be selected from phenyl, napthyl and 5, 6, 9 or 10 membered heteroaryl. R^{9a} and R^{9b} may each be selected from phenyl and 5 or 6 membered heteroaryl. R^{9a} and R^{9b} may each be selected from 5 or 6 membered heteroaryl. R^{9a} and R^{9b} may each be selected from 5 or 6 membered heteroaryl group comprising at least one nitrogen atom in the ring system.

R^{9a} and R^{9b} may each be 5 membered heteroaryl, e.g. 5 membered heteroaryl comprising at least one nitrogen atom in the ring system. It may be that the R^{9a} or R^{9b} ring system comprises at least two nitrogens in the ring system. R^{9a} and R^{9b} may be selected from pyrazole, imidazole 1,2,3-triazole and 1,2,4-triazole. Where R^{9a} or R^{9b} is a 5-membered heteroaryl comprises at least one nitrogen in the ring system, it may be that R^{9a} or R^{9b} is attached to the rest of the molecule via the nitrogen (where the heteroaryl group comprises one nitrogen in the ring system) or via one of the nitrogens (where the heteroaryl group comprises two or more nitrogens in the ring system). Alternatively, where R^{9a} or R^{9b} is a 5-membered heteroaryl comprises at least one nitrogen in the ring system, it may be that the R^{9a} or R^{9b} is attached to the rest of the molecule via a carbon atom nitrogen. In these compounds, the nitrogen (where the heteroaryl group comprises one nitrogen in the ring system) or one of the nitrogens (where the heteroaryl group comprises two or more nitrogens in the ring system) would be a convenient group to which a trigger moiety may be attached to form a prodrug that releases a compound of formula (I) when subjected to reductive conditions. Thus, it may be that the compound is a prodrug in which a trigger moiety that releases a compound of formula (I) under reductive conditions is attached to a nitrogen atom of R^{9a} or R^{9b}.

R^{9a} and R^{9b} may each be 6 membered heteroaryl, e.g. 6 membered heteroaryl comprising at least one nitrogen atom in the ring system. It may be that the R^{9a} or R^{9b} ring system comprises at least two nitrogens in the ring system. R^{9a} and R^{9b} may be selected from pyridine, pyrimidine, pyrazine and pyridazine. R^{9a} or R^{9b} may be pyrimidine, e.g. pyrimidin-2-yl.

As mentioned above, the trigger moiety may have the structure:

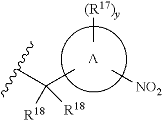

wherein ring A is a phenyl ring or a 5- or 6-membered heteroaryl ring;
R¹⁷ is independently at each occurrence selected from C₁-C₆-alkyl, C₁-C₆-haloalkyl, C₃-C₆-cycloalkyl, O—C₁-C₆-alkyl, cyano and halo;
R¹⁸ is independently at each occurrence selected from H, C₁-C₆-alkyl and C₁-C₆-haloalkyl; or the two R¹⁸ groups together form a C₃-C₆-cycloalkyl ring;
y is an integer from 0 to 3;
wherein the nitro group and the carbon attached to the two R¹⁸ groups are either attached to adjacent carbon atoms in Ring A or are attached to two carbon atoms in Ring A that are separated by two sp2 hybridised atoms selected from carbon and nitrogen.

The trigger moiety may have the structure:

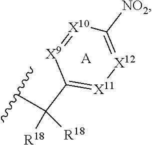

wherein X⁹, X¹⁰, X¹¹ and X¹² are selected such that Ring A is selected from phenyl, pyridine, pyrimidine, pyrazine and pyridazine any of which may be optionally substituted with from 0 to 4 R¹⁷ groups as described above. It may be that X⁹, X¹⁰, X¹¹ and X¹² are selected such that Ring A is selected from phenyl and pyridine any of which may be optionally substituted with from 0 to 4 R¹⁷ groups as described above.

The trigger moiety may have the structure:

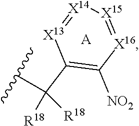

wherein X¹³, X¹⁴, X¹⁵ and X¹⁶ are selected such that Ring A is selected from phenyl, pyridine, pyrimidine, pyrazine and pyridazine any of which may be optionally substituted with from 0 to 4 R¹⁷ groups as described above. It may be that X¹³, X¹⁴, X¹⁵ and X¹⁶ are selected such that Ring A is selected from phenyl and pyridine any of which may be optionally substituted with from 0 to 4 R¹⁷ groups as described above.

The trigger moiety may have the structure:

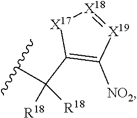

wherein X¹⁷, X¹⁸ and X¹⁹ are selected such that Ring A is selected from pyrazole, imidazole, oxazole, thiazole, isoxazole, isothiazole, furan, pyrrole, thiophene and 1,2,3-triazole any of which may be optionally substituted with from 0 to 3 R¹⁷ groups as described above. It may be that X¹⁷, X¹⁸ and X¹⁹ are selected such that Ring A is selected from imidazole and pyrazole any of which may be optionally substituted with from 0 to 3 R¹⁷ groups as described above.

The trigger moiety may have the structure:

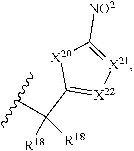

wherein X²⁰, X²¹ and X²² are selected such that Ring A is selected from imidazole, oxazole, thiazole, furan, pyrrole, thiophene, 1,2,4-triazole and 1,2,4-oxadiazole any of which may be optionally substituted with from 0 to 3 R¹⁷ groups as described above. It may be that X²⁰, X²¹ and X²² are selected such that Ring A is selected from imidazole, furan and thiophene any of which may be optionally substituted with 0 to 3 R¹⁷ groups as described above.

Exemplary trigger moieties include:

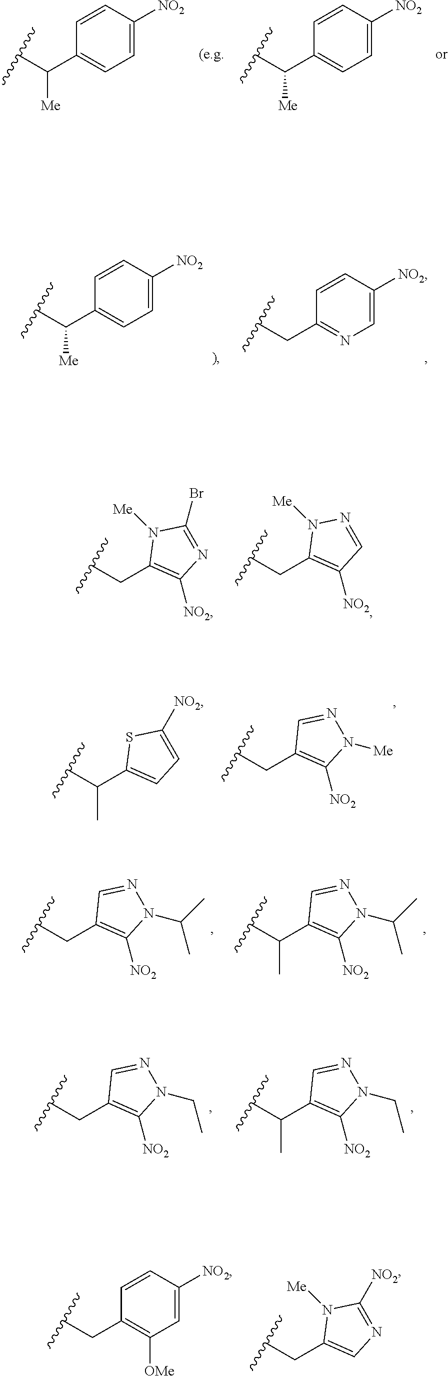

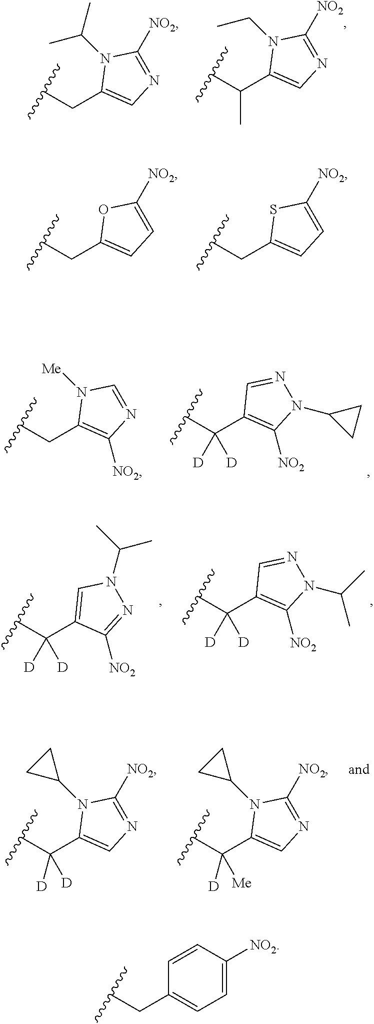

In an embodiment, the trigger moiety is selected from

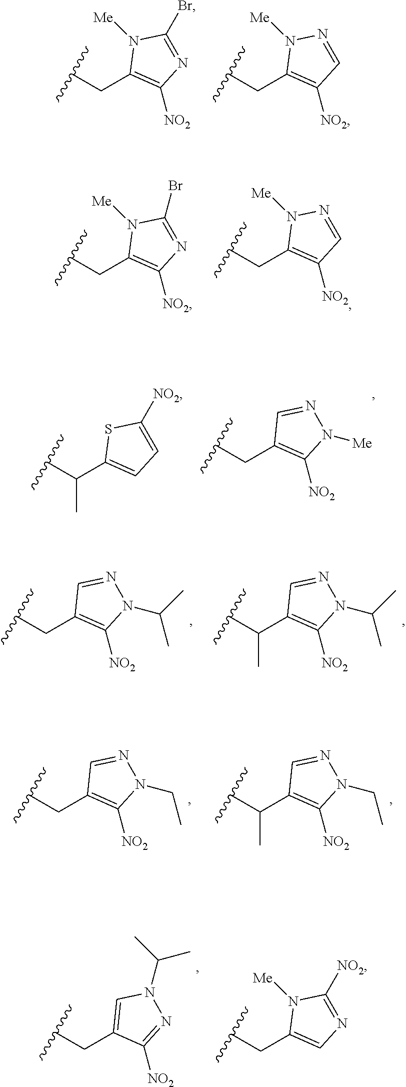

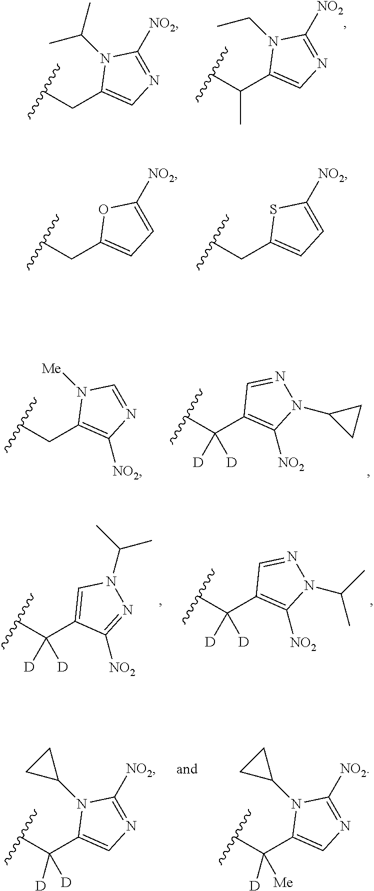

In an embodiment, the trigger moiety is selected from

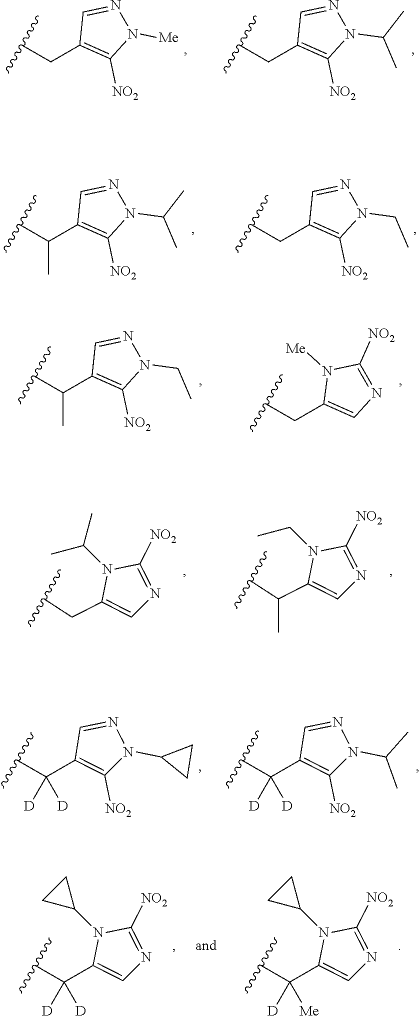

In an embodiment, the trigger moiety is selected from

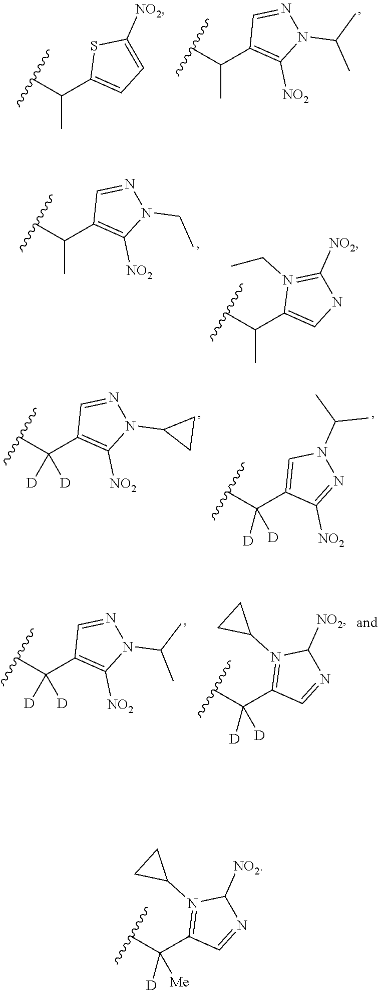

In an embodiment, the trigger moiety is selected from

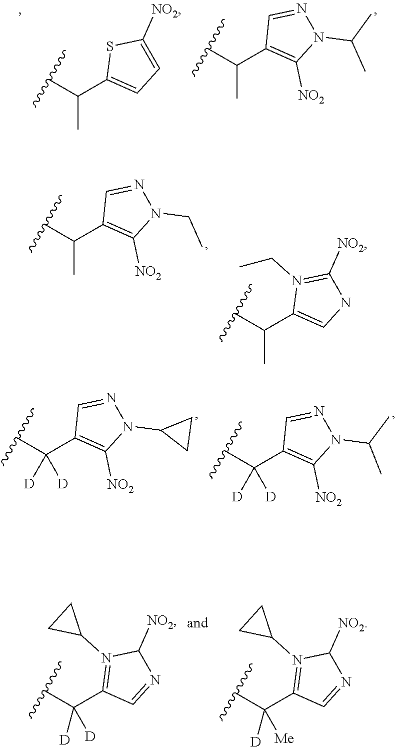

In an embodiment, the trigger moiety is selected from

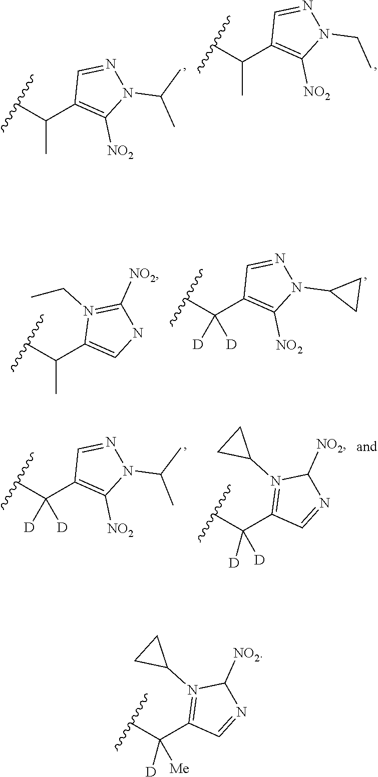

In an embodiment, the trigger moiety is selected from

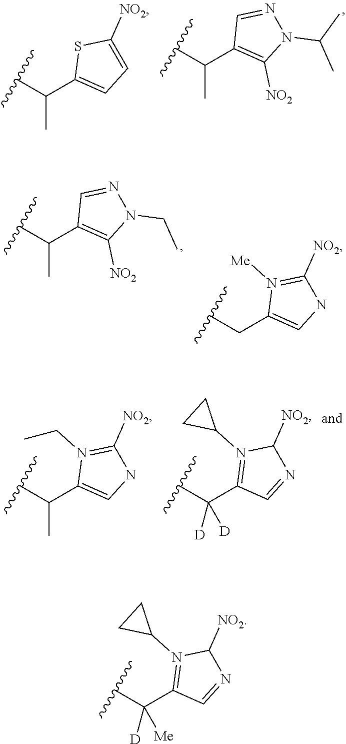

In an embodiment, the trigger moiety is selected from

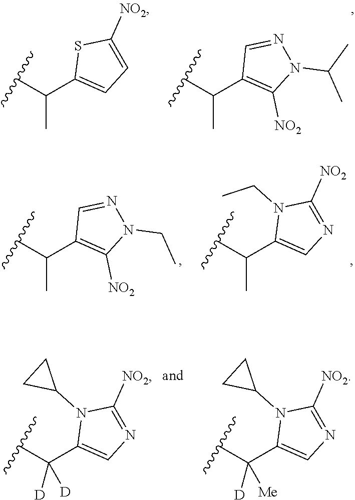 [SMILES: *C(C)c1ccc([N+](=O)[O-])s1 ; *C(C)c1cnn(C(C)C)c1[N+](=O)[O-] ; *C(C)c1cnn(CC)c1[N+](=O)[O-] ; *C(C)c1cnc([N+](=O)[O-])n1CC ; *C([2H])([2H])c1cnc([N+](=O)[O-])n1C1CC1 ; *C([2H])(C)c1cnc([N+](=O)[O-])n1C1CC1]

Particular compounds of the present invention include any one of the compounds or prodrugs exemplified in the present application, or a pharmaceutically acceptable salt or N-oxide thereof.

In some embodiments, the prodrug of the compound of formula (I) is not a compound selected from

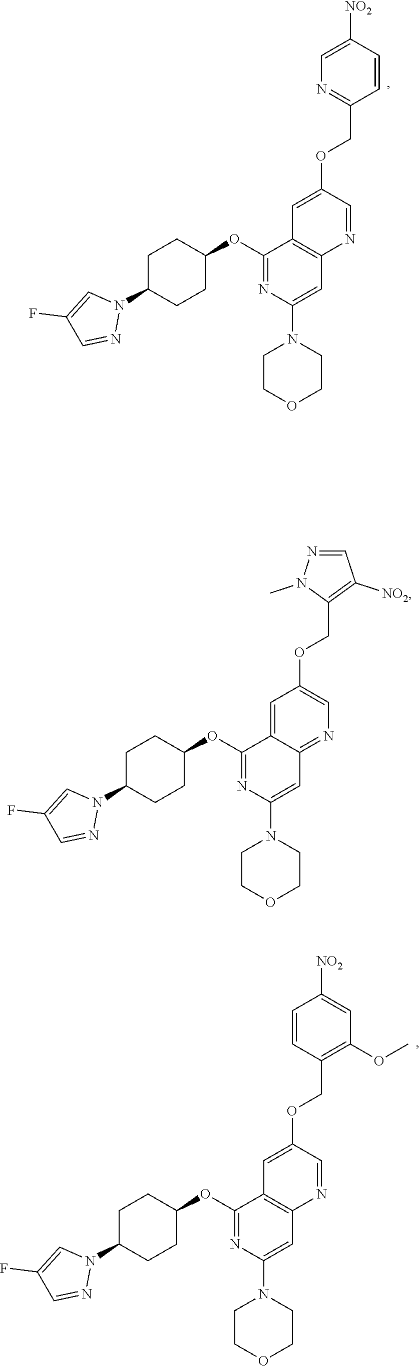 [SMILES: O=[N+]([O-])c1ccc(COc2cnc3cc(N4CCOCC4)nc(O[C@H]4CC[C@@H](n5cc(F)cn5)CC4)c3c2)nc1 ; Cn1ncc([N+](=O)[O-])c1COc1cnc2cc(N3CCOCC3)nc(O[C@H]3CC[C@@H](n4cc(F)cn4)CC3)c2c1 ; COc1cc([N+](=O)[O-])ccc1COc1cnc2cc(N3CCOCC3)nc(O[C@H]3CC[C@@H](n4cc(F)cn4)CC3)c2c1]

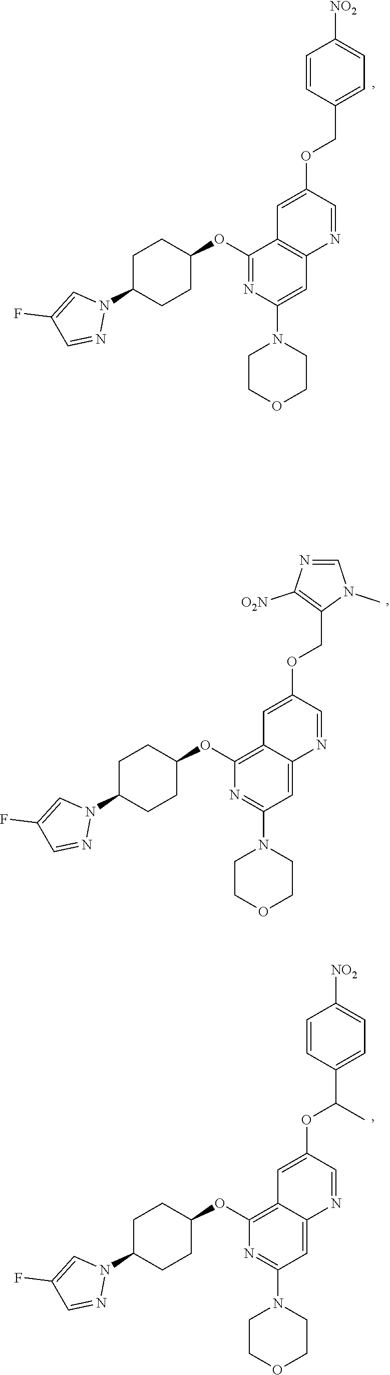 [SMILES: O=[N+]([O-])c1ccc(COc2cnc3cc(N4CCOCC4)nc(O[C@H]4CC[C@@H](n5cc(F)cn5)CC4)c3c2)cc1 ; Cn1cnc([N+](=O)[O-])c1COc1cnc2cc(N3CCOCC3)nc(O[C@H]3CC[C@@H](n4cc(F)cn4)CC3)c2c1 ; CC(Oc1cnc2cc(N3CCOCC3)nc(O[C@H]3CC[C@@H](n4cc(F)cn4)CC3)c2c1)c1ccc([N+](=O)[O-])cc1]

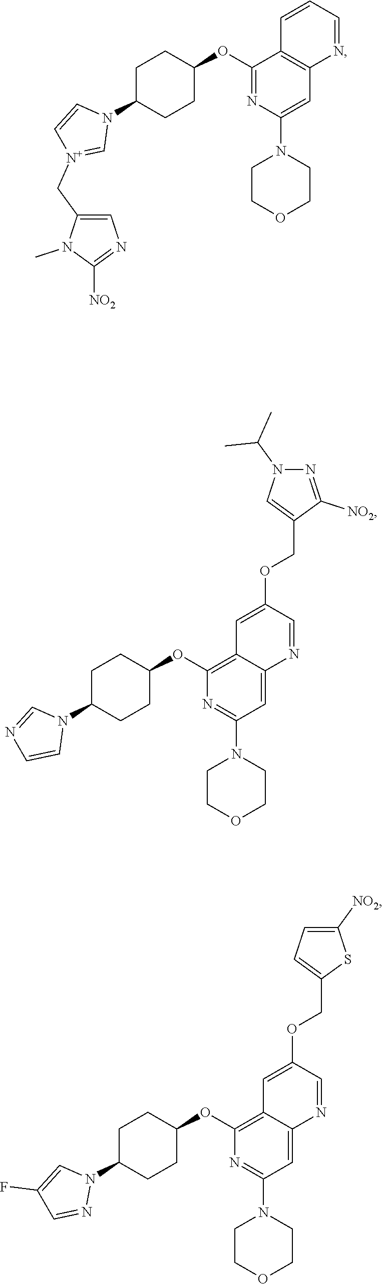 [SMILES: Cn1c(C[n+]2ccn([C@H]3CC[C@@H](Oc4nc(N5CCOCC5)cc5ncccc45)CC3)c2)cnc1[N+](=O)[O-] ; CC(C)n1cc(COc2cnc3cc(N4CCOCC4)nc(O[C@H]4CC[C@@H](n5ccnc5)CC4)c3c2)c([N+](=O)[O-])n1 ; O=[N+]([O-])c1ccc(COc2cnc3cc(N4CCOCC4)nc(O[C@H]4CC[C@@H](n5cc(F)cn5)CC4)c3c2)s1]

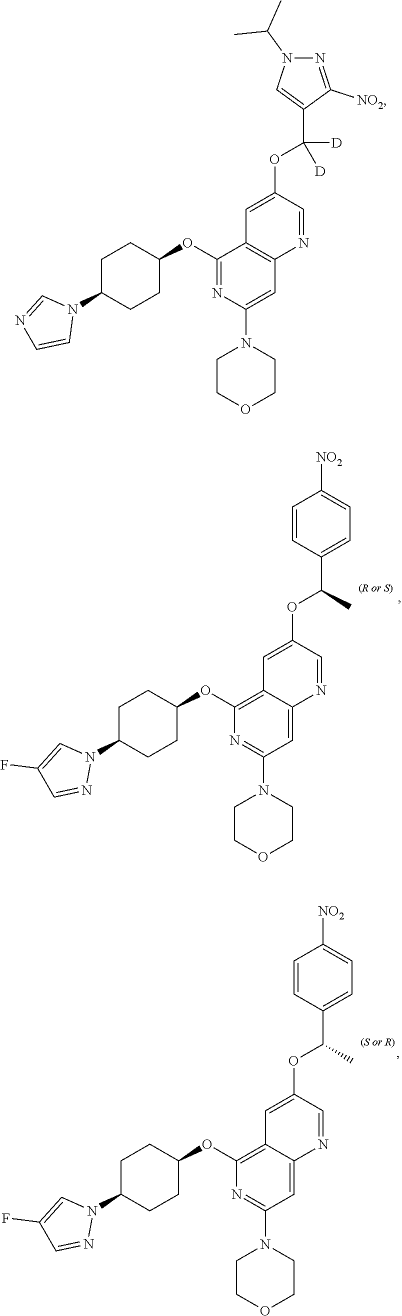 [SMILES: [2H]C([2H])(Oc1cnc2cc(N3CCOCC3)nc(O[C@H]3CC[C@@H](n4ccnc4)CC3)c2c1)c1cn(C(C)C)nc1[N+](=O)[O-] ; C[C@@H](Oc1cnc2cc(N3CCOCC3)nc(O[C@H]3CC[C@@H](n4cc(F)cn4)CC3)c2c1)c1ccc([N+](=O)[O-])cc1 ; C[C@H](Oc1cnc2cc(N3CCOCC3)nc(O[C@H]3CC[C@@H](n4cc(F)cn4)CC3)c2c1)c1ccc([N+](=O)[O-])cc1]

or a pharmaceutically acceptable salt or N-oxide thereof.

Also provided is a pharmaceutical formulation comprising a compound of the formula (I), or an aforementioned prodrug thereof or a pharmaceutically acceptable salt or N-oxide of the compound of formula (I) or the prodrug thereof, and a pharmaceutically acceptable excipient.

A further aspect provides a compound of the formula (I), or an aforementioned prodrug thereof or a pharmaceutically acceptable salt or N-oxide of the compound of formula (I) or the prodrug thereof, for use as a medicament.

Further provided is a compound of the formula (I), or an aforementioned prodrug thereof or a pharmaceutically acceptable salt or N-oxide of the compound of formula (I) or the prodrug thereof, for use in a treatment of cancer, wherein the treatment further comprises a DNA damaging chemotherapeutic agent and/or radiotherapy.

Also provided is a method of treating a cancer the method comprising administering to said subject an effective amount of a compound of the formula (I), or an aforementioned prodrug thereof or a pharmaceutically acceptable salt or N-oxide of the compound of formula (I) or the prodrug thereof, wherein the treatment further comprises a DNA damaging chemotherapeutic agent and/or radiotherapy.

Also provided is the use of a compound of formula (I), or an aforementioned prodrug thereof or a pharmaceutically acceptable salt or N-oxide of the compound of formula (I) or the prodrug thereof, for use in the manufacture of a medicament for treatment of cancer, wherein the treatment further comprises a DNA damaging chemotherapeutic agent and/or radiotherapy.

The compounds of formula (I) are DNA-PK inhibitors and are expected to enhance the effectiveness of cancer therapies that induce DNA damage in cancer cells, particularly hypoxic cancer cells. Accordingly also provided is a compound of the formula (I), or an aforementioned prodrug thereof or a pharmaceutically acceptable salt or N-oxide of the compound of formula (I) or the prodrug thereof, for use in a treatment of cancer, wherein the compound of the formula (I), or an aforementioned prodrug thereof or a pharmaceutically acceptable salt or N-oxide of the compound of formula (I) or the prodrug thereof to sensitise cancer cells to radiotherapy and/or a DNA damaging chemotherapeutic agent.

The cancer will typically be a solid cancer. For example, the cancer may be selected from: lung cancer, rectal cancer, colon cancer, liver cancer, bladder cancer, breast cancer, biliary cancer, prostate cancer, ovarian cancer, stomach cancer, bowel cancer, skin cancer, pancreatic cancer, brain cancer, cervix cancer, anal cancer or head and neck cancer. In some embodiments the cancer is head and neck cancer.

DNA damaging chemotherapeutic agents that may be used together with the compound of the formula (I), or an aforementioned prodrug thereof or a pharmaceutically acceptable salt or N-oxide of the compound of formula (I) or the prodrug thereof are well-known and include that induce DNA cross-links or function as topoisomerase inhibitors, inducing the generation of double strand-breaks in DNA. Examples of DNA damaging chemotherapeutic agents include platinum anticancer agents (e.g. cisplatin, carboplatin, oxaliplatin or picoplatin); anthracyclines (e.g. doxorubicin or daunorubicin); antifolates (e.g. methotrexate or pemetrexed); 5-fluorouracil; etoposide; gemcitabine; capecitabine; 6-mercaptopurine; 8-azaguanine; fludarabine; cladribine; vinorelbine; cyclophosphamide; taxoids (e.g. taxol, taxotere or paclitaxel), DNA-alkylating agents (e.g. nitrosoureas such as carmustine, lomustine or semustine); triazenes (e.g. dacarbazine or temozolomide); mitomycin C; or streptozotocin.

In a preferred embodiment the compound of formula (I) is used together with radiotherapy in the treatment of a cancer, wherein the compound of formula (I) act to sensitise cancer cells, particularly hypoxic cancer cells to radiotherapy. Accordingly, in a preferred embodiment there is provided a method of treating a cancer the method comprising administering to said subject an effective amount of a compound of the formula (I), or an aforementioned prodrug thereof or a pharmaceutically acceptable salt or N-oxide of the compound of formula (I) or the prodrug thereof, wherein the treatment further comprises radiotherapy

Also provided is a method of inhibiting DNA-PK activity in a human or animal subject in need of such inhibition, the method comprising administering to said subject an effective amount of a compound of the formula (I) or an aforementioned prodrug thereof or a pharmaceutically acceptable salt or N-oxide of the compound of formula (I) or the prodrug thereof.

Preferred, suitable, and optional features of any one particular aspect of the present invention are also preferred, suitable, and optional features of any other aspect.

### BRIEF DESCRIPTION OF THE DRAWINGS

FIG. 1 shows the concentration of prodrug 30 and the parent compound (Example 169) over time described in the examples. In this assay cells in stirred culture were gassed under 5% and 0.1% oxygen conditions with prodrug 30.

FIG. 2 shows the conversion of prodrugs 1-5 and 7-31 to the parent compounds in the assay described in the examples. In this assay cells in stirred culture were gassed under 5%, 1% and 0.1% oxygen conditions with the prodrug tested.

FIGS. 3-11 demonstrate cell panel activation screens in oxic (5% oxygen) and hypoxic (0.2% oxygen) cell suspensions for prodrugs 20, 1, 30, 27, 26, 24, 23, 22 and 19, respectively.

FIG. 12 shows the assessment of activation and activity of prodrugs 1, 20 and 21 in 3D spheroids assay disclosed herein. Data for the parent compound 162 is also shown.

FIG. 13 shows the hypoxic to oxic ratio observed for prodrugs 1, 2, 5, 7, 9, 10, 11, 12, 14, 17, 19, 20, 21, 30 and 31 in the 3D spheroids assay disclosed herein.

FIG. 14 shows clonogenic cell survival after tumour excision following treatment with 10Gy X-rays after treatment with prodrug 30 and parent compound 169, respectively.

FIG. 15 shows pharmacokinetics of prodrugs 20, 22 and 27 administered to mice intravenously (IV) at a dose of 10 mg/kg and per orally (PO) at a dose of 40 mg/kg.

FIG. 16 shows Western blots of tumour lysates following treatment with 10Gy X-rays after treatment with prodrug 30 and parent compound 169, respectively.

FIG. 17 shows tumour growth measurements indicating effects of prodrugs 27 and 22 after 10Gy treatment.

### DETAILED DESCRIPTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “halo” or “halogen” refers to one of the halogens, group 17 of the periodic table. In particular the term refers to fluorine, chlorine, bromine and iodine. Preferably, the term refers to fluorine or chlorine.

The term Cₘ-Cₙ refers to a group with m to n carbon atoms.

The term “C₁-C₆-alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁-C₄-alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁-C₄-alkoxy.

The term “C₁-C₆-alkoxy” refers to an alkyl group which is attached to a molecule via oxygen. This includes moieties where the alkyl part may be linear or branched and may contain 1, 2, 3, 4, 5 or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, ted-butoxy, n-pentoxy and n-hexoxy. The alkyl part of the alkoxy group may be unsubstituted or substituted by one or more substituents. Possible substituents are described below. Substituents for the alkyl group may be halogen, e.g. fluorine, chlorine, bromine and iodine, OH, C₁-C₆ alkoxy.

The term “C₁-C₆-haloalkyl” refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆-haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “C₂-C₆-alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “C₂-C₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “C₃-C₆-cycloalkyl” refers to a saturated hydrocarbon ring system containing 3, 4, 5 or 6 carbon atoms. For example, the “C₃-C₆-cycloalkyl” may be cyclopropyl, cyclobutyl, cyclopentyl, cyclohexyl, bicyclo[2.1.1]hexane or bicyclo[1.1.1]pentane.

The term “heterocyclyl”, “heterocyclic”, “heterocycle” or “heterocycloalkyl” means a non-aromatic saturated or partially saturated monocyclic or fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 3,8-diaza-bicyclo[3.2.1]octanyl, 8-aza-bicyclo[3.2.1]octanyl, 2,5-Diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4th Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

By “spiro bi-cyclic ring systems” is meant that the two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-Diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

The term “aromatic” when applied to a substituent as a whole means a single ring or polycyclic ring system with 4n+2 electrons in a conjugated π system within the ring or ring system where all atoms contributing to the conjugated π system are in the same plane.

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” refers to an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically, including the compounds of the formulae (I) to (VI), Compound List A, Compound List B and the compounds in the Examples.

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

A “-” in a substituent group denotes the point of attachment of that substituent to the rest of the molecule. Where a group is a linker group having two “-”s indicated, the “-” on the left indicates the attachment of the linker group to the bicyclic core of the molecule depicted in formula (I), either directly or via other linker groups. Likewise, the “-” on the right indicates the attachment of the linker group to groups that are further away from the bicyclic core of the molecule depicted in formula (I) than the linker group. Thus, in the group -L¹- the “-” on the left denotes the point of attachment to Y and the “-” on the right denotes the point of attachment to -L²-R^{9a} in formula (I). Likewise, in the group -L²- the “-” on the left denotes the point of attachment to -L¹- and the “-” on the right denotes the point of attachment to —R^{9a} in formula (I).

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

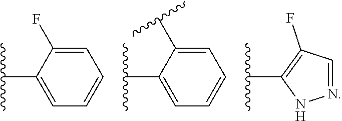

“Meta” substitution is a substitution pattern where two substituents are on carbons one carbon removed from each other, i.e with a single carbon atom between the substituted carbons. In other words, there is a substituent on the second atom away from the atom with another substituent. For example, the groups below are meta substituted.

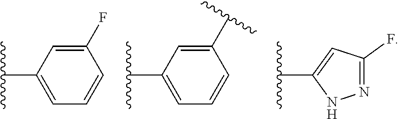

“Para” substitution is a substitution pattern where two substituents are on carbons two carbons removed from each other, i.e with two carbon atoms between the substituted carbons. In other words, there is a substituent on the third atom away from the atom with another substituent. For example, the groups below are para substituted.

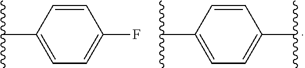

By “acyl” is meant an organic radical derived from, for example, an organic acid by the removal of the hydroxyl group, e.g. a radical having the formula R—C(O)—, where R may be selected from H, C₁₋₆-alkyl, C₃₋₈-cycloalkyl, phenyl, benzyl or phenethyl group, e.g. R is H or C₁₋₃-alkyl. In one embodiment acyl is alkyl-carbonyl. Examples of acyl groups include, but are not limited to, formyl, acetyl, propionyl and butyryl. A particular acyl group is acetyl (also represented as Ac).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 525 and, for example, is 500 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by for example, one or more of the following methods:

(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess Mps1 kinase inhibitory activity.

Compounds and salts described in this specification may be isotopically-labeled (or “radio-labeled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹⁸F and the like. The radionuclide that is used will depend on the specific application of that radio-labeled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F. In particular, one or both of the R¹⁸ moieties may be D.

It is also to be understood that certain compounds of the invention may exist in solvated as well as unsolvated forms such as, for example, hydrated forms. It is to be understood that the invention encompasses all such solvated forms that possess DNA-PK inhibitory activity.

It is also to be understood that certain compounds of the invention may exhibit polymorphism, and that the invention encompasses all such forms that possess DNA-PK inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

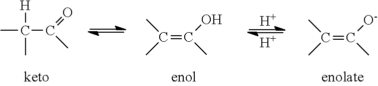

Compounds of the invention containing an amine function may also form N-oxides. A reference herein to a compound of the formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle or heteroaryl group. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

In one embodiment the compound of formula (I) is not in the form of an N-oxide.

In another embodiment the compound of formula (I) is not in the form of a salt. Alternatively, the compound of formula (I) may be in the form of a pharmaceutically acceptable salt.

The in vivo effects of a compound of the formula (I) may be exerted in part by one or more metabolites that are formed within the human or animal body after administration of a compound of the formula (I).

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃.OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

Further information on the preparation of the compounds of the invention is provided in the Examples section. The general reaction schemes and specific methods described in the Examples form a further aspect of the invention. The compounds of the invention can be made according to or analogously to the methods described in the Examples. The compounds of the invention can be made according to or analogously to the methods described in the following general synthetic schemes. The following schemes are only meant to represent examples of the invention and are in no way meant to be a limit of the invention.

Herein, the term ‘DCE’ means 1,2-dichloroethane, ‘DCM’ means dichloromethane, ‘DIPEA’ means diisopropylethylamine, ‘DMF’ means N,N-dimethylformamide, ‘DIAD’ means diisopropylazodicarboxylate, ‘EtOH’ means ethanol, ‘HCl’ means hydrochloric acid, ‘iPrOH’ means isopropanol, ‘LHMDS’ means lithium bis(trimethylsilyl)amide, ‘RuPhos Pd G1’ means chloro-(2-dicyclohexylphosphino-2′,6′-diisopropoxy-1,1′-biphenyl)[2-(2-aminoethyl)phenyl] palladium(II)-methyl-tert-butyl ether adduct, ‘tBuBrettPhos Pd G3’ means [(2-di-tert-butylphosphino-3,6-dimethoxy-2′,4′,6′-triisopropyl-1,1′-biphenyl)-2-(2′-amino-1,1′-biphenyl)] palladium(II) methanesulfonate and ‘THF’ means tetrahydrofuran.

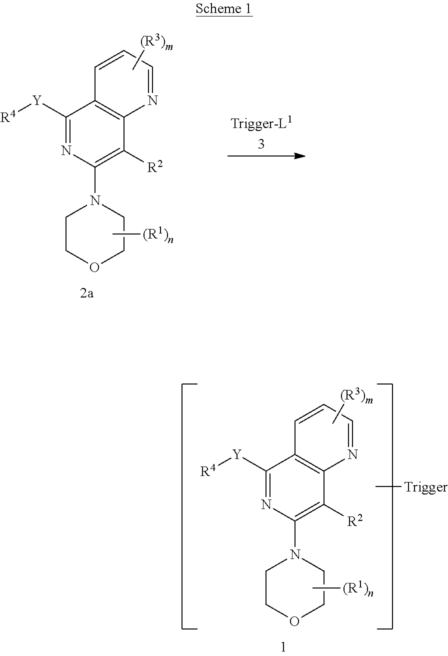

Scheme 1 illustrates methods of preparing prodrugs 1 of compounds of formula (I), wherein R¹-R⁴, Y and ‘Trigger’ are as defined in formula (I). Parent compounds 2a, wherein a suitable attachment point, such as —OH, —NH, —NH₂ or a quaternisable nitrogen is present, can be treated with intermediates 3, wherein L¹ is a suitable leaving group such as chloro or bromo, in the presence of a base such as potassium carbonate in an appropriate solvent such as DMF. Alternatively, prodrugs 1 can be prepared by reaction of parent compounds 2a and intermediates 3, wherein L¹ is hydroxy, under Mitsunobu conditions, using a suitable azodicarboxylate, phosphine and solvent (for example, DIAD, triphenylphosphine and THF, respectively).

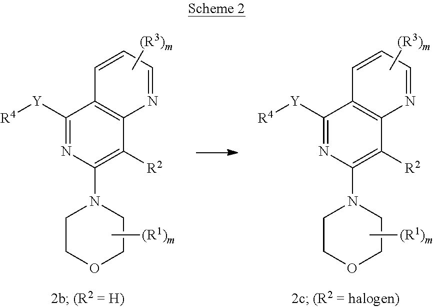

Scheme 2 illustrates methods of preparing compounds of formula (I), wherein R² is halogen, hereby represented as formula 2c, and wherein R¹, R³, R⁴ and Y are as defined in formula (I). Parent compounds 2b, wherein R² is hydrogen can be treated with a suitable halogenating agent, such as N-chlorosuccinimide in an appropriate solvent, such as DCE, to furnish parent compounds 2c.

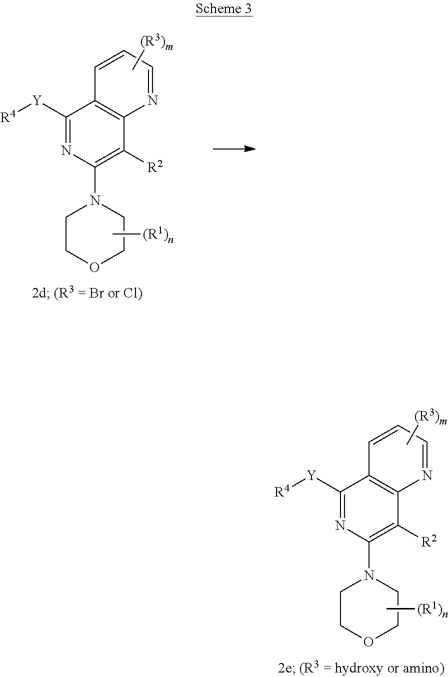

Scheme 3 illustrates methods of preparing compounds of formula (I), wherein R³ is hydroxy or amino, hereby represented as formula 2e, and wherein R¹, R², R⁴ and Y are as defined in formula (I). Parent compounds 2e, can be prepared by means of a Buchwald palladium-catalysed coupling of parent compounds of formula 2d, wherein R³ is a suitable leaving group, such as chloro or bromo, with a hydroxide salt, using a suitable palladium catalyst and solvent (for example tBuBrettPhos Pd G3 and dioxane respectively). Alternatively, parent compounds 2e, can be prepared by means of a Buchwald palladium-catalysed coupling of parent compounds of formula 2d with an amine, using a suitable palladium catalyst, base and solvent (for example tBuBrettPhos Pd G3, LHMDS and THF respectively).

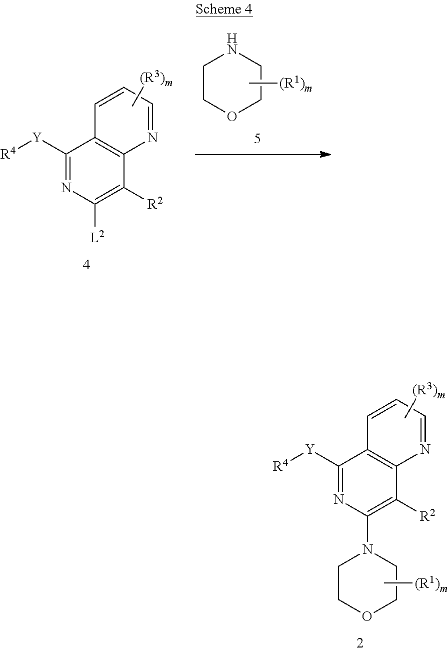

Parent compounds 2 of formula (I), wherein R¹-R⁴ and Y are as defined in formula (I) can be prepared by means of a Buchwald palladium-catalyzed coupling of intermediates of formula 4, wherein L² is a suitable leaving group such as chloro or bromo, with morpholines of formula 5, using a suitable palladium catalyst, base and solvent (for example RuPhos Pd G1, cesium carbonate and dioxane, respectively). Alternatively, parent compounds 2 of formula (I) can be prepared by heating intermediates of formula 4 in morpholines of formula 5 (Scheme 4).

Morpholines of formula 5 are commercially available or can be prepared by known methods.

Additional parent compounds 2 of formula (I) can be prepared from parent compounds 2 of formula (I) by elaboration of functional groups present. Such elaboration includes, but is not limited to, hydrolysis, reduction, oxidation, alkylation, amidation, hydroxylation, halogenation and dehydration. Such transformations may in some instances require the use of protecting groups.

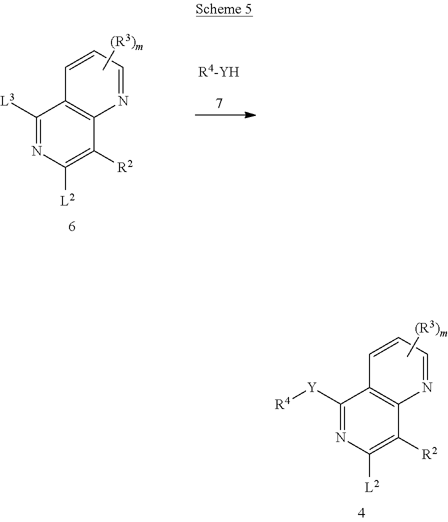

Scheme 5 illustrates methods of preparing intermediates of formula 4, wherein R²-R⁴ and Y are as defined in formula (I) and L² represents a leaving group such as chloro or bromo. Treatment of intermediates of formula 6, wherein L³ is an appropriate leaving group such as chloro or bromo, with alcohols or amines of formula 7 in the presence of a suitable base such as cesium carbonate, sodium hydride or DIPEA in an appropriate solvent such as dioxane, DMF or iPrOH, yields intermediates of formula 4.

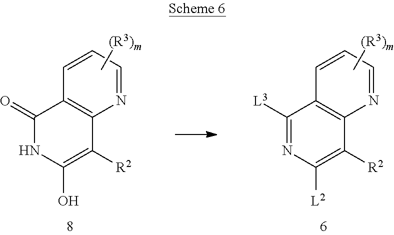

Scheme 6 illustrates a method of preparing intermediates of formula 6, wherein R² and R³ are as defined in formula (I) and L² and L³ represent suitable leaving groups such as chloro or bromo. Heating naphthyridones of formula 8 in an appropriate halogenating agent, such as phenylphosphonic dichloride or phosphorous oxychloride, furnishes intermediates of formula 6.

Naphthyridones of formula 8 are commercially available or can be prepared by known methods.

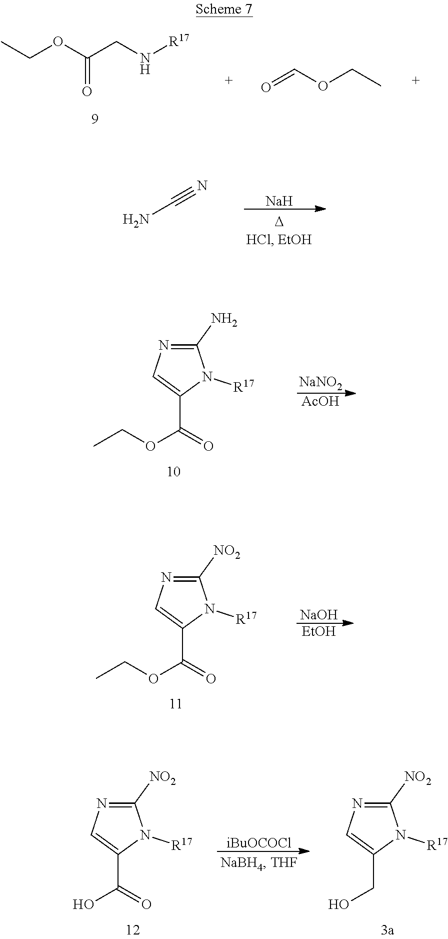

Scheme 7 illustrates methods of preparing triggers of formula 3a, wherein both R¹⁸ groups are hydrogen and R¹⁷ is as defined in formula (I). Aminoimidazoles 10 can be prepared by condensation of amino esters 9 with ethyl formate in the presence of a suitable base, such as sodium hydride, and cyclisation with cyanamide using a suitable acid and solvent (for example concentrated HCl and EtOH, respectively). Aminoimidazoles 10 can be oxidised using sodium nitrite in acetic acid to give nitroimidazoles 11. The ester group in 11 can be saponified with sodium hydroxide to furnish carboxylic acids 12 which, in turn, can be reacted with isobutyl chloroformate and reduced with a suitable reducing agent in an appropriate solvent, such as sodium borohydride and THF respectively, to furnish triggers 3a.

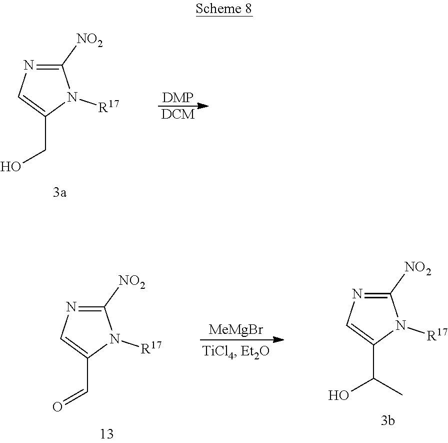

Scheme 8 illustrates methods of preparing triggers of formula 3b, wherein one R¹⁸ group is methyl, one R¹⁸ group is hydrogen and R¹⁷ is as defined in formula (I). The alcohol group in triggers 3a can be oxidised using Dess-Martin periodinane in an appropriate solvent, such as DCM, to give aldehydes 13. Aldehydes 13 can be reacted with methylmagnesium bromide in the presence of titanium tetrachloride and an appropriate solvent, such as diethyl ether, to furnish triggers 3b.

Additional triggers 3 of formula (I) can be prepared from commercially available starting materials using known methods.

It will be appreciated that where appropriate functional groups exist, compounds of various formulae or any intermediates used in their preparation may be further derivatised by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of formula (I) may be synthesized in the form of racemic mixtures of enantiomers which can be separated from one another following art-known resolution procedures. The racemic compounds of formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, tert-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

Compounds of the invention may be prepared from commercially available starting materials using the general methods illustrated herein.

The resultant compound of formula (I) from the processes defined above can be isolated and purified using techniques well known in the art.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

The processes defined herein may further comprise the step of subjecting the compound of formula (I) to a salt exchange, particularly in situations where the compound of formula (I) is formed as a mixture of different salt forms. The salt exchange suitably comprises immobilising the compound of formula II on a suitable solid support or resin, and eluting the compounds with an appropriate acid to yield a single salt of the compound of formula (I).

Certain of the intermediates described in the reaction schemes above and in the Examples herein are novel. Such a novel intermediate, or a salt thereof, particularly a pharmaceutically acceptable salt thereof form a further aspect of the invention.

### Biological Activity

The biological assays described in the accompanying example section may be used to measure the pharmacological effects of the compounds of the present invention.

### Pharmaceutical Compositions

In accordance with another aspect, the present invention provides a pharmaceutical formulation comprising a compound of formula (I), or a pharmaceutically acceptable salt or N-oxide thereof, and a pharmaceutically acceptable excipient.

Conventional procedures for the selection and preparation of suitable pharmaceutical formulations are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

In the following sections discussing uses and applications a reference to “compound of the formula (I)” is intended to encompass all of the compounds of the invention disclosed herein, for example any of the compounds of formulae (I) to (X).

The Background to the invention discusses various aspects of radiotherapy, DNA-PK inhibitors and the treatment of cancer. The disclosure of the Background of the Invention is incorporated into and forms part of the Detailed Description of the Invention.

In accordance with another aspect, the present invention provides a compound of formula (I), or an aforementioned prodrug thereof or a pharmaceutically acceptable salt or N-oxide of the compound of formula (I) or the prodrug thereof or a pharmaceutically acceptable salt or N-oxide thereof, for use as a medicament.

Further provided is a compound of the formula (I), or an aforementioned prodrug thereof or a pharmaceutically acceptable salt or N-oxide of the compound of formula (I) or the prodrug thereof, for use in a treatment of cancer, wherein the treatment further comprises radiotherapy.

Further provided is a compound of the formula (I), or an aforementioned prodrug thereof or a pharmaceutically acceptable salt or N-oxide of the compound of formula (I) or the prodrug thereof, for use in a treatment of cancer, wherein the treatment further comprises a DNA damaging chemotherapeutic agent.

DNA damaging chemotherapeutic agents that may be used together with the compound of formula (I) include for example any of those disclosed herein.

Further provided is a compound of the formula (I), or an aforementioned prodrug thereof or a pharmaceutically acceptable salt or N-oxide of the compound of formula (I) or the prodrug thereof, for use in a treatment of cancer, wherein the treatment further comprises a DNA damaging chemotherapeutic agent and radiotherapy.

Also provided is a method of treating a cancer the method comprising administering to said subject an effective amount of a compound of the formula (I), or an aforementioned prodrug thereof or a pharmaceutically acceptable salt or N-oxide of the compound of formula (I) or the prodrug thereof, wherein the treatment further comprises radiotherapy.

Also provided is the use of a compound of formula (I), or an aforementioned prodrug thereof or a pharmaceutically acceptable salt or N-oxide of the compound of formula (I) or the prodrug thereof, for use in the manufacture of a medicament for treatment of cancer, wherein the treatment further comprises radiotherapy.

The cancer will typically be a solid cancer. For example, the cancer may be selected from: lung cancer, rectal cancer, colon cancer, liver cancer, bladder cancer, breast cancer, biliary cancer, prostate cancer, ovarian cancer, stomach cancer, bowel cancer, skin cancer, pancreatic cancer, brain cancer, cervix cancer, anal cancer and head and neck cancer. In some embodiments the cancer is head and neck cancer.

### Radiotherapy

The compound of formula (I), or an aforementioned prodrug thereof or a pharmaceutically acceptable salt or N-oxide of the compound of formula (I) or the prodrug thereof may also be used be used in combination with radiotherapy. Suitable radiotherapy treatments include, for example X-ray therapy, proton beam therapy, gamma ray therapy or electron beam therapies. Radiotherapy (also described herein as “radiation therapy”, “ionizing radiation” and “IR”) techniques are well known and include conformal radiotherapy (3D CRT), intensity modulated radiation therapy (IMRT), image guided radiotherapy (IGRT), 4-dimensional radiotherapy (4D-RT) or stereotactic radiotherapy (SRT). Radiotherapy may also encompasses the use of radionuclide agents, for example ¹³¹I, ³²P, ⁹⁰Y, ⁸⁹Sr, ¹⁵³Sm or ²²³Ra. Such radionuclide therapies are well known and commercially available, for example ²²³Ra is available as an IV formulation for the treatment of cancer as AlphaRadin™ or Xofigo™. Radionuclides may be targeted to certain tissues or tumours by, for example, conjugating the radionuclide to a suitable antibody or receptor ligand protein.

According to a further aspect of the invention there is provided a compound of the formula (I), or an aforementioned prodrug thereof or a pharmaceutically acceptable salt or N-oxide of the compound of formula (I) or the prodrug thereof as defined hereinbefore for use in the treatment of cancer conjointly with radiotherapy.

According to a further aspect of the invention there is provided a method of treatment of a human or animal subject suffering from a cancer comprising administering to the subject a therapeutically effective amount of a compound of formula (I), or an aforementioned prodrug thereof or a pharmaceutically acceptable salt or N-oxide of the compound of formula (I) or the prodrug thereof simultaneously, sequentially or separately with radiotherapy.

In some embodiments the compound of formula (I), or an aforementioned prodrug thereof or a pharmaceutically acceptable salt or N-oxide of the compound of formula (I) or the prodrug thereof is administered to the subject prior to the radiotherapy. Administering the compound prior to radiotherapy advantageously sensitises the tissue to be treated (e.g. hypoxic tissue within a tumour) prior to application of radiotherapy. In other embodiments it is contemplated that the compound and the radiotherapy will be administered to the subject substantially simultaneously.

### Routes of Administration

The compounds of the invention or pharmaceutical composition comprising the active compound may be administered to a subject by any convenient route of administration, whether systemically/peripherally or topically (i.e. at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant.

### Combination Therapies for the Treatment of Cancer

The compounds of formula (I) or an aforementioned prodrug thereof or a pharmaceutically acceptable salt or N-oxide of the compound of formula (I) or the prodrug thereof may be used alone to provide an anti-cancer effect. However, the compounds of the invention are suitably used in combination with an anti-tumour agent and/or anti-tumour modality (e.g. IR), particularly anti-tumour agents and anti-tumour modalities that induce DNA damage. The compounds of formula (I) or the prodrug thereof may therefore be used in combination with one or more additional anti-tumour agent and/or modality (e.g. IR). The compounds of the invention may enable a lower dose of the additional anti-tumour agent or modality (such as IR) to be administered whilst maintaining or enhancing the anti-cancer effect of the additional agent or modality. Accordingly, the compounds of the invention may increase the therapeutic window and reduce undesirable side effects associated with the additional agent or modality.

Such anti-tumour agents may include, for example, one or more of the following categories of anti-tumour agents:

(i) antiproliferative/antineoplastic drugs and combinations thereof, such as alkylating agents (for example a platinum drug (e.g. cis-platin, oxaliplatin or carboplatin), cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), erbB2 tyrosine kinase inhibitors such as lapatinib) and antibodies to costimulatory molecules such as CTLA-4, 4-IBB and PD-I, or antibodies to cytokines (IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors; aurora kinase inhibitors and cyclin dependent kinase inhibitors such as CDK2 and/or CDK4 inhibitors; and CCR2, CCR4 or CCR6 antagonists;
(v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); toll-like receptor modulators for example TLR-7 or TLR-9 agonists; PD-1, PD-L1, PD-L2 and CTL4-A modulators (for example Nivolumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab; and
(viii) cytotoxic agents for example fludaribine (fludara), cladribine, pentostatin (Nipent™);
(ix) targeted therapies, for example PI3K inhibitors, for example idelalisib and perifosine; SMAC (second mitochondriaderived activator of caspases) mimetics, also known as Inhibitor of Apoptosis Proteins (IAP) antagonists (IAP antagonists). These agents act to supress IAPs, for example XIAP, cIAP1 and cIAP2, and thereby re-establish cellular apoptotic pathways. Particular SMAC mimetics include Birinapant (TL32711, TetraLogic Pharmaceuticals), LCL161 (Novartis), AEG40730 (Aegera Therapeutics), SM-164 (University of Michigan), LBW242 (Novartis), ML101 (Sanford-Burnham Medical Research Institute), AT-406 (Ascenta Therapeutics/University of Michigan), GDC-0917 (Genentech), AEG35156 (Aegera Therapeutic), and HGS1029 (Human Genome Sciences); and agents which target ubiquitin proteasome system (UPS), for example, bortezomib, carfilzomib, marizomib (NPI-0052), MLN9708 and p53 agonists, for example Nutlin-3A (Roche) and MI713 (Sanofi).
(xii) chimeric antigen receptors, anticancer vaccines and arginase inhibitors; and
(xiii) DNA damage response inhibitors, for example ATM, ATR, CHK1, WEE1, BER or PARP inhibitors. For example, a PARP inhibitor (e.g. olaparib, veliparib, rucaparib or niraparib, BMN-673.

The additional anti-tumour agent may be a single agent or one or more of the additional agents listed herein. In some embodiments the additional anti-tumour agent is used in combination with the compound of formula (I), or the prodrug thereof and radiotherapy. In some embodiments the additional anti-tumour agent is used in combination with the compound of formula (I), or the prodrug thereof and a DNA damaging chemotherapeutic agent.

In some embodiments the compound of formula (I), or the prodrug thereof is for use in combination with a DNA damaging chemotherapeutic agent in the treatment of a cancer. The DNA damaging chemotherapeutic agent may be, for example, an alkylating agent, an antimetabolite and/or a topoisomerase inhibitor. In certain embodiments it may be that the DNA damaging agent is an alkylating agent selected from: a platinum drug (e.g. cis-platin, oxaliplatin or carboplatin), cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine. In certain embodiments it may be that the DNA damaging agent is an antimetabolite selected from: gemcitabine, 5-fluorouracil, tegafur, raltitrexed, methotrexate, pemetrexed, cytosine arabinoside, floxuridine, cytarabine, 6-mercaptopurine, 6-thioguanine, fludarabine phosphate, pentostatine and hydroxyurea. In certain embodiments it may be that the DNA damaging agent topoisomerase inhibitor selected from epipodophyllotoxins like etoposide and teniposide, amsacrine, topotecan, irinotecan, mitoxantrone and camptothecin.

In some embodiments the compound of formula (I), or the prodrug thereof is for use concurrently with radiotherapy in the treatment of a cancer. The compound of formula (I), or the prodrug thereof, sensitises cells (e.g. tumour cells) to the radiotherapy and thus acts as a radiosensitiser. The compounds of the invention may be used in combination with various forms of radiotherapy, for example a radiotherapy described herein. In certain embodiments the radiotherapy may be an external radiation therapy or an internal radiotherapy. External radiation therapy utilises photons (e.g. X-rays), protons and/or electrons. The external radiation therapy may be administered using well-known methods, for example, 3-D conformal radiation therapy, intensity-modulated radiation therapy, image-guided radiation therapy, tomotherapy, stereotactic radiosurgery, stereotactic body radiation therapy or proton-beam therapy. Internal radiotherapy utilises a radioactive source inside the body. The internal radio therapy may take the form of a radioactive implant (brachytherapy) placed inside the body (e.g. interstitial brachytherapy or intracavity brachytherapy). The implant may take the form of radioactive pellets, seeds, sheets, wires or tubes that are placed in or close to the tumour to be treated. Internal radiotherapy may also be administered as a radioactive liquid, for example a liquid comprising radioactive iodine, radioactive strontium, radioactive phosphorus or radium 223.

The combination treatments described herein may be achieved by way of the simultaneous, sequential or separate dosing of the individual components of the treatment. Such combination products employ the compounds of this invention within a therapeutically effective dosage range described hereinbefore and the other anti-tumour agent and/or radiotherapy within its or their approved dosage range(s).

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. By way of an example, it may be that the compound of formula (I), or the prodrug thereof is administered to a subject prior to radiotherapy. In another embodiment the compound of formula (I), or the prodrug thereof is administered substantially simultaneously with radiotherapy. In another embodiment the compound of formula (I), or the prodrug thereof is administered to a subject that has received prior radiotherapy. For example, the compound of formula (I), or the prodrug thereof is administered to a subject that has been treated with radiotherapy 1 hour, 2 hours, 4 hours 8 hours, 12 hours, 1 day, 2 days, 1 week, 2 weeks or 1 month prior to administration of the compound of formula (I), or the prodrug thereof. In certain embodiments the compound of formula (I), or the prodrug thereof is for use in the treatment of a cancer in a subject prior to the subject receiving radiotherapy. For example, the compound of formula (I), or the prodrug thereof is administered to a subject 1 hour, 2 hours, 4 hours 8 hours, 12 hours, 1 day, 2 days, 1 week, 2 weeks or 1 month prior to initiating radiotherapy.

In some embodiments in which a combination treatment is used, the amount of the compound of formula (I), or the prodrug thereof, and the amount of the other pharmaceutically active agent(s) or radiotherapy are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of formula (I), or the prodrug thereof, and an approved or otherwise published dosage range(s) of the other pharmaceutically active compound(s) and/or doses of radiotherapy.

According to a further aspect of the invention there is provided a compound of formula (I), or an aforementioned prodrug thereof, or a pharmaceutically acceptable salt or N-oxide of the compound of formula (I) or the prodrug thereof, and an additional anti-tumour agent as defined hereinbefore, for use in the conjoint treatment of cancer. Optionally the compound of formula (I), or an aforementioned prodrug thereof, and the anti-tumour agent are for use in the treatment of a cancer in combination with a radiotherapy, for example a radiotherapy defined herein.

According to a further aspect of the invention there is provided a pharmaceutical product comprising a compound of formula (I), or an aforementioned prodrug thereof or a pharmaceutically acceptable salt or N-oxide of the compound of formula (I) or the prodrug thereof and an additional anti-tumour agent as defined hereinbefore for the conjoint treatment of cancer.

According to a further aspect of the invention there is provided a method of treatment of a human or animal subject suffering from a cancer comprising administering to the subject a therapeutically effective amount of formula (I), or an aforementioned prodrug thereof or a pharmaceutically acceptable salt or N-oxide of the compound of formula (I) or the prodrug, thereof simultaneously, sequentially or separately with an additional anti-tumour agent as defined hereinbefore. Optionally the method further comprises treating the subject with radiotherapy (e.g. a radiotherapy described herein). The radiotherapy may be administered to the subject simultaneously, sequentially or separately with compound of formula (I), or an aforementioned prodrug thereof and the anti-tumour agent.

According to a further aspect of the invention there is provided a compound of formula (I), or an aforementioned prodrug thereof or a pharmaceutically acceptable salt or N-oxide of the compound of formula (I) or the prodrug thereof, for use simultaneously, sequentially or separately with an additional anti-tumour agent as defined hereinbefore, in the treatment of a cancer. Optionally the compound of formula (I), or an aforementioned prodrug and the anti-tumour agent are for use in the treatment of a cancer in combination with radiotherapy (e.g. a radiotherapy defined herein). The radiotherapy may be administered to the subject simultaneously, sequentially or separately with compound of formula (I), or an aforementioned prodrug and the anti-tumour agent.

### EXAMPLES

Several methods for preparing the compounds of this invention are illustrated in the following examples. Unless otherwise noted, all starting materials were obtained from commercial suppliers and used without further purification.

Herein, the term ‘BEH’ means bridged ethylsiloxane/silica hybrid, ‘BINAP’ means (2,2′-bis(diphenylphosphino)-1,1′-binaphthyl), ‘CDCl₃’ means deuterochloroform, ‘CSH’ means charged surface hybrid, ‘DCE’ means 1,2-dichloroethane, ‘DCM’ means dichloromethane, ‘DDQ’ means 2,3-dichloro-5,6-dicyano-p-benzoquinone, DIAD′ means diisopropyl azodicarboxylate, DIPEA′ means diisopropylethylamine, ‘DMF’ means N,N-dimethylformamide, ‘DMSO’ means dimethylsulfoxide, ‘EtOAc’ means ethyl acetate, ‘EtOH’ means ethanol, ‘HATU’ means N-[(dimethylamino)-1H-1,2,3-triazolo[4,5-b]pyridin-1-ylmethylene]-N-methylmethanaminium hexafluorophosphate N-oxide, ‘HCl’ means hydrochloric acid, ‘HPLC’ means high-performance liquid chromatography, ‘PrOH’ means isopropanol, ‘ISOLUTE® SCX-2 SPE’ means ISOLUTE® silica propylsulfonic acid strong cation exchange column, ‘LC’ means liquid chromatography, ‘LCMS’ means liquid chromatography/mass spectrometry, ‘MDAP’ means mass-directed autopurification, ‘MeCN’ means acetonitrile, ‘MeOH’ means methanol, ‘Rₜ’ means retention time, RuPhos' means 2-dicyclohexylphosphino-2′,6′-diisopropoxybiphenyl, ‘RuPhos Pd G1.TBME’ means chloro-(2-dicyclohexylphosphino-2′,6′-diisopropoxy-1,1-biphenyl)[2-(2-aminoethyl)phenyl] palladium(II) methyl-tert-butyl ether adduct, ‘RuPhos Pd G3’ means (2-dicyclohexylphosphino-2′,6′-diisopropoxy-1,1-biphenyl)[2-(2′-amino-1,1′-biphenyl)] palladium(II)methanesulfonate, ‘Selectfluor®’ means 1-chloromethyl-4-fluoro-1,4-diazoniabicyclo[2.2.2]octane bis(tetrafluoroborate), ‘SFC’ means supercritical fluid chromatography, ‘tBuBrettPhos’ means 2-(di-tert-butylphosphino)-2′,4′,6′-triisopropyl-3,6-dimethoxy-1,1′-biphenyl, ‘tBuBrettPhos Pd G3’ means [(2-di-tert-butylphosphino-3,6-dimethoxy-2′,4′,6′-triisopropyl-1,1′-biphenyl)-2-(2′-amino-1,1′-biphenyl)] palladium(II) methanesulfonate, ‘TFA’ means trifluoroacetic acid, ‘THF’ means tetrahydrofuran and ‘Trt’ means trityl.

In the structures of the intermediates and the compounds of the present invention, deuterium (²H) is represented by the chemical symbol D.

When in the Examples below, intermediates, parent compounds, triggers or prodrugs were prepared according to the reaction protocol of a fully described Example, this means that the intermediate, parent compound, trigger or prodrug was prepared by an analogous reaction protocol (but not necessarily identical) as the Example referred to.

Where indicated in the Examples below, purification of intermediates, parent compounds, triggers and prodrugs was performed using the following methods:

### Reverse-Phase Preparative HPLC

Method A: Experiments were performed on a Gilson 321-H2 system linked to a Gilson 151 UV/Vis detector. LC was carried out using a Phenomenex® Kinetex 50×21.2 mm EVO C18 column, or a Phenomenex® Kinetex 250×21.2 mm EVO C18 column and an 18 ml/minute flow rate. The solvent system was a mixture of water containing 0.1% formic acid (solvent A) and MeCN containing 0.1% formic acid (solvent B), with a gradient between 95% solvent A/5% solvent B and 2% solvent A/98% solvent B over 5 to 25 minutes.

Method B: Experiments were performed on a Gilson 321-H2 system linked to a Gilson 151 UV/Vis detector. LC was carried out using a Phenomenex® Kinetex 50×21.2 mm EVO C18 column, or a Phenomenex® Kinetex 250×21.2 mm EVO C18 column and an 18 ml/minute flow rate. The solvent system was a mixture of water containing 0.1% ammonium hydroxide (solvent A) and MeCN containing 0.1% ammonium hydroxide (solvent B), with a gradient between 95% solvent A/5% solvent B and 2% solvent A/98% solvent B over 5 to 25 minutes.

### MDAP

Method A: Experiments were performed on an Agilent 1260 Infinity system linked to an Agilent 6120 single quadrupole mass spectrometer. LC was carried out using a Waters XBridge® BEH or XSelect® CSH 10×50 mm, 19×250 mm or 30×150 mm C18 column and a 20 to 60 ml/minute flow rate. The solvent system was a mixture of water containing 0.1% formic acid (solvent A) and MeCN containing 0.1% formic acid (solvent B), with a gradient between 90% solvent A/10% solvent B and 2% solvent A/98% solvent B over 15 to 25 minutes.
Method B: Experiments were performed on an Agilent 1260 Infinity system linked to an Agilent 6120 single quadrupole mass spectrometer. LC was carried out using a Waters XBridge® BEH or XSelect® CSH 10×50 mm, 19×250 mm or 30×150 mm C18 column and a 20 to 60 ml/minute flow rate. The solvent system was a mixture of water containing 0.1% ammonium hydroxide (solvent A) and MeCN containing 0.1% ammonium hydroxide (solvent B), with a gradient between 90% solvent A/10% solvent B and 2% solvent A/98% solvent B over 15 to 25 minutes.

### Preparation of Intermediates

### Example A1

### a) Preparation of Intermediate 1

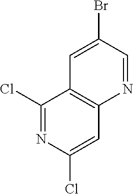 [SMILES: Clc1cc2ncc(Br)cc2c(Cl)n1]

A mixture of 3-bromo-6,8-dihydro-1,6-naphthyridine-5,7-dione (2.00 g, 8.30 mmol) and phenylphosphonic dichloride (20 ml) was heated at 110° C. for 18 hours. The mixture was cooled to ambient temperature and carefully poured into water (200 ml) with stirring. The resulting mixture was extracted with EtOAc. The organic phase was washed with saturated aqueous sodium bicarbonate solution, followed by brine, dried over sodium sulfate and concentrated in vacuo. The residue was purified by column chromatography on silica gel, eluting with a mixture of cyclohexane and EtOAc (1:0 to 3:2 by volume), to afford the desired product as a white solid (0.73 g, 32%).

¹H NMR (400 MHz, CDCl₃) δ ppm: 9.11 (d, J=2.3 Hz, 1H), 8.74 (dd, J=0.9, 2.3 Hz, 1H), 7.93 (d, J=0.9 Hz, 1H).

### Example A2

### a) Preparation of Intermediate 2

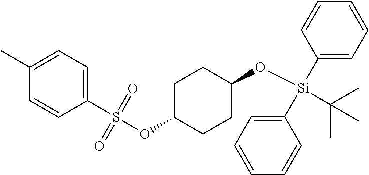 [SMILES: Cc1ccc(S(=O)(=O)O[C@H]2CC[C@H](O[Si](c3ccccc3)(c3ccccc3)C(C)(C)C)CC2)cc1]

A solution of trans-4-[[(1,1-dimethylethyl)diphenylsilyl]oxy]cyclohexanol (3.15 g, 8.90 mmol) in pyridine (10 ml) at 50° C. was treated with p-toluenesulfonyl chloride (3.40 g, 17.7 mmol) and the resulting mixture was heated at 50° C. for 18 hours. The mixture was cooled to ambient temperature and partitioned between EtOAc and water. The organic phase was washed with 1.0 M aqueous HCl solution, followed by brine, dried over sodium sulfate and concentrated in vacuo. The residue was purified by column chromatography on silica gel, eluting with a mixture of cyclohexane and EtOAc (1:0 to 5:1 by volume), to afford the desired product as a colourless oil (3.50 g, 75%).

¹H NMR (400 MHz, CDCl₃) δ ppm: 7.76 (d, J=8.4 Hz, 2H), 7.64-7.59 (m, 4H), 7.45-7.28 (m, 8H), 4.59-4.52 (m, 1H), 3.82-3.74 (m, 1H), 2.43 (s, 3H), 1.96-1.86 (m, 2H), 1.75-1.66 (m, 2H), 1.53-1.33 (m, 4H), 1.02 (s, 9H).

### b) Preparation of Intermediate 3

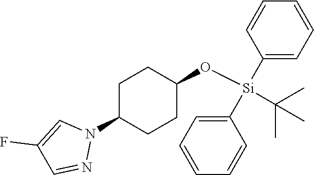 [SMILES: CC(C)(C)[Si](O[C@H]1CC[C@@H](n2cc(F)cn2)CC1)(c1ccccc1)c1ccccc1]

A solution of 4-fluoro-1H-pyrazole (0.75 g, 8.68 mmol) in DMF (35 ml) at ambient temperature was treated with sodium hydride (0.35 g, 8.68 mmol, 60% in mineral oil). After stirring for 20 minutes, a solution of intermediate 2 (3.50 g, 6.68 mmol) in DMF (15 ml) was added and the resulting mixture was heated at 100° C. for 4 hours. The mixture was cooled to ambient temperature and partitioned between EtOAc and water. The organic phase was washed with saturated aqueous sodium bicarbonate solution, followed by brine, dried over sodium sulfate and concentrated in vacuo. The residue was purified by column chromatography on silica gel, eluting with a mixture of cyclohexane and EtOAc (1:0 to 3:7 by volume), to afford the desired product as a colourless oil (1.90 g, 68%).

LCMS (Method A): Rt=1.88 min, m/z [M+H]⁺=423

### c) Preparation of Intermediate 4

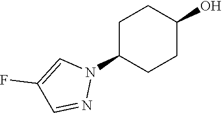 [SMILES: O[C@H]1CC[C@@H](n2cc(F)cn2)CC1]

A mixture of intermediate 3; (1.13 g, 2.68 mmol), 1.0 M tetrabutylammonium fluoride solution in THF (5.0 ml, 5.00 mmol) and THF (20 ml) was heated at reflux for 18 hours. The resulting mixture was cooled to ambient temperature and concentrated in vacuo. The residue was purified by column chromatography on silica gel, eluting with a mixture of EtOAc and MeOH (1:0 to 7:3 by volume), to afford the desired product as a colourless oil (0.39 g, 79%).

¹H NMR (400 MHz, CDCl₃) δ ppm: 7.34-7.30 (m, 2H), 4.10-4.03 (m, 2H), 2.19-2.04 (m, 2H), 1.97-1.87 (m, 4H), 1.75-1.64 (m, 2H), 1.39 (s, 1H).

### Example A3

### a) Preparation of Intermediate 5

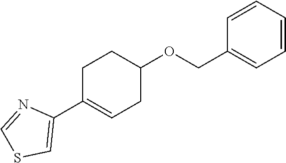 [SMILES: C1=C(c2cscn2)CCC(OCc2ccccc2)C1]

A mixture of 4-(benzyloxy)cyclohex-1-en-1-yl trifluoromethanesulfonate (1.00 g, 2.97 mmol), 4-(tributylstannyl)thiazole (1.11 g, 2.97 mmol), lithium chloride (0.19 g, 4.46 mmol), copper(I) iodide (0.11 g, 0.59 mmol) and tetrakis(triphenylphosphine)palladium(0) (0.34 g, 0.29 mmol) in dioxane (30 ml) was heated at 100° C. for 50 minutes under a nitrogen atmosphere. The resulting mixture was cooled to ambient temperature and concentrated in vacuo. The residue was purified by column chromatography on silica gel, eluting with a mixture of cyclohexane and EtOAc (1:0 to 7:3 by volume), to afford the desired product as an orange solid (0.18 g, 22%).

LCMS (Method A): Rt=1.58 min, m/z [M+H]⁺=272

Intermediate 6; was prepared according to the reaction protocol of intermediate 5 using the appropriate starting materials (Table 1).

TABLE 1

| Intermediate | Structure | Starting Materials | LCMS Data |
|---|---|---|---|
| 6 | 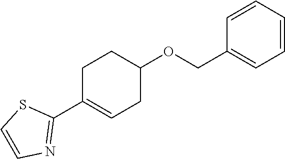 [SMILES: C1=C(c2nccs2)CCC(OCc2ccccc2)C1] | a) 4-(Benzyloxy)cyclohex-1-en- 1-yl trifluoromethanesulfonate b) 2-(Tributylstannyl)thiazole | Rt = 1.60 min, m/z [M + H]⁺ = 272 (Method A) |

### Example A4

### a) Preparation of Intermediate 7

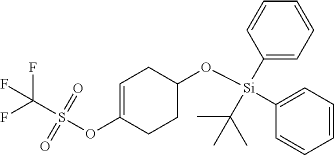 [SMILES: CC(C)(C)[Si](OC1CC=C(OS(=O)(=O)C(F)(F)F)CC1)(c1ccccc1)c1ccccc1]

A solution of 4-((tert-butyldiphenylsilyl)oxy)cyclohexan-1-one (0.67 g, 1.91 mmol) and 1,1,1-trifluoro-N-phenyl-N-((trifluoromethyl)sulfonyl)methanesulfonamide (1.02 g, 2.86 mmol) in anhydrous THF (8.0 ml) at −78° C. under an argon atmosphere was treated with 1.5 M lithium bis(trimethylsilyl)amide solution (1.90 ml, 2.86 mmol) dropwise over 10 minutes. The resulting mixture was stirred for 2 hours at −78° C., then warmed to ambient temperature over 1 hour. The reaction was quenched by addition of water and extracted with EtOAc. The organic phase was washed with brine, dried over sodium sulfate and concentrated in vacuo. The residue was purified by column chromatography on silica gel, eluting with a mixture of methyl tert-butyl ether and isohexane (1:0 to 9:1 by volume), to afford the desired product a colourless gum (0.70 g, 76%).

¹H NMR (400 MHz, CDCl₃) δ ppm: 7.67-7.63 (m, 4H), 7.45-7.35 (m, 6H), 5.59-5.56 (m, 1H), 4.05-4.00 (m, 1H), 2.62-2.54 (m, 1H), 2.25-2.18 (m, 3H), 1.90-1.81 (m, 1H), 1.75-1.67 (m, 1H), 1.05 (s, 9H).

### b) Preparation of Intermediate 8

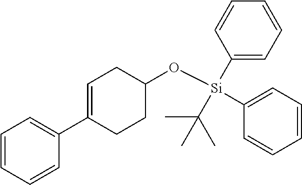 [SMILES: CC(C)(C)[Si](OC1CC=C(c2ccccc2)CC1)(c1ccccc1)c1ccccc1]

A suspension of intermediate 7; (0.15 g, 0.32 mmol), phenylboronic acid (0.039 g, 0.32 mmol), [1,1-bis(diphenylphosphino)ferrocene]dichloro palladium(II) (0.026 g, 0.032 mmol) and cesium carbonate (0.31 g, 0.96 mmol) in a mixture of dioxane (3.0 ml) and water (1.0 ml) was heated at 85° C. for 1 hour under an argon atmosphere. The resulting mixture was cooled to ambient temperature, filtered and the filtrate was partitioned between EtOAc and saturated aqueous sodium bicarbonate solution. The organic phase was dried over sodium sulfate and concentrated in vacuo. The residue was purified by column chromatography on silica gel, eluting with a mixture of cyclohexane and EtOAc (1:0 to 19:1 by volume), to afford the desired product a colourless oil (0.18 g, 89%).

¹H NMR (400 MHz, CDCl₃) δ ppm: 7.72-7.67 (m, 4H), 7.46-7.27 (m, 10H), 7.22-7.17 (m, 1H), 5.92-5.88 (m, 1H), 4.08-4.01 (m, 1H), 2.61-2.53 (m, 1H), 2.36-2.22 (m, 3H), 1.92-1.77 (m, 2H), 1.08-1.06 (m, 9H).

Intermediates 9 and 10 were prepared according to the reaction protocol of intermediate 8 using the appropriate starting materials (Table 2).

TABLE 2

| Intermediate | Structure | Starting Materials | NMR Data |
|---|---|---|---|
| 9 | 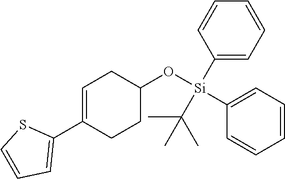 [SMILES: CC(C)(C)[Si](OC1CC=C(c2cccs2)CC1)(c1ccccc1)c1ccccc1] | a) Intermediate 7; b) 4,4,5,5-Tetramethyl-2- (thiophen-2-yl)-1,3,2- dioxaborolane | ¹H NMR (400 MHz, CDCl₃) δ ppm: 7.71- 7.65 (m, 4H), 7.43- 7.34 (m, 6H), 7.08 (dd, J = 1.0, 5.1 Hz, 1H), 6.94-6.91 (m, 1H), 6.89-6.87 (m, 1H), 5.98-5.94 (m, 1H), 4.04-3.97 (m, 1H), 2.63-2.55 (m, 1H), 2.35-2.22 (m, 3H), 1.89-1.75 (m, 2H), 1.07-1.06 (m, 9H). |
| 10 | 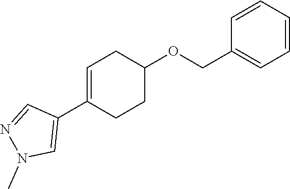 [SMILES: Cn1cc(C2=CCC(OCc3ccccc3)CC2)cn1] | a) 4- (Benzyloxy)cyclohex-1- en-1-yl trifluoromethanesulfonate b) 1-Methyl-4-(4,4,5,5- tetramethyl-1,3,2- dioxaborolan-2-yl)-1H- pyrazole | ¹H NMR (400 MHz, CDCl₃) δ ppm: 7.53- 7.50 (m, 1H), 7.39- 7.26 (m, 6H), 5.87- 5.84 (m, 1H), 4.64- 4.57 (m, 2H), 3.86 (s, 3H), 3.75-3.67 (m, 1H), 2.56-2.42 (m, 2H), 2.37-2.19 (m, 2H), 2.10-2.04 (m, 1H), 1.86-1.76 (m, 1H). |

### c) Preparation of Intermediate 11

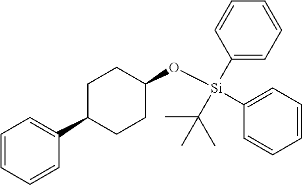 [SMILES: CC(C)(C)[Si](O[C@H]1CC[C@@H](c2ccccc2)CC1)(c1ccccc1)c1ccccc1]

A suspension of intermediate 8; (0.18 g, 0.43 mmol) and 10% palladium on carbon (0.040 g) in a mixture of DCM (5.0 ml) and MeOH (2.0 ml) was stirred under a hydrogen atmosphere for 4 hours. The resulting mixture was filtered through Celite® and the filtrate was concentrated in vacuo to afford the desired product as a colourless oil (0.18 g, 100%).

¹H NMR (400 MHz, CDCl₃) δ ppm: 7.72-7.68 (m, 4H), 7.45-7.11 (m, 11H), 4.12-4.09 (m, 0.6H), 3.71-3.63 (m, 0.4H), 2.55-2.41 (m, 1H), 2.16-2.07 (m, 1.2H), 1.97-1.92 (m, 0.8H), 1.81-1.78 (m, 2H), 1.67-1.60 (m, 1.2H), 1.59-1.39 (m, 2H), 1.37-1.26 (m, 0.8H), 1.13-1.10 (s, 5.4H), 1.08-1.05 (s, 3.6H). 3:2 mixture of cis:trans isomers.

Intermediates 12 to 15 were prepared according to the reaction protocol of intermediate 11 using the appropriate starting materials (Table 3).

TABLE 3

| | | Starting | |
|---|---|---|---|
| Intermediate | Structure | Materials | Analytical Data |
| 12 | 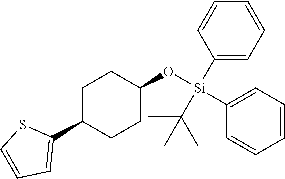 [SMILES: CC(C)(C)[Si](O[C@H]1CC[C@@H](c2cccs2)CC1)(c1ccccc1)c1ccccc1] | a) Intermediate 9; | ¹H NMR (400 MHz, CDCl₃) δ ppm: 7.69-7.63 (m, 4H), 7.45- 7.33 (m, 6H), 7.14 (dd, J = 1.2, 5.1 Hz, 0.7H), 7.07 (dd, J = 1.2, 5.1 Hz, 0.3H), 6.96 (dd, J = 3.5, 5.1 Hz, 0.7H), 6.89-6.85 (m, 1H), 6.73-6.71 (m, 0.3H), 4.08-4.04 (m, 0.7H), 3.69-3.61 (m, 0.3H), 2.86-2.75 (m, 1H), 2.13-1.24 (m, 8H), 1.09 (s, 6.3H), 1.06 (s, 2.7H). 7:3 mixture of cis:trans isomers. |
| 13 | 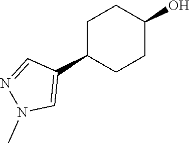 | a) Intermediate | ¹H NMR (400 MHz, CDCl₃) δ ppm: 7.34 (s, 1H), 7.16 (s, 1H), 4.03-4.00 (m, 1H), 3.86 (s, 3H), 2.62-2.52 (m, 1H), 1.82-1.61 (m, 8H), 1.56-1.52 (m, 1H). |
| 14 | 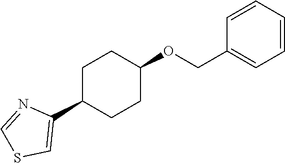 [SMILES: c1ccc(CO[C@H]2CC[C@@H](c3cscn3)CC2)cc1] | a) Intermediate 5; | ¹H NMR (300 MHz, CDCl₃) δ ppm: 8.76-8.73 (m, 1H), 7.38- 7.28 (m, 5H), 6.97-6.95 (m, 0.8H), 6.93-6.90 (m, 0.2H), 4.60 (s, 0.4H), 4.54 (s, 1.6H), 3.74- 3.68 (m, 0.8H), 3.48-3.37 (m, 0.2H), 2.97-2.80 (m, 1H), 2.24- 1.88 (m, 6H), 1.68-1.53 (m, 2H). 4:1 mixture of cis:trans isomers. |
| 15 | 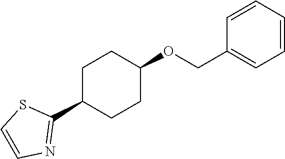 [SMILES: c1ccc(CO[C@H]2CC[C@@H](c3nccs3)CC2)cc1] | a) Intermediate 6; | Rt = 1.56, 1.59 min, m/z [M − H]⁻ = 274 (Method A) 3:1 mixture of isomers. |

### d) Preparation of Intermediate 16

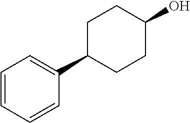 [SMILES: O[C@H]1CC[C@@H](c2ccccc2)CC1]

A mixture of intermediate 11; (0.18 g, 0.43 mmol), 1.0 M tetrabutylammonium fluoride solution in THF (0.86 ml, 0.86 mmol) and THF (4.0 ml) was heated at 70° C. for 18 hours. The resulting mixture was cooled to ambient temperature and concentrated in vacuo. The residue was purified by column chromatography on silica gel, eluting with a mixture of DCM and MeOH (1:0 to 47:3 by volume), to afford the desired product as a white gum (0.059 g, 78%).

¹H NMR (400 MHz, CDCl₃) δ ppm: 7.33-7.16 (m, 5H), 4.16-4.12 (m, 1H), 2.58-2.51 (m, 1H), 1.97-1.84 (m, 4H), 1.73-1.64 (m, 4H), 1.32-1.28 (m, 1H).

Intermediate 17 was prepared according to the reaction protocol of intermediate 16 using the appropriate starting materials (Table 4).

TABLE 4

| | | Starting | |
|---|---|---|---|
| Intermediate | Structure | Materials | NMR Data |
| 17 | 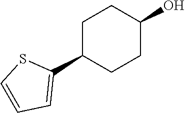 [SMILES: O[C@H]1CC[C@@H](c2cccs2)CC1] | a) Intermediate 12; | ¹H NMR (400 MHz, CDCl₃) δ ppm: 7.14-7.11 (m, 1H), 6.95-6.91 (m, 1H), 6.85-6.83 (m, 0.7H), 6.81- 6.80 (m, 0.3H), 4.08-4.05 (m, 0.7H), 4.01-3.99 (m, 0.3H), 2.89- 2.78 (m, 1H), 2.16-2.07 (m, 2H), 1.97-1.82 (m, 4H), 1.74-1.65 (m, 2H), 1.27-1.25 (m, 1H). 7:3 mixture of cis:trans isomers. |

### Example A5

### a) Preparation of Intermediate 18

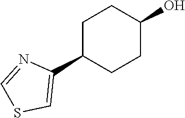 [SMILES: O[C@H]1CC[C@@H](c2cscn2)CC1]

A solution of intermediate 14; (0.19 g, 0.69 mmol) in a mixture of DCM (14 ml) and water (0.5 ml) was treated with DDQ (0.17 g, 0.76 mmol) and the resulting mixture was heated at 40° C. for 1 hour. The mixture was cooled to ambient temperature and partitioned between DCM and saturated aqueous sodium bicarbonate solution. The organic phase was dried over sodium sulfate and concentrated in vacuo. The residue was purified by column chromatography on silica gel, eluting with a mixture of cyclohexane and EtOAc (1:0 to 0:1 by volume), to afford the desired product as a colourless oil (0.046 g, 37%).

¹H NMR (300 MHz, CDCl₃) δ ppm: 8.76 (d, J=2.0 Hz, 1H), 6.97 (dd, J=0.9, 2.0 Hz, 1H), 4.12-4.07 (m, 1H), 2.96-2.85 (m, 1H), 2.04-1.37 (m, 9H).

Intermediate 19 was prepared according to the reaction protocol of intermediate 18 using the appropriate starting materials (Table 5).

TABLE 5

| | | Starting | |
|---|---|---|---|
| Intermediate | Structure | Materials | NMR Data |
| 19 | 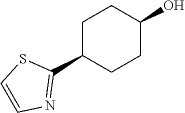 [SMILES: O[C@H]1CC[C@@H](c2nccs2)CC1] | a) Intermediate 15; | ¹H NMR (400 MHz, CDCl₃) δ ppm: 7.69 (d, J = 3.3 Hz, 1H), 7.22 (d, J = 3.3 Hz, 1H), 4.11-4.04 (m, 1H), 3.15-3.04 (m, 1H), 2.14-1.68 (m, 8H), 1.39-1.34 (m, 1H). |

### Example A6

### a) Preparation of Intermediate 20

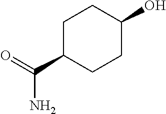

A suspension of 2-oxabicyclo[2.2.2]octan-3-one (10.0 g, 79.4 mmol) in 33% ammonia solution in water (100 ml) was stirred at ambient temperature for 18 hours. The resulting mixture was concentrated in vacuo. The residue was azeotroped with toluene and dried under high vacuum for 18 hours to afford the desired product as a white solid (11.3 g, 100%).

¹H NMR (400 MHz, DMSO-d₆) δ ppm: 7.09 (s, 1H), 6.60 (s, 1H), 4.29-4.24 (m, 1H), 3.75-3.69 (m, 1H), 2.10-2.02 (m, 1H), 1.81-1.69 (m, 2H), 1.63-1.55 (m, 2H), 1.45-1.35 (m, 4H).

### Example A7

### a) Preparation of Intermediate 21

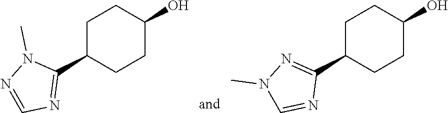

A mixture of intermediate 20 (0.50 g, 3.50 mmol) in N,N-dimethylformamide dimethyl acetal (10 ml) was heated at 110° C. for 2 hours. The resulting mixture was cooled to ambient temperature and concentrated in vacuo, azeotroping with toluene. The residue was taken up in acetic acid (10 ml), treated with methylhydrazine (1.0 ml) and the resulting mixture was heated at 90° C. for 2 hours. The mixture was cooled to ambient temperature and concentrated in vacuo. The residue was partitioned between EtOAc and saturated aqueous sodium bicarbonate solution. The organic phase was dried over sodium sulfate and concentrated in vacuo to afford the desired product as a colourless oil (0.070 g, 11%).

¹H NMR (400 MHz, DMSO-d₆) δ ppm: 7.81-7.79 (m, 1H), 5.26-5.22 (m, 0.5H), 5.10-5.07 (m, 0.5H), 3.86-3.84 (m, 3H), 2.87-2.78 (m, 1H), 2.14-1.94 (m, 4H), 1.82-1.59 (m, 5H). 1:1 mixture of regioisomers.

Intermediate 22 was prepared according to the reaction protocol of intermediate 21 using the appropriate starting materials (Table 6).

TABLE 6

| Intermediate | Structure | Starting Materials | LCMS Data |
|---|---|---|---|
| 22 | 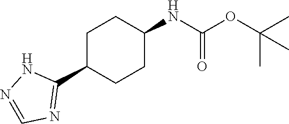 [SMILES: CC(C)(C)OC(=O)N[C@H]1CC[C@@H](c2ncn[nH]2)CC1] | a) tert-Butyl cis-4- carbamoylcyclohexyl) carbamate b) Hydrazine hydrate | Rt = 1.43 min, m/z [M − H]⁻ = 265 (Method B) |

### Example A8

### a) Preparation of Intermediate 23

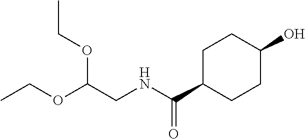 [SMILES: CCOC(CNC(=O)[C@H]1CC[C@@H](O)CC1)OCC]

A solution of cis-4-hydroxycyclohexane-1-carboxylic acid (0.25 g, 1.73 mmol), 2,2-diethoxyethan-1-amine (0.26 ml, 1.73 mmol) and DIPEA (0.90 ml, 5.19 mmol) in DMF (4.0 ml) was treated with HATU (0.86 g, 2.25 mmol). After stirring at ambient temperature for 1 hour, the resulting mixture was partitioned between EtOAc and saturated aqueous sodium bicarbonate solution. The organic phase was dried over sodium sulfate and concentrated in vacuo. The residue was purified by column chromatography on silica gel, eluting with a mixture of DCM and 2.0 M ammonia solution in MeOH (1:0 to 9:1 by volume), to afford the desired product as a brown oil (0.38 g, 84%).

¹H NMR (400 MHz, DMSO-d₆) δ ppm: 7.68 (t, J=5.8 Hz, 1H), 4.45 (t, J=5.6 Hz, 1H), 4.28-4.26 (m, 1H), 3.74-3.72 (m, 1H), 3.65-3.55 (m, 2H), 3.49-3.41 (m, 2H), 3.11-3.07 (m, 2H), 2.16-2.08 (m, 1H), 1.82-1.70 (m, 2H), 1.63-1.57 (m, 2H), 1.44-1.33 (m, 4H), 1.10 (t, J=7.1 Hz, 6H).

Intermediate 24 was prepared according to the reaction protocol of intermediate 23 using the appropriate starting materials (Table 7).

TABLE 7

| Intermediate | Structure | Starting Materials | LCMS Data |
|---|---|---|---|
| 24 | 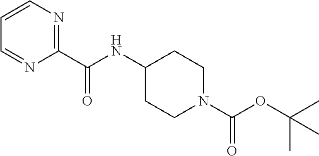 [SMILES: CC(C)(C)OC(=O)N1CCC(NC(=O)c2ncccn2)CC1] | a) 2-Pyrimidinecarboxylic acid b) Tert-butyl 4- aminopiperidine-1- carboxylate | Rt = 1.46 min, m/z [M + Na]⁺ = 329 (Method B) |

### Example A9

### a) Preparation of Intermediate 25

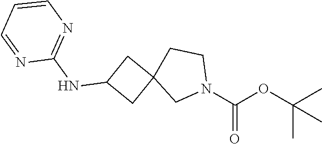 [SMILES: CC(C)(C)OC(=O)N1CCC2(CC(Nc3ncccn3)C2)C1]

A mixture of tert-butyl 2-amino-6-azaspiro[3.4]octane-6-carboxylate (0.45 g, 1.97 mmol), 2-fluoropyrimidine (0.19 g, 1.97 mmol) and triethylamine (0.55 ml, 3.94 mmol) in iPrOH (2.0 ml) was heated at 130° C. under microwave irradiation for 1 hour. The resulting mixture was cooled to ambient temperature and concentrated in vacuo. The residue was purified by flash chromatography on silica gel, eluting with a mixture of DCM and MeOH (1:0 to 47:3 by volume), to afford the desired product as a white solid (0.57 g, 95%).

LCMS (Method A): Rt=1.24 min, m/z [M-tBu]⁺=249

Intermediates 26 to 31 were prepared according to the reaction protocol of intermediate 25 using the appropriate starting materials (Table 8).

TABLE 8

| Intermediate | Structure | Starting Materials | LCMS Data |
|---|---|---|---|
| 26 | 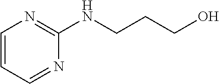 [SMILES: OCCCNc1ncccn1] | a) 3-Aminopropan-1-ol b) 2-Fluoropyrimidine | Rt = 0.76 min, m/z [M + H]⁺ = 154 (Method B) |
| 27 | 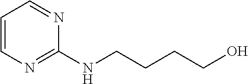 [SMILES: OCCCCNc1ncccn1] | a) 4-Aminobutan-1-ol b) 2-Fluoropyrimidine | Rt = 0.93 min, m/z [M + H]⁺ = 168 (Method B) U1110145 |
| 28 | 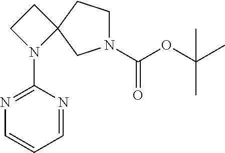 [SMILES: CC(C)(C)OC(=O)N1CCC2(CCN2c2ncccn2)C1] | a) tert-Butyl 1,6- diazaspiro[3.4]octane-6- carboxylate b) 2-Fluoropyrimidine | Rt = 1.45 min, m/z [M + H]⁺ = 291 (Method A) |
| 29 | 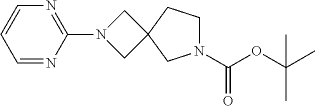 [SMILES: CC(C)(C)OC(=O)N1CCC2(C1)CN(c1ncccn1)C2] | a) tert-Butyl 2,6- diazaspiro[3.4]octane-6- carboxylate b) 2-Fluoropyrimidine | Rt = 1.16 min, m/z [M + H]⁺ = 291 (Method A) |
| 30 | 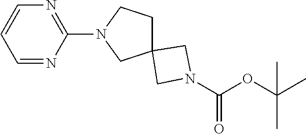 [SMILES: CC(C)(C)OC(=O)N1CC2(CCN(c3ncccn3)C2)C1] | a) tert-Butyl 2,6- diazaspiro[3.4]octane-2- carboxylate b) 2-Fluoropyrimidine | Rt = 1.17 min, m/z [M + H]⁺-tert-Bu = 235 (Method A) |
| 31 | 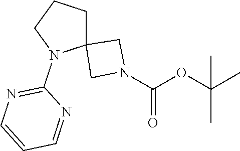 [SMILES: CC(C)(C)OC(=O)N1CC2(CCCN2c2ncccn2)C1] | a) tert-Butyl 2,5- diazaspiro[3.4]octane-2- carboxylate b) 2-Fluoropyrimidine | Rt = 1.54 min, m/z [M + H]⁺ = 291 (Method A) |

### b) Preparation of Intermediate 32

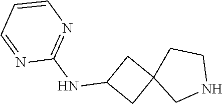 [SMILES: c1cnc(NC2CC3(CCNC3)C2)nc1]

A solution of intermediate 25 (0.57 g, 1.87 mmol) in DCM (5.0 ml) was treated with TFA (2.0 ml) and the resulting mixture was stirred at ambient temperature under a nitrogen atmosphere for 2 hours. The resulting mixture was purified on an ISOLUTE® SCX-2 SPE column, eluting with MeOH followed by 2.0 M ammonia solution in MeOH, to afford the desired product as a pale yellow oil (0.35 g, 91%).

LCMS (Method B): Rt=1.06 min, m/z [M+H]⁺=205

Intermediates 33 to 38 were prepared according to the reaction protocol of intermediate 32 using the appropriate starting materials (Table 9).

TABLE 9

| Intermediate | Structure | Starting Materials | Analytical Data |
|---|---|---|---|
| 33 | 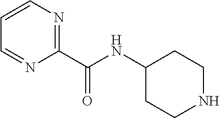 [SMILES: O=C(NC1CCNCC1)c1ncccn1] | a) Intermediate 24; | Rt = 0.13 min, m/z [M + H]⁺ = 207 (Method A) |
| 34 | 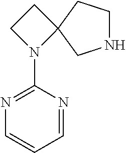 [SMILES: c1cnc(N2CCC23CCNC3)nc1] | a) Intermediate 28; | Rt = 0.30 min, m/z [M + H]⁺ = 191 (Method A) |
| 35 | 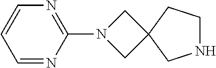 [SMILES: c1cnc(N2CC3(CCNC3)C2)nc1] | a) Intermediate 29; | Rt = 0.17 min, m/z [M + H]⁺ = 191 (Method A) |
| 36 | 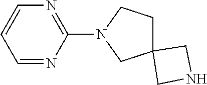 [SMILES: c1cnc(N2CCC3(CNC3)C2)nc1] | a) Intermediate 30; | Rt = 0.17 min, m/z [M + H]⁺ = 191 (Method A) |
| 37 | 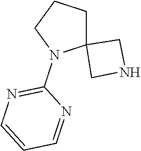 [SMILES: c1cnc(N2CCCC23CNC3)nc1] | a) Intermediate 31; | Rt = 0.40 min, m/z [M + H]⁺ = 191 (Method A) |
| 38 | 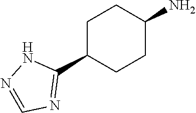 [SMILES: N[C@H]1CC[C@@H](c2ncn[nH]2)CC1] | a) Intermediate 22; | ¹H NMR (400 MHz, DMSO-d₆) δ ppm: 7.99 (s, 1H), 2.89 (tt, J = 3.4, 6.8 Hz, 1H), 2.83 (tt, J = 4.0, 7.9 Hz, 1H), 2.09- 1.99 (m, 2H), 1.68-1.54 (m, 4H), 1.43-1.36 (m, |
| | | | 2H). |

### Example A10

### a) Preparation of Intermediate 39

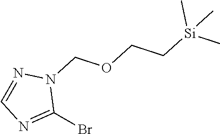

A solution of 5-bromo-1H-1,2,4-triazole (0.40 g, 2.70 mmol) and triethylamine (0.75 ml, 5.40 mmol) in DCM (5.0 mL) at 0° C. was treated with 2-(trimethylsilyl)ethoxymethyl chloride (0.58 ml, 3.24 mmol). After warming to ambient temperature for 45 minutes, the resulting mixture was partitioned between DCM and water. The organic phase was dried over magnesium sulfate and concentrated in vacuo. The residue was purified by column chromatography on silica gel, eluting with a mixture of cyclohexane and EtOAc (1:0 to 3:2 by volume), to afford the desired product as a colourless oil (0.20 g, 27%).

¹H NMR (300 MHz, CDCl₃) δ ppm: 7.93 (s, 1H), 5.51 (s, 2H), 3.70-3.63 (m, 2H), 0.97-0.91 (m, 2H), 0.02-0.03 (m, 9H).

### Example A11

### a) Preparation of Intermediate 40

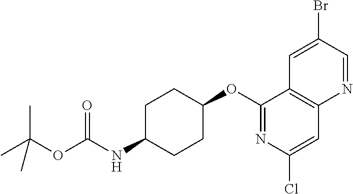 [SMILES: CC(C)(C)OC(=O)N[C@H]1CC[C@@H](Oc2nc(Cl)cc3ncc(Br)cc23)CC1]

A mixture of intermediate 1 (0.28 g, 1.00 mmol), cis-(4-hydroxycyclohexyl)carbamic acid tert-butyl ester (0.26 g, 1.20 mmol), cesium carbonate (0.49 g, 1.50 mmol) and dioxane (5.0 ml) was heated at 110° C. in a sealed tube for 18 hours. Additional portions of cis-(4-hydroxycyclohexyl)carbamic acid tert-butyl ester (0.13 g, 0.60 mmol) and cesium carbonate (0.24 g, 0.75 mmol) were added and heating was continued at 110° C. for a further 4 hours. The resulting mixture was cooled to ambient temperature and partitioned between EtOAc and water. The organic phase was dried over sodium sulfate and concentrated in vacuo. The residue was purified by column chromatography on silica gel, eluting with a mixture of cyclohexane and EtOAc (1:0 to 0:1 by volume), to afford the desired product as a pale yellow solid (0.26 g, 56%).

LCMS (Method A): Rt=1.87 min, m/z [M+H]⁺=456/458/460

Intermediates 41 to 47 were prepared according to the reaction protocol of intermediate 40 using the appropriate starting materials (Table 10).

TABLE 10

| Intermediate | Structure | Starting Materials | Analytical Data |
|---|---|---|---|
| 41 | 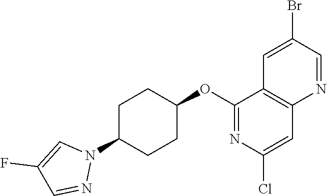 [SMILES: Fc1cnn([C@H]2CC[C@@H](Oc3nc(Cl)cc4ncc(Br)cc34)CC2)c1] | a) Intermediate 1 b) Intermediate 4 | Rt = 1.62 min, m/z [M + H]⁺ = 425/427/429 (Method A) |
| 42 | 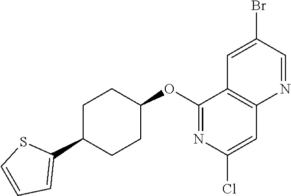 [SMILES: Clc1cc2ncc(Br)cc2c(O[C@H]2CC[C@@H](c3cccs3)CC2)n1] | a) Intermediate 1 b) Intermediate 17 | Rt = 1.97 min, m/z [M + H]⁺ = 423/425/427 (Method A) |
| 43 | 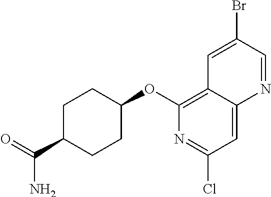 [SMILES: NC(=O)[C@H]1CC[C@@H](Oc2nc(Cl)cc3ncc(Br)cc23)CC1] | a) Intermediate 1 b) Intermediate 20 | Rt = 1.38 min, m/z [M + H]⁺ = 384/386/388 (Method A) |
| 44 | 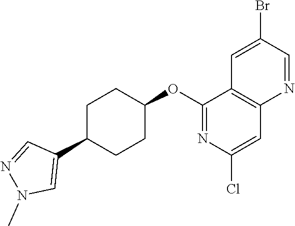 [SMILES: Cn1cc([C@H]2CC[C@@H](Oc3nc(Cl)cc4ncc(Br)cc34)CC2)cn1] | a) Intermediate 1; b) Intermediate 13 | Rt = 1.71 min, m/z [M + H]⁺ = 421/423/425 (Method A) |
| 45 | 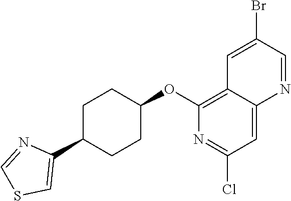 [SMILES: Clc1cc2ncc(Br)cc2c(O[C@H]2CC[C@@H](c3cscn3)CC2)n1] | a) Intermediate 1 b) Intermediate 18 | ¹H NMR (300 MHz, CDCl₃) δ ppm: 8.99 (d, J = 2.1 Hz, 1H), 8.80 (d, J = 2.1 Hz, 1H), 8.61 (dd, J = 0.9, 2.2 Hz, 1H), 7.47 (d, J = 0.8 Hz, 1H), 7.03 (dd, J = 0.9, 2.2 Hz, 1H), 5.67-5.63 (m, 1H), 3.07-2.97 (m, 1H), 2.33-2.24 (m, 2H), |
| | | | 2.15-1.83 (m, 6H). |
| 46 | 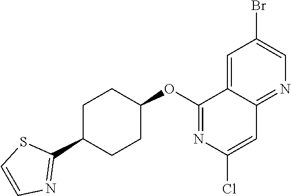 [SMILES: Clc1cc2ncc(Br)cc2c(O[C@H]2CC[C@@H](c3nccs3)CC2)n1] | a) Intermediate 1 b) Intermediate 19 | ¹H NMR (400 MHz, CDCl₃) δ ppm: 8.99 (d, J = 2.8 Hz, 1H), 8.61 (dd, J = 0.9, 2.2 Hz, 1H), 7.74 (d, J = 2.8 Hz, 1H), 7.48 (d, J = 0.8 Hz, 1H), 7.26-7.24 (m, 1H), 5.66-5.60 (m, 1H), 3.26-3.17 (m, 1H), 2.34-2.25 (m, 2H), 2.18-2.04 (m, 4H), 1.95-1.85 (m, 2H). |
| 47 | 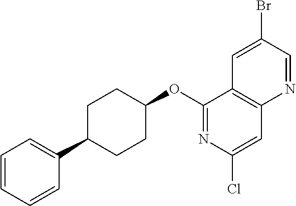 [SMILES: Clc1cc2ncc(Br)cc2c(O[C@H]2CC[C@@H](c3ccccc3)CC2)n1] | a) Intermediate 1 b) Intermediate 16 | Rt = 1.99 min, m/z [M + H]⁺ = 417/419/421 (Method A) |

### Example A12

### a) Preparation of Intermediate 48

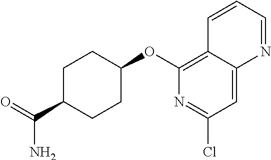 [SMILES: NC(=O)[C@H]1CC[C@@H](Oc2nc(Cl)cc3ncccc23)CC1]

A mixture of intermediate 20 (0.71 g, 4.96 mmol) in anhydrous THF (25 ml) was treated portionwise with sodium hydride (0.20 g, 5.0 mmol, 60% in mineral oil). The resulting mixture was stirred at ambient temperature for 20 minutes, then heated to 50° C. for 30 minutes. 5,7-Dichloro-1,6-naphthyridine (1.00 g, 5.02 mmol) was added and the resulting mixture was heated under reflux for 2 hours. The mixture was cooled to ambient temperature and partitioned between EtOAc and water. The organic phase was dried over sodium sulfate and concentrated in vacuo. The residue was purified by column chromatography on silica gel, eluting with a mixture of EtOAc and MeOH (1:0 to 4:1 by volume), to afford the desired product as a white solid (0.69 g, 46%).

LCMS (Method A): Rt=1.16 min, m/z [M+H]⁺=306/308

Intermediates 49 to 64 were prepared according to the reaction protocol of intermediate 48 using the appropriate starting materials (Table 11).

TABLE 11

| Intermediate | Structure | Starting Materials | Analytical Data |
|---|---|---|---|
| 49 | 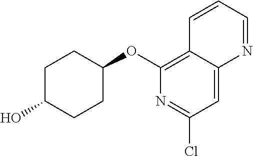 [SMILES: O[C@H]1CC[C@H](Oc2nc(Cl)cc3ncccc23)CC1] | a) 5,7-Dichloro-1,6- naphthyridine; b) trans-1,4- Cyclohexanediol; | Rt = 1.29 min, m/z [M + H]⁺ = 279/281 (Method A) |
| 50 | 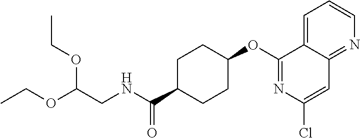 [SMILES: CCOC(CNC(=O)[C@H]1CC[C@@H](Oc2nc(Cl)cc3ncccc23)CC1)OCC] | a) 5,7-Dichloro-1,6- naphthyridine; b) Intermediate 23 | Rt = 1.65 min, m/z [M + H]⁺ = 422/424 (Method A) |
| 51 | 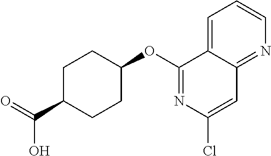 [SMILES: O=C(O)[C@H]1CC[C@@H](Oc2nc(Cl)cc3ncccc23)CC1] | a) 5,7-Dichloro-1,6- naphthyridine; b) cis-4-Hydroxy- cyclohexanecarboxylic acid; | Rt = 1.34 min, m/z [M + H]⁺ = 307/309 (Method A) |
| 52 | 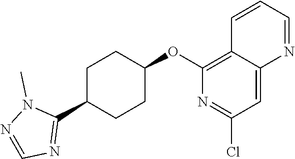 [SMILES: Cn1ncnc1[C@H]1CC[C@@H](Oc2nc(Cl)cc3ncccc23)CC1] | a) 5,7-Dichloro-1,6- naphthyridine; b) Intermediate 21 | ¹H NMR (400 MHz, CDCl₃) δ ppm: 9.00 (dd, J = 1.5, 4.3 Hz, 1H), 8.62-8.58 (m, 1H), 7.83 (s, 1H), 7.51 (d, J = 0.7 Hz, 1H), 7.45 (dd, J = 4.3, 8.4 Hz, 1H), 5.72-5.69 (m, 1H), 3.89 (s, 3H), 2.96-2.87 (m, 1H), 2.39- 2.34 (m, 2H), 2.24-2.13 (m, 2H), 1.92-1.81 (m, 4H). |
| 53 | 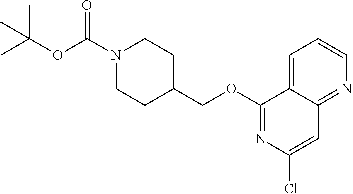 [SMILES: CC(C)(C)OC(=O)N1CCC(COc2nc(Cl)cc3ncccc23)CC1] | a) 5,7-Dichloro-1,6- naphthyridine; b) tert-Butyl 4- (hydroxymethyl)piperidine- 1-carboxylate; | Rt = 1.65 min, m/z [M + H]⁺ = 322/324 (Method A) |
| 54 | 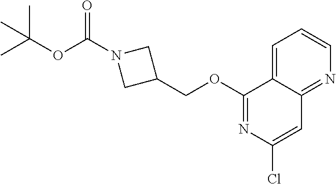 [SMILES: CC(C)(C)OC(=O)N1CC(COc2nc(Cl)cc3ncccc23)C1] | a) 5,7-Dichloro-1,6- naphthyridine; b) tert-Butyl 3- (hydroxymethyl)azetidine-1- carboxylate; | ¹H NMR (400 MHz, DMSO-d₆) δ ppm: 9.10 (dd, J = 1.5, 4.3 Hz, 1H), 8.50 (dd, J = 1.5, 8.4 Hz, 1H), 7.65 (dd, J = 4.3, 8.4 Hz, 1H), 7.60- 7.59 (m, 1H), 4.62 (d, J = 6.1 Hz, 2H), 4.07- |
| | | | 3.98 (m, 2H), |
| | | | 3.85-3.80 (m, |
| | | | 2H), 3.11-3.03 |
| | | | (m, 1H), 1.39 (s, |
| | | | 9H). |
| 55 | 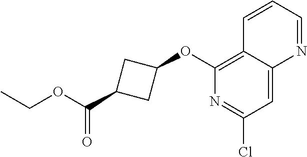 [SMILES: CCOC(=O)[C@H]1C[C@@H](Oc2nc(Cl)cc3ncccc23)C1] | a) 5,7-Dichloro-1,6- naphthyridine; b) cis-3-Hydroxy- cyclobutanecarboxylic acid ethyl ester; | Rt = 1.50 min, m/z [M + H]⁺ = 307/309 (Method A) |
| 56 | 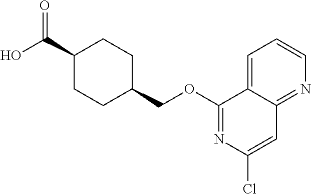 [SMILES: O=C(O)[C@H]1CC[C@@H](COc2nc(Cl)cc3ncccc23)CC1] | a) 5,7-Dichloro-1,6- naphthyridine; b) cis-4-(Hydroxymethyl)- cyclohexanecarboxylic acid; | Rt = 1.53 min, m/z [M + H]⁺ = 321/323 (Method A) |
| 57 | 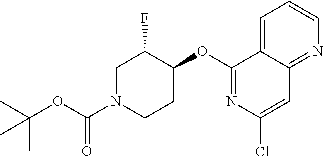 [SMILES: CC(C)(C)OC(=O)N1CC[C@H](Oc2nc(Cl)cc3ncccc23)[C@@H](F)C1] | a) 5,7-Dichloro-1,6- naphthyridine; b) tert-Butyl (3S,4S)-3- fluoro-4-hydroxypiperidine- 1-carboxylate; | Rt = 1.76 min, m/z [M + H]⁺ = 382/384 (Method A) |
| 58 | 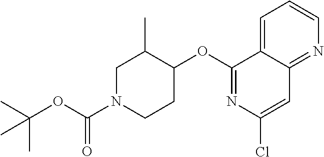 [SMILES: CC1CN(C(=O)OC(C)(C)C)CCC1Oc1nc(Cl)cc2ncccc12] | a) 5,7-Dichloro-1,6- naphthyridine; b) tert-Butyl 4-hydroxy-3- methylpiperidine-1- carboxylate; | ¹H NMR (400 MHz, CDCl₃) δ ppm: 9.03-9.00 (m, 1H), 8.51- 8.48 (m, 1H), 7.52-7.51 (m, 1H), 7.47-7.42 (m, 1H), 5.61- 5.57 (m, 0.4H), 5.19-5.10 (m, |
| | | | 0.6H), 4.10-3.69 |
| | | | (m, 2H), 3.31- |
| | | | 3.12 (m, 1.4H), |
| | | | 2.80 (bs, 0.6H), |
| | | | 2.33-2.25 (m, |
| | | | 0.6H), 2.17-2.01 |
| | | | (m, 1.4H), 1.90- |
| | | | 1.81 (m, 0.4H), |
| | | | 1.67-1.57 (m, |
| | | | 0.6H), 1.49 (s, |
| | | | 9H), 1.04-1.00 |
| | | | (m, 3H). 3:2 |
| | | | mixture of |
| | | | cis:trans |
| | | | isomers. |
| 59 | 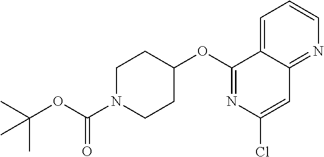 [SMILES: CC(C)(C)OC(=O)N1CCC(Oc2nc(Cl)cc3ncccc23)CC1] | a) 5,7-Dichloro-1,6- naphthyridine; b) tert-Butyl 4- hydroxypiperidine-1- carboxylate; | Rt = 1.63 min, m/z [M + H]⁺ = 364/366 (Method A) |
| 60 | 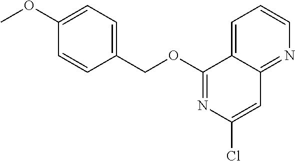 [SMILES: COc1ccc(COc2nc(Cl)cc3ncccc23)cc1] | a) 5,7-Dichloro-1,6- naphthyridine; b) (4- Methoxyphenyl)methanol; | Rt = 1.62 min, m/z [M + H]⁺ = 301/303 (Method A) |
| 61 | 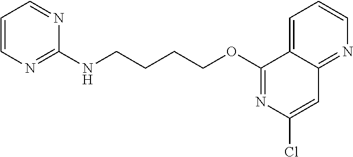 [SMILES: Clc1cc2ncccc2c(OCCCCNc2ncccn2)n1] | a) 5,7-Dichloro-1,6- naphthyridine; b) Intermediate 27 | Rt = 1.20 min, m/z [M + H]⁺ = 330/332 (Method A) |
| 62 | 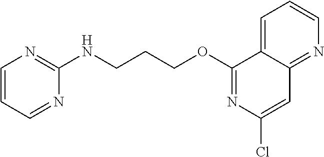 [SMILES: Clc1cc2ncccc2c(OCCCNc2ncccn2)n1] | a) 5,7-Dichloro-1,6- naphthyridine; b) Intermediate 26 | Rt = 1.15 min, m/z [M + H]⁺ = 316/318 (Method A) |
| 63 | 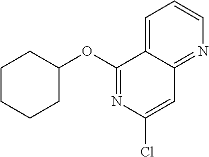 [SMILES: Clc1cc2ncccc2c(OC2CCCCC2)n1] | a) 5,7-Dichloro-1,6- naphthyridine; b) Cyclohexanol; | Rt = 1.74 min, m/z [M + H]⁺ = 263/265 (Method A) |
| 64 | 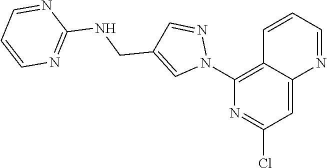 [SMILES: Clc1cc2ncccc2c(-n2cc(CNc3ncccn3)cn2)n1] | a) 5,7-Dichloro-1,6- naphthyridine b) N-((1H-Pyrazol-4- yl)methyl)pyrimidin-2-amine | Rt = 1.19 min, m/z [M + H]⁺ = 338/340 (Method A) |

### Example A13

### a) Preparation of Intermediate 65

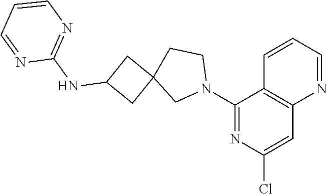 [SMILES: Clc1cc2ncccc2c(N2CCC3(CC(Nc4ncccn4)C3)C2)n1]

A mixture of 5,7-dichloro-1,6-naphthyridine (0.13 g, 0.68 mmol), intermediate 32 (0.14 g, 0.68 mmol) and triethylamine (0.19 ml, 1.36 mmol) in iPrOH (1.0 ml) was heated at 100° C. under microwave irradiation for 30 minutes. The resulting mixture was cooled to ambient temperature and concentrated in vacuo. The residue was purified by column chromatography on silica gel, eluting with a mixture of DCM and MeOH (1:0 to 47:3 by volume), to afford the desired product as a yellow oil (0.25 g, 100%).

LCMS (Method A): Rt=1.13/1.14 min, m/z [M+H]⁺=367/369

Intermediates 66 to 100 were prepared according to the reaction protocol of intermediate 65 using the appropriate starting materials (Table 12).

TABLE 12

| Intermediate | Structure | Starting Materials | LCMS Data |
|---|---|---|---|
| 66 | 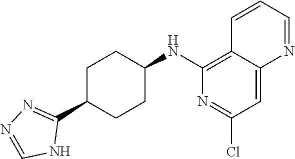 [SMILES: Clc1cc2ncccc2c(N[C@H]2CC[C@@H](c3nnc[nH]3)CC2)n1] | a) 5,7-Dichloro-1,6-naphthyridine b) Intermediate 38; | Rt = 1.04 min, m/z [M + H]⁺ = 329/331 (Method A) |
| 67 | 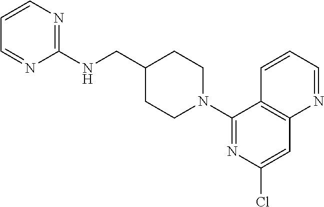 [SMILES: Clc1cc2ncccc2c(N2CCC(CNc3ncccn3)CC2)n1] | a) 5,7-Dichloro-1,6-naphthyridine b) N-(Piperidin-4- ylmethyl)pyrimidin-2-amine | Rt = 1.22 min, m/z [M + H]⁺ = 355/357 (Method A) |
| 68 | 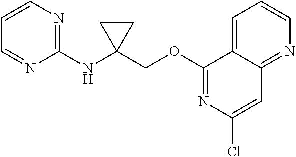 [SMILES: Clc1cc2ncccc2c(OCC2(Nc3ncccn3)CC2)n1] | a) 5,7-Dichloro-1,6-naphthyridine b) (1-(Pyrimidin-2- ylamino)cyclopropyl)methanol; | Rt = 1.19 min, m/z [M + H]⁺ = 328/330 (Method A) |
| 69 | 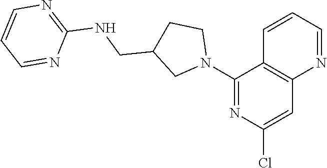 [SMILES: Clc1cc2ncccc2c(N2CCC(CNc3ncccn3)C2)n1] | a) 5,7-Dichloro-1,6-naphthyridine b) N-(Pyrrolidin-3- ylmethyl)pyrimidin-2-amine | Rt = 1.04 min, m/z [M + H]⁺ = 341/343 (Method A) |
| 70 | 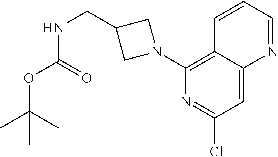 [SMILES: CC(C)(C)OC(=O)NCC1CN(c2nc(Cl)cc3ncccc23)C1] | a) 5,7-Dichloro-1,6-naphthyridine b) tert-Butyl(azetidin-3- ylmethyl)carbamate | Rt = 1.78 min, m/z [M + H]⁺ = 349/351 (Method B) |
| 71 | 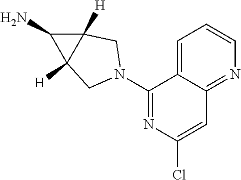 [SMILES: N[C@@H]1[C@H]2CN(c3nc(Cl)cc4ncccc34)C[C@@H]12] | a) 5,7-Dichloro-1,6-naphthyridine b) (1R,58,6S)-3- Azabicyclo[3.1.0]hexan-6-amine | Rt = 0.71 min, m/z [M + H]⁺ = 261/263 (Method A) |
| 72 | 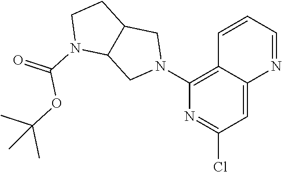 [SMILES: CC(C)(C)OC(=O)N1CCC2CN(c3nc(Cl)cc4ncccc34)CC21] | a) 5,7-Dichloro-1,6-naphthyridine b) tert-Butyl hexahydropyrrolo[3,4- b]pyrrole-1(2H)-carboxylate | Rt = 1.70 min, m/z [M + H]⁺ = 375/377 (Method A) |
| 73 | 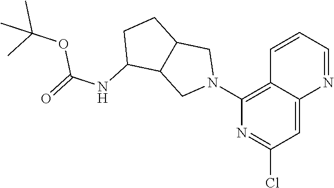 [SMILES: CC(C)(C)OC(=O)NC1CCC2CN(c3nc(Cl)cc4ncccc34)CC21] | a) 5,7-Dichloro-1,6-naphthyridine b) tert-Butyl (octahydrocyclopenta[c]pyrrol-4- yl)carbamate | Rt = 1.44 min, m/z [M + H]⁺ = 389/391 (Method A) |
| 74 | 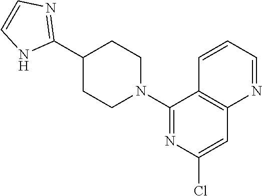 [SMILES: Clc1cc2ncccc2c(N2CCC(c3ncc[nH]3)CC2)n1] | a) 5,7-Dichloro-1,6-naphthyridine b) 4-(1H-Imidazol-2-yl)piperidine | Rt = 1.01 min, m/z [M + H]⁺ = 314/316 (Method A) |
| 75 | 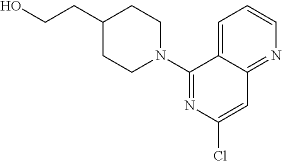 [SMILES: OCCC1CCN(c2nc(Cl)cc3ncccc23)CC1] | a) 5,7-Dichloro-1,6-naphthyridine b) 2-(Piperidin-4-yl)ethan-1-ol | Rt = 1.22 min, m/z [M + H]⁺ = 292/294 (Method A) |
| 76 | 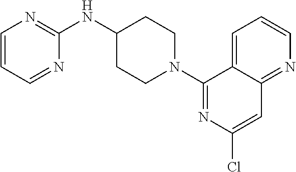 [SMILES: Clc1cc2ncccc2c(N2CCC(Nc3ncccn3)CC2)n1] | a) 5,7-Dichloro-1,6-naphthyridine b) N-(Piperidin-4-yl)pyrimidin-2- amine | Rt = 1.19 min, m/z [M + H]⁺ = 341/343 (Method A) |
| 77 | 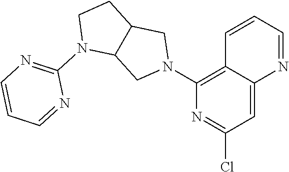 [SMILES: Clc1cc2ncccc2c(N2CC3CCN(c4ncccn4)C3C2)n1] | a) 5,7-Dichloro-1,6-naphthyridine b) 1-(Pyrimidin-2- yl)octahydropyrrolo[3,4-b]pyrrole | Rt = 1.19 min, m/z [M + H]⁺ = 353/355 (Method A) |
| 78 | 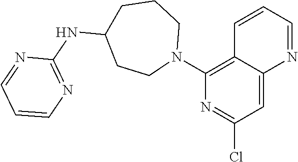 [SMILES: Clc1cc2ncccc2c(N2CCCC(Nc3ncccn3)CC2)n1] | a) 5,7-Dichloro-1,6-naphthyridine b) N-(Pyrimidin-2-yl)azepan-4- amine | Rt = 1.14 min, m/z [M + H]⁺ = 355/357 (Method A) |
| 79 | 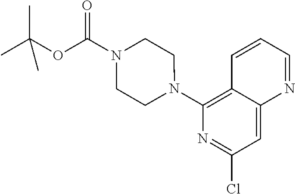 [SMILES: CC(C)(C)OC(=O)N1CCN(c2nc(Cl)cc3ncccc23)CC1] | a) 5,7-Dichloro-1,6-naphthyridine b) tert-Butyl piperazine-1- carboxylate | Rt = 1.61 min, m/z [M + H]⁺ = 349/351 (Method A) |
| 80 | 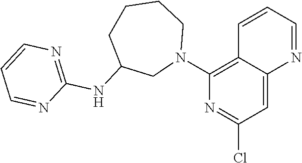 [SMILES: Clc1cc2ncccc2c(N2CCCCC(Nc3ncccn3)C2)n1] | a) 5,7-Dichloro-1,6-naphthyridine b) N-(Pyrimidin-2-yl)azepan-4- amine | Rt = 1.22 min, m/z [M + H]⁺ = 355/357 (Method A) |
| 81 | 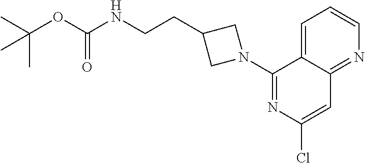 [SMILES: CC(C)(C)OC(=O)NCCC1CN(c2nc(Cl)cc3ncccc23)C1] | a) 5,7-Dichloro-1,6-naphthyridine b) tert-Butyl(2-(azetidin-3- yl)ethylcarbamate | Rt = 1.36 min, m/z [M + H]⁺ = 363/365 (Method A) |
| 82 | 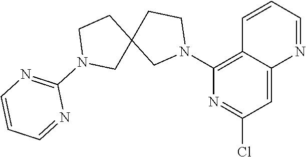 [SMILES: Clc1cc2ncccc2c(N2CCC3(CCN(c4ncccn4)C3)C2)n1] | a) 5,7-Dichloro-1,6-naphthyridine b) 2-(Pyrimidin-2-yl)-2,7- diazaspiro[4.4]nonane | Rt = 1.13 min, m/z [M + H]⁺ = 367/369 (Method A) |
| 83 | 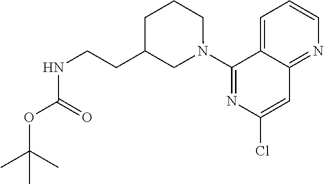 [SMILES: CC(C)(C)OC(=O)NCCC1CCCN(c2nc(Cl)cc3ncccc23)C1] | a) 5,7-Dichloro-1,6-naphthyridine b) tert-Butyl(2-(piperidin-3- yl)ethylcarbamate | Rt = 1.62 min, m/z [M + H]⁺ = 391/393 (Method A) |
| 84 | 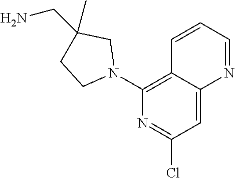 [SMILES: CC1(CN)CCN(c2nc(Cl)cc3ncccc23)C1] | a) 5,7-Dichloro-1,6-naphthyridine b) (3-Methylpyrrolidin-3- yl)methanamine hydrochloride | Rt = 0.62 min, m/z [M + H]⁺ = 277/279 (Method A) |
| 85 | 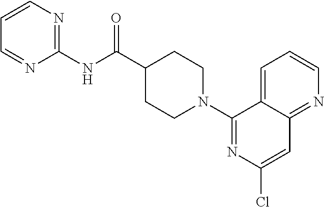 [SMILES: O=C(Nc1ncccn1)C1CCN(c2nc(Cl)cc3ncccc23)CC1] | a) 5,7-Dichloro-1,6-naphthyridine b) N-(Pyrimidin-2-yl)piperidine-4- carboxamide | Rt = 1.08 min, m/z [M + H]⁺ = 369/371 (Method A) |
| 86 | 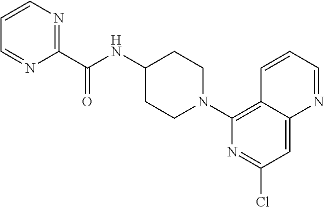 [SMILES: O=C(NC1CCN(c2nc(Cl)cc3ncccc23)CC1)c1ncccn1] | a) 5,7-Dichloro-1,6-naphthyridine b) Intermediate 33 | Rt = 1.11 min, m/z [M + H]⁺ = 369/371 (Method A) |
| 87 | 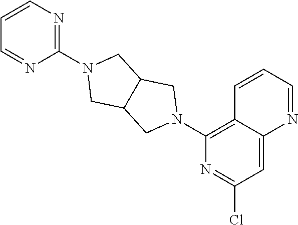 [SMILES: Clc1cc2ncccc2c(N2CC3CN(c4ncccn4)CC3C2)n1] | a) 5,7-Dichloro-1,6-naphthyridine b) 2-(Pyrimidin-2- yl)octahydropyrrolo[3,4-c]pyrrole | Rt = 1.08 min, m/z [M + H]⁺ = 353/355 (Method A) Rt = 1.22 |
| 88 | 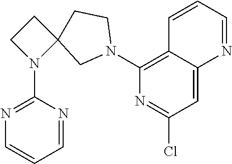 [SMILES: Clc1cc2ncccc2c(N2CCC3(CCN3c3ncccn3)C2)n1] | a) 5,7-Dichloro-1,6-naphthyridine b) Intermediate 34 | min, m/z [M + H]⁺ = 353/355 (Method A) Rt = 1.57 |
| 89 | 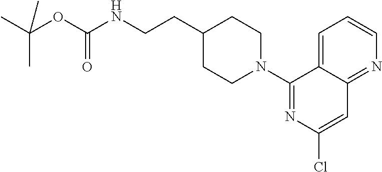 [SMILES: CC(C)(C)OC(=O)NCCC1CCN(c2nc(Cl)cc3ncccc23)CC1] | a) 5,7-Dichloro-1,6-naphthyridine b) tert-Butyl(2-(piperidin-4- yl)ethylcarbamate | [M + H]⁺ = min, m/z 391/393 (Method A) |
| 90 | 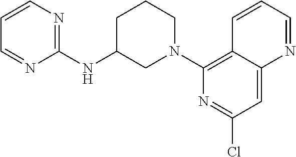 [SMILES: Clc1cc2ncccc2c(N2CCCC(Nc3ncccn3)C2)n1] | a) 5,7-Dichloro-1,6-naphthyridine b) N-(Piperidin-3-yl)pyrimidin-2- amine | Rt = 1.28 min, m/z [M + H]⁺ = 341/343 (Method A) |
| 91 | 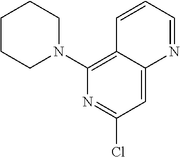 [SMILES: Clc1cc2ncccc2c(N2CCCCC2)n1] | a) 5,7-Dichloro-1,6-naphthyridine b) Piperidine | Rt = 1.48 min, m/z [M + H]⁺ = 248/250 (Method A) |
| 92 | 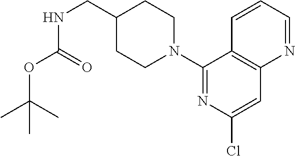 [SMILES: CC(C)(C)OC(=O)NCC1CCN(c2nc(Cl)cc3ncccc23)CC1] | a) 5,7-Dichloro-1,6-naphthyridine b) tert-Butyl(piperidin-4- ylmethyl)carbamate | Rt = 1.50 min, m/z [M + H]⁺ = 377/379 (Method A) |
| 93 | 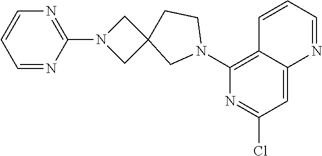 [SMILES: Clc1cc2ncccc2c(N2CCC3(CN(c4ncccn4)C3)C2)n1] | a) 5,7-Dichloro-1,6-naphthyridine b) Intermediate 35 | Rt = 1.09 min, m/z [M + H]⁺ = 353/355 (Method A) |
| 94 | 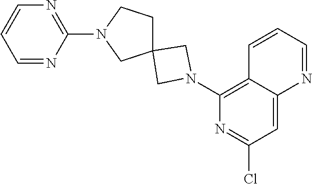 [SMILES: Clc1cc2ncccc2c(N2CC3(CCN(c4ncccn4)C3)C2)n1] | a) 5,7-Dichloro-1,6-naphthyridine b) Intermediate 36 | Rt = 1.11 min, m/z [M + H]⁺ = 353/355 (Method A) |
| 95 | 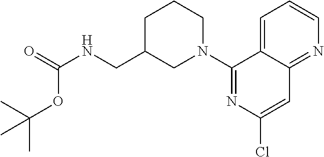 [SMILES: CC(C)(C)OC(=O)NCC1CCCN(c2nc(Cl)cc3ncccc23)C1] | a) 5,7-Dichloro-1,6-naphthyridine b) tert-Butyl(piperidin-3- ylmethyl)carbamate | Rt = 1.51 min, m/z [M + H]⁺ = 377/379 (Method A) |
| 96 | 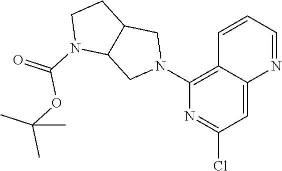 [SMILES: CC(C)(C)OC(=O)N1CCC2CN(c3nc(Cl)cc4ncccc34)CC21] | a) 5,7-Dichloro-1,6-naphthyridine b) tert-Butyl hexahydropyrrolo[3,4- b]pyrrole-1(2H)-carboxylate | Rt = 1.45 min, m/z [M + H]⁺ = 375/377 (Method A) |
| 97 | 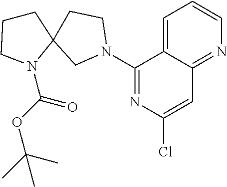 [SMILES: CC(C)(C)OC(=O)N1CCCC12CCN(c1nc(Cl)cc3ncccc13)C2] | a) 5,7-Dichloro-1,6-naphthyridine b) tert-Butyl 1,7- diazaspiro[4.4]nonane-1- carboxylate | Rt = 1.56 min, m/z [M + H]⁺ = 389/391 (Method A) |
| 98 | 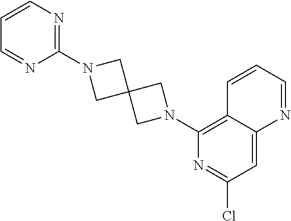 [SMILES: Clc1cc2ncccc2c(N2CC3(CN(c4ncccn4)C3)C2)n1] | a) 5,7-Dichloro-1,6-naphthyridine b) 2-(Pyrimidin-2-yl)-2,6- diazaspiro[3.3]heptane | Rt = 1.05 min, m/z [M + H]⁺ = 339/341 (Method A) |
| 99 | 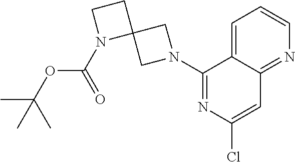 [SMILES: CC(C)(C)OC(=O)N1CCC12CN(c1nc(Cl)cc3ncccc13)C2] | a) 5,7-Dichloro-1,6-naphthyridine b) tert-Butyl 1,6- diazaspiro[3.3]heptane-1- carboxylate | Rt = 1.40 min, m/z [M + H]⁺ = 361/363 (Method A) |
| 100 | 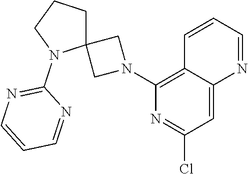 [SMILES: Clc1cc2ncccc2c(N2CC3(CCCN3c3ncccn3)C2)n1] | a) 5,7-Dichloro-1,6-naphthyridine b) Intermediate 37 | Rt = 1.27 min, m/z [M + H]⁺ = 353/355 (Method A) |

### Example A14

### a) Preparation of Intermediate 101

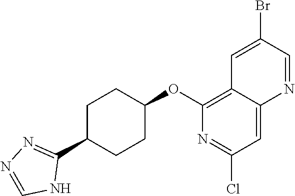 [SMILES: Clc1cc2ncc(Br)cc2c(O[C@H]2CC[C@@H](c3nnc[nH]3)CC2)n1]

A mixture of intermediate 43 (0.23 g, 0.59 mmol) in N,N-dimethylformamide dimethyl acetal (5.0 ml) was heated at 110° C. for 2 hours. The resulting mixture was cooled to ambient temperature and concentrated in vacuo, azeotroping with toluene. The residue was taken up in acetic acid (5.0 ml), treated with hydrazine hydrate (0.20 ml) and the resulting mixture was heated at 90° C. for 2 hours. The mixture was cooled to ambient temperature and concentrated in vacuo. The residue was purified by column chromatography on silica gel, eluting with a mixture of EtOAc and MeOH (1:0 to 7:3 by volume), to afford the desired product as a pale yellow solid (0.23 g, 94%).

LCMS (Method A): Rt=1.38 min, m/z [M+H]⁺=408/410

Intermediate 102 was prepared according to the reaction protocol of intermediate 101 using the appropriate starting materials (Table 13).

TABLE 13

| Intermediate | Structure | Starting Materials | LCMS Data |
|---|---|---|---|
| 102 | 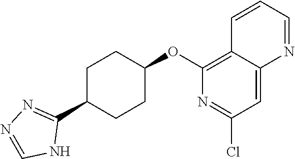 [SMILES: Clc1cc2ncccc2c(O[C@H]2CC[C@@H](c3nnc[nH]3)CC2)n1] | a) Intermediate 48 b) Hydrazine hydrate | Rt = 1.18 min, m/z [M + H]⁺ = 330/332 (Method A) |

### Example A15

### a) Preparation of Intermediate 103

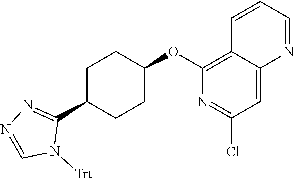

A solution of intermediate 102 (0.54 g, 1.64 mmol) and DIPEA (0.35 ml, 2.05 mmol) in DCM (20 ml) was treated with trityl chloride (0.57 g, 2.05 mmol). After stirring for 1.5 hours at ambient temperature, the mixture was partitioned between water and DCM. The organic phase was dried over magnesium sulfate and concentrated in vacuo. The residue was purified by column chromatography on silica gel, eluting with a mixture of cyclohexane and EtOAc (1:0 to 3:7 by volume), to afford the desired product as a white solid (0.77 g, 82%).

LCMS (Method B): Rt=2.59 min, m/z [M+H]⁺=572

### Example A16

### a) Preparation of Intermediate 104

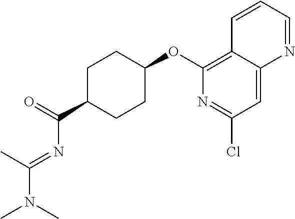 [SMILES: C/C(=N\C(=O)[C@H]1CC[C@@H](Oc2nc(Cl)cc3ncccc23)CC1)N(C)C]

A mixture of intermediate 48 (0.10 g, 0.33 mmol) in N,N-dimethylacetamide dimethyl acetal (1.0 ml) was heated at 110° C. for 2 hours. The resulting mixture was cooled to ambient temperature and concentrated in vacuo. The residue was azeotroped with toluene to afford the desired product as a brown oil (0.12 g, 100%).

LCMS (Method A): Rt=0.95 min, m/z [M+H]⁺=375/377

### b) Preparation of Intermediate 105

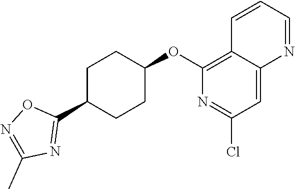 [SMILES: Cc1noc([C@H]2CC[C@@H](Oc3nc(Cl)cc4ncccc34)CC2)n1]

A stirred solution of intermediate 104 (0.060 g, 0.16 mmol) and 50% aqueous ammonium hydroxide (0.027 ml) in acetic acid (2.0 ml) was heated at 90° C. for 3 hours. The resulting mixture was cooled to ambient temperature and concentrated in vacuo. The residue was partitioned between EtOAc and saturated aqueous sodium bicarbonate solution. The organic phase was dried over magnesium sulfate and concentrated in vacuo to afford the desired product as a white solid (0.052 g, 95%).

LCMS (Method A): Rt=1.50 min, m/z [M+H]⁺=345/347

### Example A17

### a) Preparation of Intermediate 106

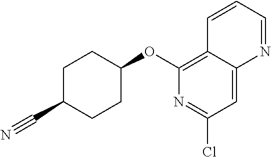 [SMILES: N#C[C@H]1CC[C@@H](Oc2nc(Cl)cc3ncccc23)CC1]

A mixture of intermediate 48 (0.12 g, 0.39 mmol) and 2,4,6-trichloro-1,3,5-triazine (0.047 g, 0.25 mmol) in DMF (1.0 ml) was stirred at ambient temperature for 30 minutes. The resulting mixture was partitioned between EtOAc and water. The organic phase was dried over sodium sulfate and concentrated in vacuo to afford the desired product as a yellow solid (0.11 g, 100%).

LCMS (Method A): Rt=1.48 min, m/z [M+H]⁺=288/290

### Example A18

### a) Preparation of Intermediate 107

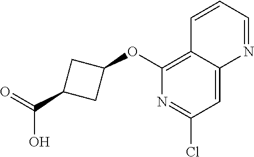 [SMILES: O=C(O)[C@H]1C[C@@H](Oc2nc(Cl)cc3ncccc23)C1]

A solution of intermediate 55 (0.027 g, 0.89 mmol) in MeOH (3.0 mL) and THF (3.0 ml) was treated with 1.0 M aqueous sodium hydroxide (2.67 ml, 2.67 mmol). After stirring at ambient temperature for 1 hour, the resulting mixture was concentrated in vacuo. The residue was acidified with 1.0 M HCl and the resulting precipitate was collected by filtration and dried under high vacuum at 40° C. for 18 hours to afford the desired product as a pale brown solid (0.15 g, 62%).

LCMS (Method A): Rt=1.33 min, m/z [M+H]⁺=394/396

### b) Preparation of Intermediate 108

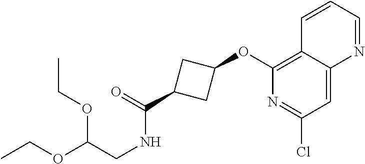 [SMILES: CCOC(CNC(=O)[C@H]1C[C@@H](Oc2nc(Cl)cc3ncccc23)C1)OCC]

A solution of intermediate 107 (0.15 g, 0.55 mmol), 2,2-diethoxyethan-1-amine (0.080 ml, 0.55 mmol) and DIPEA (0.19 ml, 1.09 mmol) in DMF (3.0 ml) was treated with HATU (0.25 g, 0.66 mmol). After stirring at ambient temperature for 3 hours, the resulting mixture was partitioned between EtOAc and water. The organic phase was dried over sodium sulfate and concentrated in vacuo. The residue was purified by flash chromatography on silica gel, eluting with a mixture of cyclohexane and EtOAc (1:0 to 0:1 by volume), to afford the desired product as an off-white solid (0.16 g, 73%).

LCMS (Method A): Rt=1.20 min, m/z [M+H]⁺=279/281

Intermediate 109 was prepared according to the reaction protocol of intermediate 108 using the appropriate starting materials (Table 14).

TABLE 14

| | | Starting | |
|---|---|---|---|
| Intermediate | Structure | Materials | NMR Data |
| 109 | 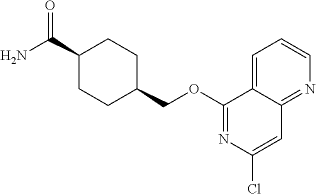 [SMILES: NC(=O)[C@H]1CC[C@@H](COc2nc(Cl)cc3ncccc23)CC1] | a) Intermediate 56; b) Ammonium chloride | ¹H NMR (400 MHz, DMSO-d₆) δ ppm: 9.09 (dd, J = 1.5, 4.3 Hz, 1H), 8.58-8.55 (m, 1H), 7.65 (dd, J = 4.3, 8.4 Hz, 1H), 7.56 (d, J = 0.8 Hz, 1H), 7.19 (s, 1H), 6.71 (s, 1H), 4.40 (d, J = 7.1 Hz, 2H), 2.34-2.26 (m, 1H), 2.12- 2.06 (m, 1H), 1.84-1.76 (m, 2H), 1.65-1.61 (m, 4H), 1.56-1.49 (m, 2H). |

### c) Preparation of Intermediate 110

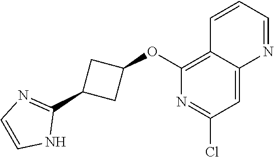 [SMILES: Clc1cc2ncccc2c(O[C@H]2C[C@@H](c3ncc[nH]3)C2)n1]

A suspension of intermediate 108 (0.24 g, 0.62 mmol) in xylene (10 ml) was treated with acetic acid (0.71 ml, 12.9 mmol) and ammonium acetate (0.24 g, 3.10 mmol). The resulting mixture was heated at 170° C. under microwave irradiation for 30 minutes. The mixture was cooled to ambient temperature and partitioned between DCM and saturated aqueous sodium bicarbonate solution. The organic phase was dried over sodium sulfate and concentrated in vacuo to afford the desired product as a beige solid (0.14 g, 77%).

LCMS (Method A): Rt=0.77 min, m/z [M+H]⁺=301/303

### d) Preparation of Intermediate 111

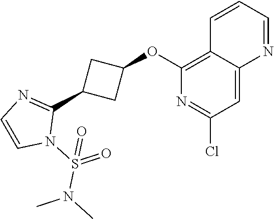 [SMILES: CN(C)S(=O)(=O)n1ccnc1[C@H]1C[C@@H](Oc2nc(Cl)cc3ncccc23)C1]

A solution of intermediate 110 (0.050 g, 0.17 mmol) and triethylamine (0.070 ml, 0.50 mmol) in DMF (1.0 ml) was treated with dimethylsulfamoyl chloride (0.036 ml, 0.33 mmol) and the resulting mixture was stirred at ambient temperature for 4 hours. Additional portions of dimethylsulfamoyl chloride (0.018 ml, 0.17 mmol) and triethylamine (0.035 ml, 0.25 mmol) were added and stirring was continued for a further 18 hours. The resulting mixture was partitioned between EtOAc and saturated aqueous sodium bicarbonate solution. The organic phase was dried over magnesium sulfate and concentrated in vacuo. The residue was purified by column chromatography on silica gel, eluting with a mixture of DCM and MeOH (1:0 to 19:1 by volume), to afford the desired product as a colourless oil (0.023 g, 34%).

LCMS (Method A): Rt=1.40 min, m/z [M+H]⁺=408/410

Intermediate 112 was prepared according to the reaction protocol of intermediate 111 using the appropriate starting materials (Table 15).

TABLE 15

| | | Starting | |
|---|---|---|---|
| Intermediate | Structure | Materials | LCMS Data |
| 112 | 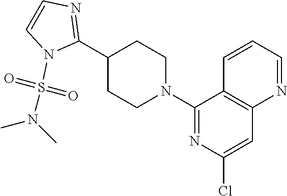 [SMILES: CN(C)S(=O)(=O)n1ccnc1C1CCN(c2nc(Cl)cc3ncccc23)CC1] | a) Intermediate 74 | Rt = 1.36 min, m/z [M + H]⁺ = 421/423 (Method A) |

### Example A19

### a) Preparation of Intermediate 113

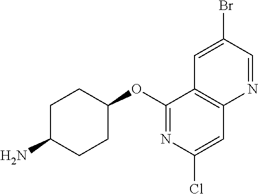 [SMILES: N[C@H]1CC[C@@H](Oc2nc(Cl)cc3ncc(Br)cc23)CC1]

A stirred solution of intermediate 40 (0.26 g, 0.56 mmol) in DCM (5.0 ml) was treated with TFA (1.0 ml). After stirring for 2 hours at ambient temperature, the resulting mixture was concentrated in vacuo and the residue was partitioned between DCM and saturated aqueous sodium bicarbonate solution. The organic phase was dried over sodium sulfate and concentrated in vacuo to afford the desired product as a white solid (0.20 g, 100%).

LCMS (Method A): Rt=0.99 min, m/z [M+H]⁺=356/358/360

Intermediates 114 to 120 were prepared according to the reaction protocol of intermediate 113 using the appropriate starting materials (Table 16).

TABLE 16

| Intermediate | Structure | Starting Materials | LCMS Data |
|---|---|---|---|
| 114 | 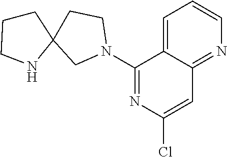 [SMILES: Clc1cc2ncccc2c(N2CCC3(CCCN3)C2)n1] | a) Intermediate 97; | Rt = 0.65 min, m/z [M + H]⁺ = 289/291 (Method A) |
| 115 | 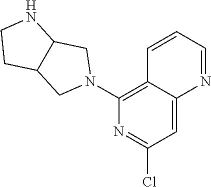 [SMILES: Clc1cc2ncccc2c(N2CC3CCNC3C2)n1] | a) Intermediate 72; | Rt = 0.65 min, m/z [M + H]⁺ = 275/277 (Method A) |
| 116 | 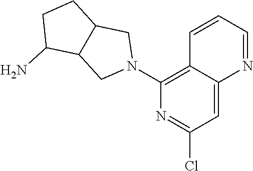 [SMILES: NC1CCC2CN(c3nc(Cl)cc4ncccc34)CC12] | a) Intermediate 73; | Rt = 0.69 min, m/z [M + H]⁺ = 289/291 (Method A) |
| 117 | 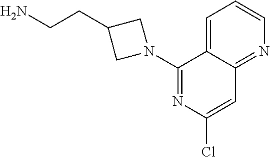 [SMILES: NCCC1CN(c2nc(Cl)cc3ncccc23)C1] | a) Intermediate 81; | Rt = 0.54 min, m/z [M + H]⁺ = 263/265 (Method A) |
| 118 | 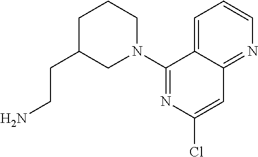 [SMILES: NCCC1CCCN(c2nc(Cl)cc3ncccc23)C1] | a) Intermediate 83; | Rt = 0.82 min, m/z [M + H]⁺ = 291/293 (Method A) |
| 119 | 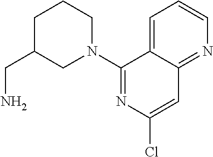 [SMILES: NCC1CCCN(c2nc(Cl)cc3ncccc23)C1] | a) Intermediate 95; | Rt = 0.85 min, m/z [M + H]⁺ = 277/279 (Method A) |
| 120 | 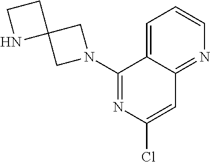 [SMILES: Clc1cc2ncccc2c(N2CC3(CCN3)C2)n1] | a) Intermediate 99; | Rt = 0.50 min, m/z [M + H]⁺ = 261/263 (Method A) |

b) Preparation of intermediate 121

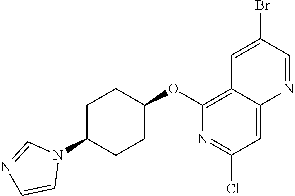 [SMILES: Clc1cc2ncc(Br)cc2c(O[C@H]2CC[C@@H](n3ccnc3)CC2)n1]

A solution of intermediate 113 (0.20 g, 0.56 mmol) in MeOH (5.0 ml) was treated with ammonium carbonate (0.029 g, 0.30 mmol), paraformaldehyde (0.018 g, 0.60 mmol) and glyoxal trimer dihydrate (0.042 g, 0.20 mmol) and the resulting mixture was stirred at ambient temperature for 18 hours. Additional portions of ammonium carbonate (0.029 g, 0.30 mmol), paraformaldehyde (0.018 g, 0.60 mmol) and glyoxal trimer dihydrate (0.042 g, 0.20 mmol) were added and stirring was continued for a further 24 hours. The resulting mixture was concentrated in vacuo and the residue was partitioned between DCM and water. The organic phase was dried over sodium sulfate and concentrated in vacuo. The residue was purified on a Biotage® KP-NH column, eluting with a mixture of isohexane and EtOAc (1:0 to 0:1 by volume), to afford the desired product as a white solid (0.13 g, 58%).

LCMS (Method A): Rt=1.08 min, m/z [M+H]⁺=407/409

### Example A20

### a) Preparation of Intermediate 122

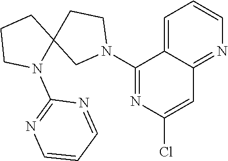 [SMILES: Clc1cc2ncccc2c(N2CCC3(CCCN3c3ncccn3)C2)n1]

A mixture of intermediate 114, (0.16 g, 0.54 mmol), 2-fluoropyrimidine (0.058 g, 0.59 mmol) and triethylamine (0.15 ml, 1.08 mmol) in iPrOH (1.0 ml) was heated at 100° C. for 18 hours in a sealed tube. The resulting mixture was cooled to ambient temperature and concentrated in vacuo. The residue was purified by column chromatography on silica gel, eluting with a mixture of DCM and MeOH (1:0 to 23:2 by volume), to afford the desired product as a pale yellow oil (0.19 g, 95%).

LCMS (Method A): Rt=1.38 min, m/z [M+H]⁺=367/369

Intermediates 123 to 129 were prepared according to the reaction protocol of intermediate 122 using the appropriate starting materials (Table 17).

TABLE 17

| Intermediate | Structure | Starting Materials | LCMS Data |
|---|---|---|---|
| 123 | 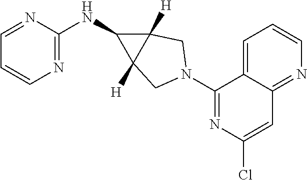 [SMILES: Clc1cc2ncccc2c(N2C[C@@H]3[C@H](C2)[C@H]3Nc2ncccn2)n1] | a) Intermediate 71; b) 2-Fluoropyrimidine | Rt = 1.46 min, m/z [M + H]⁺ = 261/263 (Method B) |
| 124 | 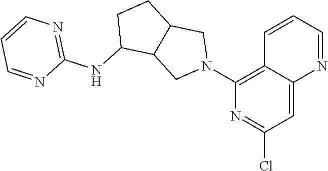 [SMILES: Clc1cc2ncccc2c(N2CC3CCC(Nc4ncccn4)C3C2)n1] | a) Intermediate 116; b) 2-Fluoropyrimidine | Rt = 1.24 min, m/z [M + H]⁺ = 367/369 (Method A) |
| 125 | 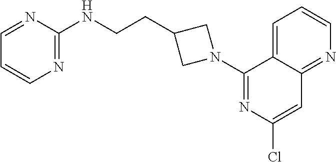 [SMILES: Clc1cc2ncccc2c(N2CC(CCNc3ncccn3)C2)n1] | a) Intermediate 117; b) 2-Fluoropyrimidine | Rt = 1.04 min, m/z [M + H]⁺ = 341/343 (Method A) |
| 126 | 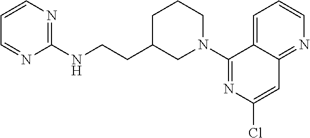 [SMILES: Clc1cc2ncccc2c(N2CCCC(CCNc3ncccn3)C2)n1] | a) Intermediate 118; b) 2-Fluoropyrimidine | Rt = 1.32 min, m/z [M + H]⁺ = 369/371 (Method A) |
| 127 | 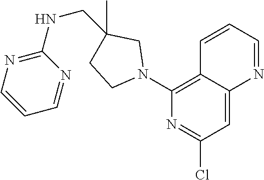 [SMILES: CC1(CNc2ncccn2)CCN(c2nc(Cl)cc3ncccc23)C1] | a) Intermediate 84; b) 2-Fluoropyrimidine | Rt = 1.17 min, m/z [M + H]⁺ = 355/357 (Method A) |
| 128 | 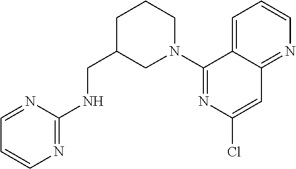 [SMILES: Clc1cc2ncccc2c(N2CCCC(CNc3ncccn3)C2)n1] | a) Intermediate 119; b) 2-Fluoropyrimidine | Rt = 1.21 min, m/z [M + H]⁺ = 355/357 (Method A) |
| 129 | 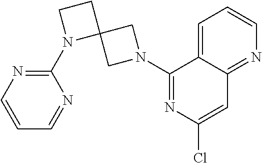 [SMILES: Clc1cc2ncccc2c(N2CC3(CCN3c3ncccn3)C2)n1] | a) Intermediate 120; b) 2-Fluoropyrimidine | Rt = 1.17 min, m/z [M + H]⁺ = 339/341 (Method A) |

### Example A21

### a) Preparation of Intermediate 130

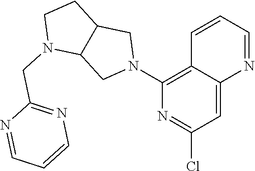 [SMILES: Clc1cc2ncccc2c(N2CC3CCN(Cc4ncccn4)C3C2)n1]

A solution of intermediate 115 (0.44 g, 1.58 mmol), 2-(chloromethyl)pyrimidine hydrochloride (0.26 g, 1.58 mmol) and DIPEA (0.81 ml, 4.74 mmol) in MeCN (10 ml) was stirred at ambient temperature for 18 hours. The resulting mixture was concentrated in vacuo. The residue was purified by column chromatography on silica gel, eluting with a mixture of DCM and MeOH (1:0 to 1:1 by volume), to afford the desired product as a pale yellow solid (0.58 g, 100%).

LCMS (Method A): Rt=0.72 min, m/z [M+H]⁺=367/369

### Example A22

### a) Preparation of Intermediate 131

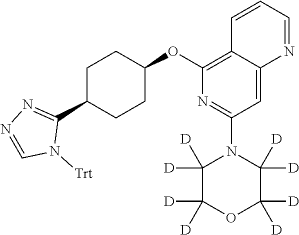

A mixture of intermediate 103 (0.090 g, 0.16 mmol), morpholine-2,2,3,3,5,5,6,6-d₈ (0.045 ml, 0.47 mmol), RuPhos Pd G1 (0.038 g, 0.047 mmol), RuPhos (0.022 g, 0.047 mmol) and cesium carbonate (0.15 g, 0.47 mmol) in dioxane (2.5 ml) was heated at 80° C. under an argon atmosphere for 3 hours. The resulting mixture was cooled to ambient temperature, filtered through Celite® and concentrated in vacuo. The residue was purified on a Biotage® KP-NH column, eluting with a mixture of cyclohexane and EtOAc (1:0 to 0:1 by volume), to afford the desired product as a yellow solid (0.085 g, 86%).

LCMS (Method B): Rt=2.39 min, m/z [M+H]⁺=631

Intermediates 132 to 135 were prepared according to the reaction protocol of intermediate 131 using the appropriate starting materials (Table 20).

TABLE 20

| Intermediate | Structure | Starting Materials | LCMS Data |
|---|---|---|---|
| 132 | 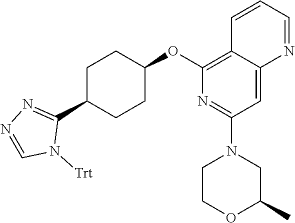 | a) Intermediate 103; b) (R)-2-Methylmorpholine hydrochloride | Rt = 2.46 min, m/z [M + H]⁺ = 637 (Method B) |
| 133 | 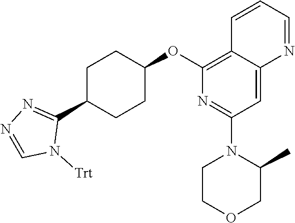 | a) Intermediate 103; b) (S)-3-Methylmorpholine | Rt = 2.44 min, m/z [M + H]⁺ = 637 (Method B) |
| 134 | 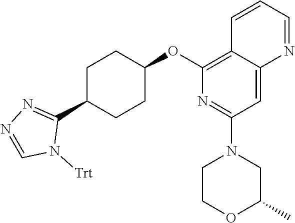 | a) Intermediate 103; b) (S)-2-Methylmorpholine | Rt = 2.45 min, m/z [M + H]⁺ = 637 (Method B) |
| 135 | 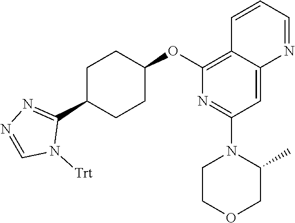 | a) Intermediate 103; b) (R)-3-Methylmorpholine | Rt = 2.43 min, m/z [M + H]⁺ = 637 (Method B) |

Preparation of Compounds of Formula (I) (‘Parent Compounds’)

### Example B1

a) Preparation of parent compound 1

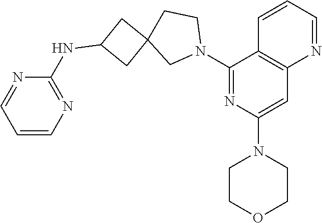 [SMILES: c1cnc(NC2CC3(CCN(c4nc(N5CCOCC5)cc5ncccc45)C3)C2)nc1]

A suspension of intermediate 65 (0.25 g, 0.68 mmol), morpholine (0.18 ml, 2.04 mmol), RuPhos Pd G1.TBME (0.056 g, 0.068 mmol), RuPhos (0.032 g, 0.068 mmol) and cesium carbonate (0.67 g, 2.04 mmol) in dioxane (5.0 ml) was heated at 85° C. for 45 minutes under an argon atmosphere. The resulting mixture was cooled to ambient temperature, filtered through Celite® and concentrated in vacuo. The residue was purified by MDAP (Method A) to afford the desired product as an orange solid (0.15 g, 52%).

LCMS (Method C): Rt=2.67, 2.74 min, m/z [M+H]⁺=418

¹H NMR (400 MHz, DMSO-d₆) δ ppm: 8.61-8.59 (m, 1H), 8.42-8.36 (m, 1H), 8.27-8.23 (m, 2H), 7.48-7.37 (m, 1H), 7.00 (dd, J=4.2, 8.3 Hz, 1H), 6.57-6.53 (m, 1H), 6.28-6.26 (m, 1H), 4.45-4.34 (m, 1H), 3.84-3.69 (m, 8H), 3.49-3.45 (m, 4H), 2.40-2.30 (m, 2H), 2.10-1.92 (m, 4H). Formic acid 0.59 equivalents. 1:1 mixture of diastereomers.

Parent compounds 2 to 53 were prepared according to the reaction protocol of parent compound 1 using morpholine and the appropriate starting material (Table 18).

TABLE 18

| Parent Compound | Structure | Starting Material |
|---|---|---|
| 2 | 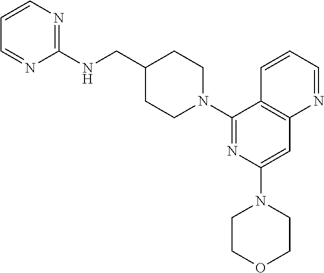 [SMILES: c1cnc(NCC2CCN(c3nc(N4CCOCC4)cc4ncccc34)CC2)nc1] | Intermediate 67 |
| 3 | 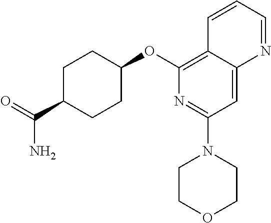 [SMILES: NC(=O)[C@H]1CC[C@@H](Oc2nc(N3CCOCC3)cc3ncccc23)CC1] | Intermediate 48 |
| 4 | 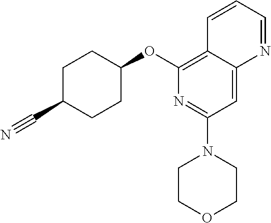 [SMILES: N#C[C@H]1CC[C@@H](Oc2nc(N3CCOCC3)cc3ncccc23)CC1] | Intermediate 106 |
| 5 | 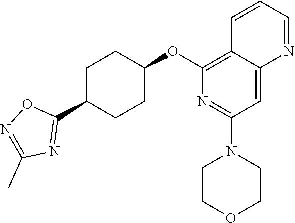 [SMILES: Cc1noc([C@H]2CC[C@@H](Oc3nc(N4CCOCC4)cc4ncccc34)CC2)n1] | Intermediate 105 |
| 6 | 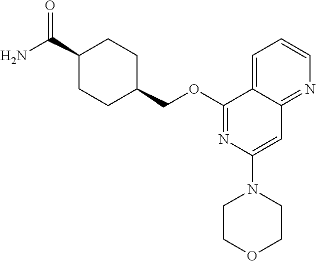 [SMILES: NC(=O)[C@H]1CC[C@@H](COc2nc(N3CCOCC3)cc3ncccc23)CC1] | Intermediate 109 |
| 7 | 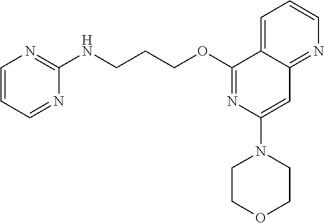 [SMILES: c1cnc(NCCCOc2nc(N3CCOCC3)cc3ncccc23)nc1] | Intermediate 62 |
| 8 | 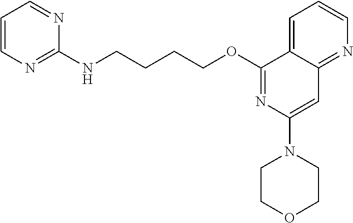 [SMILES: c1cnc(NCCCCOc2nc(N3CCOCC3)cc3ncccc23)nc1] | Intermediate 61 |
| 8 | 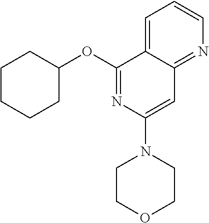 [SMILES: c1cnc2cc(N3CCOCC3)nc(OC3CCCCC3)c2c1] | Intermediate 63 |
| 10 | 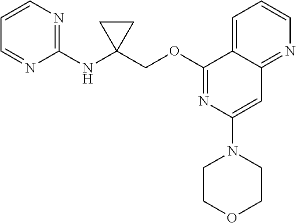 [SMILES: c1cnc(NC2(COc3nc(N4CCOCC4)cc4ncccc34)CC2)nc1] | Intermediate 68 |
| 11 | 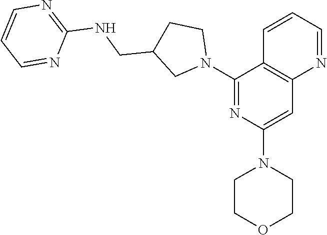 [SMILES: c1cnc(NCC2CCN(c3nc(N4CCOCC4)cc4ncccc34)C2)nc1] | Intermediate 69 |
| 12 | 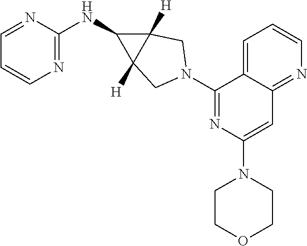 [SMILES: c1cnc(N[C@H]2[C@@H]3CN(c4nc(N5CCOCC5)cc5ncccc45)C[C@@H]32)nc1] | Intermediate 123 |
| 13 | 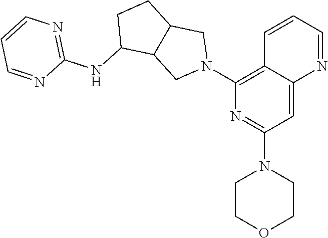 [SMILES: c1cnc(NC2CCC3CN(c4nc(N5CCOCC5)cc5ncccc45)CC32)nc1] | Intermediate 124 |
| 14 | 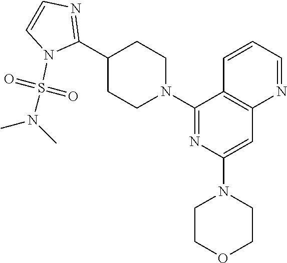 [SMILES: CN(C)S(=O)(=O)n1ccnc1C1CCN(c2nc(N3CCOCC3)cc3ncccc23)CC1] | Intermediate 112 |
| 15 | 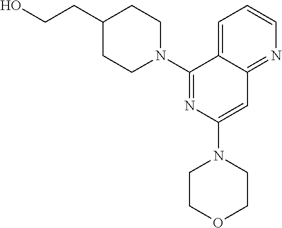 [SMILES: OCCC1CCN(c2nc(N3CCOCC3)cc3ncccc23)CC1] | Intermediate 75 |
| 16 | 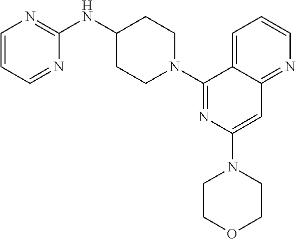 [SMILES: c1cnc(NC2CCN(c3nc(N4CCOCC4)cc4ncccc34)CC2)nc1] | Intermediate 76 |
| 17 | 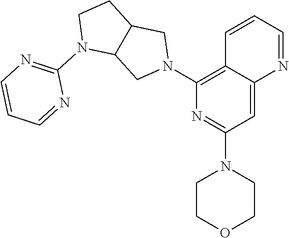 [SMILES: c1cnc(N2CCC3CN(c4nc(N5CCOCC5)cc5ncccc45)CC32)nc1] | Intermediate 77 |
| 18 | 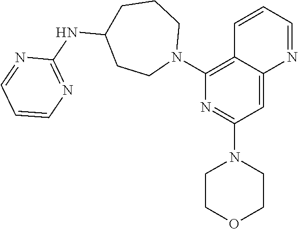 [SMILES: c1cnc(NC2CCCN(c3nc(N4CCOCC4)cc4ncccc34)CC2)nc1] | Intermediate 78 |
| 19 | 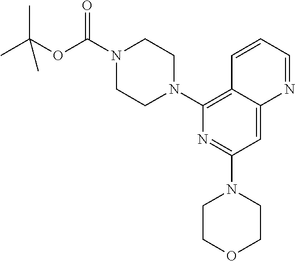 [SMILES: CC(C)(C)OC(=O)N1CCN(c2nc(N3CCOCC3)cc3ncccc23)CC1] | Intermediate 79 |
| 20 | 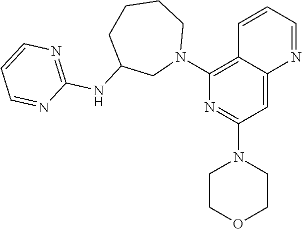 [SMILES: c1cnc(NC2CCCCN(c3nc(N4CCOCC4)cc4ncccc34)C2)nc1] | Intermediate 80 |
| 21 | 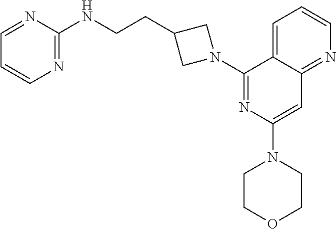 [SMILES: c1cnc(NCCC2CN(c3nc(N4CCOCC4)cc4ncccc34)C2)nc1] | Intermediate 125 |
| 22 | 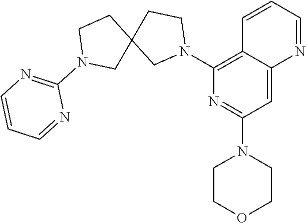 [SMILES: c1cnc(N2CCC3(CCN(c4nc(N5CCOCC5)cc5ncccc45)C3)C2)nc1] | Intermediate 82 |
| 23 | 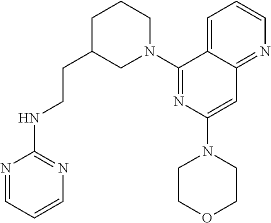 [SMILES: c1cnc(NCCC2CCCN(c3nc(N4CCOCC4)cc4ncccc34)C2)nc1] | Intermediate 126 |
| 24 | 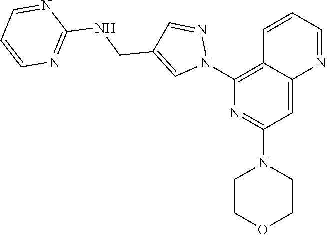 [SMILES: c1cnc(NCc2cnn(-c3nc(N4CCOCC4)cc4ncccc34)c2)nc1] | Intermediate 64 |
| 25 | 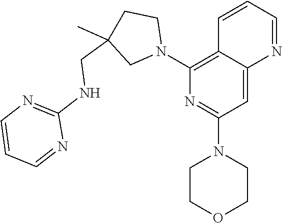 [SMILES: CC1(CNc2ncccn2)CCN(c2nc(N3CCOCC3)cc3ncccc23)C1] | Intermediate 127 |
| 26 | 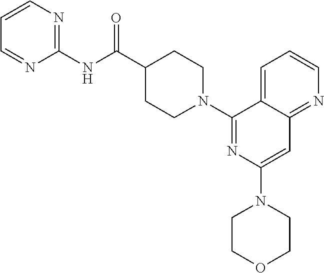 [SMILES: O=C(Nc1ncccn1)C1CCN(c2nc(N3CCOCC3)cc3ncccc23)CC1] | Intermediate 85 |
| 27 | 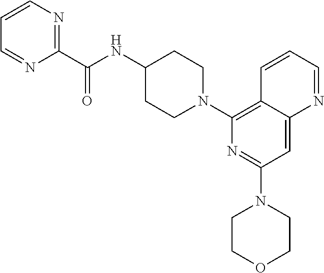 [SMILES: O=C(NC1CCN(c2nc(N3CCOCC3)cc3ncccc23)CC1)c1ncccn1] | Intermediate 86 |
| 28 | 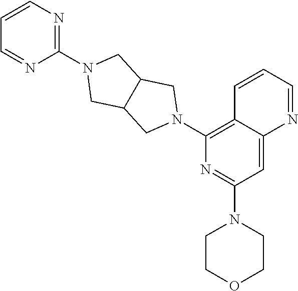 [SMILES: c1cnc(N2CC3CN(c4nc(N5CCOCC5)cc5ncccc45)CC3C2)nc1] | Intermediate 87 |
| 29 | 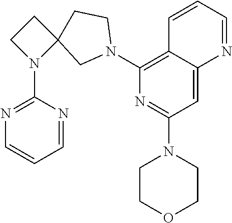 [SMILES: c1cnc(N2CCC23CCN(c2nc(N4CCOCC4)cc4ncccc24)C3)nc1] | Intermediate 88 |
| 30 | 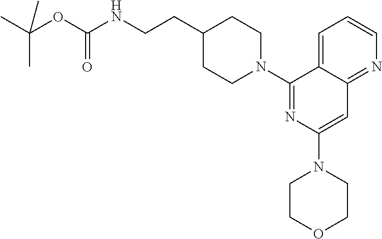 [SMILES: CC(C)(C)OC(=O)NCCC1CCN(c2nc(N3CCOCC3)cc3ncccc23)CC1] | Intermediate 89 |
| 31 | 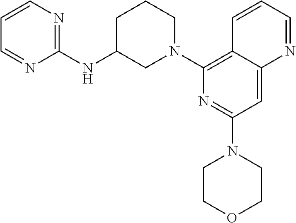 [SMILES: c1cnc(NC2CCCN(c3nc(N4CCOCC4)cc4ncccc34)C2)nc1] | Intermediate 90 |
| 32 | 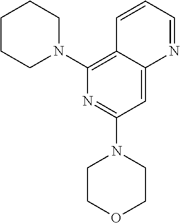 [SMILES: c1cnc2cc(N3CCOCC3)nc(N3CCCCC3)c2c1] | Intermediate 91 |
| 33 | 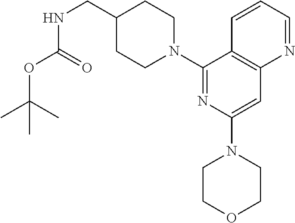 [SMILES: CC(C)(C)OC(=O)NCC1CCN(c2nc(N3CCOCC3)cc3ncccc23)CC1] | Intermediate 92 |
| 34 | 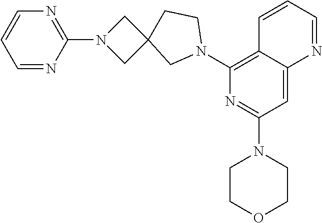 [SMILES: c1cnc(N2CC3(CCN(c4nc(N5CCOCC5)cc5ncccc45)C3)C2)nc1] | Intermediate 93 |
| 35 | 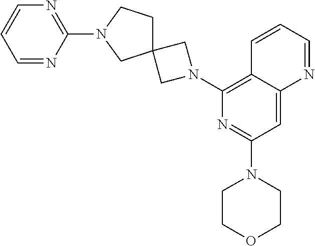 [SMILES: c1cnc(N2CCC3(C2)CN(c2nc(N4CCOCC4)cc4ncccc24)C3)nc1] | Intermediate 94 |
| 36 | 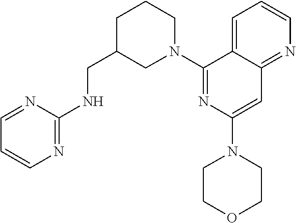 [SMILES: c1cnc(NCC2CCCN(c3nc(N4CCOCC4)cc4ncccc34)C2)nc1] | Intermediate 128 |
| 37 | 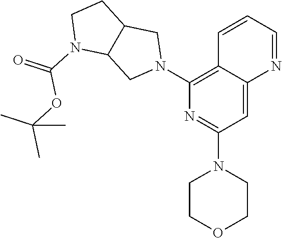 [SMILES: CC(C)(C)OC(=O)N1CCC2CN(c3nc(N4CCOCC4)cc4ncccc34)CC21] | Intermediate 96 |
| 38 | 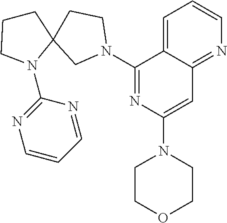 [SMILES: c1cnc(N2CCCC23CCN(c2nc(N4CCOCC4)cc4ncccc24)C3)nc1] | Intermediate 122 |
| 39 | 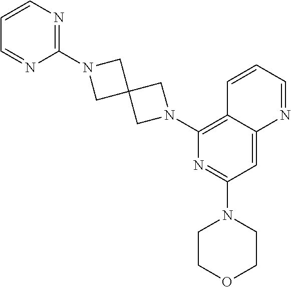 [SMILES: c1cnc(N2CC3(C2)CN(c2nc(N4CCOCC4)cc4ncccc24)C3)nc1] | Intermediate 98 |
| 40 | 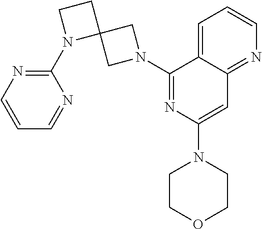 [SMILES: c1cnc(N2CCC23CN(c2nc(N4CCOCC4)cc4ncccc24)C3)nc1] | Intermediate 129 |
| 41 | 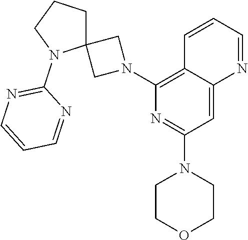 [SMILES: c1cnc(N2CCCC23CN(c2nc(N4CCOCC4)cc4ncccc24)C3)nc1] | Intermediate 100 |
| 42 | 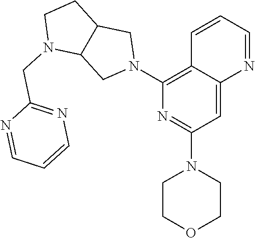 [SMILES: c1cnc(CN2CCC3CN(c4nc(N5CCOCC5)cc5ncccc45)CC32)nc1] | Intermediate 130 |
| 43 | 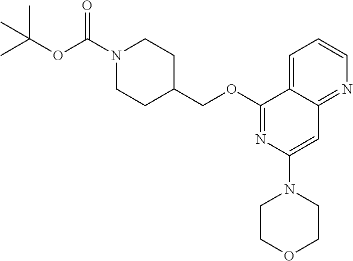 [SMILES: CC(C)(C)OC(=O)N1CCC(COc2nc(N3CCOCC3)cc3ncccc23)CC1] | Intermediate 53 |
| 44 | 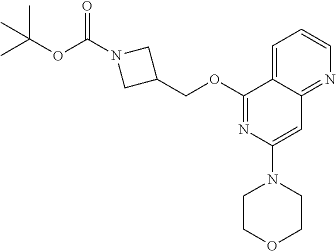 [SMILES: CC(C)(C)OC(=O)N1CC(COc2nc(N3CCOCC3)cc3ncccc23)C1] | Intermediate 54 |
| 45 | 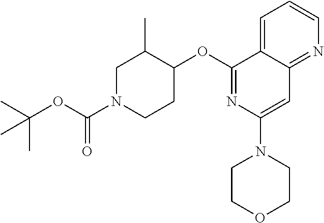 [SMILES: CC1CN(C(=O)OC(C)(C)C)CCC1Oc1nc(N2CCOCC2)cc2ncccc12] | Intermediate 58; 3:2 mixture of trans:cis diastereomers. |
| 46 | 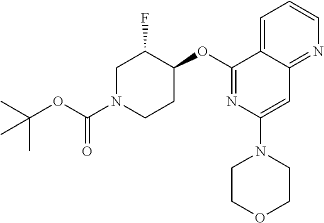 [SMILES: CC(C)(C)OC(=O)N1CC[C@H](Oc2nc(N3CCOCC3)cc3ncccc23)[C@@H](F)C1] | Intermediate 57 |
| 47 | 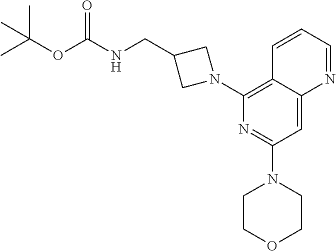 [SMILES: CC(C)(C)OC(=O)NCC1CN(c2nc(N3CCOCC3)cc3ncccc23)C1] | Intermediate 70 |
| 48 | 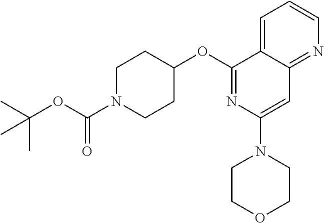 [SMILES: CC(C)(C)OC(=O)N1CCC(Oc2nc(N3CCOCC3)cc3ncccc23)CC1] | Intermediate 59 |
| 49 | 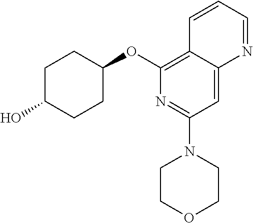 [SMILES: O[C@H]1CC[C@H](Oc2nc(N3CCOCC3)cc3ncccc23)CC1] | Intermediate 49 |
| 50 | 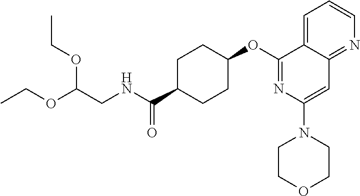 [SMILES: CCOC(CNC(=O)[C@H]1CC[C@@H](Oc2nc(N3CCOCC3)cc3ncccc23)CC1)OCC] | Intermediate 50 |
| 51 | 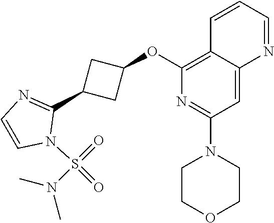 [SMILES: CN(C)S(=O)(=O)n1ccnc1[C@H]1C[C@@H](Oc2nc(N3CCOCC3)cc3ncccc23)C1] | Intermediate 111 |
| 52 | 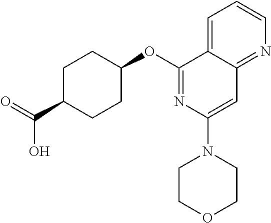 [SMILES: O=C(O)[C@H]1CC[C@@H](Oc2nc(N3CCOCC3)cc3ncccc23)CC1] | Intermediate 51 |
| 53 | 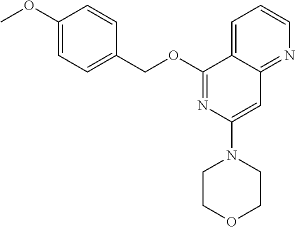 [SMILES: COc1ccc(COc2nc(N3CCOCC3)cc3ncccc23)cc1] | Intermediate 60 |

### Example B2

### a) Preparation of Parent Compound 54

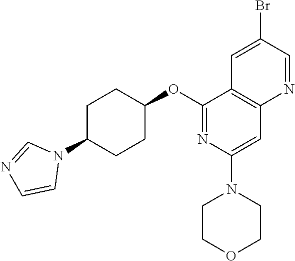 [SMILES: Brc1cnc2cc(N3CCOCC3)nc(O[C@H]3CC[C@@H](n4ccnc4)CC3)c2c1]

A mixture of intermediate 121 (0.13 g, 0.32 mmol) and morpholine (3.0 ml) was heated at 200° C. for 1 hour under microwave irradiation. The mixture was cooled to ambient temperature and concentrated in vacuo. The residue was purified on a Biotage® KP-NH column, eluting with a mixture of EtOAc and MeOH (1:0 to 4:1 by volume), to afford the desired product as a yellow solid (0.13 g, 86%).

LCMS (Method A): Rt=1.00 min, m/z [M+H]⁺=458/460

¹H NMR (400 MHz, DMSO-d₆) δ ppm: 8.80 (d, J=2.4 Hz, 1H), 8.40-8.38 (m, 1H), 7.64-7.62 (m, 1H), 7.11-7.10 (m, 1H), 7.06-7.04 (m, 1H), 6.53 (s, 1H), 5.53-5.49 (m, 1H), 4.15-4.06 (m, 1H), 3.88-3.84 (m, 4H), 3.57-3.53 (m, 4H), 2.43-2.36 (m, 2H), 2.23-2.04 (m, 4H), 1.89-1.79 (m, 2H).

Parent compounds 55 to 64 were prepared according to the reaction protocol of parent compound 54 using morpholine and the appropriate starting material (Table 19).

TABLE 19

| Parent Compound | Structure | Starting Material |
|---|---|---|
| 55 | 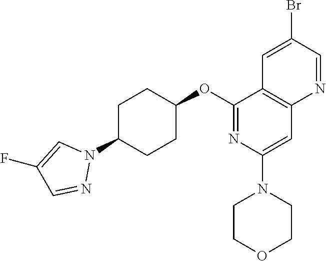 [SMILES: Fc1cnn([C@H]2CC[C@@H](Oc3nc(N4CCOCC4)cc4ncc(Br)cc34)CC2)c1] | Intermediate 41 |
| 56 | 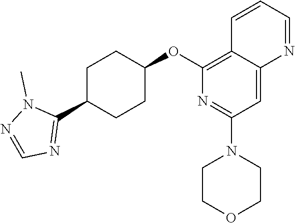 [SMILES: Cn1ncnc1[C@H]1CC[C@@H](Oc2nc(N3CCOCC3)cc3ncccc23)CC1] | Intermediate 52 |
| 57 | 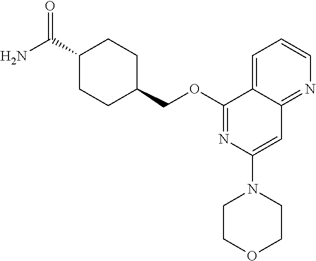 [SMILES: NC(=O)[C@H]1CC[C@H](COc2nc(N3CCOCC3)cc3ncccc23)CC1] | Intermediate 109 |
| 58 | 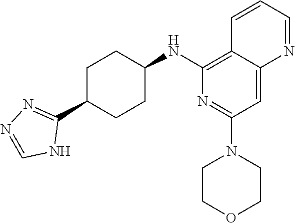 [SMILES: c1cnc2cc(N3CCOCC3)nc(N[C@H]3CC[C@@H](c4nnc[nH]4)CC3)c2c1] | Intermediate 66 |
| 59 | 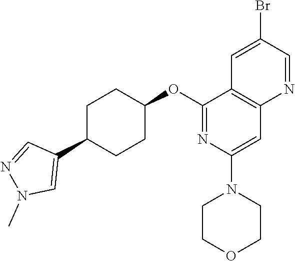 [SMILES: Cn1cc([C@H]2CC[C@@H](Oc3nc(N4CCOCC4)cc4ncc(Br)cc34)CC2)cn1] | Intermediate 44 |
| 60 | 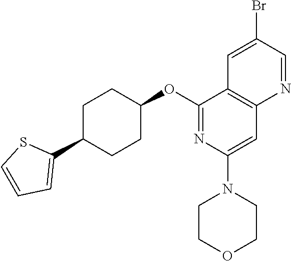 [SMILES: Brc1cnc2cc(N3CCOCC3)nc(O[C@H]3CC[C@@H](c4cccs4)CC3)c2c1] | Intermediate 42 |
| 61 | 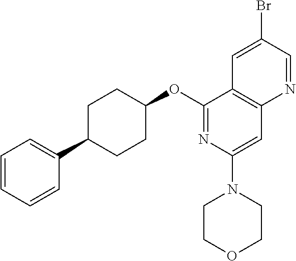 [SMILES: Brc1cnc2cc(N3CCOCC3)nc(O[C@H]3CC[C@@H](c4ccccc4)CC3)c2c1] | Intermediate 47 |
| 62 | 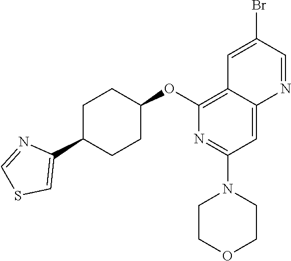 [SMILES: Brc1cnc2cc(N3CCOCC3)nc(O[C@H]3CC[C@@H](c4cscn4)CC3)c2c1] | Intermediate 45 |
| 63 | 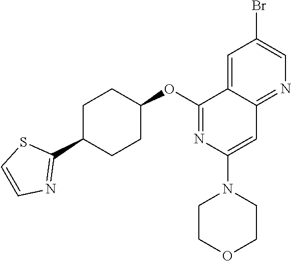 [SMILES: Brc1cnc2cc(N3CCOCC3)nc(O[C@H]3CC[C@@H](c4nccs4)CC3)c2c1] | Intermediate 46 |
| 64 | 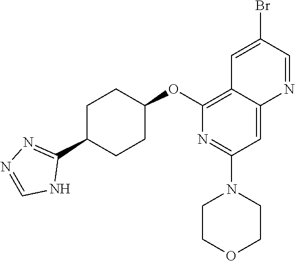 [SMILES: Brc1cnc2cc(N3CCOCC3)nc(O[C@H]3CC[C@@H](c4nnc[nH]4)CC3)c2c1] | Intermediate 101 |

### Example B3

### a) Preparation of Parent Compound 65

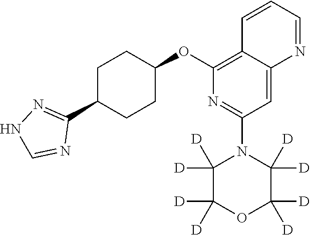 [SMILES: [2H]C1([2H])OC([2H])([2H])C([2H])([2H])N(c2cc3ncccc3c(O[C@H]3CC[C@@H](c4nc[nH]n4)CC3)n2)C1([2H])[2H]]

A solution of intermediate 131 (0.085 g, 0.14 mmol) in DCM (2.0 ml) and water (0.20 ml) was treated with TFA (2.0 ml). After stirring at ambient temperature for 3.5 hours, the resulting mixture was concentrated in vacuo. The residue was purified by ISOLUTE® SCX-2 SPE column, eluting with MeOH followed by 2.0 M ammonia solution in MeOH. Further purification by reverse phase preparative HPLC (Method B) afforded the desired product as a yellow solid (0.035 g, 67%).

LCMS (Method C): Rt=2.30 min, m/z [M+H]⁺=389

¹H NMR (400 MHz, DMSO-d₆) δ ppm: 13.66 (bs, 1H), 8.78 (dd, J=1.8, 4.3 Hz, 1H), 8.33-8.29 (m, 1H), 8.02 (s, 1H), 7.20 (dd, J=4.3, 8.2 Hz, 1H), 6.52 (d, J=0.7 Hz, 1H), 5.49-5.43 (m, 1H), 2.99-2.89 (m, 1H), 2.14-1.79 (m, 8H).

Parent compounds 66 to 69 were prepared according to the reaction protocol of parent compound 65 using the appropriate starting materials (Table 21).

TABLE 21

| Parent Compound | Structure | Starting Material |
|---|---|---|
| 66 | 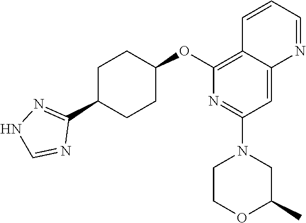 [SMILES: C[C@@H]1CN(c2cc3ncccc3c(O[C@H]3CC[C@@H](c4nc[nH]n4)CC3)n2)CCO1] | a) Intermediate 132 |
| 67 | 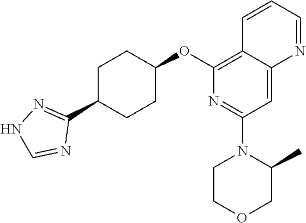 [SMILES: C[C@H]1COCCN1c1cc2ncccc2c(O[C@H]2CC[C@@H](c3nc[nH]n3)CC2)n1] | a) Intermediate 133 |
| 68 | 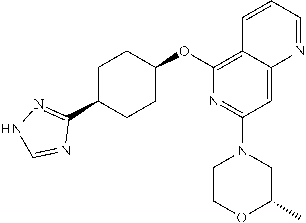 [SMILES: C[C@H]1CN(c2cc3ncccc3c(O[C@H]3CC[C@@H](c4nc[nH]n4)CC3)n2)CCO1] | a) Intermediate 134 |
| 69 | 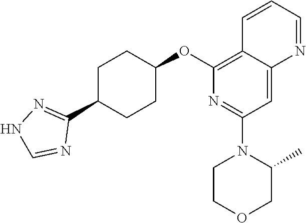 [SMILES: C[C@@H]1COCCN1c1cc2ncccc2c(O[C@H]2CC[C@@H](c3nc[nH]n3)CC2)n1] | a) Intermediate 135 |

### Example B4

### a) Preparation of Intermediate 136;

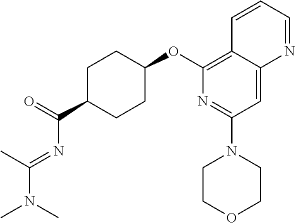 [SMILES: C/C(=N\C(=O)[C@H]1CC[C@@H](Oc2nc(N3CCOCC3)cc3ncccc23)CC1)N(C)C]

A mixture of parent compound 3 (0.10 g, 0.29 mmol) in N,N-dimethylacetamide dimethyl acetal (2.0 ml) was heated at 110° C. for 2 hours. The resulting mixture was cooled to ambient temperature and concentrated in vacuo, azeotroping with toluene, to afford the desired product as a yellow solid (0.12 g, 100%).

LCMS (Method A): Rt=0.71 min, m/z [M+H]⁺=426

Intermediates 137 and 138 were prepared according to the reaction protocol of intermediate 136 using the appropriate starting materials (Table 22).

TABLE 22

| Intermediate | Structure | Starting Materials | NMR Data |
|---|---|---|---|
| 137 | 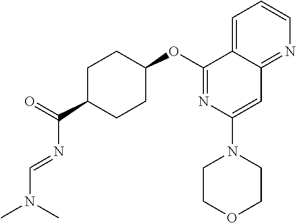 [SMILES: CN(C)/C=N/C(=O)[C@H]1CC[C@@H](Oc2nc(N3CCOCC3)cc3ncccc23)CC1] | a) Parent compound 3; b) N,N- Dimethylformamide dimethyl acetal | ¹H NMR (400 MHz, CDCl₃) δ ppm: 8.77 (dd, J = 1.8, 4.4 Hz, 1H), 8.44 (s, 1H), 8.37 (dd, J = 1.4, 8.2 Hz, 1H), 7.08 (dd, J = 4.3, 8.2 Hz, 1H), 6.52 (s, 1H), 5.50- 5.44 (m, 1H), 3.86 (dd, J = 4.9, 4.9 Hz, 4H), 3.54 (dd, J = 4.9, 4.9 Hz, 4H), 3.13 (s, 3H), 3.09 (s, 3H), 2.58-2.49 (m, 1H), 2.21-2.12 (m, |
| | | | 2H), 2.07-1.96 (m, |
| | | | 2H), 1.93-1.86 (m, |
| | | | 2H), 1.79-1.69 (m, 2H). |
| 138 | 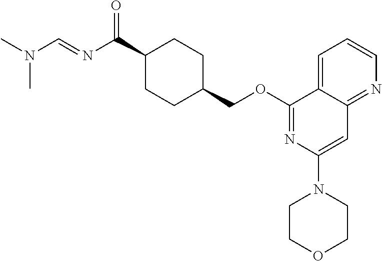 [SMILES: CN(C)/C=N/C(=O)[C@H]1CC[C@@H](COc2nc(N3CCOCC3)cc3ncccc23)CC1] | a) Parent compound 6; b) N,N- Dimethylformamide dimethyl acetal | ¹H NMR (400 MHz, DMSO-d₆) δ ppm: 8.78 (dd, J = 1.8, 4.3 Hz, 1H), 8.35 (s, 1H), 8.25 (dd, J = 1.6, 8.2 Hz, 1H), 7.18 (dd, J = 4.3, 8.2 Hz, 1H), 6.52 (s, 1H), 4.29 (d, J = 6.9 Hz, 2H), 3.74 (dd, J = 4.8, 4.8 Hz, 4H), 3.50 (dd, J = 4.9, 4.9 Hz, 4H), 3.10 (s, 3H), 2.96 (s, 3H), 2.48-2.44 (m, 1H), 2.06-1.97 (m, 2H), 1.67-1.62 (m, 2H), |
| | | | 1.57-1.39 (m, 4H). |

### b) Preparation of Parent Compound 70

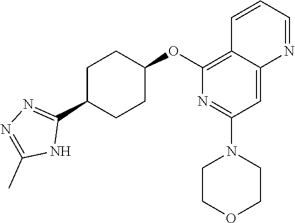 [SMILES: Cc1nnc([C@H]2CC[C@@H](Oc3nc(N4CCOCC4)cc4ncccc34)CC2)[nH]1]

A stirred solution of intermediate 136 (0.12 g, 0.29 mmol) in acetic acid (3.0 ml) at ambient temperature was treated with hydrazine hydrate (0.050 ml, 0.80 mmol). The resulting mixture was heated at 90° C. for 3 hours, then cooled to ambient temperature and concentrated in vacuo. The residue was purified by MDAP (Method A) to afford the desired product as a yellow solid (0.025 g, 22%).

LCMS (Method C): Rt=2.25 min, m/z [M+1-1]⁺=395

¹H NMR (400 MHz, DMSO-d₆) δ ppm: 13.22 (bs, 1H), 8.78 (dd, J=1.8, 4.3 Hz, 1H), 8.34-8.28 (m, 1H), 7.20 (dd, J=4.3, 8.2 Hz, 1H), 6.52 (s, 1H), 5.46-5.42 (m, 1H), 3.77-3.72 (m, 4H), 3.53-3.46 (m, 4H), 2.87-2.81 (m, 1H), 2.25 (s, 3H), 2.13-2.04 (m, 2H), 1.99-1.76 (m, 6H). Formic acid 0.75 equivalents.

Parent compounds 71 to 73 were prepared according to the reaction protocol of parent compound 70 using the appropriate starting materials (Table 23).

TABLE 23

| Parent Compound | Structure | Starting Materials |
|---|---|---|
| 71 | 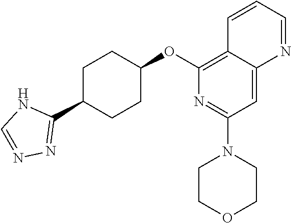 [SMILES: c1cnc2cc(N3CCOCC3)nc(O[C@H]3CC[C@@H](c4nnc[nH]4)CC3)c2c1] | a) Intermediate 137; b) Hydrazine hydrate |
| 72 | 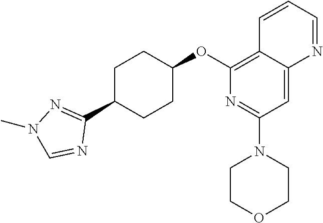 [SMILES: Cn1cnc([C@H]2CC[C@@H](Oc3nc(N4CCOCC4)cc4ncccc34)CC2)n1] | a) Intermediate 137; b) Methyl hydrazine |
| 73 | 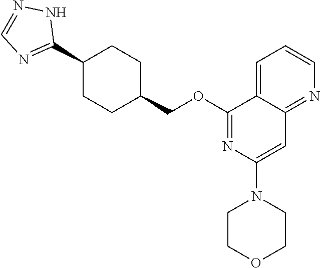 [SMILES: c1cnc2cc(N3CCOCC3)nc(OC[C@H]3CC[C@@H](c4ncn[nH]4)CC3)c2c1] | a) Intermediate 138; b) Hydrazine hydrate |

### Example B5

### a) Preparation of Parent Compound 74

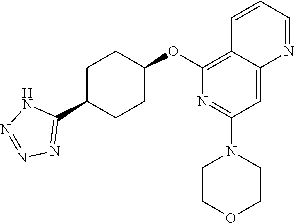 [SMILES: c1cnc2cc(N3CCOCC3)nc(O[C@H]3CC[C@@H](c4nnn[nH]4)CC3)c2c1]

A mixture of parent compound 4 (0.080 g, 0.24 mmol), sodium azide (0.046 g, 0.71 mmol) and triethylamine hydrochloride (0.098 g, 0.71 mmol) in DMF (0.75 ml) was heated under microwave irradiation at 130° C. for 2 hours, followed by 150° C. for 3 hours and 170° C. for 30 minutes. The resulting mixture was partitioned between EtOAc and water. The aqueous phase was acidified to pH 4 by addition of 1.0 M HCl and extracted with EtOAc. The combined organic phase was dried over sodium sulfate and concentrated in vacuo to afford the desired product as an orange solid (0.051 g, 56%).

LCMS (Method C): Rt=2.44 min, m/z [M+H]⁺=382

¹H NMR (400 MHz, DMSO-d₆) δ ppm: 8.79 (dd, J=1.5, 4.3 Hz, 1H), 8.35 (dd, J=1.5, 8.2 Hz, 1H), 7.20 (dd, J=4.3, 8.2 Hz, 1H), 6.53 (s, 1H), 5.75 (s, 1H), 5.51-5.46 (m, 1H), 3.77-3.73 (m, 4H), 3.53-3.49 (m, 4H), 3.24-3.17 (m, 1H), 2.15-2.07 (m, 2H), 2.03-1.83 (m, 6H).

### Example B6

### a) Preparation of Parent Compound 75

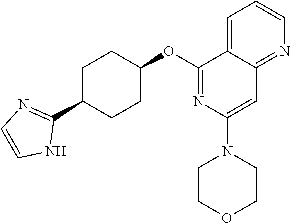 [SMILES: c1cnc2cc(N3CCOCC3)nc(O[C@H]3CC[C@@H](c4ncc[nH]4)CC3)c2c1]

A suspension of parent compound 50 (0.043 g, 0.091 mmol), acetic acid (0.10 ml, 1.82 mmol) and ammonium acetate (0.035 g, 0.46 mmol) in xylene (2.0 ml) was heated under microwave irradiation at 170° C. for 30 minutes. The resulting mixture was diluted with DCM and purified by ISOLUTE® SCX-2 SPE column, eluting with MeOH followed by 2.0 M ammonia solution in MeOH. Further purification by reverse phase preparative HPLC (Method B, followed by Method A) and trituration with diethyl ether afforded the desired product as a yellow solid (0.004 g, 12%).

LCMS (Method D): Rt=3.44 min, m/z [M+H]⁺=380

¹H NMR (400 MHz, DMSO-d₆) δ ppm: 8.80-8.77 (m, 1H), 8.36-8.32 (m, 1H), 8.28-8.24 (m, 1H), 7.22-7.17 (m, 1H), 6.90-6.84 (m, 2H), 6.54-6.52 (m, 1H), 5.48-5.43 (m, 0.75H), 5.21-5.13 (m, 0.25H), 3.78-3.72 (m, 4H), 3.55-3.48 (m, 4H), 2.86-2.73 (m, 1H), 2.28-1.92 (m, 4H), 1.88-1.62 (m, 4H). 3:1 mixture of cis:trans isomers.

### Example B7

a) Preparation of intermediate 139

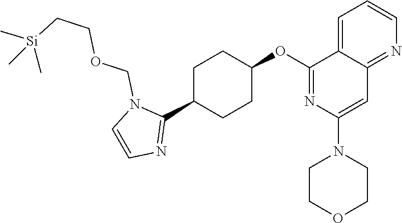 [SMILES: C[Si](C)(C)CCOCn1ccnc1[C@H]1CC[C@@H](Oc2nc(N3CCOCC3)cc3ncccc23)CC1]

A solution of parent compound 75 (0.080 g, 0.21 mmol) and triethylamine (0.059 ml, 0.42 mmol) in DCM (2.0 ml) was treated with 2-(trimethylsilyl)ethoxymethyl chloride (0.041 ml, 0.23 mmol) and the resulting mixture was stirred at ambient temperature for 5 hours. A second portion of 2-(trimethylsilyl)ethoxymethyl chloride (0.018 ml, 0.10 mmol) was added and stirring was continued for a further 30 minutes. The resulting mixture was partitioned between DCM and dilute aqueous sodium bicarbonate solution. The organic phase was dried over sodium sulfate and concentrated in vacuo. The residue was purified by column chromatography on silica gel, eluting with a mixture of DCM and 2.0 M ammonia solution in MeOH (1:0 to 93:7 by volume), to afford the desired product as a yellow oil (0.034 g, 32%).

LCMS (Method A): Rt=1.04 min, m/z [M+H]⁺=510

### b) Preparation of Intermediate 140

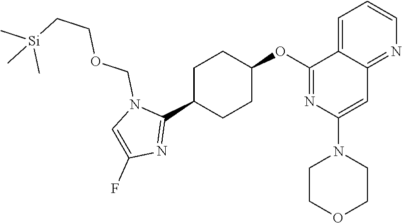 [SMILES: C[Si](C)(C)CCOCn1cc(F)nc1[C@H]1CC[C@@H](Oc2nc(N3CCOCC3)cc3ncccc23)CC1]

A solution of intermediate 139 (0.034 g, 0.067 mmol) in MeCN (1.0 ml) was treated with Selectfluor® (0.026 mg, 0.074 mmol). After stirring at ambient temperature for 19 hours, the resulting mixture was partitioned between DCM and dilute aqueous sodium bicarbonate solution. The organic phase was dried over sodium sulfate and concentrated in vacuo to afford the desired product as a yellow oil (0.031 g, 88%).

LCMS (Method A): Rt=1.22 Min, m/z [M+H]⁺=528

### c) Preparation of Parent Compound 76

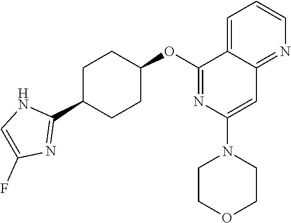 [SMILES: Fc1c[nH]c([C@H]2CC[C@@H](Oc3nc(N4CCOCC4)cc4ncccc34)CC2)n1]

A solution of intermediate 140 (0.031 g, 0.067 mmol) in DCM (2.0 ml) was treated with TFA (1.0 ml) and the resulting mixture was stirred at ambient temperature under a nitrogen atmosphere for 26 hours. The resulting mixture was purified by ISOLUTE® SCX-2 SPE column, eluting with MeOH followed by 2.0 M ammonia solution in MeOH. The residue was purified by chiral preparative SFC with the following conditions: YMC Cellulose-SC, 30/70 MeOH (0.1% DEA)/CO₂, 15 ml/min, 120 bar, 40 C, to afford the desired product as a yellow solid (0.005 g, 17%).

LCMS (Method C): Rt=2.39 min, m/z [M+H]⁺=398

¹H NMR (400 MHz, CDCl₃) δ ppm: 8.91 (dd, J=1.8, 4.3 Hz, 1H), 8.39-8.35 (m, 1H), 7.18 (dd, J=4.3, 8.3 Hz, 1H), 7.01 (m, 2H), 5.50-5.46 (m, 1H), 3.89-3.85 (m, 4H), 3.66-3.62 (m, 4H), 2.99-2.89 (m, 1H), 2.31-2.26 (m, 2H), 2.09-2.00 (m, 4H), 1.86-1.77 (m, 2H).

### Example B8

### a) Preparation of Intermediate 141

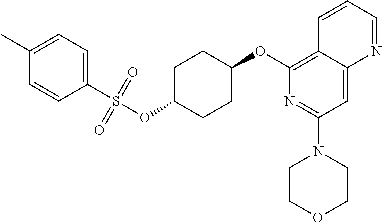 [SMILES: Cc1ccc(S(=O)(=O)O[C@H]2CC[C@H](Oc3nc(N4CCOCC4)cc4ncccc34)CC2)cc1]

A solution of parent compound 49 (0.90 g, 2.74 mmol) in pyridine (10 ml) at 0° C. was treated with p-toluenesulfonyl chloride (1.10 g, 5.74 mmol) and the resulting mixture was warmed to ambient temperature. After stirring for 18 hours, the resulting mixture was partitioned between DCM and water. The organic phase was dried over magnesium sulfate and concentrated in vacuo. The residue was purified by column chromatography on silica gel, eluting with a mixture of cyclohexane and EtOAc (19:1 to 0:1 by volume), to afford the desired product as a yellow solid (1.00 g, 76%).

LCMS (Method A): Rt=1.22 min, m/z [M+H]⁺=484

### b) Preparation of Parent Compound 77

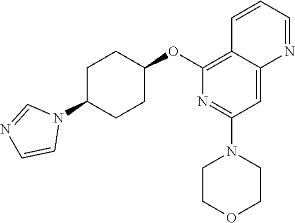 [SMILES: c1cnc2cc(N3CCOCC3)nc(O[C@H]3CC[C@@H](n4ccnc4)CC3)c2c1]

A solution of imidazole (0.015 g, 0.22 mmol) in DMF (1.0 ml) at 0° C. was treated with sodium hydride (0.09 g, 0.22 mmol, 60% in mineral oil). After stirring for 5 minutes, intermediate 141 (0.070 g, 0.15 mmol) was added and the resulting mixture was warmed to ambient temperature and then heated at 40° C. for 18 hours. The resulting mixture was cooled to ambient temperature and partitioned between EtOAc and water. The organic phase was dried over sodium sulfate and concentrated in vacuo. The residue was purified by reverse phase preparative HPLC (Method B) to afford the desired product as a yellow solid (0.012 g, 22%).

LCMS (Method D): Rt=3.70 min, m/z [M+H]⁺=380

¹H NMR (400 MHz, DMSO-d₆) δ ppm: 8.80 (dd, J=1.8, 4.3 Hz, 1H), 8.50-8.46 (m, 1H), 7.81-7.79 (m, 1H), 7.34-7.32 (m, 1H), 7.22 (dd, J=4.3, 8.2 Hz, 1H), 6.93-6.90 (m, 1H), 6.54 (s, 1H), 5.52-5.47 (m, 1H), 4.27-4.18 (m, 1H), 3.77-3.72 (m, 4H), 3.52-3.48 (m, 4H), 2.23-2.04 (m, 4H), 1.95-1.79 (m, 4H).

Parent compounds 78 to 81 were prepared according to the reaction protocol of parent compound 77 using the appropriate starting materials (Table 24).

TABLE 24

| Parent Compound | Structure | Starting Materials |
|---|---|---|
| 78 | 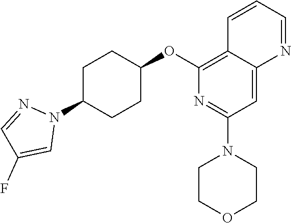 [SMILES: Fc1cnn([C@H]2CC[C@@H](Oc3nc(N4CCOCC4)cc4ncccc34)CC2)c1] | a) Intermediate 141; b) 4-Fluoro-1H-pyrazole |
| 79 | 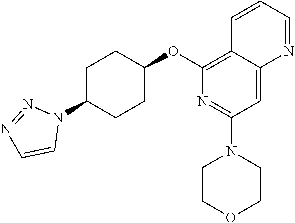 [SMILES: c1cnc2cc(N3CCOCC3)nc(O[C@H]3CC[C@@H](n4ccnn4)CC3)c2c1] | a) Intermediate 141; b) 1H-1,2,3-Triazole |
| 80 | 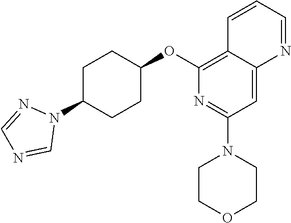 [SMILES: c1cnc2cc(N3CCOCC3)nc(O[C@H]3CC[C@@H](n4cncn4)CC3)c2c1] | a) Intermediate 141; b) 1H-1,2,4-Triazole |
| 81 | 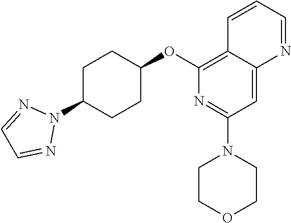 [SMILES: c1cnc2cc(N3CCOCC3)nc(O[C@H]3CC[C@@H](n4nccn4)CC3)c2c1] | a) Intermediate 141; b) 1H-1,2,3-Triazole |

### Example B9

### a) Preparation of Intermediate 142

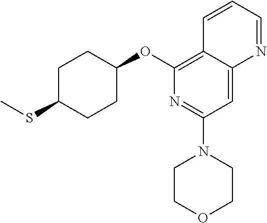 [SMILES: CS[C@H]1CC[C@@H](Oc2nc(N3CCOCC3)cc3ncccc23)CC1]

A solution of intermediate 141 (0.15 g, 0.31 mmol) in denatured EtOH (6.0 ml) was treated with sodium methanethiolate (0.043 g, 0.62 mmol) and the resulting mixture was heated at reflux for 1 hour under a nitrogen atmosphere. A second portion of sodium methanethiolate (0.043 g, 0.62 mmol) was added and heating was continued for a further 2 hours. The resulting mixture was cooled to ambient temperature and partitioned between DCM and water. The organic phase was dried over magnesium sulfate and concentrated in vacuo. The residue was purified by column chromatography on silica gel, eluting with a mixture of cyclohexane and EtOAc (1:0 to 2:3 by volume), to afford the desired product as an orange oil (0.085 g, 76%).

LCMS (Method A): Rt=1.07 min, m/z [M+H]⁺=360

### b) Preparation of Parent Compound 82

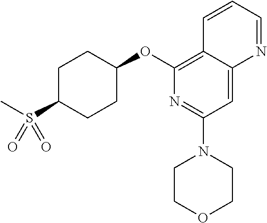 [SMILES: CS(=O)(=O)[C@H]1CC[C@@H](Oc2nc(N3CCOCC3)cc3ncccc23)CC1]

A solution of intermediate 142 (0.085 g, 0.24 mmol) in DCM (3.0 ml) was treated with TFA (0.036 ml, 0.47 mmol) at ambient temperature. After 5 minutes, 3-chloroperbenzoic acid (0.11 g, 0.47 mmol) was added and stirring was continued for a further 20 hours. The resulting mixture was partitioned between DCM and saturated aqueous sodium bicarbonate solution. The organic phase was dried over magnesium sulfate and concentrated in vacuo. The residue was purified by reverse phase preparative HPLC (Method B) to afford the desired product as a yellow solid (0.006 g, 6%).

LCMS (Method C): Rt=2.40 min, m/z [M+H]⁺=392

¹H NMR (400 MHz, DMSO-d₆) δ ppm: 8.79 (dd, J=1.5, 4.3 Hz, 1H), 8.29 (dd, J=1.5, 8.2 Hz, 1H), 7.22 (dd, J=4.3, 8.2 Hz, 1H), 6.54 (s, 1H), 5.49-5.44 (m, 1H), 3.76-3.72 (m, 4H), 3.52-3.47 (m, 4H), 3.27-3.18 (m, 1H), 2.95 (s, 3H), 2.26-2.18 (m, 2H), 2.04-1.96 (m, 2H), 1.92-1.71 (m, 4H).

### Example B10

### a) Preparation of Parent Compound 83

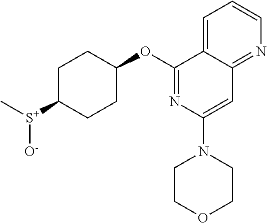 [SMILES: C[S+]([O-])[C@H]1CC[C@@H](Oc2nc(N3CCOCC3)cc3ncccc23)CC1]

A solution of intermediate 142 (0.025 g, 0.069 mmol) in DCM (2.0 ml) at 0° C. was treated with 3-chloroperbenzoic acid (0.034 g, 0.14 mmol) and the resulting mixture was warmed to ambient temperature. After stirring for 1 hour, the resulting mixture was partitioned between DCM and water. The organic phase was dried over magnesium sulfate and concentrated in vacuo. The residue was purified by reverse phase preparative HPLC (Method A) to afford the desired product as an orange solid (0.004 g, 15%).

LCMS (Method C): Rt=2.26 min, m/z [M+H]⁺=376

¹H NMR (400 MHz, DMSO-d₆) δ ppm: 8.79 (dd, J=1.5, 4.3 Hz, 1H), 8.29 (dd, J=1.5, 8.2 Hz, 1H), 7.20 (dd, J=4.3, 8.2 Hz, 1H), 6.54 (s, 1H), 5.48-5.44 (m, 1H), 3.76-3.72 (m, 4H), 3.52-3.47 (m, 4H), 2.78-2.70 (m, 1H), 2.55-2.54 (m, 3H), 2.22-2.11 (m, 2H), 1.92-1.76 (m, 6H). 1:1 mixture of diastereomers.

### Example B11

### a) Preparation of Intermediate 143

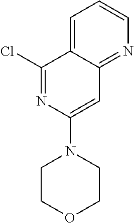 [SMILES: Clc1nc(N2CCOCC2)cc2ncccc12]

A suspension of parent compound 53 (0.50 g, 1.43 mmol) in 4.0 M HCl in dioxane (20 ml) was stirred at ambient temperature for 30 minutes. The resulting mixture was concentrated in vacuo, then suspended in phosphorus(V) oxychloride (20 ml) and heated at 90° C. for 16 hours. The resulting mixture was concentrated in vacuo and partitioned between DCM and dilute aqueous sodium bicarbonate solution. The organic phase was dried over sodium sulfate and concentrated in vacuo. The residue was purified by column chromatography on silica gel, eluting with a mixture of DCM and EtOAc (1:1 by volume) followed by DCM and 2.0 M ammonia solution in methanol (1:0 to 17:3 by volume), to afford the desired product as a yellow solid (0.29 g, 82%).

LCMS (Method B): Rt=1.71 min, m/z [M+H]⁺=250/252

b) Preparation of parent compound 84;

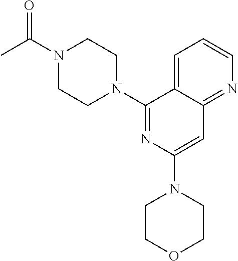 [SMILES: CC(=O)N1CCN(c2nc(N3CCOCC3)cc3ncccc23)CC1]

A suspension of intermediate 143 (0.10 g, 0.40 mmol), 1-(piperazin-1-yl)ethan-1-one (0.10 g, 0.80 mmol), RuPhos Pd G1.TBME (0.033 g, 0.04 mmol), RuPhos (0.019 g, 0.04 mmol) and cesium carbonate (0.26 g, 0.80 mmol) in dioxane (4.0 ml) was heated at 100° C. for 1 hour under an argon atmosphere. The resulting mixture was cooled to ambient temperature, filtered through Celite® and concentrated in vacuo. The residue was purified by column chromatography on silica gel, eluting with a mixture of DCM and EtOAc (1:1 by volume) followed by DCM and 2.0 M ammonia solution in methanol (1:0 to 17:3 by volume), to afford the desired product as a yellow solid (0.10 g, 76%).

LCMS (Method C): Rt=2.13 min, m/z [M+H]⁺=342

¹H NMR (400 MHz, DMSO-d₆) δ ppm: 8.75 (dd, J=1.5, 4.2 Hz, 1H), 8.24 (dd, J=1.5, 8.3 Hz, 1H), 7.16 (dd, J=4.2, 8.3 Hz, 1H), 6.57 (s, 1H), 3.76-3.71 (m, 4H), 3.69-3.66 (m, 4H), 3.53-3.48 (m, 4H), 3.40-3.35 (m, 2H), 3.33-3.30 (m, 2H), 2.05 (s, 3H).

Parent compound 85 was prepared according to the reaction protocol of parent compound 84 using the appropriate starting materials (Table 25).

TABLE 25

| Parent | | |
|---|---|---|
| Compound | Structure | Starting Materials |
| 85 | 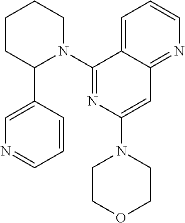 [SMILES: c1cncc(C2CCCCN2c2nc(N3CCOCC3)cc3ncccc23)c1] | a) Intermediate 143; b) 2-(3-Pyridyl) piperidine |

### Example B12

### a) Preparation of Parent Compound 86

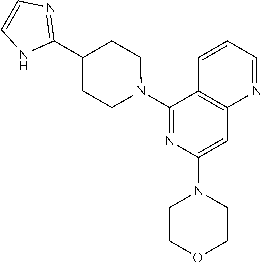 [SMILES: c1cnc2cc(N3CCOCC3)nc(N3CCC(c4ncc[nH]4)CC3)c2c1]

A mixture of parent compound 14 (0.047 g, 0.10 mmol), concentrated HCl (0.17 ml) and MeOH (5.0 ml) was heated at reflux for 2 hours. The resulting mixture was cooled to ambient temperature and concentrated in vacuo. The residue was purified by ISOLUTE® SCX-2 SPE column, eluting with MeOH followed by 2.0 M ammonia solution in MeOH, then by reverse phase preparative HPLC (Method A). Further purification by ISOLUTE® SCX-2 SPE column, eluting with MeOH followed by 2.0 M ammonia solution in MeOH and trituration with diethyl ether afforded the desired product as a pale yellow solid (0.020 g, 55%).

LCMS (Method C): Rt=1.70 min, m/z [M+H]⁺=365

¹H NMR (400 MHz, DMSO-d₆) δ ppm: 11.83 (bs, 1H), 8.72 (dd, J=1.5, 4.2 Hz, 1H), 8.16 (dd, J=1.5, 8.3 Hz, 1H), 7.15 (dd, J=4.2, 8.3 Hz, 1H), 6.91 (s, 2H), 6.52 (s, 1H), 3.90-3.83 (m, 2H), 3.76-3.71 (m, 4H), 3.53-3.48 (m, 4H), 3.12-3.03 (m, 2H), 2.98-2.89 (m, 1H), 2.05-1.95 (m, 4H).

Parent compound 87 was prepared according to the reaction protocol of parent compound 86 using the appropriate starting materials (Table 26).

TABLE 26

| Parent | | Starting |
|---|---|---|
| Compound | Structure | Materials |
| 87 | 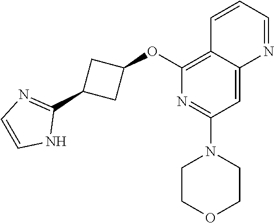 [SMILES: c1cnc2cc(N3CCOCC3)nc(O[C@H]3C[C@@H](c4ncc[nH]4)C3)c2c1] | Parent compound 51 |

### Example B13

### a) Preparation of Parent Compound 88

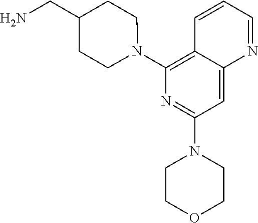 [SMILES: NCC1CCN(c2nc(N3CCOCC3)cc3ncccc23)CC1]

A solution of parent compound 33 (3.16 g, 7.40 mmol) in DCM (54 ml) was treated with TFA (9.0 ml). After stirring at ambient temperature for 1.5 hours, the resulting mixture was concentrated in vacuo. The residue was purified by ISOLUTE® SCX-2 SPE column, eluting with MeOH followed by 2.0 M ammonia solution in MeOH, to afford the desired product as a yellow solid (2.36 g, 98%).

LCMS (Method B): Rt=1.74 min, m/z [M+H]⁺=328

Parent compounds 89 to 95 were prepared according to the reaction protocol of parent compound 88 using the appropriate starting materials (Table 27).

TABLE 27

| Parent Compound | Structure | Starting Materials |
|---|---|---|
| 89 | 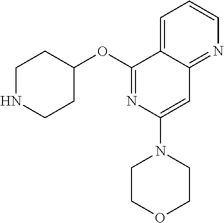 [SMILES: c1cnc2cc(N3CCOCC3)nc(OC3CCNCC3)c2c1] | Parent compound 48 |
| 90 | 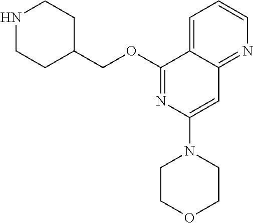 [SMILES: c1cnc2cc(N3CCOCC3)nc(OCC3CCNCC3)c2c1] | Parent compound 43 |
| 91 | 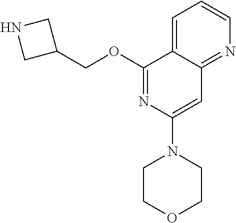 [SMILES: c1cnc2cc(N3CCOCC3)nc(OCC3CNC3)c2c1] | Parent compound 44 |
| 92 | 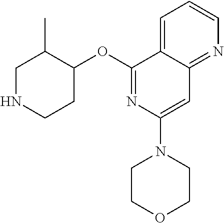 [SMILES: CC1CNCCC1Oc1nc(N2CCOCC2)cc2ncccc12] | Parent compound 45 3:2 mixture of trans:cis diastereomers. |
| 93 | 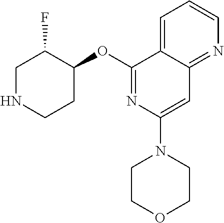 [SMILES: F[C@H]1CNCC[C@@H]1Oc1nc(N2CCOCC2)cc2ncccc12] | Parent compound 46 |
| 94 | 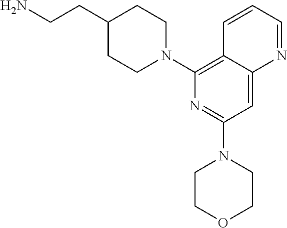 [SMILES: NCCC1CCN(c2nc(N3CCOCC3)cc3ncccc23)CC1] | Parent compound 30 |
| 95 | 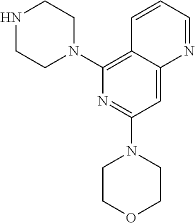 [SMILES: c1cnc2cc(N3CCOCC3)nc(N3CCNCC3)c2c1] | Parent compound 19 |

### Example B14

### a) Preparation of Parent Compounds 96 and 97

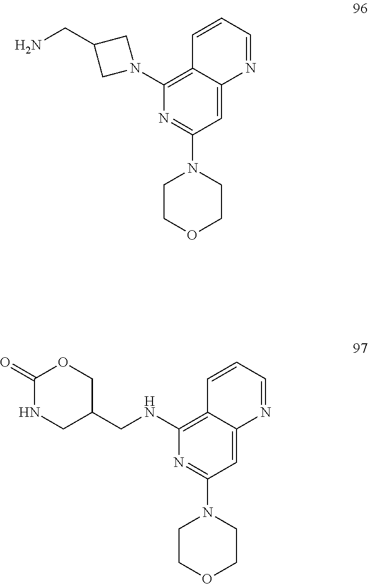 [SMILES: NCC1CN(c2nc(N3CCOCC3)cc3ncccc23)C1 ; O=C1NCC(CNc2nc(N3CCOCC3)cc3ncccc23)CO1]

A solution of parent compound 47 (1.90 g, 4.76 mmol) in DCM (30 ml) was treated with TFA (2.7 ml) and the resulting mixture was stirred at ambient temperature under a nitrogen atmosphere for 22 hours. Additional TFA (3.0 ml) was added and the resulting mixture was stirred at 40° C. for 1 hour, then cooled to ambient temperature and concentrated in vacuo. The residue was treated with TFA (6.0 ml) and the resulting mixture was stirred at ambient temperature for a further 18 hours. The mixture was concentrated in vacuo and the residue was purified by ISOLUTE® SCX-2 SPE column, eluting with MeOH followed by 2.0 M ammonia solution in MeOH. Further purification by column chromatography on silica gel, eluting with a mixture of DCM and MeOH (1:0 to 9:1 by volume), afforded parent compounds 96 as a yellow solid (0.34 g, 24%) and 97 as a yellow solid (1.10 g, 67%).

### Parent Compound 96

LCMS (Method B): Rt=1.33 min, m/z [M+H]⁺=300

### Parent Compound 97

LCMS (Method C): Rt=1.92 min, m/z [M+H]⁺=344

¹H NMR (400 MHz, DMSO-d₆) δ ppm: 8.64 (dd, J=1.6, 4.3 Hz, 1H), 8.41-8.38 (m, 1H), 7.71 (t, J=5.5 Hz, 1H), 7.16 (s, 1H), 7.06 (dd, J=4.3, 8.3 Hz, 1H), 6.16 (s, 1H), 4.25 (dd, J=2.5, 10.9 Hz, 1H), 4.07 (dd, J=7.5, 10.9 Hz, 1H), 3.74-3.69 (m, 4H), 3.52-3.42 (m, 6H), 3.30-3.27 (m, 1H), 3.08-3.03 (m, 1H), 2.45-2.33 (m, 1H).

### Example B15

### a) Preparation of Parent Compound 98

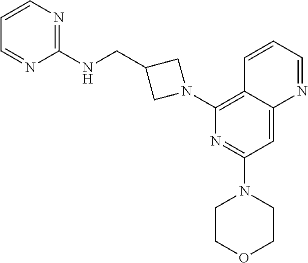 [SMILES: c1cnc(NCC2CN(c3nc(N4CCOCC4)cc4ncccc34)C2)nc1]

A mixture of parent compound 96 (0.051 g, 0.19 mmol), 2-fluoropyrimidine (0.018 g, 0.19 mmol) and triethylamine (0.079 ml, 0.56 mmol) in iPrOH (5.0 ml) was heated at 80° C. for 16 hours. The resulting mixture was cooled to ambient temperature and partitioned between EtOAc and water. The organic phase was dried over sodium sulfate and concentrated in vacuo. The residue was purified by reverse phase preparative HPLC (Method B) to afford the desired product as a yellow solid (0.002 g, 3%).

LCMS (Method C): Rt=2.38 min, m/z [M+H]⁺=378

¹H NMR (400 MHz, DMSO-d₆) δ ppm: 8.63 (dd, J=1.5, 4.2 Hz, 1H), 8.27 (d, J=4.8 Hz, 2H), 8.09 (dd, J=1.5, 8.3 Hz, 1H), 7.37 (t, J=5.9 Hz, 1H), 7.01 (dd, J=4.3, 8.3 Hz, 1H), 6.57 (t, J=4.8 Hz, 1H), 6.27 (s, 1H), 4.42-4.35 (m, 2H), 4.10 (dd, J=5.4, 8.7 Hz, 2H), 3.73-3.68 (m, 4H), 3.59-3.54 (m, 2H), 3.49-3.43 (m, 4H), 3.05-2.96 (m, 1H).

Parent compounds 99 to 130 were prepared according to the reaction protocol of parent compound 98 using the appropriate starting materials (Table 28).

TABLE 28

| Parent Compound | Structure | Starting Materials |
|---|---|---|
| 99 | 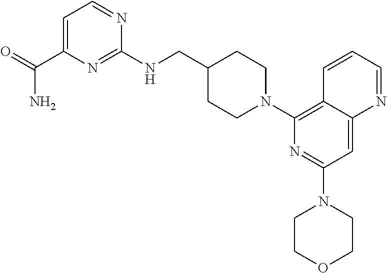 [SMILES: NC(=O)c1ccnc(NCC2CCN(c3nc(N4CCOCC4)cc4ncccc34)CC2)n1] | a) Parent compound 88 b) 2-Chloropyrimidine-4- carboxamide |
| 100 | 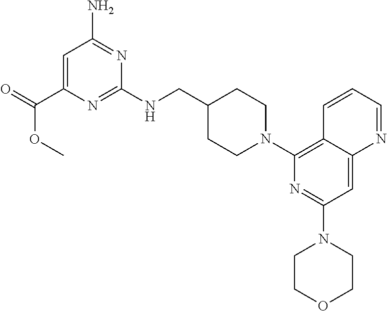 [SMILES: COC(=O)c1cc(N)nc(NCC2CCN(c3nc(N4CCOCC4)cc4ncccc34)CC2)n1] | a) Parent compound 88 b) Methyl 6-amino-2- chloropyrimidine-4-carboxylate |
| 101 | 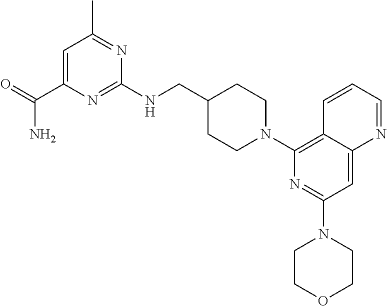 [SMILES: Cc1cc(C(N)=O)nc(NCC2CCN(c3nc(N4CCOCC4)cc4ncccc34)CC2)n1] | a) Parent compound 88 b) Methyl 2-chloro-6- methylpyrimidine-4- carboxylate |
| 102 | 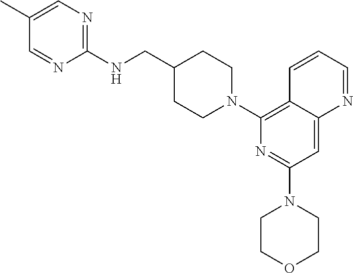 [SMILES: Cc1cnc(NCC2CCN(c3nc(N4CCOCC4)cc4ncccc34)CC2)nc1] | a) Parent compound 88 b) 2-Chloro-5-methylpyrimidine |
| 103 | 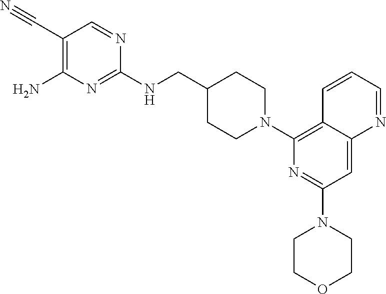 [SMILES: N#Cc1cnc(NCC2CCN(c3nc(N4CCOCC4)cc4ncccc34)CC2)nc1N] | a) Parent compound 88 b) 4-Amino-2-chloropyrimidine- 5-carbonitrile |
| 104 | 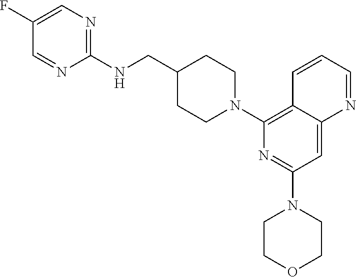 [SMILES: Fc1cnc(NCC2CCN(c3nc(N4CCOCC4)cc4ncccc34)CC2)nc1] | a) Parent compound 88; b) 2,5-Difluoropyrimidine |
| 105 | 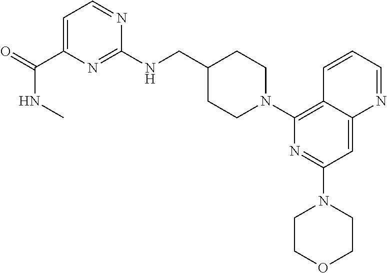 [SMILES: CNC(=O)c1ccnc(NCC2CCN(c3nc(N4CCOCC4)cc4ncccc34)CC2)n1] | a) Parent compound 88 b) 2-Chloro-N- methylpyrimidine-4- carboxamide |
| 106 | 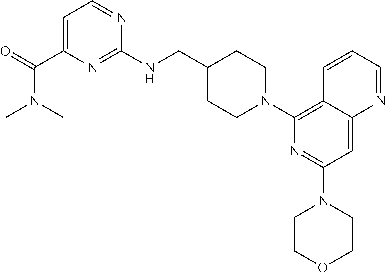 [SMILES: CN(C)C(=O)c1ccnc(NCC2CCN(c3nc(N4CCOCC4)cc4ncccc34)CC2)n1] | a) Parent compound 88 b) 2-Chloro-N,N- dimethylpyrimidine-4- carboxamide |
| 107 | 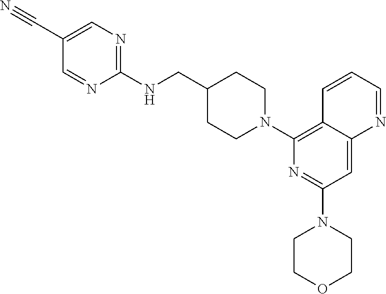 [SMILES: N#Cc1cnc(NCC2CCN(c3nc(N4CCOCC4)cc4ncccc34)CC2)nc1] | a) Parent compound 88 b) 2-Chloropyrimidine-5- carbonitrile |
| 108 | 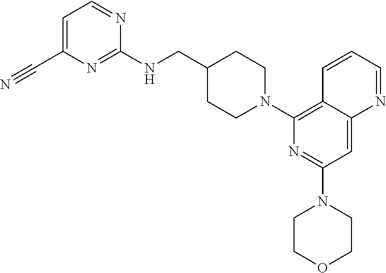 [SMILES: N#Cc1ccnc(NCC2CCN(c3nc(N4CCOCC4)cc4ncccc34)CC2)n1] | a) Parent compound 88 b) 2-Chloropyrimidine-4- carbonitrile |
| 109 | 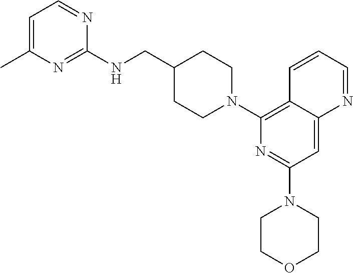 [SMILES: Cc1ccnc(NCC2CCN(c3nc(N4CCOCC4)cc4ncccc34)CC2)n1] | a) Parent compound 88 b) 2-Chloro-4-methylpyrimidine |
| 110 | 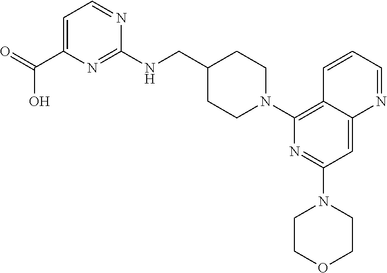 [SMILES: O=C(O)c1ccnc(NCC2CCN(c3nc(N4CCOCC4)cc4ncccc34)CC2)n1] | a) Parent compound 88 b) 2-Chloropyrimidine-4- carboxylic acid |
| 111 | 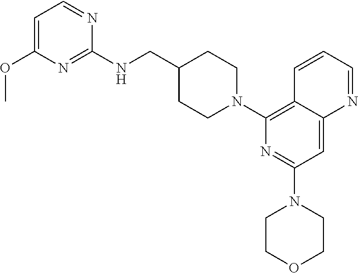 [SMILES: COc1ccnc(NCC2CCN(c3nc(N4CCOCC4)cc4ncccc34)CC2)n1] | a) Parent compound 88 b) 2-Chloro-4- methoxypyrimidine |
| 112 | 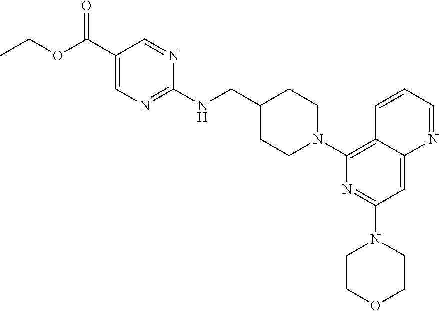 [SMILES: CCOC(=O)c1cnc(NCC2CCN(c3nc(N4CCOCC4)cc4ncccc34)CC2)nc1] | a) Parent compound 88 b) Ethyl 2-chloropyrimidine-5- carboxylate 4 |
| 113 | 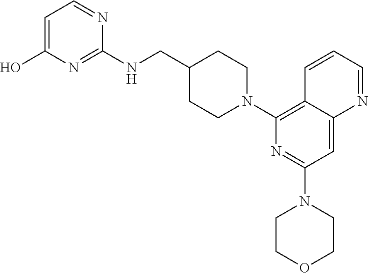 [SMILES: Oc1ccnc(NCC2CCN(c3nc(N4CCOCC4)cc4ncccc34)CC2)n1] | a) Parent compound 88 b) 2-Chloropyrimidin-4-ol |
| 114 | 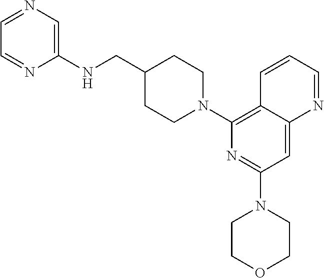 [SMILES: c1cnc2cc(N3CCOCC3)nc(N3CCC(CNc4cnccn4)CC3)c2c1] | a) Parent compound 88; b) 2-Chloropyrazine |
| 115 | 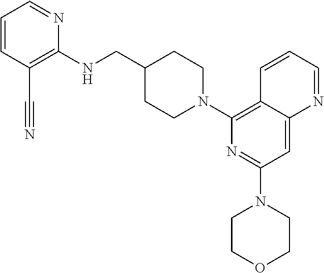 [SMILES: N#Cc1cccnc1NCC1CCN(c2nc(N3CCOCC3)cc3ncccc23)CC1] | a) Parent compound 88 b) 2-Fluoronicotinonitrile |
| 116 | 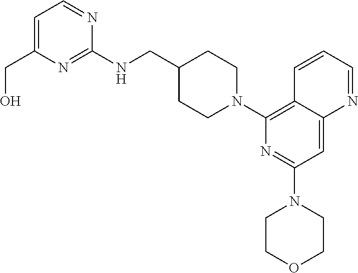 [SMILES: OCc1ccnc(NCC2CCN(c3nc(N4CCOCC4)cc4ncccc34)CC2)n1] | a) Parent compound 88 b) (2-Chloropyrimidin-4- yl)methanol |
| 117 | 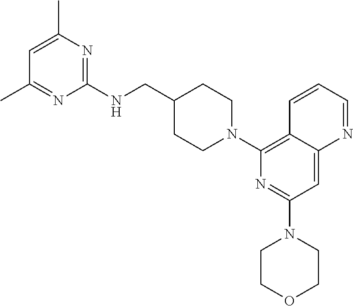 [SMILES: Cc1cc(C)nc(NCC2CCN(c3nc(N4CCOCC4)cc4ncccc34)CC2)n1] | a) Parent compound 88 b) 2-Chloro-4,6- dimethylpyrimidine |
| 118 | 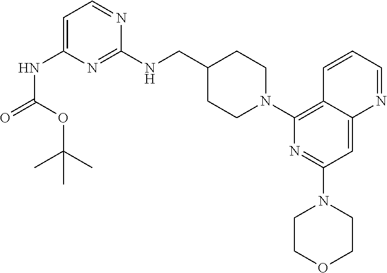 [SMILES: CC(C)(C)OC(=O)Nc1ccnc(NCC2CCN(c3nc(N4CCOCC4)cc4ncccc34)CC2)n1] | a) Parent compound 88 b) tert-Butyl (2-chloropyrimidin- 4-yl)carbamate |
| 119 | 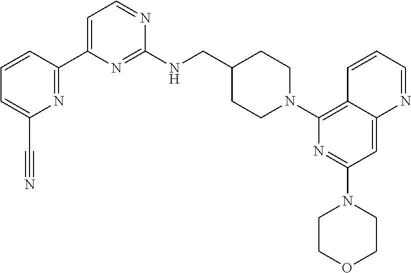 [SMILES: N#Cc1cccc(-c2ccnc(NCC3CCN(c4nc(N5CCOCC5)cc5ncccc45)CC3)n2)n1] | a) Parent compound 88 b) 6-(2-Chloropyrimidin-4- yl)picolinonitrile |
| 120 | 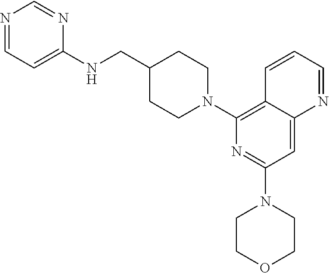 [SMILES: c1cnc2cc(N3CCOCC3)nc(N3CCC(CNc4ccncn4)CC3)c2c1] | a) Parent compound 88 b) 4-Chloropyrimidine |
| 121 | 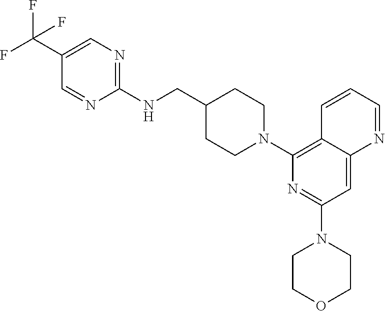 [SMILES: FC(F)(F)c1cnc(NCC2CCN(c3nc(N4CCOCC4)cc4ncccc34)CC2)nc1] | a) Parent compound 88 b) 2-Chloro-5- (trifluoromethyl)pyrimidine |
| 122 | 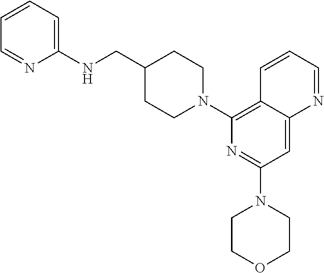 [SMILES: c1ccc(NCC2CCN(c3nc(N4CCOCC4)cc4ncccc34)CC2)nc1] | a) Parent compound 88 b) 2-Fluoropyridine |
| 123 | 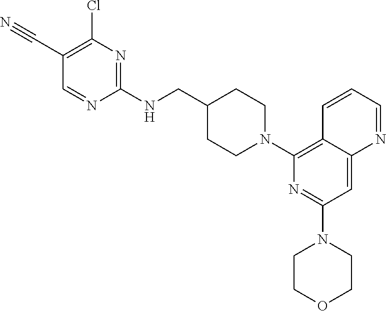 [SMILES: N#Cc1cnc(NCC2CCN(c3nc(N4CCOCC4)cc4ncccc34)CC2)nc1Cl] | a) Parent compound 88 b) 2,4-Dichloropyrimidine-5- carbonitrile |
| 124 | 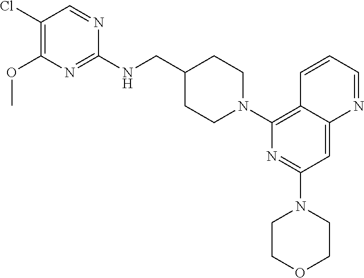 [SMILES: COc1nc(NCC2CCN(c3nc(N4CCOCC4)cc4ncccc34)CC2)ncc1Cl] | a) Parent compound 88 b) 2,5-Dichloro-4- methoxypyrimidine |
| 125 | 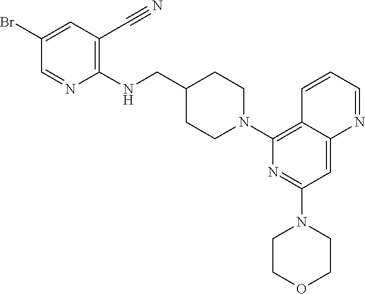 [SMILES: N#Cc1cc(Br)cnc1NCC1CCN(c2nc(N3CCOCC3)cc3ncccc23)CC1] | a) Parent compound 88 b) 5-Bromo-2- fluoronicotinonitrile |
| 126 | 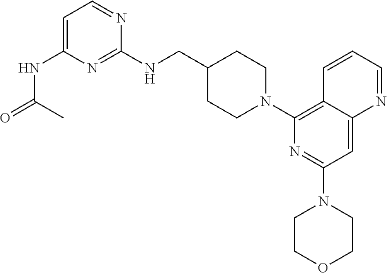 [SMILES: CC(=O)Nc1ccnc(NCC2CCN(c3nc(N4CCOCC4)cc4ncccc34)CC2)n1] | a) Parent compound 88 b) N-(2-Chloropyrimidin-4- yl)acetamide |
| 127 | 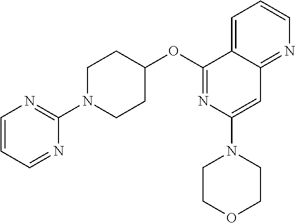 [SMILES: c1cnc(N2CCC(Oc3nc(N4CCOCC4)cc4ncccc34)CC2)nc1] | a) Parent compound 89 b) 2-Fluoropyrimidine |
| 128 | 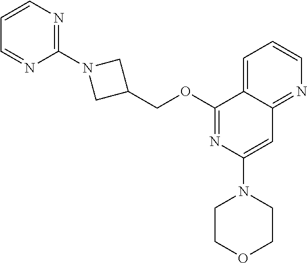 [SMILES: c1cnc(N2CC(COc3nc(N4CCOCC4)cc4ncccc34)C2)nc1] | a) Parent compound 91 b) 2-Fluoropyrimidine |
| 129 | 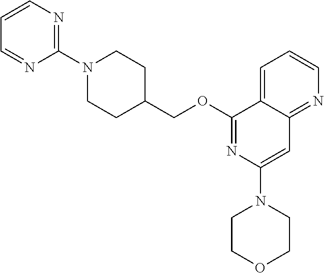 [SMILES: c1cnc(N2CCC(COc3nc(N4CCOCC4)cc4ncccc34)CC2)nc1] | a) Parent compound 90 b) 2-Fluoropyrimidine |
| 130 | 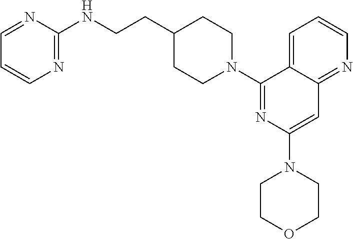 [SMILES: c1cnc(NCCC2CCN(c3nc(N4CCOCC4)cc4ncccc34)CC2)nc1] | a) Parent compound 94 b) 2-Fluoropyrimidine |

### Example B16

### a) Preparation of Parent Compound 131

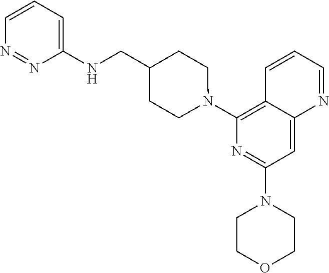 [SMILES: c1cnnc(NCC2CCN(c3nc(N4CCOCC4)cc4ncccc34)CC2)c1]

A suspension of parent compound 88 (0.080 g, 0.25 mmol), 3-bromopyridazine (0.043 g, 0.27 mmol), palladium(II) acetate (0.0022 g, 0.009 mmol), BINAP (0.0061 g, 0.009 mmol) and sodium tert-butoxide (0.059 g, 0.61 mmol) in THF (2.5 ml) was heated at reflux for 20 hours under an argon atmosphere. The resulting mixture was cooled to ambient temperature, diluted with EtOAc and filtered through Celite®. The filtrate was concentrated in vacuo and the residue was purified by reverse phase preparative HPLC (Method B) to afford the desired product as an orange solid (0.013 g, 13%).

LCMS (Method C): Rt=1.90 min, m/z [M+H]⁺=406

¹H NMR (400 MHz, DMSO-d₆) δ ppm: 8.71 (dd, J=1.5, 4.2 Hz, 1H), 8.39 (dd, J=1.4, 4.4 Hz, 1H), 8.14 (dd, J=1.5, 8.3 Hz, 1H), 7.20 (dd, J=4.4, 9.0 Hz, 1H), 7.14 (dd, J=4.2, 8.3 Hz, 1H), 6.90 (t, J=5.6 Hz, 1H), 6.81 (dd, J=1.4, 9.0 Hz, 1H), 6.50 (s, 1H), 3.87-3.79 (m, 2H), 3.75-3.70 (m, 4H), 3.53-3.46 (m, 4H), 3.36-3.27 (m, 2H), 2.99-2.89 (m, 2H), 1.91-1.83 (m, 3H), 1.55-1.45 (m, 2H).

Parent compounds 132 to 138 were prepared according to the reaction protocol of parent compound 131 using the appropriate starting materials (Table 29).

TABLE 29

| Parent Compound | Structure | Starting Materials |
|---|---|---|
| 132 | 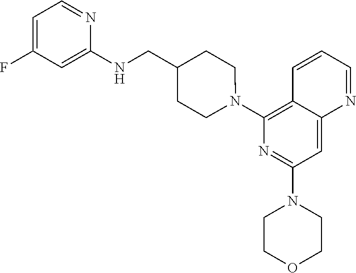 [SMILES: Fc1ccnc(NCC2CCN(c3nc(N4CCOCC4)cc4ncccc34)CC2)c1] | a) Parent compound 88 b) 2-Bromo-4-fluoropyridine |
| 133 | 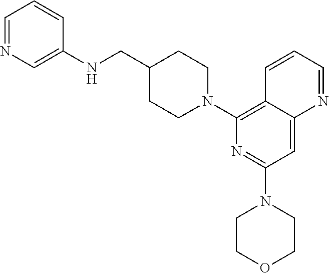 [SMILES: c1cncc(NCC2CCN(c3nc(N4CCOCC4)cc4ncccc34)CC2)c1] | a) Parent compound 88 b) 3-Bromopyridine |
| 134 | 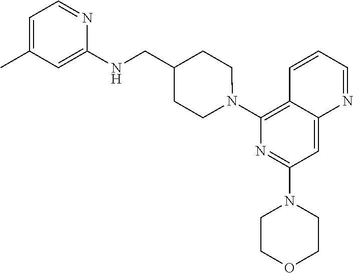 [SMILES: Cc1ccnc(NCC2CCN(c3nc(N4CCOCC4)cc4ncccc34)CC2)c1] | a) Parent compound 88 b) 2-Bromo-4-methylpyridine |
| 135 | 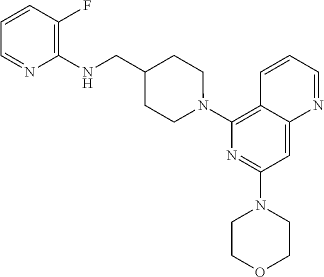 [SMILES: Fc1cccnc1NCC1CCN(c2nc(N3CCOCC3)cc3ncccc23)CC1] | a) Parent compound 88 b) 2-Bromo-3-fluoropyridine |
| 136 | 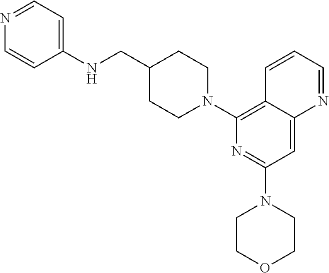 [SMILES: c1cnc2cc(N3CCOCC3)nc(N3CCC(CNc4ccncc4)CC3)c2c1] | a) Parent compound 88 b) 4-Bromopyridine |
| 137 | 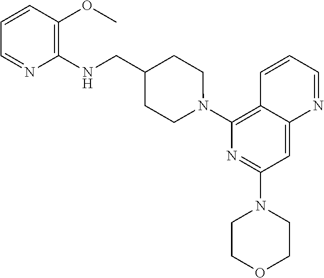 [SMILES: COc1cccnc1NCC1CCN(c2nc(N3CCOCC3)cc3ncccc23)CC1] | a) Parent compound 88 b) 2-Bromo-3-methoxypyridine |
| 138 | 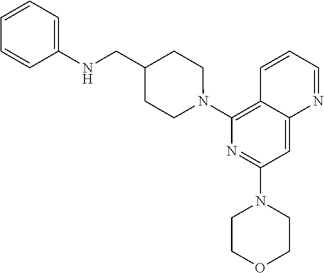 [SMILES: c1ccc(NCC2CCN(c3nc(N4CCOCC4)cc4ncccc34)CC2)cc1] | a) Parent compound 88 b) Bromobenzene |

### Example B17

### a) Preparation of Parent Compound 139

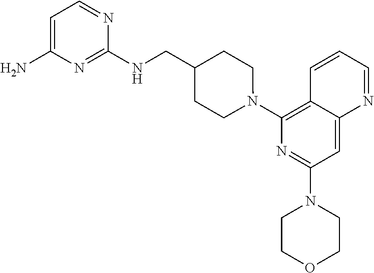 [SMILES: Nc1ccnc(NCC2CCN(c3nc(N4CCOCC4)cc4ncccc34)CC2)n1]

A solution of parent compound 118 (0.045 g, 0.086 mmol) in DCM (4.0 ml) at 0° C. was treated with TFA (0.5 ml) and the resulting mixture was warmed to ambient temperature. After stirring for 3 hours, the resulting mixture was concentrated in vacuo. The residue was purified by ISOLUTE® SCX-2 SPE column, eluting with MeOH followed by 2.0 M ammonia solution in MeOH, to afford the desired product as a yellow solid (0.026 g, 72%).

LCMS (Method C): Rt=1.99 min, m/z [M+H]⁺=421

¹H NMR (400 MHz, DMSO-d₆) δ ppm: 8.71 (dd, J=1.5, 4.2 Hz, 1H), 8.12 (dd, J=1.5, 8.3 Hz, 1H), 7.66 (d, J=5.6 Hz, 1H), 7.13 (dd, J=4.2, 8.3 Hz, 1H), 6.49 (s, 1H), 6.38 (bs, 1H), 6.21 (bs, 2H), 5.67 (d, J=5.6 Hz, 1H), 3.84-3.76 (m, 2H), 3.75-3.70 (m, 4H), 3.51-3.46 (m, 4H), 3.21-3.15 (m, 2H), 2.95-2.85 (m, 2H), 1.85-1.74 (m, 3H), 1.46-1.37 (m, 2H).

### Example B18

### a) Preparation of Parent Compound 140

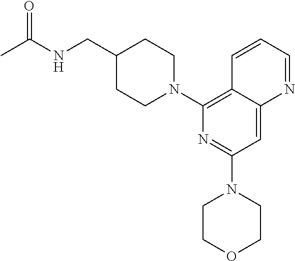 [SMILES: CC(=O)NCC1CCN(c2nc(N3CCOCC3)cc3ncccc23)CC1]

A solution of parent compound 88 (0.070 g, 0.21 mmol) in THF (2.0 ml) at 0° C. was treated with triethylamine (0.055 ml, 0.43 mmol) and acetyl chloride (0.016 ml, 0.22 mmol) and the resulting mixture was warmed to ambient temperature. After stirring for 1 hour, the resulting mixture was partitioned between DCM and water. The organic phase was dried over magnesium sulfate and concentrated in vacuo. The residue was purified by column chromatography on silica gel, eluting with a mixture of DCM and MeOH (1:0 to 9:1 by volume), to afford the desired product as a yellow solid (0.068 g, 87%).

LCMS (Method C): Rt=2.28 min, m/z [M+H]⁺=370

¹H NMR (400 MHz, DMSO-d₆) δ ppm: 8.71 (dd, J=1.5, 4.2 Hz, 1H), 8.12 (dd, J=1.5, 8.3 Hz, 1H), 7.89 (t, J=5.6 Hz, 1H), 7.13 (dd, J=4.2, 8.3 Hz, 1H), 6.50 (s, 1H), 3.84-3.76 (m, 2H), 3.75-3.70 (m, 4H), 3.52-3.47 (m, 4H), 3.05-2.99 (m, 2H), 2.94-2.85 (m, 2H), 1.82 (s, 3H), 1.80-1.75 (m, 2H), 1.67-1.60 (m, 1H), 1.46-1.33 (m, 2H).

### Example B19

### a) Preparation of Parent Compound 14103

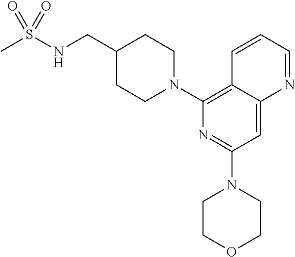 [SMILES: CS(=O)(=O)NCC1CCN(c2nc(N3CCOCC3)cc3ncccc23)CC1]

A solution of parent compound 88 (0.074 g, 0.23 mmol) in DCM (2.0 ml) was treated with DIPEA (0.046 ml, 0.27 mmol) and methanesulfonyl chloride (0.019 ml, 0.23 mmol). After stirring at ambient temperature for 1.5 hours, the resulting mixture was partitioned between DCM and saturated aqueous sodium bicarbonate solution. The organic phase was dried over magnesium sulfate and concentrated in vacuo. The residue was purified by column chromatography on silica gel, eluting with a mixture of DCM and MeOH (1:0 to 19:1 by volume). Further purification by reverse phase preparative HPLC (Method B) afforded the desired product as a yellow solid (0.048 g, 52%).

LCMS (Method C): Rt=2.46 min, m/z [M+H]⁺=406

¹H NMR (400 MHz, DMSO-d₆) δ ppm: 8.72 (dd, J=1.5, 4.2 Hz, 1H), 8.13 (dd, J=1.5, 8.3 Hz, 1H), 7.14 (dd, J=4.2, 8.3 Hz, 1H), 7.07 (t, J=5.8 Hz, 1H), 6.50 (s, 1H), 3.85-3.77 (m, 2H), 3.75-3.71 (m, 4H), 3.52-3.47 (m, 4H), 2.97-2.86 (m, 7H), 1.88-1.79 (m, 2H), 1.72-1.64 (m, 1H), 1.49-1.36 (m, 2H).

Parent compounds 142 to 144 were prepared according to the reaction protocol of parent compound 141 using the appropriate starting materials (Table 30).

TABLE 30

| Parent Compound | Structure | Starting Materials |
|---|---|---|
| 142 | 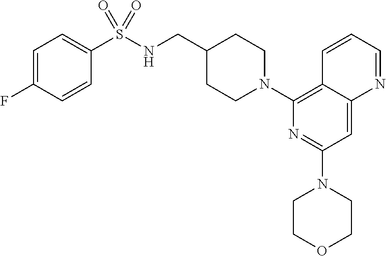 [SMILES: O=S(=O)(NCC1CCN(c2nc(N3CCOCC3)cc3ncccc23)CC1)c1ccc(F)cc1] | a) Parent compound 88 b) 4-Fluorobenzenesulfonyl chloride 2 |
| 143 | 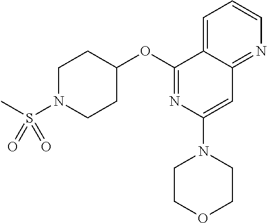 [SMILES: CS(=O)(=O)N1CCC(Oc2nc(N3CCOCC3)cc3ncccc23)CC1] | a) Parent compound 89 b) Methanesulfonyl chloride |
| 144 | 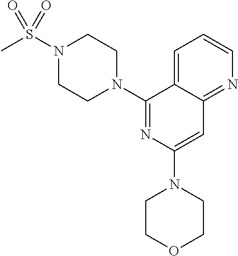 [SMILES: CS(=O)(=O)N1CCN(c2nc(N3CCOCC3)cc3ncccc23)CC1] | a) Parent compound 95 b) Methanesulfonyl chloride |

### Example B20

### a) Preparation of Parent Compound 145

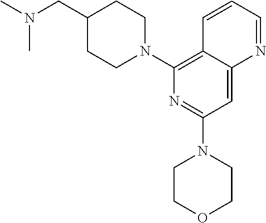 [SMILES: CN(C)CC1CCN(c2nc(N3CCOCC3)cc3ncccc23)CC1]

A solution of parent compound 88 (0.080 g, 0.24 mmol) in DCE (2.0 ml) and MeOH (2.0 ml) at 0° C. was treated with 37% aqueous formaldehyde solution (0.022 ml). After 30 minutes, sodium triacetoxyborohydride (0.10 g, 0.49 mmol) was added and the resulting mixture was stirred at 0° C. for 1.5 hours. A second portion of 37% aqueous formaldehyde solution (0.030 ml) was added, followed by sodium triacetoxyborohydride (0.10 g, 0.49 mmol) and stirring was continued for a further 1 hour. The resulting mixture was partitioned between DCM and saturated aqueous sodium bicarbonate solution. The organic phase was dried over magnesium sulfate and concentrated in vacuo. The residue was purified by reverse phase preparative HPLC (Method B) to afford the desired product as a yellow solid (0.026 g, 30%).

LCMS (Method C): Rt=1.73 min, m/z [M+H]⁺=356

¹H NMR (400 MHz, DMSO-d₆) δ ppm: 8.71 (dd, J=1.5, 4.2 Hz, 1H), 8.12 (dd, J=1.5, 8.3 Hz, 1H), 7.13 (dd, J=4.2, 8.3 Hz, 1H), 6.49 (s, 1H), 3.82-3.76 (m, 2H), 3.76-3.72 (m, 4H), 3.51-3.47 (m, 4H), 3.39-3.27 (m, 2H), 2.96-2.88 (m, 2H), 2.13 (s, 6H), 1.83-1.78 (m, 2H), 1.73-1.66 (m, 1H), 1.42-1.30 (m, 2H).

### Example B21

### a) Preparation of Parent Compound 146

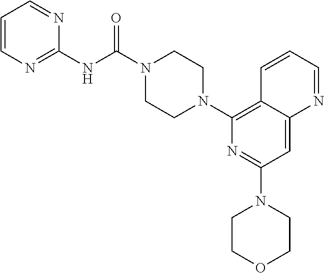 [SMILES: O=C(Nc1ncccn1)N1CCN(c2nc(N3CCOCC3)cc3ncccc23)CC1]

A solution of 2-aminopyrimidine (0.13 g, 1.34 mmol) in THF (10.0 ml) at 0° C. was treated with triphosgene (0.14 g, 0.48 mmol) and DIPEA (0.83 ml, 4.82 mmol). The resulting mixture was stirred at 0° C. for 30 minutes, then warmed to ambient temperature for a further 30 minutes. The mixture was then cooled to 0° C. and parent compound 95 (0.40 g, 1.34 mmol) was added. The resulting mixture was stirred at 0° C. for 1 hour then warmed to ambient temperature. After stirring for a further 48 hours, the mixture was partitioned between EtOAc and water. The organic phase was dried over sodium sulfate and concentrated in vacuo. The residue was purified by column chromatography on silica gel, eluting with a mixture of EtOAc and MeOH (1:0 to 3:2 by volume). Further purification by reverse phase preparative HPLC (Method B) afforded the desired product as a yellow solid (0.088 g, 16%).

LCMS (Method C): Rt=2.04 min, m/z [M+H]⁺=421

¹H NMR (400 MHz, DMSO-d₆) δ ppm: 9.61 (s, 1H), 8.75 (dd, J=1.5, 4.2 Hz, 1H), 8.55 (d, J=4.8 Hz, 2H), 8.26 (dd, J=1.5, 8.4 Hz, 1H), 7.17 (dd, J=4.2, 8.4 Hz, 1H), 7.02 (t, J=4.8 Hz, 1H), 6.58 (s, 1H), 3.76-3.71 (m, 4H), 3.71-3.66 (m, 4H), 3.54-3.49 (m, 4H), 3.41-3.36 (m, 4H).

### Example B22

### a) Preparation of Parent Compound 147

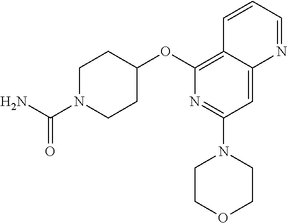 [SMILES: NC(=O)N1CCC(Oc2nc(N3CCOCC3)cc3ncccc23)CC1]

A solution of parent compound 89 (0.050 g, 0.16 mmol) in DCM (2.0 ml) was treated with DIPEA (0.057 ml, 0.32 mmol) at ambient temperature under a nitrogen atmosphere. After 5 minutes, phosgene solution in toluene (20% wt., 0.13 ml, 0.26 mmol) was added and stirring was continued for 2 hours. The resulting mixture was cooled to 0° C. and ammonium hydroxide (2.0 ml) was added. The mixture was warmed to ambient temperature and stirring was continued for a further 18 hours. The resulting mixture was concentrated in vacuo and the residue was purified by reverse phase preparative HPLC (Method B) to afford the desired product as a yellow solid (0.013 g, 23%).

LCMS (Method C): Rt=2.03 min, m/z [M+H]⁺=358

¹H NMR (400 MHz, DMSO-d₆) δ ppm: 8.79 (dd, J=1.5, 4.3 Hz, 1H), 8.31 (dd, J=1.5, 8.2 Hz, 1H), 7.19 (dd, J=4.3, 8.2 Hz, 1H), 6.54 (s, 1H), 5.98 (s, 2H), 5.42-5.34 (m, 1H), 3.77-3.72 (m, 4H), 3.66-3.58 (m, 2H), 3.52-3.47 (m, 4H), 3.30-3.24 (m, 2H), 2.00-1.92 (m, 2H), 1.75-1.64 (m, 2H).

Parent compound 148 was prepared according to the reaction protocol of parent compound 147 using the appropriate starting materials (Table 31).

TABLE 31

| Parent Compound | Structure | Starting Materials |
|---|---|---|
| 148 | 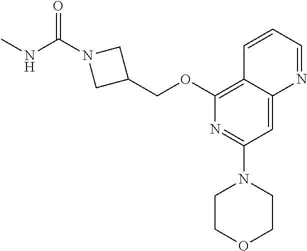 [SMILES: CNC(=O)N1CC(COc2nc(N3CCOCC3)cc3ncccc23)C1] | a) Parent compound 91 b) 2.0M Methylamine in THF |

### Example B23

### a) Preparation of Parent Compound 149

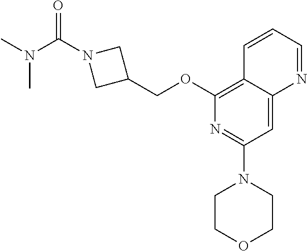 [SMILES: CN(C)C(=O)N1CC(COc2nc(N3CCOCC3)cc3ncccc23)C1]

A solution of parent compound 91 (0.075 g, 0.25 mmol) in DCM (3.0 ml) at 0° C. was treated with triethylamine (0.070 ml, 0.50 mmol) and dimethylcarbamic chloride (0.024 ml, 0.26 mmol) and the resulting mixture was warmed to ambient temperature. After stirring for 18 hours, the resulting mixture was partitioned between DCM and water. The organic phase was dried over magnesium sulfate and concentrated in vacuo. The residue was purified by reverse phase preparative HPLC (Method B) to afford the desired product as a dark yellow solid (0.032 g, 35%).

LCMS (Method C): Rt=2.38 min, m/z [M+H]⁺=372

¹H NMR (400 MHz, DMSO-d₆) δ ppm: 8.79 (dd, J=1.5, 4.3 Hz, 1H), 8.25 (dd, J=1.5, 8.2 Hz, 1H), 7.19 (dd, J=4.3, 8.2 Hz, 1H), 6.55 (s, 1H), 4.55 (d, J=6.0 Hz, 2H), 4.06-4.01 (m, 2H), 3.85-3.81 (m, 2H), 3.77-3.72 (m, 4H), 3.54-3.49 (m, 4H), 3.09-2.98 (m, 1H), 2.76 (s, 6H).

### Example B24

### a) Preparation of Parent Compound 150

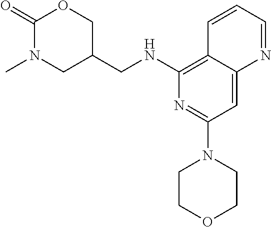 [SMILES: CN1CC(CNc2nc(N3CCOCC3)cc3ncccc23)COC1=O]

A solution of parent compound 97 (0.070 g, 0.20 mmol) in DMF (2.0 ml) at 0° C. was treated with potassium tert-butoxide (0.025 g, 0.22 mmol). After stirring for 5 minutes, methyl iodide (0.015 ml, 0.25 mmol) was added and the resulting mixture was warmed to ambient temperature. After 18 hours, the resulting mixture was concentrated in vacuo and partitioned between water and DCM. The organic phase was dried over magnesium sulfate and concentrated in vacuo. The residue was purified by reverse phase preparative HPLC (Method B) to afford the desired product as a yellow solid (0.019 g, 26%).

LCMS (Method C): Rt=2.03 min, m/z [M+H]⁺=358

¹H NMR (400 MHz, DMSO-d₆) δ ppm: 8.64 (dd, J=1.5, 4.2 Hz, 1H), 8.38 (dd, J=1.5, 8.3 Hz, 1H), 7.73 (t, J=5.6 Hz, 1H), 7.06 (dd, J=4.2, 8.3 Hz, 1H), 6.17 (s, 1H), 4.25 (dd, J=2.8, 10.7 Hz, 1H), 4.08 (dd, J=7.2, 10.7 Hz, 1H), 3.74-3.69 (m, 4H), 3.59-3.36 (m, 7H), 3.17-3.11 (m, 1H), 2.83 (s, 3H). CH hidden by DMSO solvent peak.

### Example B25

### a) Preparation of Intermediate 144

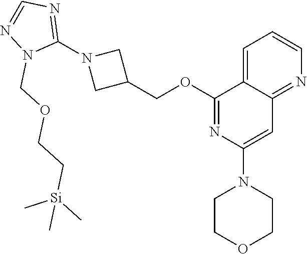 [SMILES: C[Si](C)(C)CCOCn1ncnc1N1CC(COc2nc(N3CCOCC3)cc3ncccc23)C1]

A mixture of parent compound 91 (0.050 g, 0.17 mmol), intermediate 39 (0.046 g, 0.17 mmol), RuPhos Pd G3 (0.014 g, 0.017 mmol), RuPhos (0.008 g, 0.017 mol) and cesium carbonate (0.11 g, 0.33 mmol) in DMF (2.0 ml) was heated at 85° C. for 1.5 hours under an argon atmosphere. The resulting mixture was cooled to ambient temperature and partitioned between EtOAc and water. The organic phase was dried over magnesium sulfate and concentrated in vacuo. The residue was purified by column chromatography on silica gel, eluting with a mixture of DCM and MeOH (1:0 to 19:1 by volume), to afford the desired product as a yellow solid (0.030 g, 36%).

LCMS (Method A): Rt=1.10 min, m/z [M+H]⁺=498

### b) Preparation of Parent Compound 151

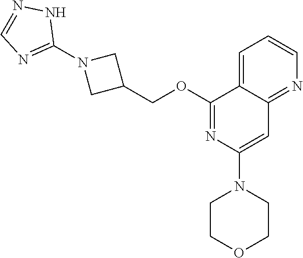 [SMILES: c1cnc2cc(N3CCOCC3)nc(OCC3CN(c4ncn[nH]4)C3)c2c1]

A solution of intermediate 144 in DCM (1.0 ml) was treated with TFA (1.0 ml) and the resulting mixture was stirred at ambient temperature for 18 hours. Additional TFA (0.5 ml) was added and after stirring for a further 2 hours, the resulting mixture was concentrated in vacuo. The residue was purified by ISOLUTE® SCX-2 SPE column, eluting with a mixture of MeOH and 2.0 M ammonia solution in MeOH (1:0 to 0:1 by volume). Further purification by preparative HPLC (Method B) afforded the desired product as a yellow solid (0.002 g, 9%).

LCMS (Method C): Rt=1.94 min, m/z [M+H]⁺=368

¹H NMR (400 MHz, DMSO-d₆) δ ppm: 13.05-12.61 (m, 1H), 8.78 (dd, J=1.5, 4.3 Hz, 1H), 8.06 (dd, J=1.5, 8.2 Hz, 1H), 7.92-7.48 (m, 1H), 7.15 (dd, J=4.3, 8.2 Hz, 1H), 6.55 (s, 1H), 4.62 (d, J=6.0 Hz, 2H), 4.12-4.05 (m, 2H), 3.89-3.83 (m, 2H), 3.76-3.72 (m, 4H), 3.54-3.49 (m, 4H), 3.28-3.22 (m, 1H).

### Example B26

### a) Preparation of Parent Compounds 152 and 153

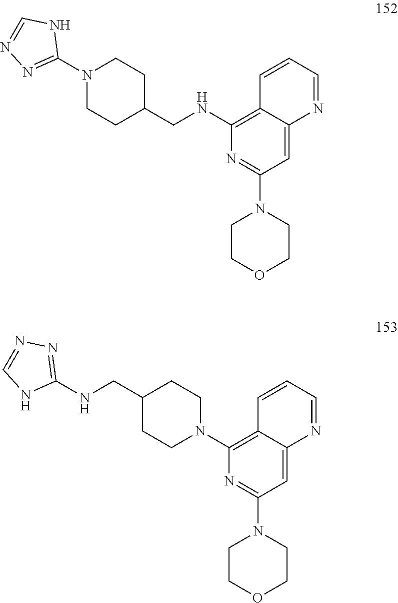 [SMILES: c1cnc2cc(N3CCOCC3)nc(NCC3CCN(c4nnc[nH]4)CC3)c2c1 ; c1cnc2cc(N3CCOCC3)nc(N3CCC(CNc4nnc[nH]4)CC3)c2c1]

A mixture of parent compound 88 (0.080 g, 0.24 mmol) and 3-bromo-4H-1,2,4-triazole (0.036 g, 0.24 mmol) was heated at 150° C. for 18 hours in a sealed tube. The resulting mixture was cooled to ambient temperature, dissolved in DMSO and filtered. Purification by MDAP (Method B) afforded parent compounds 152 as a yellow solid (0.002 g, 2%) and 153 as a yellow solid (0.010 g, 10%).

### Parent Compound 152

LCMS (Method C): Rt=2.10 min, m/z [M+H]⁺=395

¹H NMR (400 MHz, DMSO-d₆) δ ppm: 12.61 (s, 1H), 8.62 (dd, J=1.3, 4.3 Hz, 1H), 8.41 (dd, J=1.3, 8.3 Hz, 1H), 7.71 (s, 1H), 7.62 (t, J=5.5 Hz, 1H), 7.04 (dd, J=4.3, 8.3 Hz, 1H), 6.13 (s, 1H), 3.93-3.85 (m, 2H), 3.74-3.69 (m, 4H), 3.49-3.46 (m, 4H), 2.80-2.73 (m, 2H), 1.93-1.87 (m, 1H), 1.80-1.74 (m, 2H), 1.30-1.19 (m, 2H), CH₂ hidden below water peak.

### Parent Compound 153;

LCMS 859964 (Method C): Rt=1.96 min, m/z [M+H]⁺=395

¹H NMR 1025921 (400 MHz, DMSO-d₆) δ ppm: 12.08 (bs, 1H), 8.71 (dd, J=1.5, 4.2 Hz, 1H), 8.13 (dd, J=1.5, 8.3 Hz, 1H), 7.36 (bs, 1H), 7.13 (dd, J=4.2, 8.3 Hz, 1H), 6.62 (bs, 1H), 6.49 (s, 1H), 3.83-3.80 (m, 2H), 3.75-3.71 (m, 4H), 3.50-3.43 (m, 4H), 3.11-3.07 (m, 2H), 2.94-2.88 (m, 2H), 1.85-1.81 (m, 3H), 1.48-1.41 (m, 2H).

### Example B27

### a) Preparation of Parent Compound 154

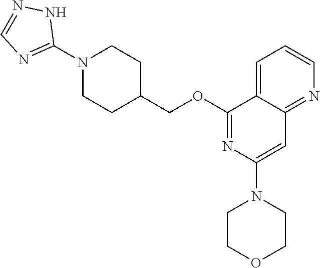 [SMILES: c1cnc2cc(N3CCOCC3)nc(OCC3CCN(c4ncn[nH]4)CC3)c2c1]

A mixture of parent compound 90 (0.10 g, 0.30 mmol) and 3-bromo-4H-1,2,4-triazole (0.045 g, 0.30 mmol) was heated at 150° C. for 18 hours in a sealed tube. The mixture was cooled to ambient temperature and purified by MDAP (Method B) to afford the desired product as a yellow solid (0.014 g, 12%).

LCMS (Method C): Rt=2.19 min, m/z [M+H]⁺=396

¹H NMR (400 MHz, DMSO-d₆) δ ppm: 12.71 (bs, 1H), 8.79 (dd, J=1.5, 4.3 Hz, 1H), 8.29 (dd, J=1.5, 8.2 Hz, 1H), 7.74 (bs, 1H), 7.19 (dd, J=4.3, 8.2 Hz, 1H), 6.53 (s, 1H), 4.33 (d, J=6.4 Hz, 2H), 3.99-3.91 (m, 2H), 3.77-3.72 (m, 4H), 3.53-3.48 (m, 4H), 2.88-2.78 (m, 2H), 2.10-2.00 (m, 1H), 1.89-1.83 (m, 2H), 1.46-1.34 (m, 2H).

Parent compounds 155 to 157 were prepared according to the reaction protocol of parent compound 154 using the appropriate starting materials (Table 32).

TABLE 32

| Parent Compound | Structure | Starting Materials |
|---|---|---|
| 155 | 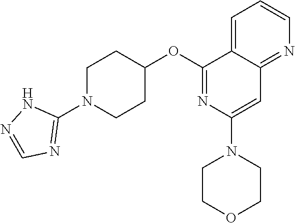 [SMILES: c1cnc2cc(N3CCOCC3)nc(OC3CCN(c4ncn[nH]4)CC3)c2c1] | a) Parent compound 89 b) 3-Bromo-4H-1,2,4-triazole |
| 156 | 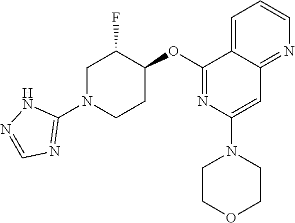 [SMILES: F[C@H]1CN(c2ncn[nH]2)CC[C@@H]1Oc1nc(N2CCOCC2)cc2ncccc12] | a) Parent compound 93 b) 3-Bromo-4H-1,2,4-triazole |
| 157 | 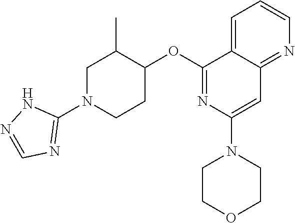 [SMILES: CC1CN(c2ncn[nH]2)CCC1Oc1nc(N2CCOCC2)cc2ncccc12] | a) Parent compound 92 b) 3-Bromo-4H-1,2,4-triazole 3:2 mixture of trans:cis diastereomers. |

### Example B28

### a) Preparation of Parent Compounds 158 and 159

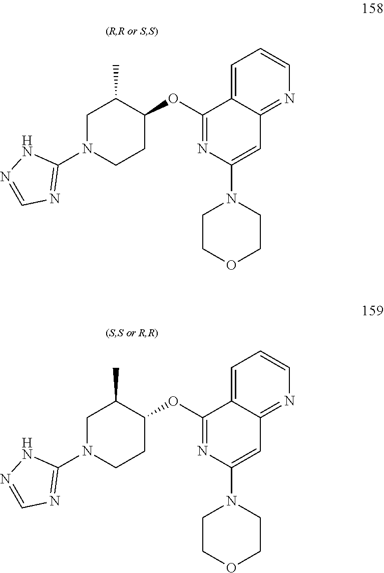 [SMILES: C[C@H]1CN(c2ncn[nH]2)CC[C@@H]1Oc1nc(N2CCOCC2)cc2ncccc12 ; C[C@@H]1CN(c2ncn[nH]2)CC[C@H]1Oc1nc(N2CCOCC2)cc2ncccc12]

Parent compound 157 (0.075 g, 0.19 mmol) was purified by chiral preparative SFC with the following conditions: YMC Amylose-C, 15/85 EtOH (0.1% DEA)/CO₂, 100 ml/min, 120 bar, 40 C. This afforded parent compound 158 (2^{nd} eluting, trans isomer; R,R or S,S) as a yellow solid (0.009 g, 12%) and parent compound 159 (3^{rd} eluting, trans isomer; S,S or R,R) as a yellow solid (0.011 g, 15%).

### Parent Compound 158

LCMS (Method C): Rt=2.22 min, m/z [M+H]⁺=396

¹H NMR (400 MHz, DMSO-d₆) δ ppm: 12.82 (bs, 1H), 8.79 (dd, J=1.5, 4.3 Hz, 1H), 8.29 (dd, J=1.5, 8.3 Hz, 1H), 7.82 (bs, 1H), 7.18 (dd, J=4.3, 8.2 Hz, 1H), 6.54 (s, 1H), 5.08-5.01 (m, 1H), 3.94-3.86 (m, 2H), 3.78-3.73 (m, 4H), 3.53-3.47 (m, 4H), 3.17-3.11 (m, 1H), 2.86 (dd, J=10.7, 12.0 Hz, 1H), 2.30-2.22 (m, 1H), 2.10-2.00 (m, 1H), 1.68-1.53 (m, 1H), 0.99 (d, J=6.6 Hz, 3H).

### Parent Compound 159

LCMS (Method C): Rt=2.22 min, m/z [M+H]⁺=396

¹H NMR (400 MHz, DMSO-d₆) δ ppm: 12.78 (bs, 1H), 8.78 (dd, J=1.5, 4.3 Hz, 1H), 8.28 (dd, J=1.5, 8.2 Hz, 1H), 7.81 (bs, 1H), 7.18 (dd, J=4.3, 8.2 Hz, 1H), 6.54 (s, 1H), 5.09-5.01 (m, 1H), 3.94-3.85 (m, 2H), 3.78-3.73 (m, 4H), 3.53-3.47 (m, 4H), 3.17-3.09 (m, 1H), 2.89-2.81 (m, 1H), 2.29-2.22 (m, 1H), 2.09-1.97 (m, 1H), 1.69-1.53 (m, 1H), 0.99 (d, J=6.6 Hz, 3H).

### b) Preparation of Parent Compound 160

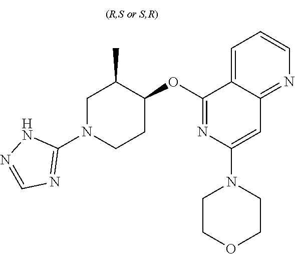 [SMILES: C[C@@H]1CN(c2ncn[nH]2)CC[C@@H]1Oc1nc(N2CCOCC2)cc2ncccc12]

Parent compound 157 (0.034 g, 0.19 mmol) was purified by chiral preparative SFC with the following conditions: LUX-Cellulose-4, 40/60 IPA (0.1% DEA)/CO₂, 15 ml/min, 120 bar, 40 C.

This afforded parent compound 160 (2^{nd} eluting, cis isomer; R,S or S,R) as a yellow solid (0.011 g, 32%).

LCMS (Method E): Rt=2.26 min, m/z [M+H]⁺=396

¹H NMR (400 MHz, DMSO-d₆) δ ppm: 12.76 (s, 1H), 8.79 (dd, J=1.5, 4.3 Hz, 1H), 8.37 (dd, J=1.5, 8.2 Hz, 1H), 7.78 (s, 1H), 7.19 (dd, J=4.3, 8.2 Hz, 1H), 6.54 (s, 1H), 5.48-5.45 (m, 1H), 3.77-3.72 (m, 4H), 3.72-3.60 (m, 2H), 3.53-3.48 (m, 4H), 3.26-3.16 (m, 2H), 2.19-2.08 (m, 2H), 1.93-1.84 (m, 1H), 0.97 (d, J=6.8 Hz, 3H).

### c) Preparation of Parent Compound 161

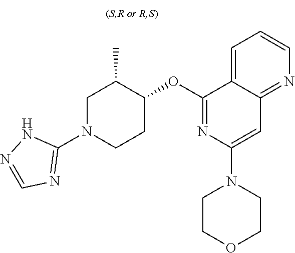 [SMILES: C[C@H]1CN(c2ncn[nH]2)CC[C@H]1Oc1nc(N2CCOCC2)cc2ncccc12]

Parent compound 157 (0.015 g, 0.38 mmol) was purified by chiral preparative SFC with the following conditions: YMC Amylose-C, 15/85 EtOH (0.1% DEA)/CO₂, 100 ml/min, 120 bar, 40 C. This afforded parent compound 161 (4^{th} eluting, cis isomer; S,R or R,S) as a yellow solid (0.09 g, 60%).

LCMS (Method E): Rt=2.26 min, m/z [M+H]⁺=396

¹H NMR (400 MHz, DMSO-d₆) δ ppm: 12.73 (bs, 1H), 8.79 (dd, J=1.5, 4.3 Hz, 1H), 8.37 (dd, J=1.5, 8.2 Hz, 1H), 7.78 (bs, 1H), 7.19 (dd, J=4.3, 8.2 Hz, 1H), 6.54 (s, 1H), 5.48-5.45 (m, 1H), 3.77-3.72 (m, 4H), 3.72-3.61 (m, 2H), 3.53-3.48 (m, 4H), 3.26-3.15 (m, 2H), 2.19-2.08 (m, 2H), 1.94-1.85 (m, 1H), 0.97 (d, J=6.8 Hz, 3H).

### Example B29

### a) Preparation of Parent Compound 162

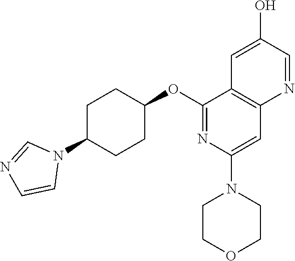 [SMILES: Oc1cnc2cc(N3CCOCC3)nc(O[C@H]3CC[C@@H](n4ccnc4)CC3)c2c1]

A solution of parent compound 54 (0.13 g, 0.27 mmol), tBuBrettPhos Pd G3 (0.012 g, 0.014 mmol) and tBuBrettPhos (0.007 g, 0.014 mmol) in dioxane (0.5 ml) was treated with a solution of potassium hydroxide (0.045 g, 0.83 mmol) in water (0.099 ml) under an argon atmosphere. The resulting mixture was heated at 80° C. for 24 hours. A second portion of tBuBrettPhos Pd G3 (0.012 g, 0.014 mmol) and tBuBrettPhos (0.007 g, 0.014 mmol) were added and heating was continued for 5 hours at 80° C., followed by a further 2 hours at 90° C. The resulting mixture was cooled to ambient temperature and concentrated in vacuo. The residue was purified on a Biotage® KP-NH column, eluting with a mixture of EtOAc and MeOH (1:0 to 1:1 by volume), to afford the desired product as a yellow solid (0.056 g, 53%).

LCMS (Method D): Rt=2.59 min, m/z [M+H]⁺=396

¹H NMR (400 MHz, DMSO-d₆) δ ppm: 10.09 (bs, 1H), 8.53 (d, J=2.8 Hz, 1H), 7.76-7.74 (m, 1H), 7.66 (d, J=2.8 Hz, 1H), 7.28-7.26 (m, 1H), 6.92-6.91 (m, 1H), 6.50 (s, 1H), 5.48-5.43 (m, 1H), 4.28-4.19 (m, 1H), 3.77-3.72 (m, 4H), 3.42-3.37 (m, 4H), 2.22-2.13 (m, 2H), 2.11-1.94 (m, 4H), 1.89-1.80 (m, 2H).

Parent compounds 163 to 168 were prepared according to the reaction protocol of parent compound 162 using the appropriate starting materials (Table 33).

TABLE 33

| Parent Compound | Structure | Starting Materials |
|---|---|---|
| 163 | 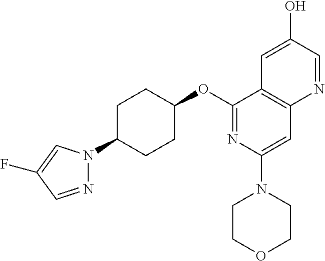 [SMILES: Oc1cnc2cc(N3CCOCC3)nc(O[C@H]3CC[C@@H](n4cc(F)cn4)CC3)c2c1] | Parent compound 55 |
| 164 | 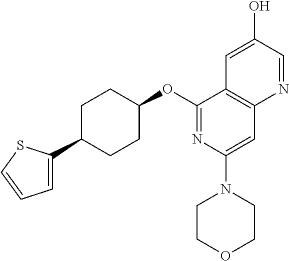 [SMILES: Oc1cnc2cc(N3CCOCC3)nc(O[C@H]3CC[C@@H](c4cccs4)CC3)c2c1] | Parent compound 60 |
| 165 | 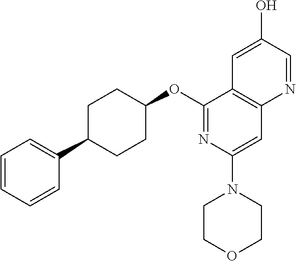 [SMILES: Oc1cnc2cc(N3CCOCC3)nc(O[C@H]3CC[C@@H](c4ccccc4)CC3)c2c1] | Parent compound 61 |
| 166 | 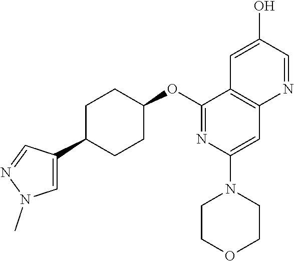 [SMILES: Cn1cc([C@H]2CC[C@@H](Oc3nc(N4CCOCC4)cc4ncc(O)cc34)CC2)cn1] | Parent compound 59 |
| 167 | 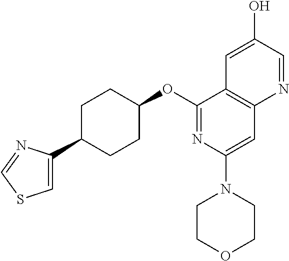 [SMILES: Oc1cnc2cc(N3CCOCC3)nc(O[C@H]3CC[C@@H](c4cscn4)CC3)c2c1] | Parent compound 62 |
| 168 | 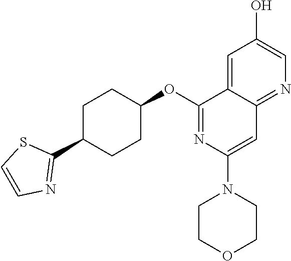 [SMILES: Oc1cnc2cc(N3CCOCC3)nc(O[C@H]3CC[C@@H](c4nccs4)CC3)c2c1] | Parent compound 63 |

### Example B30

### a) Preparation of Intermediate 145

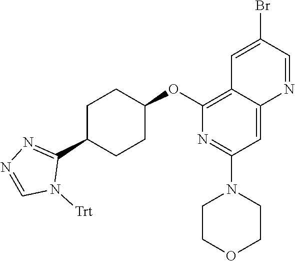

A solution of parent compound 64 (0.21 g, 0.46 mmol) in chloroform (5.0 ml) was treated with DIPEA (0.10 ml, 0.60 mmol) and trityl chloride (0.14 g, 0.50 mmol). After stirring at ambient temperature for 1 hour, the resulting mixture was concentrated in vacuo. The residue was purified on a Biotage® KP-NH column, eluting with a mixture of isohexane and EtOAc (1:0 to 3:2 by volume), to afford the desired product as a yellow solid (0.23 g, 71%).

LCMS (Method A): Rt=2.05 min, m/z [M+H]⁺=701/703

### b) Preparation of Intermediate 146

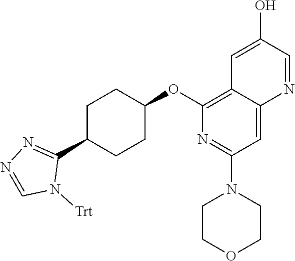

A solution of intermediate 145 (0.23 g, 0.33 mmol), tBuBrettPhos Pd G3 (0.008 g, 0.016 mmol) and tBuBrettPhos (0.014 g, 0.016 mmol) in dioxane (0.7 ml) was treated with a solution of potassium hydroxide (0.054 g, 0.98 mmol) in water (0.12 ml) under an argon atmosphere. The resulting mixture was heated at 80° C. for 18 hours. A second portion of tBuBrettPhos Pd G3 (0.004 g, 0.008 mmol) and tBuBrettPhos (0.007 g, 0.008 mmol) were added and heating was continued for a further 2 hours at 80° C. The resulting mixture was cooled to ambient temperature and concentrated in vacuo. The residue was purified on a Biotage® KP-NH column, eluting with a mixture of EtOAc and MeOH (1:0 to 1:1 by volume), to afford the desired product as a yellow foam (0.14 g, 66%).

LCMS (Method A): Rt=1.53 min, m/z [M+H]⁺=639

### c) Preparation of Parent Compound 169

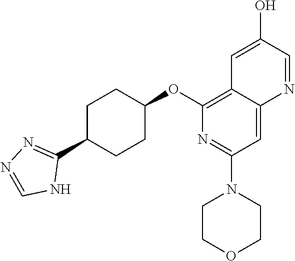 [SMILES: Oc1cnc2cc(N3CCOCC3)nc(O[C@H]3CC[C@@H](c4nnc[nH]4)CC3)c2c1]

A stirred solution of intermediate 146 (0.098 g, 0.15 mmol) in DCM (5.0 ml) was treated with triethylsilane (0.10 ml, 1.35 mmol) and TFA (0.20 ml, 1.25 mmol). After stirring at ambient temperature for 30 minutes, the reaction mixture was treated with DIPEA (0.50 ml, 2.87 mmol) and stirring was continued for a further 10 minutes. The resulting mixture was concentrated in vacuo and the residue was purified on a Biotage® KP-NH column, eluting with a mixture of EtOAc and MeOH (1:0 to 1:1 by volume), to afford the desired product as a yellow solid (0.038 g, 62%).

LCMS (Method C): Rt=2.48 min, m/z [M+H]⁺=397

¹H NMR 1027589 (400 MHz, DMSO-d₆) δ ppm: 13.67 (bs, 1H), 10.04 (bs, 1H), 8.52 (d, J=2.8 Hz, 1H), 8.04 (bs, 1H), 7.59 (d, J=2.8 Hz, 1H), 6.48 (s, 1H), 5.47-5.41 (m, 1H), 3.77-3.72 (m, 4H), 3.43-3.37 (m, 4H), 2.97-2.87 (m, 1H), 2.14-2.06 (m, 2H), 2.00-1.77 (m, 6H).

### Example B31

### a) Preparation of Compound 170

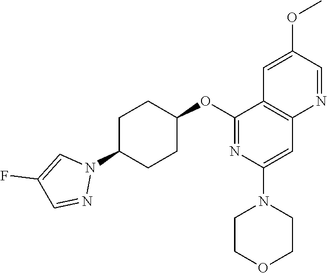 [SMILES: COc1cnc2cc(N3CCOCC3)nc(O[C@H]3CC[C@@H](n4cc(F)cn4)CC3)c2c1]

A suspension of parent compound 163 (0.020 g, 0.048 mmol) and potassium carbonate (0.013 g, 0.097 mmol) in DMF (1.0 ml) was treated with iodomethane (0.003 ml, 0.053 mmol). After stirring at ambient temperature for 1 hour, a second portion of iodomethane (0.002 ml, 0.025 mmol) was added and stirring was continued for a further 1 hour. The resulting mixture was partitioned between EtOAc and saturated aqueous sodium bicarbonate solution. The organic phase was dried over sodium sulfate and concentrated in vacuo. The residue was purified by ISOLUTE® SCX-2 SPE column, eluting with a mixture of MeOH and 2.0 M ammonia solution in MeOH (1:0 to 0:1 by volume). Further purification by column chromatography on silica gel, eluting with a mixture of DCM and 2.0 M ammonia solution in MeOH (1:0 to 24:1 by volume), afforded the desired product as a yellow solid (0.018 g, 88%).

LCMS (Method C): Rt=4.08 min, m/z [M+H]⁺=428

¹H NMR (400 MHz, DMSO-d₆) δ ppm: 8.64 (d, J=3.0 Hz, 1H), 8.00 (d, J=4.6 Hz, 1H), 7.66 (d, J=3.0 Hz, 1H), 7.47 (d, J=4.4 Hz, 1H), 6.56 (s, 1H), 5.45-5.40 (m, 1H), 4.27-4.22 (m, 1H), 3.92 (s, 3H), 3.77-3.72 (m, 4H), 3.46-3.41 (m, 4H), 2.21-2.13 (m, 4H), 1.95-1.81 (m, 4H).

### Example B32

### a) Preparation of Parent Compound 171

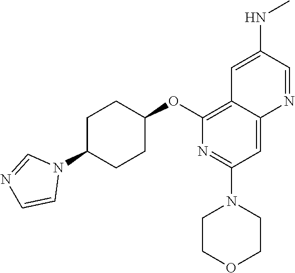 [SMILES: CNc1cnc2cc(N3CCOCC3)nc(O[C@H]3CC[C@@H](n4ccnc4)CC3)c2c1]

A solution of parent compound 54 (0.015 g, 0.033 mmol) and tBuBrettPhos Pd G3 (0.003 g, 0.0033 mmol) in 2.0 M methylamine solution in THF (0.025 ml, 0.050 mmol) was treated with 1.5 M lithium bis(trimethylsilyl)amide solution in THF (0.055 ml, 0.083 mmol). After stirring at ambient temperature for 1 hour, the resulting mixture was concentrated in vacuo. The residue was purified by reverse phase preparative HPLC (Method B) to afford the desired product as a yellow solid (0.002 g, 16%).

LCMS (Method C): Rt=2.05 min, m/z [M+H]⁺=409

¹H NMR (400 MHz, DMSO-d₆) δ ppm: 8.46 (d, J=2.9 Hz, 1H), 7.75-7.74 (m, 1H), 7.26-7.25 (m, 1H), 7.12 (d, J=2.8 Hz, 1H), 6.92-6.91 (m, 1H), 6.45 (s, 1H), 6.12 (q, J=5.0 Hz, 1H), 5.45-5.42 (m, 1H), 4.28-4.20 (m, 1H), 3.77-3.72 (m, 4H), 3.39-3.34 (m, 4H), 2.78 (d, J=5.0 Hz, 3H), 2.21-2.14 (m, 2H), 2.11-2.02 (m, 2H), 1.96-1.92 (m, 2H), 1.87-1.80 (m, 2H).

### Example B33

### a) Preparation of Parent Compound 172

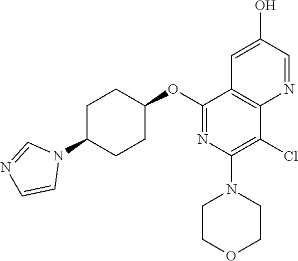 [SMILES: Oc1cnc2c(Cl)c(N3CCOCC3)nc(O[C@H]3CC[C@@H](n4ccnc4)CC3)c2c1]

A solution of parent compound 162 (0.020 g, 0.051 mmol) in DCE (1.0 ml) was treated with N-chlorosuccinimide (0.008 g, 0.061 mmol). After stirring at ambient temperature for 1 hour, the resulting mixture was partitioned between water and DCM. The organic phase was dried over sodium sulfate and concentrated in vacuo. The residue was purified by column chromatography on silica gel, eluting with a mixture of DCM and 2.0 M ammonia solution in MeOH (1:0 to 9:1 by volume), to afford the desired product as a pale yellow solid (0.003 g, 13%).

LCMS (Method C): Rt=2.73 min, m/z [M+H]⁺=430

¹H NMR (400 MHz, DMSO-d₆) δ ppm: 10.57 (s, 1H), 8.72 (d, J=2.9 Hz, 1H), 7.78 (d, J=2.9 Hz, 1H), 7.77-7.75 (m, 1H), 7.29-7.27 (m, 1H), 6.93-6.91 (m, 1H), 5.50-5.45 (m, 1H), 4.29-4.20 (m, 1H), 3.79-3.76 (m, 4H), 3.31-3.27 (m, 4H), 2.25-2.17 (m, 2H), 2.08-1.95 (m, 4H), 1.91-1.82 (m, 2H).

### Example B34

### a) Preparation of Parent Compound 173

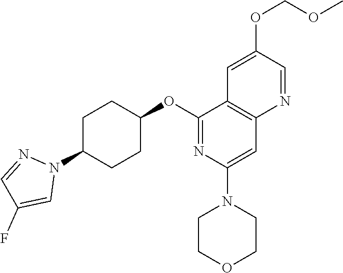 [SMILES: COCOc1cnc2cc(N3CCOCC3)nc(O[C@H]3CC[C@@H](n4cc(F)cn4)CC3)c2c1]

A suspension of parent compound 163 (0.041 g, 0.10 mmol), chloromethyl methyl ether (0.014 g, 0.17 mmol) and potassium carbonate (0.028 g, 0.20 mmol) in DMF (0.5 ml) was stirred at ambient temperature for 18 hours. The resulting mixture was partitioned between EtOAc and water. The organic phase was dried over sodium sulfate and concentrated in vacuo. The residue was purified on a Biotage® KP-NH column, eluting with a mixture of isohexane and EtOAc (1:0 to 0:1 by volume), to afford the desired product as a yellow oil (0.040 g, 89%).

¹H NMR (400 MHz, CDCl₃) δ ppm: 8.68 (d, J=2.9 Hz, 1H), 7.94 (dd, J=0.7, 2.9 Hz, 1H), 7.41 (dd, J=0.7, 4.6 Hz, 1H), 7.33 (dd, J=0.7, 4.4 Hz, 1H), 6.57 (d, J=0.7 Hz, 1H), 5.55-5.50 (m, 1H), 5.29 (s, 2H), 4.26-4.17 (m, 1H), 3.89-3.85 (m, 4H), 3.55 (s, 3H), 3.52-3.48 (m, 4H), 2.38-2.30 (m, 2H), 2.22-2.05 (m, 4H), 1.86-1.76 (m, 2H).

LCMS (Method A): Rt=1.23 min, m/z [M+H]⁺=458

### Example B35

### a) Preparation of Parent Compound 174

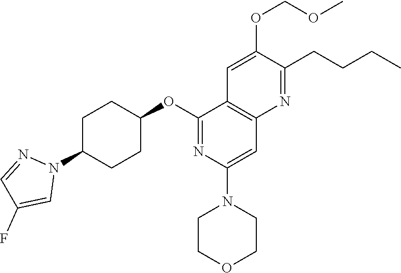 [SMILES: CCCCc1nc2cc(N3CCOCC3)nc(O[C@H]3CC[C@@H](n4cc(F)cn4)CC3)c2cc1OCOC]

A solution of parent compound 173 (0.040 g, 0.090 mmol) in anhydrous THF (0.5 ml) at −78° C. under an argon atmosphere was treated with a 2.5 M solution of n-butyllithium in hexanes (0.087 ml, 0.18 mmol). After stirring for 30 minutes, N-fluorobenzenesulfonimide (0.055 g, 0.18 mmol) was added and the resulting mixture was warmed to ambient temperature. After stirring for 18 hours, the resulting mixture was partitioned between EtOAc and water. The organic phase was dried over sodium sulfate and concentrated in vacuo. The residue was purified on a Biotage® KP-NH column, eluting with a mixture of isohexane and EtOAc (1:0 to 2:1 by volume), to afford the desired product as a yellow oil (0.021 g, 50%).

LCMS (Method A): Rt=1.34 min, m/z [M+H]⁺=514

¹H NMR (400 MHz, CDCl₃) δ ppm: 7.94-7.88 (m, 1H), 7.42 (dd, J=0.6, 4.6 Hz, 1H), 7.32 (dd, J=0.6, 4.4 Hz, 1H), 6.58 (s, 1H), 5.54-5.49 (m, 1H), 5.32 (s, 2H), 4.27-4.17 (m, 1H), 3.88-3.84 (m, 4H), 3.54 (s, 3H), 3.51-3.46 (m, 4H), 2.98 (t, J=7.6 Hz, 2H), 2.36-2.28 (m, 2H), 2.23-2.02 (m, 4H), 1.83-1.69 (m, 4H), 1.50-1.42 (m, 2H), 0.96 (t, J=7.3 Hz, 3H).

### Example B36

### a) Preparation of Parent Compound 175

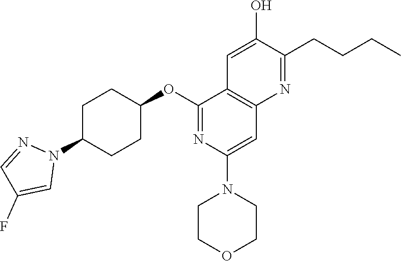 [SMILES: CCCCc1nc2cc(N3CCOCC3)nc(O[C@H]3CC[C@@H](n4cc(F)cn4)CC3)c2cc1O]

A mixture of parent compound 174 (0.020 g, 0.030 mmol) and concentrated HCl (0.1 ml) in MeOH (2.0 ml) was heated at 65° C. for 2 hours. The resulting mixture was cooled to ambient temperature and purified by ISOLUTE® SCX-2 SPE column, eluting with a mixture of MeOH and 2.0 M ammonia solution in MeOH (1:0 to 0:1 by volume). Further purification by reverse phase preparative HPLC (Method B) afforded the desired product as a yellow solid (0.007 g, 38%).

LCMS (Method C): Rt=3.68 min, m/z [M+H]⁺=470

¹H NMR (400 MHz, DMSO-d₆) δ ppm: 9.99 (s, 1H), 7.93 (dd, J=0.6, 4.6 Hz, 1H), 7.58 (s, 1H), 7.48 (dd, J=0.6, 4.4 Hz, 1H), 6.45 (s, 1H), 5.43-5.38 (m, 1H), 4.26-4.19 (m, 1H), 3.76-3.71 (m, 4H), 3.41-3.36 (m, 4H), 2.85-2.79 (m, 2H), 2.18-2.04 (m, 4H), 1.99-1.91 (m, 2H), 1.89-1.78 (m, 2H), 1.72-1.63 (m, 2H), 1.40-1.32 (m, 2H), 0.91 (t, J=7.4 Hz, 3H).

### Preparation of Trigger Moiety Precursors (‘Triggers’)

### Example C1

### a) Preparation of Intermediate 147

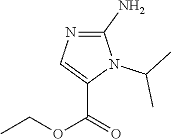

A stirred solution of ethyl isopropylglycinate (5.07 g, 34.9 mmol) in ethyl formate (30 ml) at 0° C. under a nitrogen atmosphere was treated portionwise with sodium hydride (2.09 g, 52.5 mmol, 60% in mineral oil) over 15 minutes. The mixture was warmed to ambient temperature and stirring was continued for 18 hours. The resulting mixture was concentrated in vacuo and the residue was triturated with hexane. After decanting the hexane layer, the remaining residue was taken up in EtOH (20 ml) and treated with concentrated HCl (3.0 ml). The resulting solution was heated at reflux for 1.5 hours, then cooled to ambient temperature, filtered and the filtrate was concentrated in vacuo. The residue was taken up in 10% aqueous acetic acid (50 ml) and treated with cyanamide (2.93 g, 69.6 mmol) and sodium acetate (5.72 g, 69.7 mmol). The resulting solution was heated at 100° C. for 1.5 hours. The mixture was then cooled to 5° C. and acidified with concentrated HCl to pH 1. The resulting mixture was treated portionwise with potassium carbonate until pH 8-9 and partitioned between EtOAc and water. The organic phase was dried over sodium sulfate and concentrated in vacuo. The residue was purified by column chromatography on silica gel, eluting with a mixture of DCM and 2.0 M ammonia solution in MeOH (1:0 to 15:1 by volume), to afford the desired product as a brown gum (0.46 g, 7%).

LCMS (Method A): Rt=0.68 min, m/z [M+H]⁺=198

Intermediate 148 was prepared according to the reaction protocol of intermediate 147 using the appropriate starting materials (Table 34).

TABLE 34

| Inter- | | Starting | LCMS |
|---|---|---|---|
| mediate | Structure | Materials | Data |
| 148 | 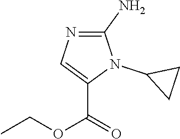 | a) Cyclopropyl glycinate 8 | Rt = 0.61 min, m/z [M + H]⁺ = 196 (Method A) |

### b) Preparation of Intermediate 149

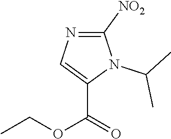 [SMILES: CCOC(=O)c1cnc([N+](=O)[O-])n1C(C)C]

A stirred mixture of intermediate 147 (0.55 g, 2.77 mmol) in acetic acid (3.0 ml) at 0° C. was treated dropwise with a solution of sodium nitrite (1.26 g, 18.3 mmol) in water (3.0 ml). The mixture was warmed to ambient temperature and stirring was continued for 2 hours. The resulting mixture was partitioned between DCM and water. The organic phase was dried over sodium sulfate and concentrated in vacuo. The residue was purified by column chromatography on silica gel, eluting with a mixture of DCM and MeOH (1:0 to 7:3 by volume), to afford the desired product as a yellow gum (0.16 g, 25%).

¹H NMR (300 MHz, CDCl₃) δ ppm: 7.69 (s, 1H), 5.57 (hept, J=7.0 Hz, 1H), 4.39 (q, J=7.1 Hz, 2H), 1.65 (d, J=7.0 Hz, 6H), 1.41 (t, J=7.1 Hz, 3H).

Intermediates 150 to 153 were prepared according to the reaction protocol of intermediate 149 using the appropriate starting materials (Table 35).

TABLE 35

| Inter- | | Starting | Analytical |
|---|---|---|---|
| mediate | Structure | Materials | Data |
| 150 | 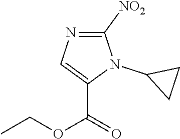 [SMILES: CCOC(=O)c1cnc([N+](=O)[O-])n1C1CC1] | a) Intermediate 148 | LCMS (Method A) Rt = 1.50 min, m/z [M + H]⁺ = 226 |
| 151 | 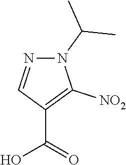 | a) 5-Amino-1- isopropyl-1H- pyrazole-4- carboxylic acid | ¹H NMR (300 MHz, CDCl₃) δ ppm: 13.47 (s, 1H), 8.04 (s, 1H), 4.75 (hept, J = 6.5 Hz, 1H), 1.44 (d, J = 6.5 Hz, 6H). |
| 152 | 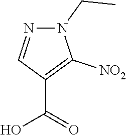 | a) 1 -Ethyl-5- nitro-1 H- pyrazole-4- carboxylic acid | ¹H NMR (300 MHz, CDCl₃) δ ppm: 7.97 (s, 1H), 4.39 (q, J = 7.5 Hz, 2H), 1.53 (t, J = 7.5 Hz, 3H). |
| 153 | 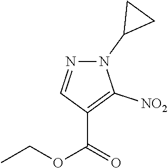 [SMILES: CCOC(=O)c1cnn(C2CC2)c1[N+](=O)[O-]] | a) Ethyl 1- cyclopropyl- 5-nitro-1H- pyrazole- 4-carboxylate | LCMS (Method A) Rt = 1.71 min, m/z [M + H]⁺ = 226 |

### c) Preparation of Intermediate 154

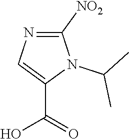

A stirred solution of intermediate 149 (0.12 g, 0.52 mmol) in EtOH (0.5 ml) at ambient temperature was treated dropwise with a solution of sodium hydroxide (0.21 g, 5.15 mmol) in water (0.5 ml). After stirring for 2 hours, the resulting mixture was acidified to pH 3 by addition of 2.0 M aqueous HCl and partitioned between EtOAc and water. The organic phase was dried over sodium sulfate and concentrated in vacuo to afford the desired product as a white solid (0.097 g, 94%).

LCMS (Method A): Rt=1.10 min, m/z=198

Intermediate 155 was prepared according to the reaction protocol of intermediate 154 using the appropriate starting materials (Table 36).

TABLE 36

| | | Starting | |
|---|---|---|---|
| Intermediate | Structure | Materials | LCMS Data |
| 155 | 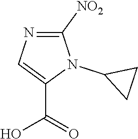 [SMILES: O=C(O)c1cnc([N+](=O)[O-])n1C1CC1] | a) Intermediate 150 | Rt = 0.99 min, m/z [M + H]⁺ = 198 (Method A) |

### d) Preparation of Trigger 1

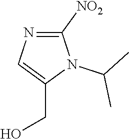

A stirred solution of intermediate 154 (0.065 g, 0.33 mmol) and triethylamine (0.045 ml, 0.65 mmol) in anhydrous THF (1.5 ml) under an argon atmosphere at −10° C. was treated with isobutyl chloroformate (0.085 ml, 0.65 mmol). After stirring at −5° C. for 30 minutes, the resulting mixture was treated with sodium borohydride (0.065 g, 1.71 mmol). Water (1.3 ml) was then carefully added and stirring was continued for 10 minutes. The resulting mixture was partitioned between EtOAc and water. The organic phase was dried over sodium sulfate and concentrated in vacuo. The residue was purified by column chromatography on silica gel, eluting with a mixture of isohexane and EtOAc (1:0 to 0:1 by volume), to afford the desired product as a colourless oil, which solidified on standing (0.040 g, 67%).

LCMS (Method A): Rt=0.93 min, m/z [M+H]⁺=186

### Example C2

### Preparation of Trigger 2

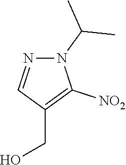

A stirred solution of intermediate 151 (0.27 g, 1.34 mmol) in anhydrous THF (9.0 ml) at ambient temperature was treated with borane dimethylsulfide (0.15 ml, 1.54 mmol). The resulting mixture was heated at reflux for 1.5 hours. The mixture was cooled to ambient temperature and treated sequentially with MeOH (0.7 ml), water (0.7 ml) and 1.0 M HCl. After stirring at ambient temperature for 10 minutes, the resulting mixture was partitioned between EtOAc and water. The organic phase was dried over sodium sulfate and concentrated in vacuo to afford the desired product as a pale yellow oil (0.18 g, 73%).

LCMS (Method A): Rt=1.01 min, m/z [M+H]⁺=186

Trigger 3 was prepared according to the reaction protocol of trigger 2 using the appropriate starting materials (Table 37).

TABLE 37

| | | Starting | |
|---|---|---|---|
| Trigger | Structure | Materials | LCMS Data |
| 3 | 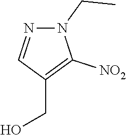 | a) Intermediate 152 | Rt = 0.95 min, m/z [M + H]⁺ = 172 (Method A) |

### Example C3

### Preparation of Intermediate 156

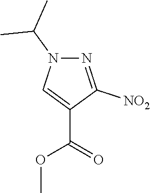

A stirred mixture of methyl 3-nitro-1H-pyrazole-4-carboxylate (0.25 g, 1.46 mmol) and potassium carbonate (0.61 g, 4.38 mmol) in DMF (5.0 ml) under an argon atmosphere was treated dropwise with 2-bromopropane (0.21 ml, 2.19 mmol). The resulting mixture was stirred at 50° C. for 2.5 hours. The mixture was cooled to ambient temperature and partitioned between EtOAc and saturated aqueous sodium bicarbonate solution. The organic phase was dried over sodium sulfate and concentrated in vacuo. The residue was purified by column chromatography on silica gel, eluting with a mixture of isohexane and EtOAc (1:0 to 0:1 by volume), to afford the desired product as a colourless oil (0.28 g, 90%).

¹H NMR (300 MHz, CDCl₃) δ ppm: 7.93 (s, 1H), 4.55 (hept, J=6.6 Hz, 1H), 3.87 (s, 3H), 1.57 (d, J=6.6 Hz, 6H).

Preparation of Trigger 4

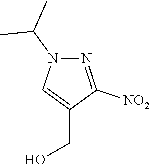

A stirred solution of intermediate 156 (0.14 g, 0.66 mmol) in MeOH (2.0 ml) at 0° C. was treated with sodium borohydride (0.050 g, 1.31 mmol) and the resulting mixture was stirred at ambient temperature for 18 hours. A second portion of sodium borohydride (0.075 g, 1.98 mmol) was added and the mixture was stirred at 50° C. for 2 hours. A third portion of sodium borohydride (0.075 g, 1.98 mmol) was added and stirring was continued at 50° C. for a further 2 hours. The resulting mixture was cooled to 0° C. and partitioned between EtOAc and water. The organic phase was dried over sodium sulfate and concentrated in vacuo. The residue was purified by column chromatography on silica gel, eluting with a mixture of isohexane and EtOAc (1:0 to 0:1 by volume), to afford the desired product as a white solid (0.040 g, 33%).

¹H NMR (400 MHz, CDCl₃) δ ppm: 7.54 (s, 1H), 4.82 (s, 2H), 4.57 (hept, J=6.7 Hz, 1H), 2.48 (s, 1H), 1.56 (d, J=6.7 Hz, 6H).

### Example C4

### Preparation of Intermediate 157

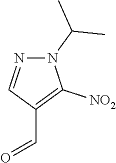

A stirred solution of trigger 2 (0.15 g, 0.81 mmol) in DCM (5.0 ml) was treated with Dess-Martin periodinane (0.41 g, 0.97 mmol). After stirring for 1.5 hours at ambient temperature, the resulting mixture was partitioned between DCM and saturated aqueous sodium bicarbonate solution. The organic phase was dried over magnesium sulfate and concentrated in vacuo. The residue was purified by column chromatography on silica gel, eluting with a mixture of isohexane and EtOAc (1:0 to 1:1 by volume), to afford the desired product as a dark yellow oil (0.15 g, 100%).

¹H NMR (300 MHz, CDCl₃) δ ppm: 10.24 (s, 1H), 8.03 (s, 1H), 5.40-5.27 (m, 1H), 1.59 (d, J=6.7 Hz, 6H).

Intermediate 158 was prepared according to the reaction protocol of intermediate 157 using the appropriate starting materials (Table 38).

TABLE 38

| | | Starting | |
|---|---|---|---|
| Intermediate | Structure | Materials | NMR Data |
| 158 | 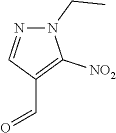 | a) Trigger 3 | ¹H NMR (300 MHz, CDCl₃) δ ppm: 10.29 (s, 1H), 8.02 (s, 1H), 4.67 (q, J = 7.1 Hz, 2H), 1.56 (t, J = 7.1 Hz, 3H). |

### Preparation of Trigger 5

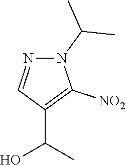

A solution of 1.0 M titanium tetrachloride solution in DCM (1.0 ml, 1.0 mmol) and diethyl ether (9.0 ml) at −78° C. under an argon atmosphere was treated with 1.4 M methyl magnesium bromide solution in THF and toluene (0.79 ml, 1.11 mmol, 1:3 by volume). The resulting mixture was stirred at −78° C. for 30 minutes. A solution of intermediate 157; (0.15 g, 0.82 mmol) in diethyl ether (1.0 ml) was added and the resulting mixture was stirred at −78° C. for a further 2.5 hours. The mixture was quenched with saturated aqueous ammonium chloride solution and partitioned between ice-water and EtOAc. The organic phase was dried over magnesium sulfate and concentrated in vacuo. The residue was purified by column chromatography on silica gel, eluting with a mixture of isohexane and EtOAc (1:0 to 7:3 by volume), to afford the desired product as a yellow oil (0.022 g, 13%).

LCMS (Method A): Rt=1.01 min, m/z [M+H]⁺=200

Triggers 6 and 7 were prepared according to the reaction protocol of trigger 5 using the appropriate starting materials (Table 39).

TABLE 39

| | | Starting | |
|---|---|---|---|
| Trigger | Structure | Materials | LCMS Data |
| 6 | 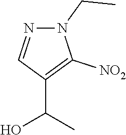 | a) Intermediate 158 | Rt = 1.14 min, m/z [M + H]⁺ = 186 (Method A) |
| 7 | 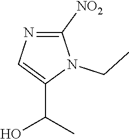 | a) 1 -Ethyl-2-nitro- 1H-imidazole-5- carbaldehyde | Rt = 0.91 min, m/z [M + H]⁺ = 186 (Method A) |

### Example C5

### Preparation of Intermediate 159

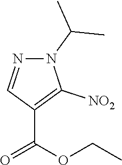 [SMILES: CCOC(=O)c1cnn(C(C)C)c1[N+](=O)[O-]]

A solution of intermediate 151 (0.11 g, 0.55 mmol) in EtOH (8.5 ml) was treated with concentrated sulfuric acid (0.085 ml) and the resulting mixture was heated at reflux for 22 hours. The mixture was cooled to ambient temperature and concentrated in vacuo. The residue was partitioned between EtOAc and saturated aqueous sodium bicarbonate solution. The organic phase was dried over magnesium sulfate and concentrated in vacuo. The residue was purified by column chromatography on silica gel, eluting with a mixture of isohexane and EtOAc (1:0 to 7:3 by volume), to afford the desired product as a colourless oil (0.055 g, 44%).

LCMS (Method A): Rt=1.31 min, m/z [M+H]⁺=228

### Example C6

### a) Preparation of Trigger 8

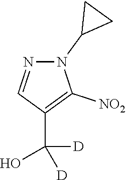 [SMILES: [2H]C([2H])(O)c1cnn(C2CC2)c1[N+](=O)[O-]]

A stirred solution of intermediate 153 (0.062 g, 0.28 mmol) in methanol-d₄ (1.5 ml) was treated with sodium borodeuteride (0.046 g, 1.10 mmol) and the resulting mixture was stirred at ambient temperature for 2.5 hours. A second portion of sodium borodeuteride (0.046 g, 1.10 mmol) was added and stirring was continued for 18 hours. A third portion of sodium borodeuteride (0.046 g, 1.10 mmol) was added and stirring was continued for a further 3 hours. The resulting mixture was partitioned between EtOAc and water. The organic phase was dried over sodium sulfate and concentrated in vacuo. The residue was purified by column chromatography on silica gel, eluting with a mixture of isohexane and EtOAc (1:0 to 3:7 by volume), to afford the desired product as a yellow oil (0.020 g, 39%).

¹H NMR (400 MHz, CDCl₃) δ ppm: 7.02 (s, 1H), 3.55-3.48 (m, 1H), 2.27-2.27 (m, 1H), 1.31-1.25 (m, 2H), 1.04-1.00 (m, 2H).

Triggers 9 and 10 were prepared according to the reaction protocol of trigger 8 using the appropriate starting materials (Table 40).

TABLE 40

| | | Starting | |
|---|---|---|---|
| Trigger | Structure | Materials | NMR Data |
| 9 | 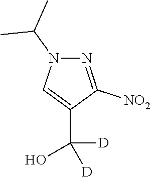 | a) Intermediate 156 | ¹H NMR (300 MHz, CDCl₃) δ ppm: 7.54 (s, 1H), 4.57 (hept, J = 6.6 Hz, 1H), 2.48 (s, 1H) 1.56 (d, J = 6.6 Hz, 6H). |
| 10 | 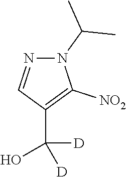 | a) Intermediate 159 | ¹H NMR (300 MHz, CDCl₃) δ ppm: 7.62 (s, 1H), 5.40 (hept, J = 6.9 Hz, 1H), 2.11 (s, 1H), 1.54 (d, J = 6.9 Hz, 6H). |

### Example C7

### a) Preparation of Trigger 11

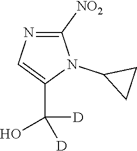 [SMILES: [2H]C([2H])(O)c1cnc([N+](=O)[O-])n1C1CC1]

A stirred solution of intermediate 155 (0.090 g, 0.46 mmol) and triethylamine (0.13 ml, 0.91 mmol) in anhydrous THF (1.5 ml) under an argon atmosphere at −15° C. was treated with isobutyl chloroformate (0.12 ml, 0.65 mmol). The resulting mixture was stirred at −5° C. for 10 minutes, then treated with sodium borodeuteride (0.10 g, 2.39 mmol). Deuterium oxide (0.083 ml, 4.60 mmol) was carefully added and stirring was continued for 10 minutes. The resulting mixture was partitioned between EtOAc and water. The organic phase was dried over sodium sulfate and concentrated in vacuo. The residue was purified by column chromatography on silica gel, eluting with a mixture of isohexane and EtOAc (1:0 to 0:1 by volume), to afford the desired product as a pale yellow oil, which solidified on standing (0.063 g, 74%).

LCMS (Method A): Rt=0.83 min, m/z [M+H]⁺=186

### Example C8

### Preparation of Intermediate 160

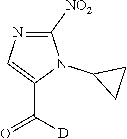 [SMILES: [2H]C(=O)c1cnc([N+](=O)[O-])n1C1CC1]

A stirred solution of trigger 11 (0.070 g, 0.38 mmol) in DCM (1.0 ml) was treated with 0.3 M Dess-Martin periodinane solution in DCM (2.5 mL, 0.75 mmol). After stirring at ambient temperature for 0.5 hours, the resulting mixture was partitioned between DCM and saturated aqueous sodium bicarbonate solution. The organic phase was dried over sodium sulfate and concentrated in vacuo. The residue was purified by column chromatography on silica gel, eluting with a mixture of isohexane and EtOAc (1:0 to 1:1 by volume), to afford the desired product as a colourless oil (0.064 g, 93%).

LCMS (Method A): Rt=0.99 min, m/z [M+H]⁺=183

### Preparation of Trigger 12

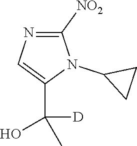

A solution of 1.0 M titanium tetrachloride solution in DCM (1.0 ml, 1.0 mmol) at −78° C. under an argon atmosphere was treated with 3.0 M methyl magnesium bromide solution in diethyl ether (0.35 ml, 1.0 mmol). The resulting mixture was stirred at −78° C. for 10 minutes. A solution of intermediate 160 (0.063 g, 0.35 mmol) in THF (2.0 ml) was added and the resulting mixture was warmed to 0° C. over 30 minutes. The resulting mixture was partitioned between DCM and water. The organic phase was dried over sodium sulfate and concentrated in vacuo. The residue was purified by column chromatography on silica gel, eluting with a mixture of isohexane and EtOAc (1:0 to 0:1 by volume), to afford the desired product as a yellow oil (0.023 g, 33%).

LCMS (Method A): Rt=0.92 min, m/z [M+H]⁺=199

### Preparation of Prodrugs

### Example D1

### a) Preparation of Prodrug 1

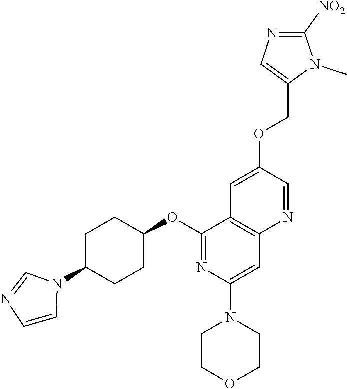 [SMILES: Cn1c(COc2cnc3cc(N4CCOCC4)nc(O[C@H]4CC[C@@H](n5ccnc5)CC4)c3c2)cnc1[N+](=O)[O-]]

A mixture of parent compound 162 (0.026 g, 0.065 mmol), 5-(bromomethyl)-1-methyl-2-nitro-1H-imidazole (0.017 g, 0.075 mmol) and potassium carbonate (0.018 g, 0.13 mmol) in DMF (0.5 ml) was stirred at ambient temperature for 2 hours. A second portion of 5-(bromomethyl)-1-methyl-2-nitro-1H-imidazole (0.010 g, 0.045 mmol) was added and stirring was continued for a further 1 hour. The resulting mixture was partitioned between EtOAc and saturated aqueous sodium bicarbonate solution. The organic phase was dried over sodium sulfate and concentrated in vacuo. The residue was purified on a Biotage® KP-NH column, eluting with a mixture of EtOAc and MeOH (1:0 to 4:1 by volume). Further purification by MDAP (Method B) afforded the desired product as a yellow solid (0.012 g, 53%).

LCMS (Method C): Rt=3.90 min, m/z [M+H]⁺=535

¹H NMR (400 MHz, DMSO-d₆) δ ppm: 8.68 (d, J=2.9 Hz, 1H), 7.91 (d, J=2.9 Hz, 1H), 7.79 (dd, J=1.0, 1.0 Hz, 1H), 7.36 (s, 1H), 7.34 (dd, J=1.0, 1.0 Hz, 1H), 6.90 (dd, J=1.0, 1.0 Hz, 1H), 6.56 (s, 1H), 5.48-5.47 (m, 3H), 4.24 (tt, J=3.6, 11.0 Hz, 1H), 4.00 (s, 3H), 3.77-3.72 (m, 4H), 3.48-3.42 (m, 4H), 2.24-2.11 (m, 4H), 1.98-1.82 (m, 4H).

Prodrugs 2 to 14 were prepared according to the reaction protocol of prodrug 1 using the appropriate starting materials (Table 41).

TABLE 41

| Prodrug | Structure | Starting Materials |
|---|---|---|
| 2 | 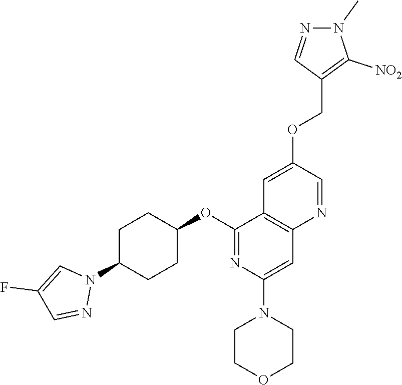 [SMILES: Cn1ncc(COc2cnc3cc(N4CCOCC4)nc(O[C@H]4CC[C@@H](n5cc(F)cn5)CC4)c3c2)c1[N+](=O)[O-]] | a) Parent compound 163 b) 4-(Bromomethyl)-1-methyl- 5-nitro-1H-pyrazole |
| 3 | 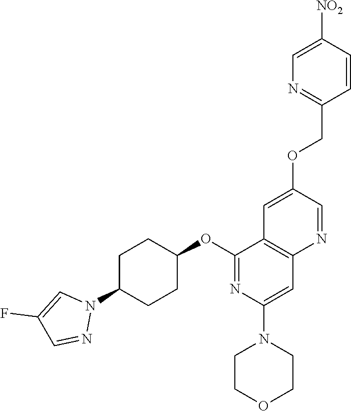 [SMILES: O=[N+]([O-])c1ccc(COc2cnc3cc(N4CCOCC4)nc(O[C@H]4CC[C@@H](n5cc(F)cn5)CC4)c3c2)nc1] | a) Parent compound 163 b) 2-(Bromomethyl)-5- nitropyridine |
| 4 | 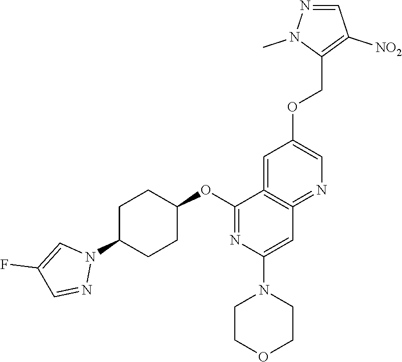 | a) Parent compound 163 b) 5-(Bromomethyl)-1-methyl- 4-nitro-1H-pyrazole |
| 5 | 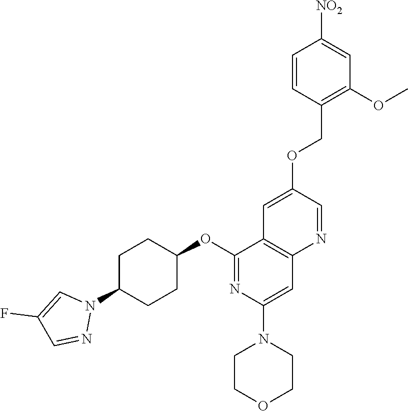 [SMILES: COc1cc([N+](=O)[O-])ccc1COc1cnc2cc(N3CCOCC3)nc(O[C@H]3CC[C@@H](n4cc(F)cn4)CC3)c2c1] | a) Parent compound 163 b) 1-(Bromomethyl)-2- methoxy-4-nitrobenzene |
| 6 | 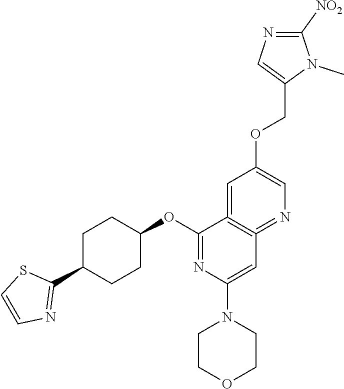 | a) Parent compound 168 b) 5-(Bromomethyl)-1-methyl- 2-nitro-1H-imidazole |
| 7 | 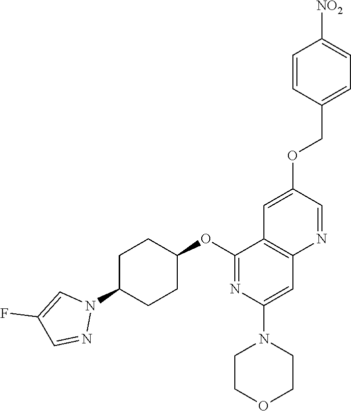 [SMILES: O=[N+]([O-])c1ccc(COc2cnc3cc(N4CCOCC4)nc(O[C@H]4CC[C@@H](n5cc(F)cn5)CC4)c3c2)cc1] | a) Parent compound 163 b) 1-(Bromomethyl)-4- nitrobenzene |
| 8 | 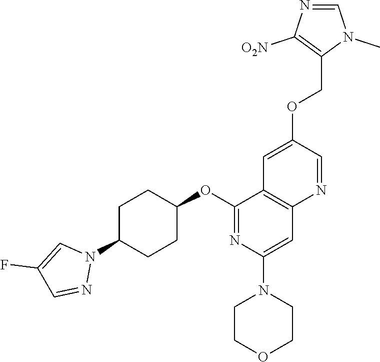 [SMILES: Cn1cnc([N+](=O)[O-])c1COc1cnc2cc(N3CCOCC3)nc(O[C@H]3CC[C@@H](n4cc(F)cn4)CC3)c2c1] | a) Parent compound 163 b) 5-(Chloromethyl)-1-methyl- 4-nitro-1H-imidazole |
| 9 | 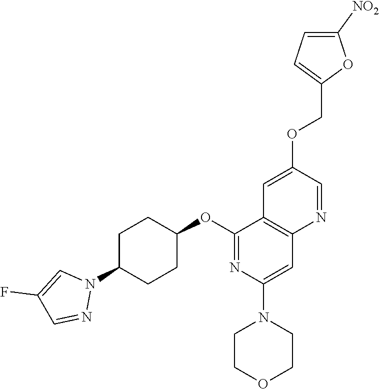 [SMILES: O=[N+]([O-])c1ccc(COc2cnc3cc(N4CCOCC4)nc(O[C@H]4CC[C@@H](n5cc(F)cn5)CC4)c3c2)o1] | a) Parent compound 163 b) 2-(Bromomethyl)-5- nitrofuran |
| 10 | 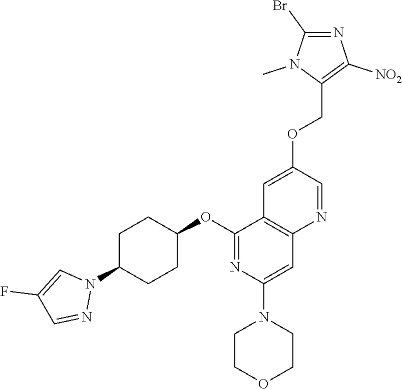 [SMILES: Cn1c(Br)nc([N+](=O)[O-])c1COc1cnc2cc(N3CCOCC3)nc(O[C@H]3CC[C@@H](n4cc(F)cn4)CC3)c2c1] | a) Parent compound 163 b) 2-Bromo-5-(bromomethyl)- 1-methyl-4-nitro-1H-imidazole |
| 11 | 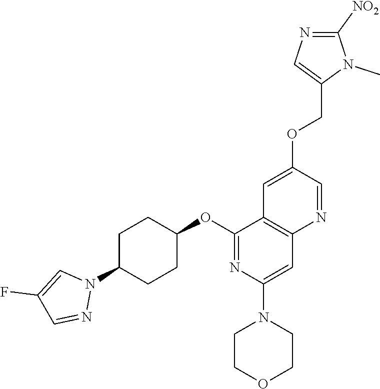 [SMILES: Cn1c(COc2cnc3cc(N4CCOCC4)nc(O[C@H]4CC[C@@H](n5cc(F)cn5)CC4)c3c2)cnc1[N+](=O)[O-]] | a) Parent compound 163 b) 5-(Bromomethyl)-1-methyl- 2-nitro-1H-imidazole |
| 12 | 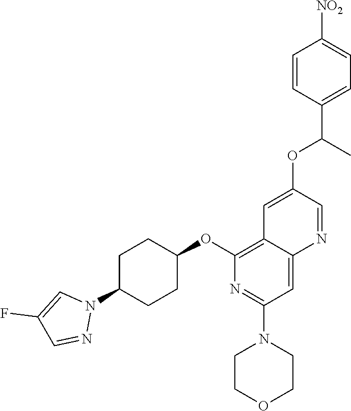 [SMILES: CC(Oc1cnc2cc(N3CCOCC3)nc(O[C@H]3CC[C@@H](n4cc(F)cn4)CC3)c2c1)c1ccc([N+](=O)[O-])cc1] | a) Parent compound 163 b) 1-(1-Bromoethyl)-4- nitrobenzene |
| 13 | 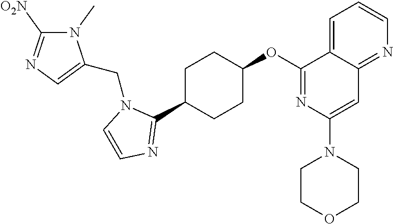 [SMILES: Cn1c(Cn2ccnc2[C@H]2CC[C@@H](Oc3nc(N4CCOCC4)cc4ncccc34)CC2)cnc1[N+](=O)[O-]] | a) Parent compound 75 b) 5-(Bromomethyl)-1-methyl- 2-nitro-1H-imidazole |
| 14 | 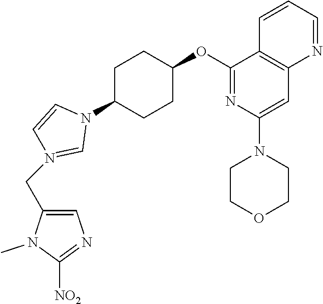 | a) Parent compound 77 b) 5-(Bromomethyl)-1-methyl- 2-nitro-1H-imidazole |

### Example D2

a) Preparation of prodrug 15

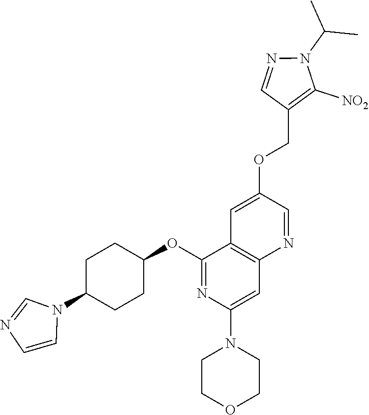 [SMILES: CC(C)n1ncc(COc2cnc3cc(N4CCOCC4)nc(O[C@H]4CC[C@@H](n5ccnc5)CC4)c3c2)c1[N+](=O)[O-]]

A stirred solution of parent compound 162 (0.033 g, 0.083 mmol), trigger 2 (0.023 g, 0.013 mmol) and triphenylphosphine (0.044 g, 0.17 mmol) in anhydrous THF (0.15 ml) was treated with DIAD (0.033 ml, 0.17 mmol). After stirring for 1.5 hours at ambient temperature, the resulting mixture was partitioned between DCM and water. The organic phase was dried over sodium sulfate and concentrated in vacuo. The residue was purified by column chromatography on silica gel, eluting with a mixture of DCM and MeOH (1:0 to 9:1 by volume). Further purification by reverse phase preparative HPLC (Method B) afforded the desired product as a yellow solid (0.011 g, 24%).

LCMS (Method C): Rt=3.27 min, m/z [M+H]⁺=563

¹H NMR (400 MHz, DMSO-d₆) δ ppm: 8.69 (d, J=3.0 Hz, 1H), 7.91 (s, 1H), 7.82 (d, J=3.0 Hz, 1H), 7.75 (s, 1H), 7.29 (s, 1H), 6.89 (s, 1H), 6.56 (s, 1H), 5.49-5.46 (m, 1H), 5.41 (s, 2H), 5.31 (hept, J=6.5 Hz, 1H), 4.27-4.20 (m, 1H), 3.77-3.73 (m, 4H), 3.47-3.42 (m, 4H), 2.23-2.06 (m, 4H), 1.96-1.82 (m, 4H), 1.46 (d, J=6.5 Hz, 6H).

Prodrugs 16 to 27 were prepared according to the reaction protocol of prodrug 15 using the appropriate starting materials (Table 42).

TABLE 42

| Prodrug | Structure | Starting Materials |
|---|---|---|
| 16 | 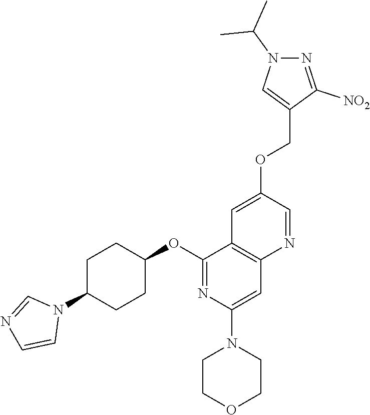 [SMILES: CC(C)n1cc(COc2cnc3cc(N4CCOCC4)nc(O[C@H]4CC[C@@H](n5ccnc5)CC4)c3c2)c([N+](=O)[O-])n1] | a) Parent compound 162 b) Trigger 4 |
| 17 | 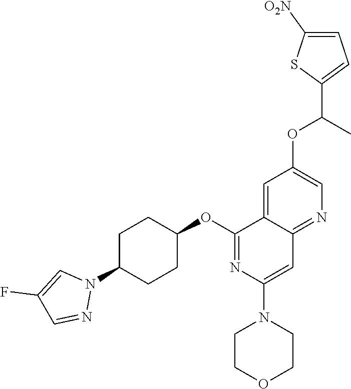 [SMILES: CC(Oc1cnc2cc(N3CCOCC3)nc(O[C@H]3CC[C@@H](n4cc(F)cn4)CC3)c2c1)c1ccc([N+](=O)[O-])s1] | a) Parent compound 163 b) 1-(5-Nitrothiophen-2- yl)ethan-1-ol |
| 18 | 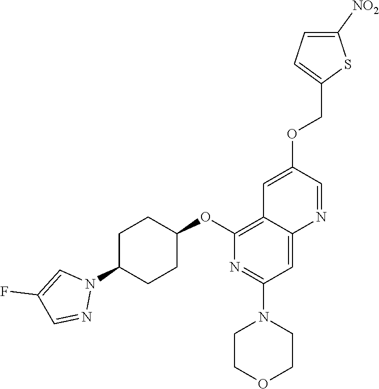 [SMILES: O=[N+]([O-])c1ccc(COc2cnc3cc(N4CCOCC4)nc(O[C@H]4CC[C@@H](n5cc(F)cn5)CC4)c3c2)s1] | a) Parent compound 163 b) (5-Nitrothiophen-2- yl)methanol |
| 19 | 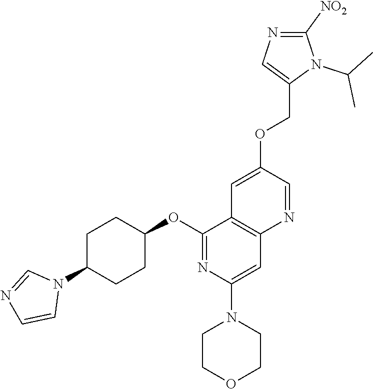 [SMILES: CC(C)n1c(COc2cnc3cc(N4CCOCC4)nc(O[C@H]4CC[C@@H](n5ccnc5)CC4)c3c2)cnc1[N+](=O)[O-]] | a) Parent compound 162 b) Trigger 1 |
| 20 | 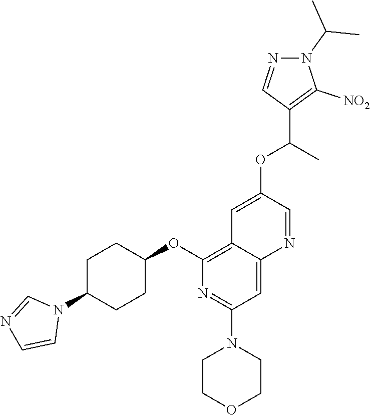 [SMILES: CC(Oc1cnc2cc(N3CCOCC3)nc(O[C@H]3CC[C@@H](n4ccnc4)CC3)c2c1)c1cnn(C(C)C)c1[N+](=O)[O-]] | a) Parent compound 162 b) Trigger 5 |
| 21 | 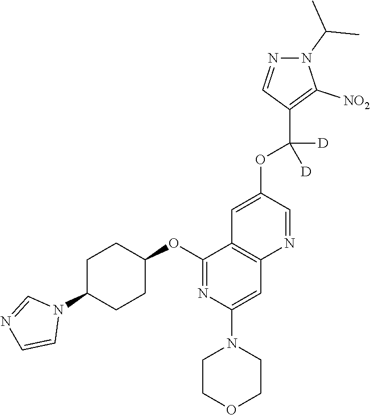 [SMILES: [2H]C([2H])(Oc1cnc2cc(N3CCOCC3)nc(O[C@H]3CC[C@@H](n4ccnc4)CC3)c2c1)c1cnn(C(C)C)c1[N+](=O)[O-]] | a) Parent compound 162 b) Trigger 10 |
| 22 | 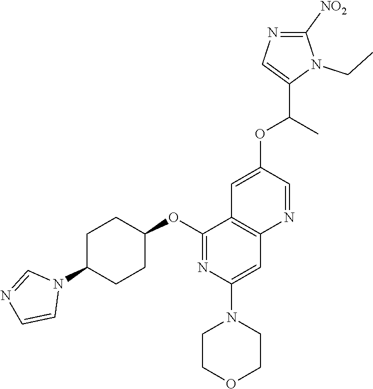 [SMILES: CCn1c(C(C)Oc2cnc3cc(N4CCOCC4)nc(O[C@H]4CC[C@@H](n5ccnc5)CC4)c3c2)cnc1[N+](=O)[O-]] | a) Parent compound 162 b) Trigger 7 |
| 23 | 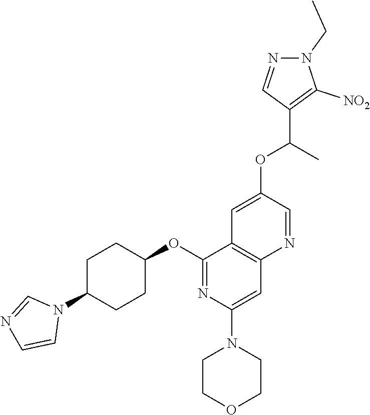 [SMILES: CCn1ncc(C(C)Oc2cnc3cc(N4CCOCC4)nc(O[C@H]4CC[C@@H](n5ccnc5)CC4)c3c2)c1[N+](=O)[O-]] | a) Parent compound 162 b) Trigger 6 |
| 24 | 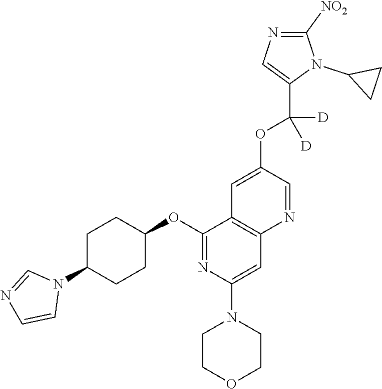 [SMILES: [2H]C([2H])(Oc1cnc2cc(N3CCOCC3)nc(O[C@H]3CC[C@@H](n4ccnc4)CC3)c2c1)c1cnc([N+](=O)[O-])n1C1CC1] | a) Parent compound 162 b) Trigger 11 |
| 25 | 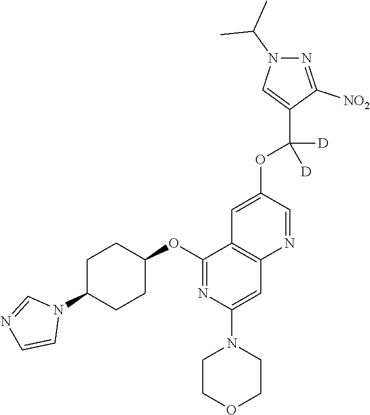 [SMILES: [2H]C([2H])(Oc1cnc2cc(N3CCOCC3)nc(O[C@H]3CC[C@@H](n4ccnc4)CC3)c2c1)c1cn(C(C)C)nc1[N+](=O)[O-]] | a) Parent compound 162 b) Trigger 9 |
| 26 | 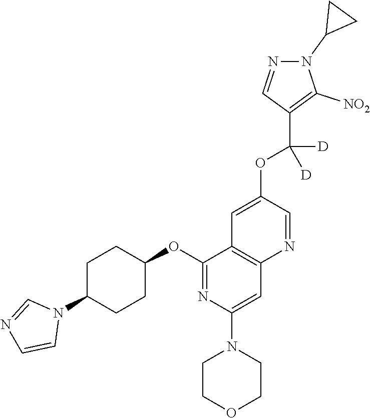 [SMILES: [2H]C([2H])(Oc1cnc2cc(N3CCOCC3)nc(O[C@H]3CC[C@@H](n4ccnc4)CC3)c2c1)c1cnn(C2CC2)c1[N+](=O)[O-]] | a) Parent compound 162 b) Trigger 8 |
| 27 | 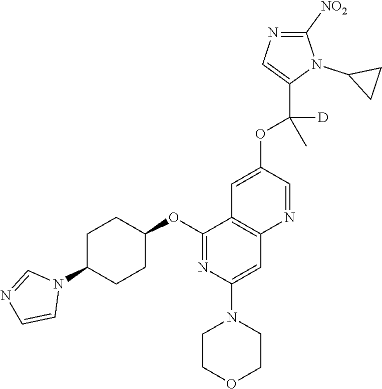 [SMILES: [2H]C(C)(Oc1cnc2cc(N3CCOCC3)nc(O[C@H]3CC[C@@H](n4ccnc4)CC3)c2c1)c1cnc([N+](=O)[O-])n1C1CC1] | a) Parent compound 162 b) Trigger 12 |

### Example D3

### a) Preparation of Prodrugs 28 and 29

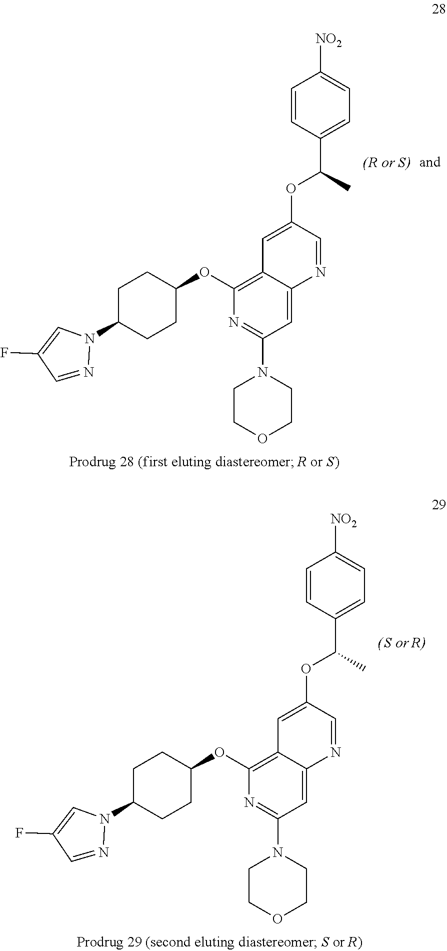 [SMILES: C[C@@H](Oc1cnc2cc(N3CCOCC3)nc(O[C@H]3CC[C@@H](n4cc(F)cn4)CC3)c2c1)c1ccc([N+](=O)[O-])cc1 ; C[C@H](Oc1cnc2cc(N3CCOCC3)nc(O[C@H]3CC[C@@H](n4cc(F)cn4)CC3)c2c1)c1ccc([N+](=O)[O-])cc1]

Prodrug 12 (0.028 g, 0.051 mmol), was purified by chiral preparative SFC with the following conditions: column, Phenomenex Lux® 5u Cellulose-4, 250×21.2 mm, 5 μm; mobile phase, CO₂ (45%), MeOH (0.1% DEA) (55%); detector, UV 255 nm. This afforded prodrug 28 (first eluting diastereomer; R or S) as a yellow solid (0.008 g, 28%) and prodrug 29 (second eluting diastereomer; S or R) as a yellow solid (0.008 g, 28%).

### Prodrug 28

LCMS (Method C): Rt=5.14 min, m/z [M+H]⁺=563

¹H NMR (400 MHz, DMSO-d₆) δ ppm: 8.67 (d, J=3.0 Hz, 1H), 8.11 (d, J=8.7 Hz, 2H), 8.03 (d, J=4.5 Hz, 1H), 7.77 (d, J=8.7 Hz, 2H), 7.57-7.54 (m, 2H), 6.48 (s, 1H), 5.87 (q, J=6.4 Hz, 1H), 5.43-5.38 (m, 1H), 4.26-4.19 (m, 1H), 3.74-3.69 (m, 4H), 3.42-3.37 (m, 4H), 2.25-2.11 (m, 2H), 2.03-1.76 (m, 6H), 1.66 (d, J=6.4 Hz, 3H).

### Prodrug 29

LCMS (Method C): Rt=5.14 min, m/z [M+H]⁺=563

¹H NMR (400 MHz, DMSO-d₆) δ ppm: 8.67 (d, J=3.1 Hz, 1H), 8.11 (d, J=8.7 Hz, 2H), 8.03 (d, J=4.6 Hz, 1H), 7.77 (d, J=8.7 Hz, 2H), 7.56-7.54 (m, 2H), 6.48 (s, 1H), 5.87 (q, J=6.4 Hz, 1H), 5.42-5.38 (m, 1H), 4.27-4.19 (m, 1H), 3.74-3.69 (m, 4H), 3.42-3.36 (m, 4H), 2.25-2.11 (m, 2H), 2.03-1.77 (m, 6H), 1.66 (d, J=6.4 Hz, 3H).

### Example D4

### a) Preparation of Intermediate 161

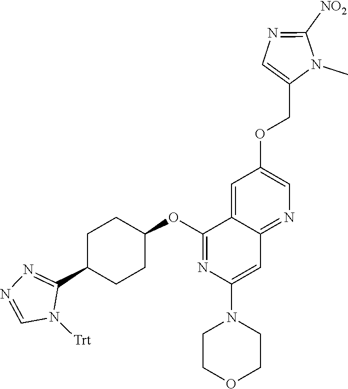

A mixture of intermediate 146 (0.043 g, 0.067 mmol), 5-(bromomethyl)-1-methyl-2-nitro-1H-imidazole (0.015 g, 0.067 mmol) and potassium carbonate (0.019 g, 0.13 mmol) in DMF (1.0 ml) was stirred at ambient temperature for 1 hour. The resulting mixture was partitioned between EtOAc and saturated aqueous sodium bicarbonate solution. The organic phase was washed with brine, dried over sodium sulfate and concentrated in vacuo to afford the desired product as a yellow oil (0.051 g, 77%).

LCMS (Method B): Rt=2.35 min, m/z [M+H]⁺=778

Intermediate 162 was prepared according to the reaction protocol of intermediate 161 using the appropriate starting materials (Table 43).

TABLE 43

| | | Starting | |
|---|---|---|---|
| Intermediate | Structure | Materials | LCMS Data |
| 162 | 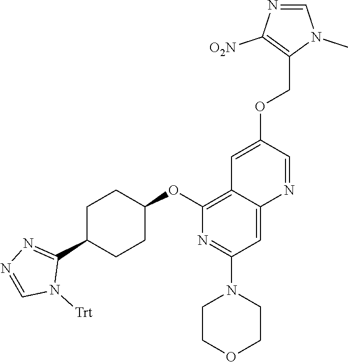 | a) Intermediate 146 b) 5- (Chloromethyl)-1 - methyl-4-nitro- 1H-imidazole | Rt = 2.26 min, m/z [M + H]+ = 778 (Method B) |

### b) Preparation of Prodrug 30

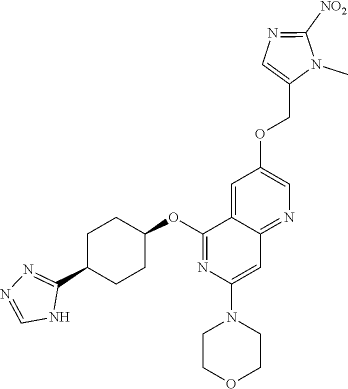 [SMILES: Cn1c(COc2cnc3cc(N4CCOCC4)nc(O[C@H]4CC[C@@H](c5nnc[nH]5)CC4)c3c2)cnc1[N+](=O)[O-]]

A stirred solution of intermediate 161 (0.051 g, 0.068 mmol) in DCM (2.0 ml) was treated with TFA (1.0 ml). After stirring at ambient temperature for 15 minutes, the mixture was diluted with toluene and concentrated in vacuo. The residue was purified by column chromatography on silica gel, eluting with a mixture of DCM and 2.0 M ammonia in MeOH (1:0 to 9:1 by volume). Further purification by reverse phase preparative HPLC (Method B) afforded the desired product as a yellow solid (0.006 g, 17%).

LCMS (Method D): Rt=3.53 min, m/z [M+H]⁺=536

¹H NMR (400 MHz, DMSO-d₆) δ ppm: 13.65 (s, 1H), 8.67 (d, J=2.9 Hz, 1H), 8.02 (s, 1H), 7.82 (d, J=2.9 Hz, 1H), 7.37 (s, 1H), 6.55 (s, 1H), 5.43-5.41 (m, 3H), 3.98 (s, 3H), 3.77-3.73 (m, 4H), 3.48-3.42 (m, 4H), 2.97-2.97 (m, 1H), 2.13-2.02 (m, 4H), 1.93-1.83 (m, 4H).

Prodrug 31 was prepared according to the reaction protocol of prodrug 30 using the appropriate starting materials (Table 44).

TABLE 44

| Prodrug | Structure | Starting Materials |
|---|---|---|
| 31 | 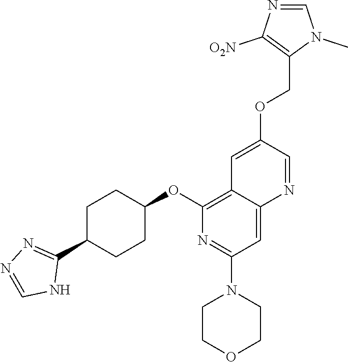 [SMILES: Cn1cnc([N+](=O)[O-])c1COc1cnc2cc(N3CCOCC3)nc(O[C@H]3CC[C@@H](c4nnc[nH]4)CC3)c2c1] | a) Intermediate 162 |

### Analytical Methods

### LCMS

Mass Spectrometry (LCMS) experiments to determine retention times and associated mass ions were performed using the following methods:

Method A: Experiments were performed on a Waters Acquity QDa mass spectrometer linked to a Waters Acquity H-Class quaternary pump LC system with a photodiode array detector. The spectrometer had an electrospray source operating in positive and negative ion mode. LC was carried out using a Waters Acquity 1.7 μm UPLC CSH 50×2.1 mm C18 column and a 1 ml/minute flow rate. The initial solvent system was 97% water containing 0.1% formic acid (solvent A) and 3% MeCN containing 0.1% formic acid (solvent B), with a gradient up to 1% solvent A and 99% solvent B over 1.5 minutes. The final solvent system was held constant for a further 0.4 minute.
Method B: Experiments were performed on a Waters Acquity QDa mass spectrometer linked to a Waters Acquity H-Class quaternary pump LC system with a photodiode array detector. The spectrometer had an electrospray source operating in positive and negative ion mode. LC was carried out using a Waters XBridge® 2.5 μm BEH 50×2.1 mm C18 column and a 1 ml/minute flow rate. The initial solvent system was 97% water containing 0.1% ammonium hydroxide (solvent A) and 3% MeCN containing 0.1% ammonium hydroxide (solvent B) for the first 0.2 minute, followed by a gradient up to 5% solvent A and 95% solvent B over the next 2 minutes. The final solvent system was held constant for a further 0.5 minute.
Method C: Experiments were performed on a Waters Micromass ZQ2000 quadrupole mass spectrometer linked to a Waters Acquity binary pump UPLC system with a photodiode array detector. The spectrometer had an electrospray source operating in positive and negative ion mode. LC was carried out using an Acquity 1.7 μm UPLC BEH 100×2.1 mm C18 column and a 0.4 ml/minute flow rate. The initial solvent system was 95% water containing 0.1% formic acid (solvent A) and 5% MeCN containing 0.1% formic acid (solvent B) for the first 0.4 minute, followed by a gradient up to 5% solvent A and 95% solvent B over the next 5.6 minutes. The final solvent system was held constant for a further 0.8 minute.
Method D: Experiments were performed on a Waters Micromass ZQ2000 quadrupole mass spectrometer linked to a Waters Acquity binary pump UPLC system with a photodiode array detector. The spectrometer had an electrospray source operating in positive and negative ion mode. LC was carried out using an Acquity 1.7 μm UPLC BEH 100×2.1 mm C18 column and a 0.4 ml/minute flow rate. The initial solvent system was 95% water containing 0.1% ammonium hydroxide (solvent A) and 5% MeCN containing 0.1% ammonium hydroxide (solvent B) for the first 0.4 minute, followed by a gradient up to 5% solvent A and 95% solvent B over the next 5.6 minutes. The final solvent system was held constant for a further 0.8 minute.
Method E: Experiments were performed on a Waters Quattro Micromass tandem quadrupole mass spectrometer linked to a Waters Acquity i-Class quaternary pump UPLC system with a photodiode array detector. The spectrometer had an electrospray source operating in positive and negative ion mode. LC was carried out using an Acquity 1.7 μm UPLC BEH 100×2.1 mm C18 column and a 0.4 ml/minute flow rate. The initial solvent system was 95% water containing 0.1% formic acid (solvent A) and 5% MeCN containing 0.1% formic acid (solvent B) for the first 0.4 minute, followed by a gradient up to 5% solvent A and 95% solvent B over the next 5.6 minutes. The final solvent system was held constant for a further 0.8 minute.
Method F: Experiments were performed on a Waters Micromass ZQ2000 quadrupole mass spectrometer linked to an Agilent HP1100 quaternary pump LC system with a photodiode array detector. The spectrometer had an electrospray source operating in positive and negative ion mode. LC was carried out using a Phenomenex® Gemini 3 μm 4.6×30 mm NX-C18 column and a 2 ml/minute flow rate. The initial solvent system was 95% water containing 0.1% ammonium hydroxide (solvent A) and 5% MeCN containing 0.1% ammonium hydroxide (solvent B) for the first 0.3 minute, followed by a gradient up to 5% solvent A and 95% solvent B over the next 4 minutes. The final solvent system was held constant for a further 1 minute.

### NMR Data

The NMR experiments herein were carried out using a Varian Unity Inova spectrometer with standard pulse sequences, operating at 400 MHz at ambient temperature. Chemical shifts (δ) are reported in parts per million (ppm) downfield from tetramethylsilane (TMS), which was used as internal standard.

The values of acid content (e.g. formic acid or acetic acid) in the compounds as provided herein, are those obtained experimentally and may vary when using different analytical methods. The content of formic acid or acetic acid reported herein was determined by ¹H NMR integration. Compounds with an acid content of below 0.5 equivalents may be considered as free bases.

### Parent Compound 2

LCMS (Method C): Rt=2.57 min, m/z [M+H]⁺=406

¹H NMR (400 MHz, DMSO-d₆) δ ppm: 8.71 (dd, J=1.5, 4.2 Hz, 1H), 8.25 (d, J=4.8 Hz, 2H), 8.12 (dd, J=1.5, 8.3 Hz, 1H), 7.25 (t, J=5.9 Hz, 1H), 7.13 (dd, J=4.2, 8.3 Hz, 1H), 6.54 (t, J=4.8 Hz, 1H), 6.50 (s, 1H), 3.84-3.80 (m, 2H), 3.75-3.70 (m, 4H), 3.51-3.47 (m, 4H), 3.28-3.22 (m, 2H), 2.95-2.86 (m, 2H), 1.86-1.78 (m, 3H), 1.51-1.40 (m, 2H).

### Parent Compound 3

LCMS (Method C): Rt=2.31 min, m/z [M+H]⁺=357

¹H NMR (400 MHz, DMSO-d₆) δ ppm: 8.79 (dd, J=1.8, 4.3 Hz, 1H), 8.30 (ddd, J=0.6, 1.8, 8.3 Hz, 1H), 7.26-7.19 (m, 2H), 6.73 (s, 1H), 6.52 (s, 1H), 5.42-5.38 (m, 1H), 3.76-3.72 (m, 4H), 3.51-3.47 (m, 4H), 2.29-2.19 (m, 1H), 2.11-2.03 (m, 2H), 1.84-1.59 (m, 6H).

### Parent Compound 4

LCMS (Method C): Rt=2.80 min, m/z [M+H]⁺=339

¹H NMR (400 MHz, DMSO-d₆) δ ppm: 8.79 (dd, J=1.5, 4.3 Hz, 1H), 8.35 (dd, J=1.5, 8.2 Hz, 1H), 7.20 (dd, J=4.3, 8.2 Hz, 1H), 6.54 (s, 1H), 5.32-5.26 (m, 1H), 3.76-3.73 (m, 4H), 3.51-3.46 (m, 4H), 3.02-2.99 (m, 1H), 1.99-1.84 (m, 8H).

### Parent Compound 5

LCMS (Method C): Rt=3.04 min, m/z [M+H]⁺=396

¹H NMR (400 MHz, DMSO-d₆) δ ppm: 8.78 (dd, J=1.5, 4.3 Hz, 1H), 8.30 (dd, J=1.5, 8.2 Hz, 1H), 7.19 (dd, J=4.3, 8.2 Hz, 1H), 6.53 (s, 1H), 5.48-5.42 (m, 1H), 3.77-3.72 (m, 4H), 3.52-3.47 (m, 4H), 3.25-3.16 (m, 1H), 2.33 (s, 3H), 2.13-2.04 (m, 2H), 2.04-1.95 (m, 4H), 1.93-1.85 (m, 2H).

### Parent Compound 6

LCMS (Method C): Rt=2.54 min, m/z [M+H]⁺=371

¹H NMR (400 MHz, DMSO-d₆) δ ppm: 8.78 (dd, J=1.5, 4.3 Hz, 1H), 8.27 (dd, J=1.5, 8.3 Hz, 1H), 7.20-7.16 (m, 2H), 6.69 (bs, 1H), 6.52 (s, 1H), 4.37 (d, J=7.1 Hz, 2H), 3.77-3.72 (m, 4H), 3.53-3.49 (m, 4H), 2.31-2.23 (m, 1H), 2.09-2.01 (m, 1H), 1.85-1.74 (m, 2H), 1.64-1.47 (m, 6H).

Parent compound 7

LCMS (Method C): Rt=2.31 min, m/z [M+H]⁺=367

¹H NMR (400 MHz, DMSO-d₆) δ ppm: 8.78 (dd, J=1.8, 4.3 Hz, 1H), 8.26 (dd, J=1.8, 8.2 Hz, 1H), 8.23 (d, J=5.4 Hz, 2H), 7.25 (t, J=5.4 Hz, 1H), 7.19 (dd, J=4.3, 8.2 Hz, 1H), 6.55-6.51 (m, 2H), 4.51 (t, J=6.4 Hz, 2H), 3.74-3.70 (m, 4H), 3.50-3.45 (m, 6H), 2.11-2.03 (m, 2H).

### Parent Compound 8

LCMS (Method C): Rt=2.44 min, m/z [M+H]⁺=381

¹H NMR (400 MHz, DMSO-d₆) δ ppm: 8.78 (dd, J=1.8, 4.3 Hz, 1H), 8.26-8.22 (m, 3H), 7.20-7.14 (m, 2H), 6.54-6.51 (m, 2H), 4.47 (t, J=6.4 Hz, 2H), 3.75-3.71 (m, 4H), 3.52-3.48 (m, 4H), 3.38-3.34 (m, 2H), 1.91-1.81 (m, 2H), 1.77-1.67 (m, 2H).

### Parent Compound 9 (Formic Acid 0.70 Equivalents)

LCMS (Method C): Rt=3.57 min, m/z [M+H]⁺=314

¹H NMR (400 MHz, DMSO-d₆) δ ppm: 8.78 (dd, J=1.5, 4.3 Hz, 1H), 8.26 (dd, J=1.5, 8.2 Hz, 1H), 7.18 (dd, J=4.3, 8.2 Hz, 1H), 6.51 (s, 1H), 5.25-5.16 (m, 1H), 3.77-3.72 (m, 4H), 3.52-3.44 (m, 4H), 2.03-1.94 (m, 2H), 1.82-1.72 (m, 2H), 1.69-1.34 (m, 6H).

### Parent Compound 10

LCMS (Method C): Rt=2.44 min, m/z [M+H]⁺=379

¹H NMR (400 MHz, DMSO-d₆) δ ppm: 8.78 (dd, J=1.5, 4.3 Hz, 1H), 8.37 (dd, J=1.5, 8.3 Hz, 1H), 8.29 (d, J=4.6 Hz, 2H), 7.71 (s, 1H), 7.20 (dd, J=4.3, 8.2 Hz, 1H), 6.62 (t, J=4.6 Hz, 1H), 6.47 (s, 1H), 4.60 (s, 2H), 3.71-3.66 (m, 4H), 3.41-3.36 (m, 4H), 1.02-0.97 (m, 2H), 0.86-0.81 (m, 2H).

### Parent Compound 11 (Formic Acid 0.63 Equivalents)

LCMS (Method C): Rt=2.53 min, m/z [M+H]⁺=392

¹H NMR (400 MHz, DMSO-d₆) δ ppm: 8.60 (dd, J=1.6, 4.2 Hz, 1H), 8.40-8.38 (m, 1H), 8.25 (d, J=4.8 Hz, 2H), 7.36 (t, J=5.8 Hz, 1H), 6.99 (dd, J=4.2, 8.3 Hz, 1H), 6.55 (t, J=4.8 Hz, 1H), 6.26 (s, 1H), 3.87-3.75 (m, 3H), 3.73-3.68 (m, 4H), 3.62 (dd, J=6.8, 10.9 Hz, 1H), 3.49-3.44 (m, 4H), 3.43-3.29 (m, 2H), 2.61-2.53 (m, 1H), 2.11-2.01 (m, 1H), 1.81-1.70 (m, 1H).

### Parent Compound 12

LCMS (Method C): Rt=2.43 min, m/z [M+H]⁺=390

¹H NMR (400 MHz, DMSO-d₆) δ ppm: 8.61 (dd, J=1.5, 4.2 Hz, 1H), 8.45-8.41 (m, 1H), 8.29 (d, J=4.8 Hz, 2H), 7.46 (d, J=3.2 Hz, 1H), 7.02 (dd, J=4.2, 8.3 Hz, 1H), 6.61 (t, J=4.8 Hz, 1H), 6.30 (s, 1H), 4.21-4.17 (m, 2H), 3.93-3.87 (m, 2H), 3.75-3.70 (m, 4H), 3.49-3.44 (m, 4H), 2.41-2.39 (m, 1H), 1.91-1.85 (m, 2H).

### Parent Compound 13

LCMS (Method C): Rt=2.82 min, m/z [M+H]⁺=418

¹H NMR (400 MHz, DMSO-d₆) δ ppm: 8.61 (dd, J=1.6, 4.2 Hz, 1H), 8.46-8.42 (m, 1H), 8.27 (d, J=4.8 Hz, 2H), 7.40 (d, J=7.4 Hz, 1H), 7.01 (dd, J=4.2, 8.3 Hz, 1H), 6.56 (t, J=4.8 Hz, 1H), 6.31 (s, 1H), 4.38-4.31 (m, 1H), 3.89 (dd, J=7.5, 11.4 Hz, 1H), 3.79 (dd, J=6.1, 11.4 Hz, 1H), 3.73-3.69 (m, 4H), 3.57 (dd, J=3.3, 11.2 Hz, 1H), 3.52 (dd, J=8.9, 11.2 Hz, 1H), 3.48-3.42 (m, 4H), 3.07-2.98 (m, 1H), 2.84-2.78 (m, 1H), 1.93-1.75 (m, 3H), 1.62-1.54 (m, 1H).

### Parent Compound 14

LCMS (Method C): Rt=2.98 min, m/z [M+H]⁺=472

¹H NMR (400 MHz, DMSO-d₆) δ ppm: 8.73 (dd, J=1.5, 4.2 Hz, 1H), 8.19 (dd, J=1.5, 8.3 Hz, 1H), 7.48 (d, J=1.7 Hz, 1H), 7.15 (dd, J=4.2, 8.3 Hz, 1H), 7.03 (d, J=1.7 Hz, 1H), 6.53 (s, 1H), 3.93-3.86 (m, 2H), 3.76-3.71 (m, 4H), 3.53-3.49 (m, 4H), 3.41-3.34 (m, 1H), 3.10-3.00 (m, 2H), 2.88 (s, 6H), 2.14-1.95 (m, 4H).

### Parent Compound 15

LCMS (Method C): Rt=2.48 min, m/z [M+H]⁺=343

¹H NMR (400 MHz, DMSO-d₆) δ ppm: 8.71 (dd, J=1.5, 4.2 Hz, 1H), 8.12 (dd, J=1.5, 8.3 Hz, 1H), 7.12 (dd, J=4.2, 8.3 Hz, 1H), 6.49 (s, 1H), 4.38 (t, J=5.1 Hz, 1H), 3.83-3.75 (m, 2H), 3.75-3.72 (m, 4H), 3.53-3.45 (m, 6H), 2.95-2.85 (m, 2H), 1.80-1.75 (m, 2H), 1.67-1.59 (m, 1H), 1.49-1.38 (m, 4H).

### Parent Compound 16

LCMS (Method C): Rt=2.52 min, m/z [M+H]⁺=392

¹H NMR (400 MHz, DMSO-d₆) δ ppm: 8.72 (dd, J=1.5, 4.2 Hz, 1H), 8.28 (d, J=4.7 Hz, 2H), 8.14 (dd, J=1.5, 8.3 Hz, 1H), 7.21 (d, J=7.7 Hz, 1H), 7.15 (dd, J=4.3, 8.3 Hz, 1H), 6.56 (t, J=4.7 Hz, 1H), 6.52 (s, 1H), 4.01-3.92 (m, 1H), 3.87-3.79 (m, 2H), 3.76-3.71 (m, 4H), 3.53-3.50 (m, 4H), 3.12-3.02 (m, 2H), 2.02-1.99 (m, 2H), 1.82-1.70 (m, 2H).

### Parent Compound 17

LCMS (Method C): Rt=2.68 min, m/z [M+H]⁺=404

¹H NMR (400 MHz, DMSO-d₆) δ ppm: 8.61 (dd, J=1.5, 4.2 Hz, 1H), 8.37-8.33 (m, 3H), 7.02 (dd, J=4.2, 8.3 Hz, 1H), 6.64 (t, J=4.8 Hz, 1H), 6.31 (s, 1H), 4.58-4.52 (m, 1H), 4.11-3.93 (m, 3H), 3.81-3.73 (m, 2H), 3.72-3.68 (m, 4H), 3.65-3.56 (m, 1H), 3.45-3.40 (m, 4H), 3.17-3.09 (m, 1H), 2.16-2.06 (m, 1H), 2.00-1.91 (m, 1H).

### Parent Compound 18

LCMS (Method C): Rt=2.58 Min, m/z [M+H]⁺=406

¹H NMR (400 MHz, DMSO-d₆) δ ppm: 8.63 (dd, J=1.5, 4.2 Hz, 1H), 8.26-8.23 (m, 3H), 7.14 (d, J=7.9 Hz, 1H), 7.03 (dd, J=4.2, 8.3 Hz, 1H), 6.53 (t, J=4.7 Hz, 1H), 6.35 (s, 1H), 4.03-3.95 (m, 1H), 3.92-3.83 (m, 2H), 3.74-3.69 (m, 4H), 3.67-3.56 (m, 2H), 3.51-3.45 (m, 4H), 2.18-2.13 (m, 1H), 2.03-1.85 (m, 4H), 1.64-1.54 (m, 1H).

### Parent Compound 19

LCMS (Method C): Rt=3.30 min, m/z [M+H]⁺=400

¹H NMR (400 MHz, DMSO-d₆) δ ppm: 8.74 (dd, J=1.5, 4.2 Hz, 1H), 8.22 (dd, J=1.5, 8.3 Hz, 1H), 7.15 (dd, J=4.2, 8.3 Hz, 1H), 6.56 (s, 1H), 3.76-3.71 (m, 4H), 3.60-3.53 (m, 4H), 3.53-3.47 (m, 4H), 3.33-3.28 (m, 4H), 1.43 (s, 9H).

### Parent Compound 20 4

LCMS (Method C): Rt=2.71 min, m/z [M+H]⁺=406

¹H NMR (400 MHz, DMSO-d₆) δ ppm: 8.63 (dd, J=1.5, 4.2 Hz, 1H), 8.38-8.36 (m, 1H), 8.26 (d, J=4.7 Hz, 2H), 7.14 (d, J=8.2 Hz, 1H), 7.02 (dd, J=4.2, 8.3 Hz, 1H), 6.55 (t, J=4.7 Hz, 1H), 6.34 (s, 1H), 4.41-4.32 (m, 1H), 4.04 (dd, J=4.0, 14.0 Hz, 1H), 3.89-3.80 (m, 1H), 3.74-3.59 (m, 6H), 3.41-3.36 (m, 4H), 2.00-1.78 (m, 4H), 1.65-1.55 (m, 1H), 1.50-1.38 (m, 1H).

### Parent Compound 21

LCMS (Method C): Rt=2.41 min, m/z [M+H]⁺=392

¹H NMR (400 MHz, DMSO-d₆) δ ppm: 8.63 (dd, J=1.5, 4.2 Hz, 1H), 8.25 (d, J=4.7 Hz, 2H), 8.09 (dd, J=1.5, 8.3 Hz, 1H), 7.16 (t, J=5.8 Hz, 1H), 7.01 (dd, J=4.2, 8.3 Hz, 1H), 6.54 (t, J=4.7 Hz, 1H), 6.26 (s, 1H), 4.46-4.40 (m, 2H), 3.99 (dd, J=5.8, 8.6 Hz, 2H), 3.73-3.69 (m, 4H), 3.48-3.44 (m, 4H), 3.31-3.25 (m, 2H), 2.85-2.75 (m, 1H), 1.92-1.84 (m, 2H).

### Parent Compound 22

LCMS (Method C): Rt=2.63 min, m/z [M+H]⁺=418

¹H NMR (400 MHz, DMSO-d₆) δ ppm: 8.60 (dd, J=1.5, 4.2 Hz, 1H), 8.41-8.39 (m, 1H), 8.32 (d, J=4.7 Hz, 2H), 7.00 (dd, J=4.2, 8.4 Hz, 1H), 6.59 (t, J=4.7 Hz, 1H), 6.29 (s, 1H), 3.94-3.87 (m, 2H), 3.79-3.77 (m, 2H), 3.73-3.70 (m, 4H), 3.64-3.55 (m, 2H), 3.54-3.51 (m, 2H), 3.49-3.45 (m, 4H), 2.06-1.98 (m, 4H).

### Parent Compound 23

LCMS (Method C): Rt=2.75 min, m/z [M+H]⁺=420

¹H NMR (400 MHz, DMSO-d₆) δ ppm: 8.70 (dd, J=1.5, 4.2 Hz, 1H), 8.23 (d, J=4.8 Hz, 2H), 8.13 (dd, J=1.5, 8.3 Hz, 1H), 7.15-7.07 (m, 2H), 6.52 (t, J=4.8 Hz, 1H), 6.49 (s, 1H), 3.76-3.69 (m, 6H), 3.50-3.46 (m, 4H), 2.98-2.89 (m, 1H), 2.70-2.62 (m, 1H), 1.96-1.86 (m, 2H), 1.80-1.66 (m, 2H), 1.58-1.44 (m, 2H), 1.24-1.13 (m, 1H). CH₂ hidden by H₂O solvent peak.

### Parent Compound 24 03

LCMS (Method C): Rt=2.62 min, m/z [M+H]⁺=389

¹H NMR (400 MHz, DMSO-d₆) δ ppm: 9.26 (dd, J=1.5, 8.7 Hz, 1H), 8.87 (dd, J=1.5, 4.2 Hz, 1H), 8.61 (d, J=0.7 Hz, 1H), 8.32 (d, J=4.8 Hz, 2H), 7.88 (d, J=0.7 Hz, 1H), 7.58 (t, J=6.0 Hz, 1H), 7.29 (dd, J=4.2, 8.7 Hz, 1H), 7.02 (s, 1H), 6.61 (t, J=4.8 Hz, 1H), 4.47 (d, J=6.0 Hz, 2H), 3.80-3.75 (m, 4H), 3.62-3.57 (m, 4H).

### Parent Compound 25

LCMS (Method C): Rt=2.69 min, m/z [M+H]⁺=406

¹H NMR (400 MHz, DMSO-d₆) δ ppm: 8.59 (dd, J=1.5, 4.2 Hz, 1H), 8.40-8.36 (m, 1H), 8.19 (d, J=4.6 Hz, 2H), 7.34 (t, J=6.4 Hz, 1H), 6.98 (dd, J=4.2, 8.4 Hz, 1H), 6.51 (t, J=4.6 Hz, 1H), 6.25 (s, 1H), 3.90-3.84 (m, 2H), 3.80-3.75 (m, 1H), 3.73-3.68 (m, 4H), 3.46-3.35 (m, 7H), 1.98-1.89 (m, 1H), 1.76-1.64 (m, 1H), 1.11 (s, 3H).

### Parent Compound 26

LCMS (Method C): Rt=2.27 min, m/z [M+H]⁺=420

¹H NMR (400 MHz, DMSO-d₆) δ ppm: 10.61 (s, 1H), 8.73 (dd, J=1.5, 4.2 Hz, 1H), 8.66 (d, J=4.8 Hz, 2H), 8.20-8.17 (m, 1H), 7.20-7.13 (m, 2H), 6.53 (s, 1H), 3.90-3.83 (m, 2H), 3.76-3.71 (m, 4H), 3.53-3.48 (m, 4H), 3.02-2.86 (m, 3H), 1.97-1.87 (m, 4H).

### Parent Compound 27

LCMS (Method C): Rt=2.30 min, m/z [M+H]⁺=420

¹H NMR (400 MHz, DMSO-d₆) δ ppm: 8.96 (d, J=4.9 Hz, 2H), 8.81 (d, J=8.2 Hz, 1H), 8.73 (dd, J=1.5, 4.2 Hz, 1H), 8.14 (dd, J=1.5, 8.3 Hz, 1H), 7.68 (t, J=4.9 Hz, 1H), 7.16 (dd, J=4.2, 8.3 Hz, 1H), 6.52 (s, 1H), 4.13-4.06 (m, 1H), 3.89-3.82 (m, 2H), 3.76-3.71 (m, 4H), 3.54-3.48 (m, 4H), 3.15-3.06 (m, 2H), 1.97-1.89 (m, 4H).

### Parent Compound 28

LCMS (Method C): Rt=2.51 min, m/z [M+H]⁺=404

¹H NMR (400 MHz, DMSO-d₆) δ ppm: 8.60 (dd, J=1.5, 4.2 Hz, 1H), 8.44-8.42 (m, 1H), 8.34 (d, J=4.8 Hz, 2H), 6.98 (dd, J=4.2, 8.3 Hz, 1H), 6.61 (t, J=4.8 Hz, 1H), 6.29 (s, 1H), 4.11-4.03 (m, 2H), 3.82-3.68 (m, 8H), 3.52-3.43 (m, 6H), 3.14-3.07 (m, 2H).

### Parent Compound 29

LCMS (Method C): Rt=2.80 min, m/z [M+H]⁺=404

¹H NMR (400 MHz, DMSO-d₆) δ ppm: 8.61 (dd, J=1.5, 4.2 Hz, 1H), 8.37 (dd, J=1.5, 8.3 Hz, 1H), 8.33 (d, J=4.8 Hz, 2H), 7.00 (dd, J=4.2, 8.3 Hz, 1H), 6.66 (t, J=4.8 Hz, 1H), 6.30 (s, 1H), 4.51 (d, J=10.5 Hz, 1H), 3.99-3.82 (m, 5H), 3.73-3.68 (m, 4H), 3.49-3.45 (m, 4H), 2.94-2.84 (m, 1H), 2.45-2.29 (m, 2H), 2.08-2.00 (m, 1H).

### Parent Compound 30

LCMS (Method C): Rt=3.53 min, m/z [M+H]⁺=442

¹H NMR (400 MHz, DMSO-d₆) δ ppm: 8.71 (dd, J=1.5, 4.2 Hz, 1H), 8.12 (dd, J=1.5, 8.3 Hz, 1H), 7.12 (dd, J=4.2, 8.3 Hz, 1H), 6.80 (t, J=5.4 Hz, 1H), 6.49 (s, 1H), 3.82-3.75 (m, 2H), 3.75-3.72 (m, 4H), 3.51-3.47 (m, 4H), 3.04-2.95 (m, 2H), 2.94-2.83 (m, 2H), 1.82-1.75 (m, 2H), 1.56-1.49 (m, 1H), 1.43-1.32 (m, 13H).

### Parent Compound 31

LCMS (Method C): Rt=2.64 min, m/z [M+H]⁺=392

¹H NMR (400 MHz, DMSO-d₆) δ ppm: 8.71 (dd, J=1.7, 4.2 Hz, 1H), 8.34-8.29 (m, 3H), 7.22 (d, J=7.6 Hz, 1H), 7.16 (dd, J=4.2, 8.3 Hz, 1H), 6.59 (t, J=4.7 Hz, 1H), 6.49 (s, 1H), 4.18-4.09 (m, 1H), 3.99-3.92 (m, 1H), 3.75-3.70 (m, 5H), 3.55-3.47 (m, 4H), 3.02-2.93 (m, 1H), 2.80 (dd, J=9.6, 12.1 Hz, 1H), 2.06-1.98 (m, 1H), 1.93-1.86 (m, 1H), 1.81-1.71 (m, 1H), 1.67-1.55 (m, 1H).

### Parent Compound 32

LCMS (Method C): Rt=3.08 min, m/z [M+H]⁺=299

¹H NMR (400 MHz, DMSO-d₆) δ ppm: 8.71 (dd, J=1.5, 4.2 Hz, 1H), 8.13 (dd, J=1.5, 8.3 Hz, 1H), 7.13 (dd, J=4.2, 8.3 Hz, 1H), 6.50 (s, 1H), 3.75-3.71 (m, 4H), 3.52-3.47 (m, 4H), 3.37-3.31 (m, 4H), 1.76-1.68 (m, 4H), 1.65-1.59 (m, 2H).

### Parent Compound 33

LCMS (Method C): Rt=3.32 min, m/z [M+H]⁺=428

¹H NMR (400 MHz, DMSO-d₆) δ ppm: 8.71 (dd, J=1.5, 4.2 Hz, 1H), 8.12 (dd, J=1.5, 8.3 Hz, 1H), 7.13 (dd, J=4.2, 8.3 Hz, 1H), 6.91 (t, J=5.8 Hz, 1H), 6.50 (s, 1H), 3.83-3.75 (m, 2H), 3.74-3.70 (m, 4H), 3.51-3.47 (m, 4H), 2.94-2.84 (m, 4H), 1.78-1.71 (m, 2H), 1.65-1.53 (m, 1H), 1.44-1.31 (m, 11H).

### Parent Compound 34

LCMS (Method C): Rt=2.53 min, m/z [M+H]⁺=404

¹H NMR (400 MHz, DMSO-d₆) δ ppm: 8.61 (dd, J=1.5, 4.2 Hz, 1H), 8.42-8.38 (m, 1H), 8.35 (d, J=4.8 Hz, 2H), 7.01 (dd, J=4.2, 8.4 Hz, 1H), 6.67 (t, J=4.8 Hz, 1H), 6.29 (s, 1H), 4.09-4.00 (m, 6H), 3.88-3.81 (m, 2H), 3.74-3.69 (m, 4H), 3.50-3.46 (m, 4H), 2.25-2.19 (m, 2H).

### Parent Compound 35

LCMS (Method C): Rt=2.57 min, m/z [M+H]⁺=404

¹H NMR (400 MHz, DMSO-d₆) δ ppm: 8.64 (dd, J=1.4, 4.2 Hz, 1H), 8.35 (d, J=4.8 Hz, 2H), 8.13 (dd, J=1.4, 8.4 Hz, 1H), 7.01 (dd, J=4.2, 8.4 Hz, 1H), 6.61 (t, J=4.8 Hz, 1H), 6.30 (s, 1H), 4.38-4.30 (m, 4H), 3.76-3.68 (m, 6H), 3.60-3.55 (m, 2H), 3.50-3.45 (m, 4H), 2.29-2.23 (m, 2H).

### Parent Compound 36

LCMS (Method C): Rt=2.63 min, m/z [M+H]⁺=406

¹H NMR (400 MHz, DMSO-d₆) δ ppm: 8.69 (dd, J=1.4, 4.2 Hz, 1H), 8.25 (d, J=4.8 Hz, 2H), 8.06 (dd, J=1.4, 8.3 Hz, 1H), 7.29 (t, J=6.0 Hz, 1H), 7.04 (dd, J=4.2, 8.3 Hz, 1H), 6.54 (t, J=4.8 Hz, 1H), 6.47 (s, 1H), 3.84-3.76 (m, 1H), 3.71-3.66 (m, 5H), 3.46-3.41 (m, 4H), 3.30-3.22 (m, 2H), 3.00-2.90 (m, 1H), 2.72 (dd, J=10.4, 12.7 Hz, 1H), 2.15-2.09 (m, 1H), 1.90-1.68 (m, 3H), 1.29-1.17 (m, 1H).

### Parent Compound 37

LCMS (Method C): Rt=3.34 min, m/z [M+H]⁺=426

¹H NMR (400 MHz, DMSO-d₆) δ ppm: 8.63 (dd, J=1.6, 4.2 Hz, 1H), 8.39-8.34 (m, 1H), 7.03 (dd, J=4.2, 8.4 Hz, 1H), 6.33 (s, 1H), 4.23-4.22 (m, 1H), 3.95-3.85 (m, 3H), 3.74-3.69 (m, 5H), 3.52-3.38 (m, 6H), 3.07-2.96 (m, 1H), 2.06-1.94 (m, 1H), 1.87-1.73 (m, 1H), 1.39 (s, 9H).

### Parent Compound 38

LCMS (Method C): Rt=3.08 min, m/z [M+H]⁺=418

¹H NMR (400 MHz, DMSO-d₆) δ ppm: 8.60 (dd, J=1.5, 4.2 Hz, 1H), 8.39-8.35 (m, 3H), 6.98 (dd, J=4.2, 8.4 Hz, 1H), 6.64 (t, J=4.8 Hz, 1H), 6.29 (s, 1H), 4.75 (d, J=9.8 Hz, 1H), 3.99-3.80 (m, 2H), 3.73-3.68 (m, 5H), 3.62-3.49 (m, 2H), 3.49-3.39 (m, 5H), 2.08-2.03 (m, 2H), 1.92-1.85 (m, 2H), 1.64-1.58 (m, 1H).

### Parent Compound 39

LCMS (Method C): Rt=2.42 min, m/z [M+H]⁺=390

¹H NMR (400 MHz, DMSO-d₆) δ ppm: 8.65 (dd, J=1.5, 4.2 Hz, 1H), 8.35 (d, J=4.8 Hz, 2H), 8.10 (dd, J=1.5, 8.4 Hz, 1H), 7.04 (dd, J=4.2, 8.4 Hz, 1H), 6.69 (t, J=4.8 Hz, 1H), 6.31 (s, 1H), 4.54 (s, 4H), 4.25 (s, 4H), 3.74-3.69 (m, 4H), 3.50-3.46 (m, 4H).

### Parent Compound 40

LCMS (Method C): Rt=2.68 min, m/z [M+H]⁺=390

¹H NMR (400 MHz, DMSO-d₆) δ ppm: 8.65 (dd, J=1.7, 4.3 Hz, 1H), 8.32 (d, J=4.7 Hz, 2H), 8.13-8.09 (m, 1H), 7.01 (dd, J=4.3, 8.3 Hz, 1H), 6.66 (t, J=4.7 Hz, 1H), 6.30 (s, 1H), 5.03 (d, J=9.8 Hz, 2H), 4.47 (d, J=9.8 Hz, 2H), 3.96-3.90 (m, 2H), 3.73-3.68 (m, 4H), 3.52-3.44 (m, 4H), 2.71-2.65 (m, 2H).

### Parent Compound 41

LCMS (Method C): Rt=2.96 min, m/z [M+H]⁺=404

¹H NMR (400 MHz, DMSO-d₆) δ ppm: 8.63 (dd, J=1.5, 4.3 Hz, 1H), 8.21 (bs, 2H), 8.11 (dd, J=1.5, 8.3 Hz, 1H), 6.99 (dd, J=4.3, 8.3 Hz, 1H), 6.59 (t, J=4.8 Hz, 1H), 6.28 (s, 1H), 5.16 (d, J=8.4 Hz, 2H), 4.23 (d, J=8.4 Hz, 2H), 3.72-3.67 (m, 4H), 3.63-3.57 (m, 2H), 3.48-3.44 (m, 4H), 2.45-2.41 (m, 2H), 1.92-1.83 (m, 2H).

### Parent Compound 42

LCMS (Method C): Rt=1.68 min, m/z [M+H]⁺=418

¹H NMR (400 MHz, DMSO-d₆) δ ppm: 8.75 (d, J=4.9 Hz, 2H), 8.61 (dd, J=1.6, 4.2 Hz, 1H), 8.36-8.34 (m, 1H), 7.38 (t, J=4.9 Hz, 1H), 7.01 (dd, J=4.2, 8.4 Hz, 1H), 6.29 (s, 1H), 4.03 (d, J=13.4 Hz, 1H), 3.83-3.60 (m, 9H), 3.48-3.42 (m, 4H), 3.40-3.25 (m, 1H), 3.18-3.10 (m, 1H), 2.90-2.80 (m, 1H), 2.59-2.53 (m, 1H), 2.06-1.96 (m, 1H), 1.74-1.63 (m, 1H).

### Parent Compound 43

LCMS (Method A): Rt=1.20 min, m/z [M+H]⁺=429

¹H NMR (400 MHz, DMSO-d₆) δ ppm: 8.79 (dd, J=1.8, 4.3 Hz, 1H), 8.27 (ddd, J=0.6, 1.8, 8.2 Hz, 1H), 7.19 (dd, J=4.3, 8.2 Hz, 1H), 6.53 (s, 1H), 4.31 (d, J=6.3 Hz, 2H), 4.02-3.95 (m, 2H), 3.76-3.72 (m, 4H), 3.53-3.48 (m, 4H), 2.82-2.69 (m, 2H), 2.06-2.00 (m, 1H), 1.82-1.78 (m, 2H), 1.40 (s, 9H), 1.28-1.17 (m, 2H).

### Parent Compound 44

LCMS (Method A): Rt=1.04 min, m/z [M+H]⁺=401

¹H NMR (400 MHz, DMSO-d₆) δ ppm: 8.79 (dd, J=1.7, 4.3 Hz, 1H), 8.24-8.21 (m, 1H), 7.18 (dd, J=4.3, 8.2 Hz, 1H), 6.56 (s, 1H), 4.57 (d, J=5.9 Hz, 2H), 4.04-3.95 (m, 2H), 3.80-3.72 (m, 6H), 3.54-3.50 (m, 4H), 3.09-3.02 (m, 1H), 1.38 (s, 9H).

### Parent Compound 45

LCMS (Method F): Rt=2.54 min, m/z [M+H]⁺=429

¹H NMR (400 MHz, DMSO-d₆) δ ppm: 8.79 (dd, J=1.7, 4.3 Hz, 1H), 8.37 (dd, J=1.7, 8.2 Hz, 0.4H), 8.30 (dd, J=1.7, 8.2 Hz, 0.6H), 7.22-7.17 (m, 1H), 6.54 (s, 1H), 5.45-5.42 (m, 0.4H), 5.05-5.00 (m, 0.6H), 3.86-3.79 (m, 1.2H), 3.77-3.72 (m, 4H), 3.68-3.59 (m, 0.8H), 3.53-3.45 (m, 4H), 3.28-3.13 (m, 1.2H), 3.02-2.78 (m, 0.8H), 2.21-2.16 (m, 0.6H), 2.08-1.88 (m, 1.4H), 1.81-1.72 (m, 0.6H), 1.56-1.46 (m, 0.4H), 1.42 (s, 9H), 0.97-0.90 (m, 3H). 3:2 mixture of trans:cis diastereomers.

### Parent Compound 46

LCMS (Method A): Rt=1.27 min, m/z [M+H]⁺=433

¹H NMR (400 MHz, CDCl₃) δ ppm: 8.81 (dd, J=1.8, 4.3 Hz, 1H), 8.28 (dd, J=1.8, 8.2 Hz, 1H), 7.11 (dd, J=4.3, 8.2 Hz, 1H), 6.59 (s, 1H), 5.55-5.48 (m, 1H), 4.86-4.71 (m, 1H), 3.89-3.85 (m, 4H), 3.84-3.75 (m, 2H), 3.63-3.52 (m, 6H), 2.34-2.25 (m, 1H), 1.89-1.83 (m, 1H).

### Parent Compound 47

LCMS (Method B): Rt=1.67 min, m/z [M+H]⁺=400

### Parent Compound 48

LCMS (Method C): Rt=3.56 min, m/z [M+H]⁺=415

¹H NMR (400 MHz, DMSO-d₆) δ ppm: 8.79 (dd, J=1.5, 4.3 Hz, 1H), 8.31 (dd, J=1.5, 8.2 Hz, 1H), 7.19 (dd, J=4.3, 8.2 Hz, 1H), 6.54 (s, 1H), 5.43-5.36 (m, 1H), 3.76-3.72 (m, 4H), 3.66-3.58 (m, 2H), 3.51-3.47 (m, 4H), 3.41-3.35 (m, 2H), 2.00-1.97 (m, 2H), 1.78-1.68 (m, 2H), 1.42 (s, 9H).

### Parent Compound 49

LCMS (Method B): Rt=1.53 min, m/z [M+H]⁺=330

¹H NMR (400 MHz, DMSO-d₆) δ ppm: 8.77 (dd, J=1.7, 4.4 Hz, 1H), 8.23 (dd, J=1.7, 8.2 Hz, 1H), 7.17 (dd, J=4.4, 8.2 Hz, 1H), 6.51 (s, 1H), 5.14 (tt, J=4.4, 8.6 Hz, 1H), 4.58 (d, J=4.3 Hz, 1H), 3.77-3.72 (m, 4H), 3.61 (dtt, J=4.3, 8.6, 8.7 Hz, 1H), 3.52-3.47 (m, 4H), 2.14-2.09 (m, 2H), 1.92-1.87 (m, 2H), 1.75-1.72 (m, 2H), 1.20-1.11 (m, 2H).

### Parent Compound 50

LCMS (Method B): Rt=0.98 min, m/z [M+H]⁺=473

¹H NMR (400 MHz, CDCl₃) δ ppm: 8.79 (dd, J=1.5, 4.4 Hz, 1H), 8.36 (dd, J=1.5, 8.2 Hz, 1H), 7.10 (dd, J=4.4, 8.2 Hz, 1H), 6.53 (s, 1H), 5.75 (t, J=5.6 Hz, 1H), 5.52-5.47 (m, 1H), 4.52 (t, J=5.3 Hz, 1H), 3.89-3.83 (m, 4H), 3.78-3.66 (m, 4H), 3.62-3.46 (m, 4H), 3.42 (dd, J=5.3, 5.6 Hz, 2H), 2.31-2.20 (m, 3H), 2.08-1.92 (m, 2H), 1.85-1.62 (m, 4H), 1.22 (t, J=6.9 Hz, 6H).

### Parent Compound 51

LCMS (Method A): Rt=1.02 min, m/z [M+H]⁺=459

### Parent Compound 52

LCMS (Method B): Rt=1.04 min, m/z [M+H]⁺=358

### Parent Compound 53

LCMS (Method C): Rt=3.39 min, m/z [M+H]⁺=352

¹H NMR (400 MHz, DMSO-d₆) δ ppm: 8.78 (dd, J=1.5, 4.3 Hz, 1H), 8.26 (dd, J=1.5, 8.3 Hz, 1H), 7.48-7.44 (m, 2H), 7.17 (dd, J=4.3, 8.3 Hz, 1H), 6.97-6.94 (m, 2H), 6.55 (s, 1H), 5.47 (s, 2H), 3.76-3.74 (m, 7H), 3.56-3.52 (m, 4H).

### Parent Compound 55

LCMS (Method A): Rt=1.51 min, m/z [M+H]⁺=476/478

¹H NMR (400 MHz, CDCl₃) δ ppm: 8.78 (d, J=2.4 Hz, 1H), 8.42-8.40 (m, 1H), 7.39 (d, J=4.6 Hz, 1H), 7.36 (d, J=4.4 Hz, 1H), 6.51 (s, 1H), 5.49-5.46 (m, 1H), 4.22-4.14 (m, 1H), 3.88-3.84 (m, 4H), 3.57-3.53 (m, 4H), 2.40-2.33 (m, 2H), 2.21-2.04 (m, 4H), 1.89-1.79 (m, 2H).

### Parent Compound 56

LCMS (Method C): Rt=2.56 min, m/z [M+H]⁺=395

¹H NMR (400 MHz, DMSO-d₆) δ ppm: 8.80 (dd, J=1.5, 4.3 Hz, 1H), 8.30 (dd, J=1.5, 8.2 Hz, 1H), 7.78 (s, 1H), 7.23 (dd, J=4.3, 8.2 Hz, 1H), 6.53 (s, 1H), 5.51-5.46 (m, 1H), 3.84 (s, 3H), 3.77-3.72 (m, 4H), 3.53-3.48 (m, 4H), 3.14-3.06 (m, 1H), 2.22-2.14 (m, 2H), 2.03-1.71 (m, 6H).

### Parent Compound 57 02

LCMS (Method C): Rt=2.44 min, m/z [M+H]⁺=371

¹H NMR (400 MHz, DMSO-d₆) δ ppm: 8.79 (dd, J=1.5, 4.3 Hz, 1H), 8.28 (dd, J=1.5, 8.2 Hz, 1H), 7.21-7.17 (m, 2H), 6.66 (bs, 1H), 6.53 (s, 1H), 4.26 (d, J=6.2 Hz, 2H), 3.77-3.72 (m, 4H), 3.53-3.48 (m, 4H), 2.13-2.02 (m, 1H), 1.95-1.87 (m, 2H), 1.85-1.76 (m, 3H), 1.45-1.31 (m, 2H), 1.20-1.07 (m, 2H).

### Parent Compound 58 (Formic Acid 0.70 Equivalents)

LCMS (Method C): Rt=2.35 min, m/z [M+H]⁺=380

¹H NMR (400 MHz, DMSO-d₆) δ ppm: 13.66 (bs, 1H), 8.60 (dd, J=1.6, 4.3 Hz, 1H), 8.51-8.46 (m, 1H), 7.84 (bs, 1H), 7.09 (d, J=6.7 Hz, 1H), 7.00 (dd, J=4.3, 8.3 Hz, 1H), 6.13 (s, 1H), 4.19-4.09 (m, 1H), 3.75-3.70 (m, 4H), 3.49-3.43 (m, 4H), 3.06-2.94 (m, 1H), 2.27-2.16 (m, 2H), 1.87-1.73 (m, 6H).

### Parent Compound 59

LCMS (Method A): Rt=1.60 min, m/z [M+H]⁺=472/474

### Parent Compound 60

LCMS (Method A): Rt=1.90 min, m/z [M+H]⁺=474/476

¹H NMR (400 MHz, CDCl₃) δ ppm: 8.78-8.77 (m, 1H), 8.44-8.40 (m, 1H), 7.19-7.15 (m, 1H), 7.00-6.86 (m, 2H), 6.50 (s, 1H), 5.53-5.48 (m, 0.7H), 5.22-5.13 (m, 0.3H), 3.90-3.84 (m, 4H), 3.58-3.53 (m, 4H), 3.05-2.86 (m, 1H), 2.32-2.23 (m, 2.6H), 2.03-1.65 (m, 5.4H). 7:3 mixture of cis:trans diastereomers.

### Parent Compound 61 L

CMS (Method A): Rt=1.93 min, m/z [M+H]⁺=468/470

¹H NMR (400 MHz, CDCl₃) δ ppm: 8.79-8.78 (m, 1H), 8.46-8.44 (m, 1H), 7.39-7.21 (m, 5H), 6.50 (s, 1H), 5.54-5.51 (m, 1H), 3.89-3.84 (m, 4H), 3.59-3.54 (m, 4H), 2.74-2.63 (m, 1H), 2.36-2.30 (m, 2H), 2.00-1.65 (m, 6H).

### Parent Compound 62

LCMS (Method A): Rt=1.67 min, m/z [M+H]⁺=475/477

¹H NMR (400 MHz, CDCl₃) δ ppm: 8.80 (d, J=2.2 Hz, 1H), 8.76 (d, J=2.2 Hz, 1H), 8.42-8.40 (m, 1H), 7.04-7.02 (m, 1H), 6.50-6.48 (m, 1H), 5.54-5.49 (m, 1H), 3.89-3.83 (m, 4H), 3.59-3.53 (m, 4H), 3.06-2.96 (m, 1H), 2.34-2.27 (m, 2H), 2.12-1.78 (m, 6H).

### Parent Compound 63

LCMS (Method A): Rt=1.68 min, m/z [M+H]⁺=475/477

¹H NMR (400 MHz, CDCl₃) δ ppm: 8.77 (d, J=2.4 Hz, 1H), 8.41 (dd, J=0.7, 2.4 Hz, 1H), 7.73 (d, J=3.4 Hz, 1H), 7.25 (d, J=3.4 Hz, 1H), 6.50 (s, 1H), 5.53-5.48 (m, 1H), 3.88-3.83 (m, 4H), 3.58-3.53 (m, 4H), 3.25-3.14 (m, 1H), 2.35-2.25 (m, 2H), 2.16-2.06 (m, 4H), 1.92-1.79 (m, 2H).

### Parent Compound 64

LCMS (Method A): Rt=1.18 min, m/z [M+H]⁺=459/461

¹H NMR (400 MHz, DMSO-d₆) δ ppm: 13.69 (bs, 1H), 8.83-8.82 (m, 1H), 8.40 (bs, 1H), 7.88 (bs, 1H), 6.54 (s, 1H), 5.45-5.41 (m, 1H), 3.76-3.73 (m, 4H), 3.55-3.50 (m, 4H), 2.98-2.90 (m, 1H), 2.14-2.07 (m, 2H), 2.05-1.94 (m, 2H), 1.89-1.80 (m, 4H).

### Parent Compound 66

LCMS (Method E): Rt=2.55 min, m/z [M+H]⁺=395

¹H NMR (400 MHz, DMSO-d₆) δ ppm: 13.66 (bs, 1H), 8.78 (dd, J=1.8, 4.3 Hz, 1H), 8.34-8.28 (m, 1H), 7.95 (bs, 1H), 7.19 (dd, J=4.3, 8.2 Hz, 1H), 6.53 (s, 1H), 5.49-5.43 (m, 1H), 4.16-4.07 (m, 2H), 3.94 (dd, J=2.4, 11.5 Hz, 1H), 3.65-3.55 (m, 2H), 2.96-2.82 (m, 2H), 2.58-2.52 (m, 1H), 2.14-1.79 (m, 8H), 1.19 (d, J=6.2 Hz, 3H).

### Parent Compound 67

LCMS (Method E): Rt=2.53 min, m/z [M+H]⁺=395

¹H NMR (400 MHz, DMSO-d₆) δ ppm: 13.66 (bs, 1H), 8.77 (dd, J=1.8, 4.3 Hz, 1H), 8.33-8.27 (m, 1H), 7.84 (bs, 1H), 7.17 (dd, J=4.3, 8.2 Hz, 1H), 6.45 (s, 1H), 5.48-5.42 (m, 1H), 4.47-4.39 (m, 1H), 3.97 (dd, J=3.0, 11.6 Hz, 1H), 3.84-3.74 (m, 2H), 3.67 (dd, J=3.0, 11.6 Hz, 1H), 3.53 (dt, J=3.0, 11.6 Hz, 1H), 3.13 (dt, J=3.8, 12.5 Hz, 1H), 2.98-2.90 (m, 1H), 2.12-1.79 (m, 8H), 1.16 (d, J=6.7 Hz, 3H).

### Parent Compound 68

LCMS (Method E): Rt=2.55 min, m/z [M+H]⁺=395

¹H NMR (400 MHz, DMSO-d₆) δ ppm: 13.65 (bs, 1H), 8.78 (dd, J=1.8, 4.3 Hz, 1H), 8.32-8.30 (m, 1H), 8.03 (bs, 1H), 7.19 (dd, J=4.3, 8.2 Hz, 1H), 6.53 (s, 1H), 5.49-5.43 (m, 1H), 4.16-4.07 (m, 2H), 3.94 (dd, J=2.5, 11.4 Hz, 1H), 3.65-3.55 (m, 2H), 2.98-2.82 (m, 2H), 2.59-2.53 (m, 1H), 2.14-1.79 (m, 8H), 1.19 (d, J=6.2 Hz, 3H).

### Parent Compound 69

LCMS (Method E): Rt=2.53 min, m/z [M+H]⁺=395

¹H NMR (400 MHz, DMSO-d₆) δ ppm: 13.67 (bs, 1H), 8.76 (dd, J=1.8, 4.3 Hz, 1H), 8.31-8.27 (m, 1H), 7.93 (bs, 1H), 7.17 (dd, J=4.3, 8.2 Hz, 1H), 6.45 (s, 1H), 5.48-5.43 (m, 1H), 4.45-4.40 (m, 1H), 4.00-3.94 (m, 1H), 3.84-3.74 (m, 2H), 3.70-3.65 (m, 1H), 3.57-3.49 (m, 1H), 3.17-3.08 (m, 1H), 2.98-2.89 (m, 1H), 2.13-1.93 (m, 4H), 1.89-1.80 (m, 4H), 1.16 (d, J=6.7 Hz, 3H).

### Parent Compound 71

LCMS (Method C): Rt=2.42 min, m/z [M+H]⁺=381

¹H NMR (400 MHz, DMSO-d₆) δ ppm: 13.67 (bs, 1H), 8.79 (dd, J=1.8, 4.3 Hz, 1H), 8.35-8.31 (m, 1H), 7.95 (bs, 1H), 7.20 (dd, J=4.3, 8.2 Hz, 1H), 6.53 (s, 1H), 5.48-5.44 (m, 1H), 3.77-3.72 (m, 4H), 3.53-3.47 (m, 4H), 2.98-2.89 (m, 1H), 2.14-1.78 (m, 8H).

### Parent Compound 72

LCMS (Method C): Rt=2.62 min, m/z [M+H]⁺=395

¹H NMR (400 MHz, DMSO-d₆) δ ppm: 8.78 (dd, J=1.8, 4.3 Hz, 1H), 8.31 (s, 1H), 8.26 (dd, J=1.8, 8.2 Hz, 1H), 7.20 (dd, J=4.3, 8.2 Hz, 1H), 6.52 (s, 1H), 5.46-5.41 (m, 1H), 3.81 (s, 3H), 3.77-3.72 (m, 4H), 3.52-3.48 (m, 4H), 2.87-2.79 (m, 1H), 2.13-2.05 (m, 2H), 2.02-1.77 (m, 6H).

### Parent Compound 73

LCMS (Method C): Rt=2.48 min, m/z [M+H]⁺=395

¹H NMR (400 MHz, DMSO-d₆) δ ppm: 13.60 (bs, 1H), 8.78 (dd, J=1.5, 4.3 Hz, 1H), 8.24 (dd, J=1.5, 8.2 Hz, 1H), 7.95 (bs, 1H), 7.18 (dd, J=4.3, 8.2 Hz, 1H), 6.52 (s, 1H), 4.34 (d, J=6.8 Hz, 2H), 3.75-3.71 (m, 4H), 3.51-3.47 (m, 4H), 3.04-2.98 (m, 1H), 2.10-2.02 (m, 3H), 1.78-1.64 (m, 4H), 1.58-1.49 (m, 2H).

### Parent Compound 78

LCMS (Method E): Rt=3.23 min, m/z [M+H]⁺=398

¹H NMR (400 MHz, DMSO-d₆) δ ppm: 8.80 (dd, J=1.5, 4.3 Hz, 1H), 8.39 (dd, J=1.5, 8.2 Hz, 1H), 8.01 (dd, J=0.7, 4.6 Hz, 1H), 7.47 (dd, J=0.7, 4.4 Hz, 1H), 7.22 (dd, J=4.3, 8.2 Hz, 1H), 6.54 (s, 1H), 5.49-5.44 (m, 1H), 4.28-4.20 (m, 1H), 3.77-3.72 (m, 4H), 3.52-3.48 (m, 4H), 2.22-2.08 (m, 4H), 1.97-1.81 (m, 4H).

### Parent Compound 79

LCMS (Method E): Rt=2.62 min, m/z [M+H]⁺=381

¹H NMR (400 MHz, DMSO-d₆) δ ppm: 8.80 (dd, J=1.5, 4.3 Hz, 1H), 8.43 (dd, J=1.5, 8.2 Hz, 1H), 8.27 (d, J=1.0 Hz, 1H), 7.75 (d, J=1.0 Hz, 1H), 7.22 (dd, J=4.3, 8.2 Hz, 1H), 6.54 (s, 1H), 5.53-5.48 (m, 1H), 4.75-4.66 (m, 1H), 3.77-3.73 (m, 4H), 3.53-3.48 (m, 4H), 2.27-2.17 (m, 4H), 2.05-1.87 (m, 4H).

### Parent Compound 80

LCMS (Method E): Rt=2.54 min, m/z [M+H]⁺=381

¹H NMR (400 MHz, DMSO-d₆) δ ppm: 8.80 (dd, J=1.5, 4.3 Hz, 1H), 8.63 (s, 1H), 8.38 (dd, J=1.5, 8.2 Hz, 1H), 7.98 (s, 1H), 7.22 (dd, J=4.3, 8.2 Hz, 1H), 6.54 (s, 1H), 5.51-5.45 (m, 1H), 4.50-4.43 (m, 1H), 3.77-3.73 (m, 4H), 3.53-3.48 (m, 4H), 2.23-2.15 (m, 4H), 2.08-1.83 (m, 4H).

### Parent Compound 81

LCMS (Method E): Rt=3.01 min, m/z [M+H]⁺=381

¹H NMR (400 MHz, DMSO-d₆) δ ppm: 8.80 (dd, J=1.5, 4.3 Hz, 1H), 8.31 (dd, J=1.5, 8.2 Hz, 1H), 7.79 (s, 2H), 7.22 (dd, J=4.3, 8.2 Hz, 1H), 6.54 (s, 1H), 5.49-5.43 (m, 1H), 4.76-4.67 (m, 1H), 3.77-3.73 (m, 4H), 3.53-3.49 (m, 4H), 2.34-2.13 (m, 4H), 2.08-1.88 (m, 4H).

### Parent Compound 85

LCMS (Method C): Rt=2.26 min, m/z [M+H]⁺=376

¹H NMR (400 MHz, DMSO-d₆) δ ppm: 8.76 (dd, J=1.5, 4.2 Hz, 1H), 8.50-8.48 (m, 1H), 8.45-8.41 (m, 1H), 8.23 (dd, J=1.6, 4.7 Hz, 1H), 7.63-7.59 (m, 1H), 7.24 (dd, J=4.2, 8.3 Hz, 1H), 7.14-7.11 (m, 1H), 6.49 (s, 1H), 4.59 (dd, J=3.1, 10.5 Hz, 1H), 3.67-3.50 (m, 5H), 3.41-3.34 (m, 2H), 3.27-3.20 (m, 2H), 2.88-2.80 (m, 1H), 1.98-1.68 (m, 5H), 1.61-1.54 (m, 1H).

### Parent Compound 87

LCMS (Method C): Rt=1.69 min, m/z [M+H]⁺=352

¹H NMR (400 MHz, DMSO-d₆) δ ppm: 11.80 (s, 1H), 8.79 (dd, J=1.5, 4.3 Hz, 1H), 8.28 (dd, J=1.5, 8.2 Hz, 1H), 7.20 (dd, J=4.3, 8.2 Hz, 1H), 6.98 (bs, 1H), 6.79 (bs, 1H), 6.55 (s, 1H), 5.33-5.24 (m, 1H), 3.77-3.72 (m, 4H), 3.55-3.50 (m, 4H), 3.28-3.21 (m, 1H), 2.93-2.84 (m, 2H), 2.47-2.40 (m, 2H).

Parent compound 89

LCMS (Method C): Rt=1.54 min, m/z [M+H]⁺=315

¹H NMR (400 MHz, DMSO-d₆) δ ppm: 8.78 (dd, J=1.5, 4.3 Hz, 1H), 8.27 (dd, J=1.5, 8.2 Hz, 1H), 7.18 (dd, J=4.3, 8.2 Hz, 1H), 6.52 (s, 1H), 5.28-5.20 (m, 1H), 3.76-3.72 (m, 4H), 3.51-3.46 (m, 4H), 3.03-2.95 (m, 2H), 2.68-2.60 (m, 2H), 2.24 (bs, 1H), 2.04-1.96 (m, 2H), 1.68-1.57 (m, 2H).

### Parent Compound 90

LCMS (Method A): Rt=0.56 min, m/z [M+H]⁺=329

¹H NMR (400 MHz, DMSO-d₆) δ ppm: 8.78 (dd, J=1.9, 4.3 Hz, 1H), 8.27 (ddd, J=0.7, 1.9, 8.2 Hz, 1H), 7.19 (dd, J=4.3, 8.2 Hz, 1H), 6.52 (s, 1H), 4.27 (d, J=6.3 Hz, 2H), 3.76-3.72 (m, 4H), 3.52-3.48 (m, 4H), 2.98-2.93 (m, 2H), 2.49-2.44 (m, 2H), 2.23 (s, 1H), 1.97-1.86 (m, 1H), 1.75-1.70 (m, 2H), 1.28-1.17 (m, 2H).

### Parent Compound 91

LCMS (Method B): Rt=1.72 min, m/z [M+H]⁺=301

¹H NMR (400 MHz, DMSO-d₆) δ ppm: 8.79 (dd, J=1.5, 4.3 Hz, 1H), 8.27 (dd, J=1.5, 8.2 Hz, 1H), 7.19 (dd, J=4.3, 8.2 Hz, 1H), 6.54 (s, 1H), 4.57 (d, J=6.7 Hz, 2H), 3.77-3.73 (m, 4H), 3.57 (dd, J=7.6, 7.7 Hz, 2H), 3.54-3.50 (m, 4H), 3.40 (dd, J=6.8, 7.7 Hz, 2H), 3.09 (ttt, J=6.7, 6.8, 7.6 Hz, 1H).

### Parent Compound 92

LCMS (Method B): Rt=1.55 min, m/z [M+H]⁺=329

¹H NMR (400 MHz, DMSO-d₆) δ ppm: 8.80-8.77 (m, 1H), 8.32 (dd, J=1.5, 8.2 Hz, 0.4H), 8.27 (dd, J=1.6, 8.1 Hz, 0.6H), 7.22-7.16 (m, 1H), 6.52 (s, 1H), 5.39 (ddd, J=3.0, 3.0, 5.6 Hz, 0.4H), 4.87 (ddd, J=4.3, 9.9, 9.9 Hz, 0.6H), 3.77-3.72 (m, 4H), 3.51-3.45 (m, 4H), 3.02-2.97 (m, 1.2H), 2.84-2.56 (m, 2H), 2.31 (dd, J=10.4, 12.5 Hz, 0.6H), 2.18-2.14 (m, 0.4H), 2.01-1.69 (m, 2H), 1.49-1.39 (m, 0.8H), 0.93-0.88 (m, 3H). 3:2 mixture of trans:cis diastereomers.

### Parent Compound 93

LCMS (Method A): Rt=0.29 min, m/z [M+H]⁺=333

¹H NMR (400 MHz, DMSO-d₆) δ ppm: 8.80 (dd, J-1.8, 4.3 Hz, 1H), 8.30 (dd, J-1.8, 8.2 Hz, 1H), 7.20 (dd, J-4.3, 8.2 Hz, 1H), 6.56 (s, 1H), 5.41-5.31 (m, 1H), 4.68 (dddd, J-4.6, 8.0, 8.7, 50.6 Hz, 1H), 3.77-3.73 (m, 4H), 3.53-3.47 (m, 4H), 3.29-3.21 (m, 1H), 2.93-2.88 (m, 1H), 2.72-2.56 (m, 2H), 2.22 (ddd, J-4.6, 7.8, 16.8 Hz, 1H), 1.59-1.49 (m, 1H).

### Parent Compound 94

LCMS (Method A): Rt=0.73 min, m/z [M+H]⁺=342

### Parent Compound 95

LCMS (Method D): Rt=2.94 min, m/z [M+H]⁺=300

¹H NMR (400 MHz, DMSO-d₆) δ ppm: 8.72 (dd, J=1.4, 4.2 Hz, 1H), 8.17 (dd, J=1.4, 8.3 Hz, 1H), 7.13 (dd, J=4.2, 8.3 Hz, 1H), 6.51 (s, 1H), 3.75-3.71 (m, 4H), 3.52-3.48 (m, 4H), 3.29-3.24 (m, 4H), 2.93-2.88 (m, 4H).

### Parent Compound 99

LCMS (Method C): Rt=2.55 min, m/z [M+H]⁺=449

¹H NMR (400 MHz, DMSO-d₆) δ ppm: 8.68 (dd, J=1.8, 4.2 Hz, 1H), 8.45 (d, J=4.8 Hz, 1H), 8.11 (ddd, J=0.8, 1.8, 8.3 Hz, 1H), 7.69 (s, 1H), 7.43 (s, 1H), 7.14-7.07 (m, 2H), 7.04 (d, J=4.8 Hz, 1H), 6.47 (s, 1H), 3.84-3.80 (m, 2H), 3.74-3.70 (m, 4H), 3.52-3.48 (m, 4H), 3.38 (dd, J=6.2, 6.2 Hz, 2H), 3.01-2.93 (m, 2H), 1.94-1.83 (m, 3H), 1.56-1.46 (m, 2H).

### Parent Compound 100

LCMS (Method C): Rt=2.15 min, m/z [M+H]⁺=479

¹H NMR (400 MHz, DMSO-d₆) δ ppm: 8.71 (dd, J=1.7, 4.2 Hz, 1H), 8.14-8.11 (m, 1H), 7.13 (dd, J=4.2, 8.3 Hz, 1H), 7.00 (bs, 1H), 6.69 (bs, 2H), 6.49 (s, 1H), 6.30 (s, 1H), 3.82-3.78 (m, 5H), 3.75-3.71 (m, 4H), 3.51-3.46 (m, 4H), 3.25-3.20 (m, 2H), 2.95-2.86 (m, 2H), 1.85-1.73 (m, 3H), 1.48-1.41 (m, 2H).

### Parent Compound 101

LCMS (Method C): Rt=2.70 min, m/z [M+H]⁺=463

¹H NMR (400 MHz, DMSO-d₆) δ ppm: 8.71 (dd, J=1.7, 4.2 Hz, 1H), 8.13-8.11 (m, 1H), 7.98 (bs, 1H), 7.67 (bs, 1H), 7.41 (bs, 1H), 7.13 (dd, J=4.2, 8.3 Hz, 1H), 6.96 (s, 1H), 6.49 (s, 1H), 3.85-3.77 (m, 2H), 3.75-3.70 (m, 4H), 3.51-3.46 (m, 4H), 3.41-3.35 (m, 2H), 2.96-2.87 (m, 2H), 2.32 (s, 3H), 1.87-1.78 (m, 3H), 1.53-1.47 (m, 2H).

### Parent Compound 102

LCMS (Method C): Rt=2.64 min, m/z [M+H]⁺=420

¹H NMR (400 MHz, DMSO-d₆) δ ppm: 8.71 (dd, J=1.7, 4.2 Hz, 1H), 8.14-8.09 (m, 3H), 7.13 (dd, J=4.2, 8.3 Hz, 1H), 7.00 (t, J=6.0 Hz, 1H), 6.49 (s, 1H), 3.84-3.79 (m, 2H), 3.75-3.72 (m, 4H), 3.51-3.46 (m, 4H), 3.26-3.20 (m, 2H), 2.95-2.85 (m, 2H), 2.05 (s, 3H), 1.85-1.77 (m, 3H), 1.48-1.39 (m, 2H).

### Parent Compound 103

LCMS (Method C): Rt=2.50 min, m/z [M+H]⁺=446

¹H NMR (400 MHz, DMSO-d₆) δ ppm: 8.71 (dd, J=1.6, 4.2 Hz, 1H), 8.25 (s, 0.4H), 8.15 (s, 0.6H), 8.14-8.09 (m, 1H), 7.64 (t, J=5.6 Hz, 0.6H), 7.47 (t, J=5.6 Hz, 0.4H), 7.18 (bs, 1H), 7.13 (dd, J=4.2, 8.3 Hz, 1H), 7.02 (bs, 1H), 6.50 (s, 1H), 3.84-3.79 (m, 2H), 3.75-3.71 (m, 4H), 3.51-3.46 (m, 4H), 3.27-3.20 (m, 2H), 2.94-2.85 (m, 2H), 1.85-1.79 (m, 3H), 1.44-1.43 (m, 2H). 3:2 mixture of rotamers.

Parent compound 104

LCMS (Method C): Rt=3.10 min, m/z [M+H]⁺=424

¹H NMR (400 MHz, DMSO-d₆) δ ppm: 8.71 (dd, J=1.5, 4.2 Hz, 1H), 8.34 (d, J=0.9 Hz, 2H), 8.12 (dd, J=1.5, 8.3 Hz, 1H), 7.36 (t, J=5.9 Hz, 1H), 7.13 (dd, J=4.2, 8.3 Hz, 1H), 6.49 (s, 1H), 3.84-3.76 (m, 2H), 3.75-3.70 (m, 4H), 3.51-3.47 (m, 4H), 3.25-3.19 (m, 2H), 2.95-2.86 (m, 2H), 1.85-1.78 (m, 3H), 1.48-1.39 (m, 2H).

Parent compound 105

LCMS (Method C): Rt=2.67 min, m/z [M+H]⁺=463

¹H NMR (400 MHz, DMSO-d₆) δ ppm: 8.71 (dd, J=1.5, 4.2 Hz, 1H), 8.58-8.28 (m, 2H), 8.12 (dd, J=1.5, 8.3 Hz, 1H), 7.48-7.28 (bm, 1H), 7.13 (dd, J=4.2, 8.3 Hz, 1H), 7.03 (d, J=4.8 Hz, 1H), 6.49 (s, 1H), 3.87-3.78 (m, 2H), 3.75-3.70 (m, 4H), 3.51-3.46 (m, 4H), 3.44-3.38 (m, 2H), 2.98-2.87 (m, 2H), 2.82 (d, J=4.8 Hz, 3H), 1.89-1.84 (m, 3H), 1.53-1.46 (m, 2H).

### Parent Compound 106

LCMS (Method C): Rt=2.65 min, m/z [M+H]⁺=477

¹H NMR (400 MHz, DMSO-d₆) δ ppm: 8.71 (dd, J=1.5, 4.2 Hz, 1H), 8.41-8.35 (m, 1H), 8.12 (dd, J=1.5, 8.3 Hz, 1H), 7.52 (t, J=5.9 Hz, 1H), 7.13 (dd, J=4.2, 8.3 Hz, 1H), 6.58 (d, J=4.8 Hz, 1H), 6.49 (s, 1H), 3.84-3.80 (m, 2H), 3.75-3.70 (m, 4H), 3.51-3.46 (m, 4H), 3.30-3.22 (m, 2H), 2.97-2.89 (m, 8H), 1.87-1.78 (m, 3H), 1.50-1.41 (m, 2H).

### Parent Compound 107

LCMS (Method C): Rt=2.93 min, m/z [M+H]⁺=431

¹H NMR (400 MHz, DMSO-d₆) δ ppm: 8.73-8.64 (m, 3H), 8.46 (t, J=6.0 Hz, 1H), 8.12 (dd, J=1.5, 8.3 Hz, 1H), 7.13 (dd, J=4.2, 8.3 Hz, 1H), 6.50 (s, 1H), 3.82-3.79 (m, 2H), 3.75-3.72 (m, 4H), 3.51-3.46 (m, 4H), 2.96-2.86 (m, 2H), 1.87-1.77 (m, 3H), 1.50-1.41 (m, 2H). CH₂ hidden by H₂O solvent peak.

### Parent Compound 108

LCMS (Method E): Rt=3.17 min, m/z [M+H]⁺=431

¹H NMR (400 MHz, DMSO-d₆) δ ppm: 8.71 (dd, J=1.7, 4.2 Hz, 1H), 8.61-8.53 (m, 1H), 8.15-8.10 (m, 1H), 8.05-7.96 (m, 1H), 7.13 (dd, J=4.2, 8.4 Hz, 1H), 7.08 (d, J=4.9 Hz, 1H), 6.50 (s, 1H), 3.84-3.79 (m, 2H), 3.75-3.71 (m, 4H), 3.52-3.46 (m, 4H), 3.30-3.22 (m, 2H), 2.97-2.89 (m, 2H), 1.87-1.77 (m, 3H), 1.47-1.43 (m, 2H).

### Parent Compound 109

LCMS (Method C): Rt=2.46 min, m/z [M+H]⁺=420

¹H NMR (400 MHz, DMSO-d₆) δ ppm: 8.71 (dd, J=1.7, 4.3 Hz, 1H), 8.14-8.11 (m, 2H), 7.15-7.09 (m, 2H), 6.49 (s, 1H), 6.43 (d, J=5.0 Hz, 1H), 3.84-3.76 (m, 2H), 3.75-3.71 (m, 4H), 3.51-3.46 (m, 4H), 3.28-3.22 (m, 2H), 2.96-2.86 (m, 2H), 2.23 (s, 3H), 1.86-1.77 (m, 3H), 1.50-1.39 (m, 2H).

### Parent Compound 110

LCMS (Method E): Rt=2.56 min, m/z [M+H]⁺=450

¹H NMR (400 MHz, DMSO-d₆) δ ppm: 8.71 (dd, J=1.5, 4.2 Hz, 1H), 8.36 (d, J=4.8 Hz, 1H), 8.12 (dd, J=1.5, 8.3 Hz, 1H), 7.39 (s, 1H), 7.13 (dd, J=4.2, 8.3 Hz, 1H), 6.89 (d, J=4.8 Hz, 1H), 6.49 (s, 1H), 3.84-3.77 (m, 2H), 3.75-3.70 (m, 4H), 3.52-3.46 (m, 4H), 3.33-3.24 (m, 2H), 2.96-2.86 (m, 2H), 1.88-1.79 (m, 3H), 1.51-1.42 (m, 2H).

### Parent Compound 111

LCMS (Method E): Rt=2.35 min, m/z [M+H]⁺=436

¹H NMR (400 MHz, DMSO-d₆) δ ppm: 8.71 (dd, J=1.5, 4.2 Hz, 1H), 8.12 (dd, J=1.5, 8.3 Hz, 1H), 8.00 (d, J=4.8 Hz, 1H), 7.19 (s, 1H), 7.13 (dd, J=4.2, 8.3 Hz, 1H), 6.50 (s, 1H), 5.99 (d, J=4.8 Hz, 1H), 3.84-3.79 (m, 5H), 3.74-3.70 (m, 4H), 3.51-3.46 (m, 4H), 3.29-3.22 (m, 2H), 2.96-2.86 (m, 2H), 1.87-1.78 (m, 3H), 1.48-1.41 (m, 2H).

### Parent Compound 112

LCMS (Method C): Rt=3.26 min, m/z [M+H]⁺=478

¹H NMR (400 MHz, DMSO-d₆) δ ppm: 8.77-8.68 (m, 3H), 8.25 (t, J=6.0 Hz, 1H), 8.12 (dd, J=1.5, 8.3 Hz, 1H), 7.13 (dd, J=4.2, 8.3 Hz, 1H), 6.50 (s, 1H), 4.26 (q, J=7.1 Hz, 2H), 3.85-3.77 (m, 2H), 3.75-3.69 (m, 4H), 3.51-3.46 (m, 4H), 3.37-3.34 (m, 2H), 2.96-2.86 (m, 2H), 1.88-1.77 (m, 3H), 1.51-1.41 (m, 2H), 1.29 (t, J=7.1 Hz, 3H).

### Parent Compound 113

LCMS (Method C): Rt=2.19 min, m/z [M+H]⁺=422

¹H NMR (400 MHz, DMSO-d₆) δ ppm: 10.77 (bs, 1H), 8.71 (dd, J=1.5, 4.2 Hz, 1H), 8.13 (dd, J=1.5, 8.3 Hz, 1H), 7.58 (d, J=6.4 Hz, 1H), 7.13 (dd, J=4.2, 8.3 Hz, 1H), 6.78 (bs, 1H), 6.50 (s, 1H), 5.51 (d, J=6.4 Hz, 1H), 3.86-3.78 (m, 2H), 3.75-3.70 (m, 4H), 3.50-3.46 (m, 4H), 3.28-3.21 (m, 2H), 2.98-2.87 (m, 2H), 1.83-1.74 (m, 3H), 1.49-1.40 (m, 2H).

### Parent Compound 114

LCMS (Method C): Rt=2.65 min, m/z [M+H]⁺=406

¹H NMR (400 MHz, DMSO-d₆) δ ppm: 8.71 (dd, J=1.5, 4.2 Hz, 1H), 8.13 (dd, J=1.5, 8.3 Hz, 1H), 7.94 (d, J=1.6 Hz, 1H), 7.91 (dd, J=1.6, 2.8 Hz, 1H), 7.62 (d, J=2.8 Hz, 1H), 7.19-7.11 (m, 2H), 6.50 (s, 1H), 3.86-3.79 (m, 2H), 3.75-3.70 (m, 4H), 3.51-3.47 (m, 4H), 3.27-3.22 (m, 2H), 2.97-2.88 (m, 2H), 1.88-1.81 (m, 3H), 1.54-1.46 (m, 2H).

### Parent Compound 115

LCMS (Method C): Rt=3.22 min, m/z [M+H]⁺=430

¹H NMR (400 MHz, DMSO-d₆) δ ppm: 8.71 (dd, J=1.5, 4.2 Hz, 1H), 8.28 (dd, J=2.0, 4.9 Hz, 1H), 8.12 (dd, J=1.5, 8.3 Hz, 1H), 7.88 (dd, J=2.0, 7.6 Hz, 1H), 7.22 (t, J=5.8 Hz, 1H), 7.13 (dd, J=4.3, 8.2 Hz, 1H), 6.63 (dd, J=4.9, 7.6 Hz, 1H), 6.50 (s, 1H), 3.85-3.77 (m, 2H), 3.75-3.70 (m, 4H), 3.50-3.48 (m, 4H), 3.42-3.36 (m, 2H), 2.96-2.86 (m, 2H), 1.97-1.89 (m, 1H), 1.84-1.76 (m, 2H), 1.51-1.38 (m, 2H).

### Parent Compound 116

LCMS (Method C): Rt=2.31 min, m/z [M+H]⁺=436

¹H NMR (400 MHz, DMSO-d₆) δ ppm: 8.71 (dd, J=1.5, 4.2 Hz, 1H), 8.24 (d, J=4.9 Hz, 1H), 8.12 (dd, J=1.5, 8.3 Hz, 1H), 7.17-7.11 (m, 2H), 6.65 (d, J=4.9 Hz, 1H), 6.49 (s, 1H), 5.34 (t, J=5.8 Hz, 1H), 4.32 (d, J=5.8 Hz, 2H), 3.84-3.76 (m, 2H), 3.74-3.70 (m, 4H), 3.51-3.46 (m, 4H), 3.28-3.22 (m, 2H), 2.95-2.87 (m, 2H), 1.86-1.80 (m, 3H), 1.45-1.43 (m, 2H).

### Parent Compound 117

LCMS (Method C): Rt=2.43 min, m/z [M+H]⁺=434

¹H NMR (400 MHz, DMSO-d₆) δ ppm: 8.71 (dd, J=1.5, 4.2 Hz, 1H), 8.14-8.10 (m, 1H), 7.13 (dd, J=4.2, 8.3 Hz, 1H), 6.97 (t, J=5.8 Hz, 1H), 6.49 (s, 1H), 6.32 (s, 1H), 3.84-3.76 (m, 2H), 3.74-3.70 (m, 4H), 3.52-3.46 (m, 4H), 3.28-3.22 (m, 2H), 2.96-2.86 (m, 2H), 2.18 (s, 6H), 1.86-1.78 (m, 3H), 1.47-1.37 (m, 2H).

### Parent Compound 118

LCMS (Method B): Rt=2.03 min, m/z [M+H]⁺=521

### Parent Compound 119

LCMS (Method C): Rt=3.46 min, m/z [M+H]⁺=508

¹H NMR (400 MHz, DMSO-d₆) δ ppm: 8.71 (dd, J=1.5, 4.2 Hz, 1H), 8.68-8.56 (m, 1H), 8.50 (bs, 1H), 8.27 (t, J=7.9 Hz, 1H), 8.19-8.12 (m, 2H), 7.58-7.54 (m, 1H), 7.42 (d, J=5.0 Hz, 1H), 7.13 (dd, J=4.2, 8.3 Hz, 1H), 6.49 (s, 1H), 3.86-3.79 (m, 2H), 3.74-3.69 (m, 4H), 3.51-3.46 (m, 4H), 3.43-3.38 (m, 2H), 2.96-2.90 (m, 2H), 1.93-1.84 (m, 3H), 1.57-1.45 (m, 2H).

### Parent Compound 120

LCMS (Method C): Rt=1.98 min, m/z [M+H]⁺=406

¹H NMR (400 MHz, DMSO-d₆) δ ppm: 8.71 (dd, J=1.5, 4.2 Hz, 1H), 8.39 (s, 1H), 8.13 (dd, J=1.5, 8.3 Hz, 1H), 8.00 (bs, 1H), 7.49-7.46 (m, 1H), 7.13 (dd, J=4.2, 8.3 Hz, 1H), 6.51-6.49 (m, 2H), 3.85-3.78 (m, 2H), 3.75-3.70 (m, 4H), 3.51-3.47 (m, 4H), 3.30-3.24 (m, 2H), 2.97-2.88 (m, 2H), 1.86-1.78 (m, 3H), 1.51-1.45 (m, 2H).

### Parent Compound 121

LCMS (Method C): Rt=3.53 min, m/z [M+H]⁺=474

¹H NMR (400 MHz, DMSO-d₆) δ ppm: 8.71 (dd, J=1.5, 4.2 Hz, 1H), 8.66-8.57 (m, 2H), 8.21 (t, J=6.0 Hz, 1H), 8.12 (dd, J=1.5, 8.3 Hz, 1H), 7.13 (dd, J=4.2, 8.3 Hz, 1H), 6.50 (s, 1H), 3.85-3.77 (m, 2H), 3.75-3.70 (m, 4H), 3.51-3.46 (m, 4H), 2.96-2.87 (m, 2H), 1.88-1.78 (m, 3H), 1.50-1.41 (m, 2H). CH₂ hidden by H₂O solvent peak.

Parent compound 122

LCMS (Method C): Rt=2.01 min, m/z [M+H]⁺=405

¹H NMR (400 MHz, DMSO-d₆) δ ppm: 8.71 (dd, J=1.5, 4.2 Hz, 1H), 8.13 (dd, J=1.5, 8.3 Hz, 1H), 7.96-7.93 (m, 1H), 7.33 (ddd, J=1.8, 6.9, 8.5 Hz, 1H), 7.13 (dd, J=4.2, 8.3 Hz, 1H), 6.57 (t, J=5.7 Hz, 1H), 6.50-6.41 (m, 3H), 3.86-3.78 (m, 2H), 3.75-3.70 (m, 4H), 3.51-3.47 (m, 4H), 3.25-3.19 (m, 2H), 2.96-2.87 (m, 2H), 1.89-1.79 (m, 3H), 1.52-1.41 (m, 2H).

### Parent Compound 123

LCMS (Method C): Rt=3.38 min, m/z [M+H]⁺=465

¹H NMR (400 MHz, DMSO-d₆) δ ppm: 8.73-8.69 (m, 2H), 8.15-8.11 (m, 1H), 7.14 (dd, J=4.2, 8.3 Hz, 1H), 7.02 (s, 1H), 6.50 (s, 1H), 3.86-3.78 (m, 2H), 3.75-3.70 (m, 4H), 3.52-3.46 (m, 4H), 2.98-2.90 (m, 2H), 1.84-1.77 (m, 3H), 1.50-1.47 (m, 2H). CH₂ hidden by H₂O solvent peak.

### Parent Compound 124

LCMS (Method C): Rt=3.39 min, m/z [M+H]⁺=470/472

¹H NMR (400 MHz, DMSO-d₆) δ ppm: 8.71 (dd, J=1.5, 4.2 Hz, 1H), 8.15-8.09 (m, 2H), 7.53-7.44 (m, 1H), 7.13 (dd, J=4.2, 8.3 Hz, 1H), 6.50 (s, 1H), 3.90 (s, 3H), 3.85-3.79 (m, 2H), 3.74-3.70 (m, 4H), 3.51-3.46 (m, 4H), 3.29-3.20 (m, 2H), 2.96-2.86 (m, 2H), 1.86-1.77 (m, 3H), 1.51-1.38 (m, 2H).

### Parent Compound 125

LCMS (Method C): Rt=3.83 min, m/z [M+H]⁺=508/510

¹H NMR (400 MHz, DMSO-d₆) δ ppm: 8.71 (dd, J=1.5, 4.2 Hz, 1H), 8.36 (d, J=2.5 Hz, 1H), 8.18 (d, J=2.5 Hz, 1H), 8.12 (dd, J=1.5, 8.3 Hz, 1H), 7.48 (t, J=5.7 Hz, 1H), 7.13 (dd, J=4.2, 8.3 Hz, 1H), 6.50 (s, 1H), 3.84-3.77 (m, 2H), 3.75-3.70 (m, 4H), 3.51-3.46 (m, 4H), 3.38-3.33 (m, 2H), 2.95-2.86 (m, 2H), 1.96-1.88 (m, 1H), 1.80-1.74 (m, 2H), 1.50-1.38 (m, 2H).

### Parent Compound 126

LCMS (Method C): Rt=2.22 min, m/z [M+H]⁺=463

¹H NMR (400 MHz, DMSO-d₆) δ ppm: 10.20 (s, 1H), 8.71 (dd, J=1.7, 4.2 Hz, 1H), 8.14-8.10 (m, 2H), 7.18 (d, J=5.4 Hz, 1H), 7.13 (dd, J=4.2, 8.3 Hz, 1H), 7.02 (bs, 1H), 6.49 (s, 1H), 3.85-3.78 (m, 2H), 3.75-3.70 (m, 4H), 3.52-3.46 (m, 4H), 3.28-3.22 (m, 2H), 2.96-2.87 (m, 2H), 2.09 (s, 3H), 1.86-1.78 (m, 3H), 1.46-1.39 (m, 2H).

### Parent Compound 127

LCMS (Method C): Rt=2.98 min, m/z [M+H]⁺=393

¹H NMR (400 MHz, DMSO-d₆) δ ppm: 8.79 (dd, J=1.8, 4.3 Hz, 1H), 8.38 (d, J=4.8 Hz, 2H), 8.33 (dd, J=1.8, 8.2 Hz, 1H), 7.19 (dd, J=4.3, 8.2 Hz, 1H), 6.63 (t, J=4.8 Hz, 1H), 6.56 (s, 1H), 5.54-5.48 (m, 1H), 4.14-4.07 (m, 2H), 3.82-3.74 (m, 6H), 3.54-3.50 (m, 4H), 2.10-2.03 (m, 2H), 1.86-1.77 (m, 2H).

### Parent Compound 128

LCMS (Method C): Rt=2.38 min, m/z [M+H]⁺=379

¹H NMR (400 MHz, DMSO-d₆) δ ppm: 8.77 (dd, J=1.8, 4.3 Hz, 1H), 8.35 (d, J=4.8 Hz, 2H), 8.07 (dd, J=1.8, 8.2 Hz, 1H), 7.12 (dd, J=4.3, 8.2 Hz, 1H), 6.68 (t, J=4.8 Hz, 1H), 6.55 (s, 1H), 4.66 (d, J=6.1 Hz, 2H), 4.24-4.18 (m, 2H), 3.98 (dd, J=5.3, 8.9 Hz, 2H), 3.76-3.73 (m, 4H), 3.54-3.49 (m, 4H), 3.26-3.19 (m, 1H).

### Parent Compound 129

LCMS (Method C): Rt=3.09 min, m/z [M+H]⁺=407

¹H NMR (400 MHz, DMSO-d₆) δ ppm: 8.79 (dd, J=1.5, 4.3 Hz, 1H), 8.34 (d, J=4.7 Hz, 2H), 8.28 (dd, J=1.5, 8.2 Hz, 1H), 7.19 (dd, J=4.3, 8.2 Hz, 1H), 6.58 (t, J=4.7 Hz, 1H), 6.54 (s, 1H), 4.77-4.70 (m, 2H), 4.33 (d, J=6.5 Hz, 2H), 3.76-3.72 (m, 4H), 3.53-3.49 (m, 4H), 2.91 (dt, J=2.2, 12.7 Hz, 2H), 2.21-2.13 (m, 1H), 1.93-1.88 (m, 2H), 1.36-1.24 (m, 2H).

### Parent Compound 130

LCMS (Method C): Rt=2.63 min, m/z [M+H]⁺=420

¹H NMR (400 MHz, DMSO-d₆) δ ppm: 8.71 (dd, J=1.5, 4.2 Hz, 1H), 8.25 (d, J=4.8 Hz, 2H), 8.13 (dd, J=1.5, 8.3 Hz, 1H), 7.15-7.11 (m, 2H), 6.53 (t, J=4.8 Hz, 1H), 6.50 (s, 1H), 3.83-3.78 (m, 2H), 3.76-3.72 (m, 4H), 3.51-3.47 (m, 4H), 3.38-3.34 (m, 2H), 2.94-2.85 (m, 2H), 1.86-1.79 (m, 2H), 1.62-1.40 (m, 5H).

### Parent Compound 132

LCMS (Method C): Rt=2.17 min, m/z [M+H]⁺=423

¹H NMR (400 MHz, DMSO-d₆) δ ppm: 8.71 (dd, J=1.5, 4.2 Hz, 1H), 8.13 (dd, J=1.5, 8.3 Hz, 1H), 7.97 (dd, J=5.9, 9.8 Hz, 1H), 7.13 (dd, J=4.2, 8.3 Hz, 1H), 6.90 (t, J=5.7 Hz, 1H), 6.50 (s, 1H), 6.38-6.33 (m, 1H), 6.26 (dd, J=2.4, 12.3 Hz, 1H), 3.86-3.78 (m, 2H), 3.75-3.70 (m, 4H), 3.51-3.47 (m, 4H), 3.26-3.20 (m, 2H), 2.97-2.87 (m, 2H), 1.87-1.78 (m, 3H), 1.51-1.41 (m, 2H).

### Parent Compound 133

LCMS (Method C): Rt=2.04 min, m/z [M+H]⁺=405

¹H NMR (400 MHz, DMSO-d₆) δ ppm: 8.71 (dd, J=1.5, 4.2 Hz, 1H), 8.14 (dd, J=1.5, 8.3 Hz, 1H), 7.99 (d, J=2.8 Hz, 1H), 7.73 (dd, J=1.4, 4.6 Hz, 1H), 7.14 (dd, J=4.2, 8.3 Hz, 1H), 7.07 (dd, J=4.6, 8.3 Hz, 1H), 6.92 (ddd, J=1.4, 2.8, 8.3 Hz, 1H), 6.50 (s, 1H), 5.95 (t, J=5.7 Hz, 1H), 3.87-3.80 (m, 2H), 3.75-3.70 (m, 4H), 3.52-3.47 (m, 4H), 3.05-2.89 (m, 4H), 1.93-1.78 (m, 3H), 1.55-1.44 (m, 2H).

### Parent Compound 134

LCMS (Method C): Rt=2.15 min, m/z [M+H]⁺=419

¹H NMR (400 MHz, DMSO-d₆) δ ppm: 8.71 (dd, J=1.5, 4.2 Hz, 1H), 8.13 (dd, J=1.5, 8.3 Hz, 1H), 7.82-7.80 (m, 1H), 7.13 (dd, J=4.2, 8.3 Hz, 1H), 6.50 (s, 1H), 6.45 (t, J=5.5 Hz, 1H), 6.30-6.28 (m, 2H), 3.85-3.77 (m, 2H), 3.75-3.70 (m, 4H), 3.51-3.47 (m, 4H), 3.23-3.17 (m, 2H), 2.96-2.86 (m, 2H), 2.13 (s, 3H), 1.88-1.77 (m, 3H), 1.49-1.41 (m, 2H).

### Parent Compound 135

LCMS (Method C): Rt=2.61 min, m/z [M+H]⁺=423

¹H NMR (400 MHz, DMSO-d₆) δ ppm: 8.71 (dd, J=1.5, 4.2 Hz, 1H), 8.12 (dd, J=1.5, 8.3 Hz, 1H), 7.82-7.80 (m, 1H), 7.30 (ddd, J=1.4, 7.7, 12.0 Hz, 1H), 7.13 (dd, J=4.2, 8.3 Hz, 1H), 6.74-6.67 (m, 1H), 6.51-6.46 (m, 2H), 3.85-3.77 (m, 2H), 3.75-3.70 (m, 4H), 3.52-3.46 (m, 4H), 2.96-2.86 (m, 2H), 1.94-1.87 (m, 1H), 1.86-1.79 (m, 2H), 1.51-1.38 (m, 2H). CH₂ hidden by H₂O solvent peak.

### Parent Compound 136

LCMS (Method D): Rt=4.00 min, m/z [M+H]⁺=405

¹H NMR (400 MHz, DMSO-d₆) δ ppm: 8.71 (dd, J=1.5, 4.2 Hz, 1H), 8.13 (dd, J=1.5, 8.3 Hz, 1H), 8.01-7.98 (m, 2H), 7.14 (dd, J=4.2, 8.3 Hz, 1H), 6.60 (t, J=5.7 Hz, 1H), 6.52-6.49 (m, 3H), 3.87-3.79 (m, 2H), 3.75-3.70 (m, 4H), 3.52-3.47 (m, 4H), 3.07-3.01 (m, 2H), 2.98-2.87 (m, 2H), 1.90-1.78 (m, 3H), 1.54-1.42 (m, 2H).

### Parent Compound 137

LCMS (Method C): Rt=2.08 min, m/z [M+H]⁺=435

¹H NMR (400 MHz, DMSO-d₆) δ ppm: 8.71 (dd, J=1.5, 4.2 Hz, 1H), 8.14-8.11 (m, 1H), 7.57 (dd, J=1.4, 5.1 Hz, 1H), 7.13 (dd, J=4.2, 8.3 Hz, 1H), 6.96 (dd, J=1.4, 7.8 Hz, 1H), 6.50 (s, 1H), 6.46 (dd, J=5.1, 7.8 Hz, 1H), 6.01 (t, J=5.9 Hz, 1H), 3.85-3.76 (m, 5H), 3.75-3.70 (m, 4H), 3.51-3.46 (m, 4H), 2.95-2.86 (m, 2H), 1.94-1.86 (m, 1H), 1.84-1.75 (m, 2H), 1.50-1.39 (m, 2H). CH₂ hidden by H₂O solvent peak.

### Parent Compound 138

LCMS (Method C): Rt=3.40 min, m/z [M+H]⁺=404

¹H NMR (400 MHz, DMSO-d₆) δ ppm: 8.71 (dd, J=1.5, 4.2 Hz, 1H), 8.14 (dd, J=1.5, 8.3 Hz, 1H), 7.14 (dd, J=4.2, 8.3 Hz, 1H), 7.09-7.02 (m, 2H), 6.60-6.57 (m, 2H), 6.52-6.47 (m, 2H), 5.66 (t, J=5.7 Hz, 1H), 3.87-3.80 (m, 2H), 3.75-3.70 (m, 4H), 3.52-3.47 (m, 4H), 3.01-2.88 (m, 4H), 1.93-1.78 (m, 3H), 1.54-1.42 (m, 2H).

### Parent Compound 142 03

LCMS (Method C): Rt=3.27 min, m/z [M+H]⁺=486

¹H NMR (400 MHz, DMSO-d₆) δ ppm: 8.71 (dd, J=1.5, 4.2 Hz, 1H), 8.09 (dd, J=1.5, 8.3 Hz, 1H), 7.90-7.85 (m, 2H), 7.75 (bs, 1H), 7.48-7.42 (m, 2H), 7.12 (dd, J=4.2, 8.3 Hz, 1H), 6.49 (s, 1H), 3.80-3.70 (m, 6H), 3.51-3.46 (m, 4H), 2.90-2.81 (m, 2H), 2.74-2.69 (m, 2H), 1.79-1.74 (m, 2H), 1.64-1.57 (m, 1H), 1.41-1.29 (m, 2H).

### Parent Compound 143

LCMS (Method C): Rt=2.51 min, m/z [M+H]⁺=393

¹H NMR (400 MHz, DMSO-d₆) δ ppm: 8.80 (dd, J=1.8, 4.4 Hz, 1H), 8.39-8.36 (m, 1H), 7.21 (dd, J=4.4, 8.2 Hz, 1H), 6.55 (s, 1H), 5.42-5.35 (m, 1H), 3.77-3.72 (m, 4H), 3.53-3.48 (m, 4H), 3.42-3.21 (m, 4H), 2.93 (s, 3H), 2.15-2.06 (m, 2H), 1.98-1.88 (m, 2H).

### Parent Compound 144

LCMS (Method C): Rt=2.35 min, m/z [M+H]⁺=378

¹H NMR (400 MHz, DMSO-d₆) δ ppm: 8.76 (dd, J=1.5, 4.2 Hz, 1H), 8.21 (dd, J=1.5, 8.3 Hz, 1H), 7.17 (dd, J=4.2, 8.3 Hz, 1H), 6.58 (s, 1H), 3.76-3.71 (m, 4H), 3.54-3.48 (m, 4H), 3.47-3.45 (m, 4H), 3.38-3.33 (m, 4H), 2.95 (s, 3H).

### Parent Compound 148

LCMS (Method C): Rt=2.07 min, m/z [M+H]⁺=358

¹H NMR (400 MHz, DMSO-d₆) δ ppm: 8.79 (dd, J=1.5, 4.3 Hz, 1H), 8.22 (dd, J=1.5, 8.2 Hz, 1H), 7.19 (dd, J=4.3, 8.2 Hz, 1H), 6.55 (s, 1H), 6.22-6.17 (m, 1H), 4.56 (d, J=6.1 Hz, 2H), 3.96-3.90 (m, 2H), 3.77-3.67 (m, 6H), 3.54-3.49 (m, 4H), 3.06-2.98 (m, 1H), 2.54 (d, J=4.4 Hz, 3H).

### Parent Compound 155

LCMS (Method C): Rt=2.10 min, m/z [M+H]⁺=382

¹H NMR (400 MHz, DMSO-d₆) δ ppm: 12.79 (bs, 1H), 8.78 (dd, J=1.5, 4.3 Hz, 1H), 8.30 (dd, J=1.5, 8.2 Hz, 1H), 7.79 (bs, 1H), 7.18 (dd, J=4.3, 8.2 Hz, 1H), 6.54 (s, 1H), 5.45-5.38 (m, 1H), 3.77-3.73 (m, 4H), 3.71-3.62 (m, 2H), 3.53-3.47 (m, 4H), 3.41-3.35 (m, 2H), 2.12-2.05 (m, 2H), 1.91-1.80 (m, 2H).

### Parent Compound 156

LCMS (Method C): Rt=2.20 min, m/z [M+H]⁺=400

¹H NMR (400 MHz, DMSO-d₆) δ ppm: 12.97 (bs, 1H), 8.80 (dd, J=1.5, 4.3 Hz, 1H), 8.33 (dd, J=1.5, 8.2 Hz, 1H), 8.00 (bs, 1H), 7.19 (dd, J=4.3, 8.2 Hz, 1H), 6.59 (s, 1H), 5.59-5.50 (m, 1H), 5.01-4.83 (m, 1H), 4.04-3.93 (m, 1H), 3.78-3.73 (m, 5H), 3.56-3.47 (m, 5H), 3.42-3.36 (m, 1H), 2.34-2.25 (m, 1H), 1.86-1.75 (m, 1H).

### Parent Compound 157

LCMS (Method C): Rt=2.23 min, m/z [M+H]⁺=396

¹H NMR (400 MHz, DMSO-d₆) δ ppm: 13.05 (bs, 0.4H), 12.60 (bs, 0.6H), 8.80-8.77 (m, 1H), 8.40-8.37 (m, 0.4H), 8.31-8.26 (m, 0.6H), 8.17 (bs, 0.4H), 7.48 (bs, 0.6H), 7.21-7.16 (m, 1H), 6.54 (s, 1H), 5.49-5.45 (m, 0.4H), 5.05-5.04 (m, 0.6H), 3.95-3.84 (m, 1.2H), 3.78-3.72 (m, 4H), 3.70-3.61 (m, 0.8H), 3.53-3.47 (m, 4H), 3.24-3.09 (m, 1.2H), 2.94-2.79 (m, 0.8H), 2.17-2.00 (m, 2H), 1.94-1.85 (m, 0.4H), 1.68-1.58 (m, 0.6H), 1.01-0.96 (m, 3H). 3:2 mixture of trans:cis diastereomers.

### Parent Compound 163

LCMS (Method C): Rt=3.23 min, m/z [M+H]⁺=414

¹H NMR (400 MHz, DMSO-d₆) δ ppm: 10.04 (s, 1H), 8.53 (d, J=2.9 Hz, 1H), 7.96 (dd, J=0.7, 4.6 Hz, 1H), 7.62 (dd, J=0.7, 2.9 Hz, 1H), 7.48 (dd, J=0.7, 4.4 Hz, 1H), 6.50 (s, 1H), 5.45-5.40 (m, 1H), 4.28-4.20 (m, 1H), 3.77-3.72 (m, 4H), 3.42-3.39 (m, 4H), 2.21-2.05 (m, 4H), 1.96-1.80 (m, 4H).

### Parent Compound 164

LCMS (Method C): Rt=4.12 min, m/z [M+H]⁺=412

¹H NMR (400 MHz, DMSO-d₆) δ ppm: 10.03 (s, 1H), 8.53-8.51 (m, 1H), 7.58-7.57 (m, 0.7H), 7.51-7.50 (m, 0.3H), 7.35-7.32 (m, 1H), 6.99-6.92 (m, 2H), 6.48 (s, 1H), 5.48-5.44 (m, 0.7H), 5.18-5.11 (m, 0.3H), 3.77-3.72 (m, 4H), 3.42-3.36 (m, 4H), 3.04-2.91 (m, 1H), 2.30-2.24 (m, 0.7H), 2.17-2.12 (m, 1.9H), 1.96-1.62 (m, 5.4H).

### Parent Compound 165

LCMS (Method C): Rt=4.24 min, m/z [M+H]⁺=406

¹H NMR (400 MHz, DMSO-d₆) δ ppm: 10.05 (s, 1H), 8.54-8.53 (m, 1H), 7.68-7.66 (m, 1H), 7.35-7.18 (m, 5H), 6.49 (s, 1H), 5.56-5.47 (m, 1H), 3.77-3.72 (m, 4H), 3.43-3.38 (m, 4H), 2.73-2.65 (m, 1H), 2.20-2.16 (m, 2H), 1.91-1.71 (m, 6H).

### Parent Compound 166

LCMS (Method C): Rt=3.20 min, m/z [M+H]⁺=410

¹H NMR (400 MHz, DMSO-d₆) δ ppm: 10.00 (bs, 1H), 8.51 (d, J=2.9 Hz, 1H), 7.57 (dd, J=0.7, 3.0 Hz, 1H), 7.53-7.50 (m, 1H), 7.33-7.30 (m, 1H), 6.48-6.47 (m, 1H), 5.46-5.42 (m, 1H), 3.78 (s, 3H), 3.76-3.72 (m, 4H), 3.42-3.37 (m, 4H), 2.65-2.59 (m, 1H), 2.09-2.07 (m, 2H), 1.83-1.70 (m, 6H).

### Parent Compound 167

LCMS (Method C): Rt=3.30 min, m/z [M+H]⁺=413

¹H NMR (400 MHz, DMSO-d₆) δ ppm: 10.08 (bs, 1H), 9.04 (d, J=2.0 Hz, 1H), 8.51 (d, J=2.9 Hz, 1H), 7.58-7.56 (m, 1H), 7.38-7.36 (m, 1H), 6.48 (s, 1H), 5.49-5.44 (m, 1H), 3.77-3.72 (m, 4H), 3.42-3.37 (m, 4H), 2.97-2.89 (m, 1H), 2.18-2.10 (m, 2H), 1.96-1.79 (m, 6H).

### Parent Compound 168

LCMS (Method C): Rt=3.28 min, m/z [M+H]⁺=413

¹H NMR (400 MHz, DMSO-d₆) δ ppm: 10.03 (bs, 1H), 8.52-8.51 (m, 1H), 7.74-7.73 (m, 1H), 7.61-7.60 (m, 1H), 7.58-7.56 (m, 1H), 6.49 (s, 1H), 5.48-5.44 (m, 1H), 3.77-3.72 (m, 4H), 3.41-3.37 (m, 4H), 3.27-3.16 (m, 1H), 2.20-2.12 (m, 2H), 2.05-1.83 (m, 6H).

### Prodrug 2

LCMS (Method D): Rt=4.96 min, m/z [M+H]⁺=553

¹H NMR (400 MHz, DMSO-d₆) δ ppm: 8.69 (d, J=3.0 Hz, 1H), 7.97 (d, J=4.5 Hz, 1H), 7.87 (s, 1H), 7.79 (d, J=3.0 Hz, 1H), 7.44 (d, J=4.3 Hz, 1H), 6.56 (s, 1H), 5.45-5.41 (m, 1H), 5.40 (s, 2H), 4.28-4.20 (m, 1H), 4.17 (s, 3H), 3.78-3.72 (m, 4H), 3.47-3.42 (m, 4H), 2.23-2.12 (m, 4H), 2.00-1.83 (m, 4H).

### Prodrug 3 04

LCMS (Method C): Rt=4.60 min, m/z [M+H]⁺=550

¹H NMR (400 MHz, DMSO-d₆) δ ppm: 9.36 (dd, J=0.4, 2.7 Hz, 1H), 8.78 (d, J=3.0 Hz, 1H), 8.64 (dd, J=2.7, 8.7 Hz, 1H), 7.98 (dd, J=0.6, 4.6 Hz, 1H), 7.92 (d, J=8.7 Hz, 1H), 7.80 (d, J=3.0 Hz, 1H), 7.48 (dd, J=0.6, 4.4 Hz, 1H), 6.56 (s, 1H), 5.54 (s, 2H), 5.44-5.40 (m, 1H), 4.29-4.20 (m, 1H), 3.77-3.72 (m, 4H), 3.46-3.41 (m, 4H), 2.20-2.09 (m, 4H), 2.00-1.79 (m, 4H).

### Prodrug 4

LCMS (Method E): Rt=4.42 min, m/z [M+H]⁺=553

¹H NMR (400 MHz, DMSO-d₆) δ ppm: 8.69 (d, J=3.0 Hz, 1H), 8.33 (s, 1H), 7.99 (dd, J=0.7, 4.6 Hz, 1H), 7.87 (d, J=3.0 Hz, 1H), 7.45 (dd, J=0.7, 4.4 Hz, 1H), 6.56 (s, 1H), 5.71 (s, 2H), 5.42-5.39 (m, 1H), 4.29-4.19 (m, 1H), 4.02 (s, 3H), 3.77-3.72 (m, 4H), 3.48-3.43 (m, 4H), 2.21-2.12 (m, 4H), 1.99-1.82 (m, 4H).

### Prodrug 5

LCMS (Method D): Rt=5.53 min, m/z [M+H]⁺=579

¹H NMR (400 MHz, DMSO-d₆) δ ppm: 8.72 (d, J=3.0 Hz, 1H), 7.97 (dd, J=0.6, 4.6 Hz, 1H), 7.88 (dd, J=2.2, 8.3 Hz, 1H), 7.83 (d, J=2.2 Hz, 1H), 7.76 (d, J=8.3 Hz, 1H), 7.74 (d, J=3.0 Hz, 1H), 7.45 (dd, J=0.6, 4.4 Hz, 1H), 6.55 (s, 1H), 5.44-5.41 (m, 1H), 5.35 (s, 2H), 4.27-4.19 (m, 1H), 3.95 (s, 3H), 3.77-3.72 (m, 4H), 3.46-3.41 (m, 4H), 2.20-2.07 (m, 4H), 1.98-1.82 (m, 4H).

### Prodrug 6 4

LCMS (Method D): Rt=4.61 min, m/z [M+H]⁺=552

¹H NMR (400 MHz, DMSO-d₆) δ ppm: 8.67 (d, J=2.9 Hz, 1H), 7.82 (d, J=2.9 Hz, 1H), 7.72 (d, J=3.3 Hz, 1H), 7.60 (d, J=3.3 Hz, 1H), 7.35 (s, 1H), 6.55 (s, 1H), 5.47-5.46 (m, 1H), 5.42 (s, 2H), 3.98 (s, 3H), 3.78-3.74 (m, 4H), 3.48-3.44 (m, 4H), 3.26-3.19 (m, 1H), 2.16-1.87 (m, 8H).

### Prodrug 7

LCMS (Method C): Rt=4.99 min, m/z [M+H]⁺=549

¹H NMR (400 MHz, DMSO-d₆) δ ppm: 8.76-8.72 (m, 1H), 8.24 (d, J=8.5 Hz, 2H), 8.01 (d, J=4.2 Hz, 1H), 7.80 (d, J=8.5 Hz, 2H), 7.75 (d, J=2.3 Hz, 1H), 7.48 (d, J=4.2 Hz, 1H), 6.55 (s, 1H), 5.48-5.40 (m, 3H), 4.27-4.22 (m, 1H), 3.78-3.71 (m, 4H), 3.47-3.43 (m, 4H), 2.21-2.07 (m, 4H), 1.99-1.82 (m, 4H).

### Prodrug 8

LCMS (Method C): Rt=3.91 min, m/z [M+H]⁺=553

¹H NMR (400 MHz, DMSO-d₆) δ ppm: 8.68 (d, J=3.0 Hz, 1H), 8.00 (dd, J=0.7, 4.6 Hz, 1H), 7.92 (s, 1H), 7.85 (d, J=3.0 Hz, 1H), 7.45 (dd, J=0.7, 4.4 Hz, 1H), 6.55 (s, 1H), 5.70 (s, 2H), 5.41-5.37 (m, 1H), 4.29-4.20 (m, 1H), 3.83 (s, 3H), 3.77-3.72 (m, 4H), 3.47-3.42 (m, 4H), 2.23-2.14 (m, 4H), 1.97-1.82 (m, 4H).

### Prodrug 9

LCMS (Method D): Rt=4.97 min, m/z [M+H]⁺=539

¹H NMR (400 MHz, DMSO-d₆) δ ppm: 8.70 (d, J=3.0 Hz, 1H), 7.99 (dd, J=0.6, 4.6 Hz, 1H), 7.85 (d, J=3.0 Hz, 1H), 7.66 (d, J=3.7 Hz, 1H), 7.46 (dd, J=0.6, 4.4 Hz, 1H), 7.06 (d, J=3.7 Hz, 1H), 6.56 (s, 1H), 5.45-5.43 (m, 3H), 4.28-4.21 (m, 1H), 3.77-3.72 (m, 4H), 3.47-3.44 (m, 4H), 2.22-2.13 (m, 4H), 1.96-1.81 (m, 4H).

### Prodrug 10

LCMS (Method C): Rt=4.38 min, m/z [M+H]⁺=631/633

¹H NMR (400 MHz, DMSO-d₆) δ ppm: 8.67 (d, J=3.0 Hz, 1H), 7.99 (dd, J=0.6, 4.6 Hz, 1H), 7.86 (d, J=3.0 Hz, 1H), 7.45 (dd, J=0.6, 4.4 Hz, 1H), 6.55 (s, 1H), 5.72 (s, 2H), 5.41-5.37 (m, 1H), 4.28-4.21 (m, 1H), 3.78 (s, 3H), 3.77-3.72 (m, 4H), 3.47-3.42 (m, 4H), 2.23-2.14 (m, 4H), 1.99-1.83 (m, 4H).

### Prodrug 11

LCMS (Method D): Rt=4.57 min, m/z [M+H]⁺=553

¹H NMR (400 MHz, DMSO-d₆) δ ppm: 8.68 (d, J=3.0 Hz, 1H), 8.02 (dd, J=0.6, 4.6 Hz, 1H), 7.88 (d, J=3.0 Hz, 1H), 7.46 (dd, J=0.6, 4.4 Hz, 1H), 7.39 (s, 1H), 6.56 (s, 1H), 5.47-5.43 (m, 3H), 4.26 (tt, J=3.8, 10.6 Hz, 1H), 4.00 (s, 3H), 3.77-3.72 (m, 4H), 3.47-3.42 (m, 4H), 2.22-2.16 (m, 4H), 1.97-1.83 (m, 4H).

### Prodrug 12

LCMS (Method C): Rt=5.18 min, m/z [M+H]⁺=563

¹H NMR (400 MHz, DMSO-d₆) δ ppm: 8.67 (d, J=3.0 Hz, 1H), 8.11 (d, J=8.8 Hz, 2H), 8.03 (dd, J=0.7, 4.6 Hz, 1H), 7.77 (d, J=8.8 Hz, 2H), 7.56-7.54 (m, 2H), 6.48 (s, 1H), 5.87 (q, J=6.4 Hz, 1H), 5.42-5.38 (m, 1H), 4.28-4.18 (m, 1H), 3.74-3.69 (m, 4H), 3.42-3.36 (m, 4H), 2.25-2.11 (m, 2H), 2.03-1.74 (m, 6H), 1.66 (d, J=6.4 Hz, 3H).

### Prodrug 13

LCMS (Method C): Rt=3.73 min, m/z [M+H]⁺=519

¹H NMR (400 MHz, DMSO-d₆) δ ppm: 8.80 (dd, J=1.9, 4.3 Hz, 1H), 8.31 (ddd, J=0.6, 1.9, 8.2 Hz, 1H), 7.23 (dd, J=4.3, 8.2 Hz, 1H), 7.02 (d, J=1.3 Hz, 1H), 6.86-6.85 (m, 2H), 6.53 (s, 1H), 5.48-5.44 (m, 1H), 5.41 (s, 2H), 3.88 (s, 3H), 3.77-3.72 (m, 4H), 3.52-3.47 (m, 4H), 2.98 (tt, J=3.4, 10.9 Hz, 1H), 2.21-2.12 (m, 2H), 2.05-1.93 (m, 2H), 1.84-1.75 (m, 2H), 1.68-1.59 (m, 2H).

### Prodrug 14 (Formic Acid 1.5 Equivalents)

LCMS (Method C): Rt=2.04 min, m/z [M+H]⁺=519

¹H NMR (400 MHz, DMSO-d₆) δ ppm: 9.46-9.43 (m, 1H), 8.81 (dd, J=1.8, 4.3 Hz, 1H), 8.51 (s, 1H), 8.48 (dd, J=1.8, 8.2 Hz, 1H), 8.06 (dd, J=1.8, 1.8 Hz, 1H), 7.84 (dd, J=1.8, 1.8 Hz, 1H), 7.39 (s, 1H), 7.21 (dd, J=4.3, 8.2 Hz, 1H), 6.55 (s, 1H), 5.68 (s, 2H), 5.53-5.49 (m, 1H), 4.48 (tt, J=3.9, 11.2 Hz, 1H), 3.92 (s, 3H), 3.77-3.72 (m, 4H), 3.53-3.47 (m, 4H), 2.25-2.04 (m, 6H), 1.90-1.82 (m, 2H).

### Prodrug 16

LCMS (Method C): Rt=3.04 min, m/z [M+H]⁺=563

¹H NMR (400 MHz, DMSO-d₆) δ ppm: 8.70 (d, J=3.0 Hz, 1H), 8.29 (s, 1H), 7.83 (d, J=3.0 Hz, 1H), 7.76 (s, 1H), 7.27 (s, 1H), 6.90 (s, 1H), 6.57 (s, 1H), 5.50-5.46 (m, 1H), 5.42 (s, 2H), 4.66 (hept, J=6.7 Hz, 1H), 4.28-4.20 (m, 1H), 3.78-3.73 (m, 4H), 3.48-3.42 (m, 4H), 2.22-2.07 (m, 4H), 1.95-1.81 (m, 4H), 1.46 (d, J=6.7 Hz, 6H).

### Prodrug 17

LCMS (Method D): Rt=5.49 min, m/z [M+H]⁺=569

¹H NMR (400 MHz, DMSO-d₆) δ ppm: 8.68 (d, J=3.0 Hz, 1H), 7.99 (dd, J=0.6, 4.6 Hz, 1H), 7.90 (d, J=4.2 Hz, 1H), 7.77 (d, J=3.0 Hz, 1H), 7.51 (dd, J=0.6, 4.4 Hz, 1H), 7.36 (d, J=4.2 Hz, 1H), 6.52 (s, 1H), 6.11 (q, J=6.3 Hz, 1H), 5.44-5.40 (m, 1H), 4.27-4.20 (m, 1H), 3.76-3.71 (m, 4H), 3.45-3.40 (m, 4H), 2.27-2.00 (m, 4H), 1.96-1.78 (m, 4H), 1.74 (d, J=6.3 Hz, 3H).

### Prodrug 18

LCMS (Method D): Rt=5.30 min, m/z [M+H]⁺=555

¹H NMR (400 MHz, DMSO-d₆) δ ppm: 8.71 (d, J=3.0 Hz, 1H), 8.02 (d, J=4.2 Hz, 1H), 8.00 (dd, J=0.7, 4.6 Hz, 1H), 7.81 (d, J=3.0 Hz, 1H), 7.47 (dd, J=0.7, 4.4 Hz, 1H), 7.38 (d, J=4.2 Hz, 1H), 6.56 (s, 1H), 5.60 (s, 2H), 5.45-5.41 (m, 1H), 4.25 (tt, J=3.7, 10.7 Hz, 1H), 3.77-3.72 (m, 4H), 3.47-3.42 (m, 4H), 2.20-2.12 (m, 4H), 1.99-1.83 (m, 4H).

### Prodrug 19

LCMS (Method C): Rt=3.06 min, m/z [M+H]⁺=563

¹H NMR (400 MHz, DMSO-d₆) δ ppm: 8.70 (d, J=2.9 Hz, 1H), 7.89 (d, J=2.9 Hz, 1H), 7.79-7.77 (m, 1H), 7.35 (s, 1H), 7.33-7.31 (m, 1H), 6.90-6.89 (m, 1H), 6.57 (s, 1H), 5.52-5.46 (m, 3H), 5.10 (hept, J=7.0 Hz, 1H), 4.29-4.21 (m, 1H), 3.77-3.72 (m, 4H), 3.47-3.42 (m, 4H), 2.24-2.10 (m, 4H), 1.99-1.82 (m, 4H), 1.57 (d, J=7.0 Hz, 6H).

### Prodrug 20

LCMS (Method C): Rt=3.32 min, m/z [M+H]⁺=577

¹H NMR (400 MHz, DMSO-d₆) δ ppm: 8.63 (d, J=3.0 Hz, 1H), 7.86 (s, 1H), 7.74 (dd, J=1.1, 1.1 Hz, 1H), 7.62 (d, J=3.0 Hz, 1H), 7.28 (dd, J=1.1, 1.1 Hz, 1H), 6.92 (dd, J=1.1, 1.1 Hz, 1H), 6.52 (s, 1H), 5.98 (q, J=6.3 Hz, 1H), 5.41-5.39 (m, 1H), 5.20 (hept, J=6.6 Hz, 1H), 4.22 (tt, J=3.8, 11.3 Hz, 1H), 3.75-3.71 (m, 4H), 3.44-3.39 (m, 4H), 2.24-1.79 (m, 8H), 1.69 (d, J=6.3 Hz, 3H), 1.42 (d, J=6.6 Hz, 3H), 1.35 (d, J=6.6 Hz, 3H).

### Prodrug 21

LCMS (Method C): Rt=3.26 min, m/z [M+H]⁺=565

¹H NMR (400 MHz, DMSO-d₆) δ ppm: 8.69 (d, J=3.0 Hz, 1H), 7.90 (s, 1H), 7.82 (dd, J=0.6, 3.0 Hz, 1H), 7.75 (dd, J=1.1, 1.1 Hz, 1H), 7.29 (dd, J=1.1, 1.1 Hz, 1H), 6.89 (dd, J=1.1, 1.1 Hz, 1H), 6.56 (s, 1H), 5.49-5.45 (m, 1H), 5.30 (hept, J=6.5 Hz, 1H), 4.24 (tt, J=3.7, 11.3 Hz, 1H), 3.77-3.73 (m, 4H), 3.47-3.42 (m, 4H), 2.23-2.05 (m, 4H), 1.94-1.80 (m, 4H), 1.46 (d, J=6.5 Hz, 6H).

### Prodrug 22

LCMS (Method C): Rt=2.92 min, m/z [M+H]⁺=563

¹H NMR (400 MHz, DMSO-d₆) δ ppm: 8.68 (d, J=3.0 Hz, 1H), 7.87 (d, J=3.0 Hz, 1H), 7.78-7.76 (m, 1H), 7.39 (s, 1H), 7.31-7.30 (m, 1H), 6.91-6.91 (m, 1H), 6.54 (s, 1H), 6.06 (q, J=6.3 Hz, 1H), 5.48-5.44 (m, 1H), 4.50-4.33 (m, 2H), 4.26-4.19 (m, 1H), 3.76-3.72 (m, 4H), 3.46-3.41 (m, 4H), 2.18-2.07 (m, 4H), 1.96-1.93 (m, 2H), 1.87-1.81 (m, 2H), 1.74 (d, J=6.3 Hz, 3H), 1.35 (t, J=7.1 Hz, 3H).

### Prodrug 23

LCMS (Method C): Rt=3.10 min, m/z [M+H]⁺=563

¹H NMR (400 MHz, DMSO-d₆) δ ppm: 8.63 (d, J=3.0 Hz, 1H), 7.85 (s, 1H), 7.75-7.74 (m, 1H), 7.63 (d, J=3.0 Hz, 1H), 7.29-7.28 (m, 1H), 6.93-6.91 (m, 1H), 6.51 (s, 1H), 6.01 (q, J=6.4 Hz, 1H), 5.40-5.36 (m, 1H), 4.46 (q, J=7.2 Hz, 2H), 4.26-4.17 (m, 1H), 3.74-3.71 (m, 4H), 3.44-3.39 (m, 4H), 2.24-1.78 (m, 8H), 1.69 (d, J=6.4 Hz, 3H), 1.33 (t, J=7.2 Hz, 3H).

### Prodrug 24

LCMS (Method D): Rt=4.09 min, m/z [M+H]⁺=563

¹H NMR (400 MHz, DMSO-d₆) δ ppm: 8.72 (d, J=3.1 Hz, 1H), 7.91 (dd, J=0.6, 3.1 Hz, 1H), 7.79-7.78 (m, 1H), 7.33-7.33 (m, 1H), 7.30 (s, 1H), 6.90-6.89 (m, 1H), 6.57 (s, 1H), 5.51-5.47 (m, 1H), 4.24 (tt, J=3.6, 11.1 Hz, 1H), 3.77-3.73 (m, 4H), 3.64-3.57 (m, 1H), 3.47-3.43 (m, 4H), 2.24-2.14 (m, 4H), 1.99-1.84 (m, 4H), 1.12-1.05 (m, 4H).

### Prodrug 25

LCMS (Method C): Rt=3.02 min, m/z [M+H]⁺=565

¹H NMR (400 MHz, DMSO-d₆) δ ppm: 8.69 (d, J=3.1 Hz, 1H), 8.28 (s, 1H), 7.82 (dd, J=0.6, 3.1 Hz, 1H), 7.76-7.75 (m, 1H), 7.27-7.26 (m, 1H), 6.89-6.89 (m, 1H), 6.56 (s, 1H), 5.49-5.47 (m, 1H), 4.65 (hept, J=6.7 Hz, 1H), 4.23 (tt, J=3.6, 11.2 Hz, 1H), 3.77-3.73 (m, 4H), 3.47-3.42 (m, 4H), 2.21-2.06 (m, 4H), 1.94-1.81 (m, 4H), 1.45 (d, J=6.7 Hz, 6H).

### Prodrug 26

LCMS (Method C): Rt=3.12 min, m/z [M+H]⁺=563

¹H NMR (400 MHz, DMSO-d₆) δ ppm: 8.68 (d, J=3.0 Hz, 1H), 7.84 (s, 1H), 7.82 (d, J=3.0 Hz, 1H), 7.75 (dd, J=1.1, 1.1 Hz, 1H), 7.29 (dd, J=1.1, 1.1 Hz, 1H), 6.90 (dd, J=1.1, 1.1 Hz, 1H), 6.56 (s, 1H), 5.49-5.45 (m, 1H), 4.28-4.20 (m, 1H), 4.20-4.13 (m, 1H), 3.77-3.72 (m, 4H), 3.47-3.42 (m, 4H), 2.21-2.06 (m, 4H), 1.97-1.80 (m, 4H), 1.22-1.09 (m, 4H).

### Prodrug 27

LCMS (Method D): Rt=4.19 min, m/z [M+H]⁺=576

¹H NMR (400 MHz, DMSO-d₆) δ ppm: 8.70 (d, J=3.0 Hz, 1H), 7.87 (d, J=3.0 Hz, 1H), 7.76 (dd, J=1.1, 1.1 Hz, 1H), 7.30 (s, 2H), 7.29 (dd, J=1.1, 1.1 Hz, 2H), 6.90 (dd, J=1.1, 1.1 Hz, 1H), 6.55 (s, 1H), 5.49-5.43 (m, 1H), 4.27-4.18 (m, 1H), 3.77-3.72 (m, 4H), 3.55-3.48 (m, 1H), 3.46-3.42 (m, 4H), 2.26-2.05 (m, 4H), 1.97-1.82 (m, 4H), 1.75 (s, 3H), 1.16-0.94 (m, 4H).

### Prodrug 31

LCMS (Method E): Rt=2.86 min, m/z [M+H]⁺=536

¹H NMR (400 MHz, DMSO-d₆) δ ppm: 13.64 (s, 1H), 8.67 (d, J=2.9 Hz, 1H), 8.37 (s, 1H), 7.92 (s, 1H), 7.80 (d, J=2.9 Hz, 1H), 6.54 (s, 1H), 5.64 (s, 2H), 5.37-5.34 (m, 1H), 3.81 (s, 3H), 3.78-3.72 (m, 4H), 3.48-3.42 (m, 4H), 2.96-2.96 (m, 1H), 2.14-2.01 (m, 4H), 1.93-1.81 (m, 4H).

### Biological Activity

### Biological Assay a

### Inhibition of DNA-PK Kinase Activity

Assay buffer was 50 mM Hepes pH 7.5 containing 0.1 mg/ml BSA (albumin from bovine serum), 50 μM DTT (dithiothreitol), 20 mM MgCl₂ and 10 μg/ml calf thymus DNA (activation buffer). Kinase reaction was carried out in OptiPlate 384 plates (PerkinElmer 6007299). All compounds tested were dissolved in dimethyl sulfoxide (DMSO) and further dilutions were made in assay buffer. Final DMSO concentration was 1% (v/v). Pre-incubation consisted of compound (1% DMSO in control and blank wells) and 24 ng/well DNA-PK kinase to allow the compounds to bind the enzyme (with buffer in the blank wells) for 15 minutes; after this time 150 μM Adenosine-5′-triphosphate (ATP) and 0.2 μg/μl DNA-PK substrate were added to commence the kinase reaction. Incubations were carried out for 1 hour at 25° C. Kinase activity was determined by using ADP-Glo™ reagent from Promega according to the manufacturer's instructions. The light generated was measured using a luminometer (EnVision, PerkinElmer). Signal obtained in the wells containing blank samples was subtracted from all other wells and IC₅₀ values were determined by fitting a sigmoidal curve to % inhibition of control versus Log₁₀ compound concentration.

### Biological Assay B

### Effect of Compounds on Phospho-DNA-PK in FaDu Cells

All compounds tested were dissolved in DMSO and further dilutions were made in culture medium. Final DMSO concentration was 0.3% (v/v). The human FaDu cells (ATCC) were cultured in MEM medium supplemented with Glutamax and 10% fetal calf serum (PAA). Cells were routinely maintained at densities of 0.2×10⁶ cells per ml at 37° C. in a humidified 5% CO₂, 95% air atmosphere. Cells were passaged twice a week, splitting back to obtain the low density. Cells were seeded in 96 well plates (Corning 3904) at 1×10⁶ cells per ml media in a volume of 50 μl per well. Seeded cells were incubated at 37° C. in a humidified 5% CO₂, 95% air atmosphere for 24 hours. Compounds were added (25 μl) to the cells and pre-incubated for 1 hour. 1 μg/ml DNA-damaging agent Neocarzinostatin (NCS) was added (25 μl) to a final volume of 100 μl and incubated for 2 hours at 37° C. in a humidified 5% CO₂, 95% air atmosphere. After this period of time plates were removed from the incubator and the 100 μl dilutions were removed from the wells gently, prior to the addition of 75 μl cold RIPA lysis buffer with protease and phosphatase inhibitors to achieve the cells lysis, followed by shaking on a plate shaker at 4° C. for 30 minutes. At the end of this incubation, lysate was assessed for phospho-DNA-PK levels by sandwich immune-assay carried out in anti-mouse antibody coated MesoScale plates. For each experiment, controls (containing DMSO and NCS but not test compound) and blanks (containing NCS and 3 μM parent compound 163, a test concentration known to give full inhibition) were run in parallel. Signal obtained in the wells containing blank samples was subtracted from all other wells and IC₅₀ values were determined by fitting a sigmoidal curve to % inhibition of control versus Logo compound concentration.

### Biological Assay C

### Stirred Cell Suspension Assay

All compounds tested were dissolved in DMSO at 10 mM/L and further dilutions were made in culture medium. Final DMSO concentration was <0.3% (v/v). The human FaDu and HCT116 cells (ATCC) were cultured in MEM medium and 10% fetal calf serum (PAA). Cells were routinely maintained at densities of 0.2×10e6 cells per ml and incubated at 37° C. in a humidified 5% CO₂, 95% air atmosphere. Cells were passaged twice a week, splitting back to obtain the low density. At the time of experiment, cells were trypsinized, counted and suspended at a density of 1% cells/volume in glass vials containing 2 ml stirred media and gassed at different pO₂. After 30 minutes, test compounds were added at 25 μM/L and 150 μl samples were removed at intervals up to 4 hours. Each sample was split to test for (1) free drug and (2) total extraction and analysed using high performance liquid chromatography to measure the release of the parent inhibitor from the hypoxia activated prodrug.

Data for the parent compounds of the invention in Biological assays A and B above are provided in Table 45 (the values in Table 45 are averaged values over all measurements on all batches of a parent compound).

TABLE 45

| Parent compound | ADP-Glo IC₅₀ (nM) | MSD FaDu IC₅₀ (nM) |
|---|---|---|
| 1 | 82 | 832 |
| 2 | 39 | 743 |
| 3 | 9 | 361 |
| 4 | 37 | 1834 |
| 5 | 21 | 1371 |
| 6 | 74 | 2008 |
| 7 | 415 | |
| 8 | 279 | 6655 |
| 9 | 468 | |
| 10 | <30000 | |
| 11 | 50 | 3502 |
| 12 | 70 | 2552 |
| 13 | 71 | 2844 |
| 14 | 73 | 4470 |
| 15 | 108 | 2154 |
| 16 | 139 | 18633 |
| 17 | 148 | 4450 |
| 18 | 149 | 4641 |
| 19 | 159 | 6558 |
| 20 | 159 | 8868 |
| 21 | 164 | 7959 |
| 22 | 198 | 1551 |
| 23 | 211 | |
| 24 | 213 | 9346 |
| 25 | 224 | |
| 26 | 226 | |
| 27 | 274 | |
| 28 | 342 | |
| 29 | 345 | |
| 30 | 369 | |
| 31 | 492 | |
| 32 | 523 | |
| 33 | 532 | |
| 34 | 588 | |
| 35 | 720 | |
| 36 | 949 | |
| 37 | 1302 | |
| 38 | <30000 | |
| 39 | <30000 | |
| 40 | <30000 | |
| 41 | <30000 | |
| 42 | 2265 | |
| 43 | | |
| 44 | | |
| 45 | | |
| 46 | | |
| 47 | | |
| 48 | 328 | |
| 49 | | |
| 50 | | |
| 51 | | |
| 52 | | |
| 53 | 242 | |
| 54 | 3 | 182 |
| 55 | | |
| 56 | 12 | 238 |
| 57 | 120 | 2456 |
| 58 | 27 | 1587 |
| 59 | | |
| 60 | | |
| 61 | | |
| 62 | | |
| 63 | | |
| 64 | | |
| 65 | 2 | 210 |
| 66 | 3 | 265 |
| 67 | 32 | 891 |
| 68 | 48 | |
| 69 | 261 | 5049 |
| 70 | 1 | 113 |
| 71 | 2 | 62 |
| 72 | 11 | 218 |
| 73 | 55 | 1728 |
| 74 | 3 | 759 |
| 75 | 5 | 57 |
| 76 | 11 | 143 |
| 77 | 6 | 104 |
| 78 | 4 | 195 |
| 79 | 6 | 373 |
| 80 | 7 | 240 |
| 81 | 10 | 507 |
| 82 | 141 | 1888 |
| 83 | 169 | |
| 84 | 188 | 3843 |
| 85 | 2031 | |
| 86 | 223 | |
| 87 | 51 | 1156 |
| 88 | | |
| 89 | 361 | |
| 90 | | |
| 91 | | |
| 92 | | |
| 93 | | |
| 94 | | |
| 95 | 5436 | |
| 96 | | |
| 97 | 27 | 3328 |
| 98 | 36 | 702 |
| 99 | 15 | 724 |
| 100 | 32 | 2365 |
| 101 | 32 | 2126 |
| 102 | 39 | 905 |
| 103 | 45 | 3703 |
| 104 | 50 | 3019 |
| 105 | 51 | 1159 |
| 106 | 52 | 11390 |
| 107 | 66 | 2373 |
| 108 | 69 | 3378 |
| 109 | 76 | 4831 |
| 110 | 88 | |
| 111 | 88 | 2143 |
| 112 | 112 | 2032 |
| 113 | 114 | 6600 |
| 114 | 117 | 9670 |
| 115 | 120 | 7991 |
| 116 | 124 | |
| 117 | 125 | 1932 |
| 118 | | |
| 119 | 136 | 2412 |
| 120 | 140 | 3182 |
| 121 | 143 | 3518 |
| 122 | 185 | 4359 |
| 123 | 232 | |
| 124 | 247 | |
| 125 | 504 | |
| 126 | 676 | |
| 127 | 203 | 13823 |
| 128 | 30 | 1386 |
| 129 | 44 | 1313 |
| 130 | 48 | 2529 |
| 131 | 63 | 7311 |
| 132 | 82 | 10557 |
| 133 | 155 | 11437 |
| 134 | 155 | |
| 135 | 178 | |
| 136 | 340 | |
| 137 | 457 | |
| 138 | 501 | |
| 139 | 126 | |
| 140 | 257 | |
| 141 | 370 | |
| 142 | 808 | |
| 143 | 110 | 1802 |
| 144 | 117 | 2889 |
| 145 | 3442 | |
| 146 | 232 | |
| 147 | 340 | |
| 148 | 171 | 4916 |
| 149 | 244 | |
| 150 | 66 | 1919 |
| 151 | 82 | 1693 |
| 152 | 19 | 5789 |
| 153 | 30 | 2487 |
| 154 | 26 | 1070 |
| 155 | 134 | 7865 |
| 156 | 93 | 875 |
| 157 | 160 | 2998 |
| 158 | 102 | 10039 |
| 159 | 116 | |
| 160 | 493 | |
| 161 | 125 | 2886 |
| 162 | 2 | 112 |
| 163 | 2 | 134 |
| 164 | 58 | 3740 |
| 165 | 31 | 1174 |
| 166 | 4 | 190 |
| 167 | 4 | 219 |
| 168 | 5 | 272 |
| 169 | 1 | 319 |
| 170 | 8 | 232 |
| 171 | 10 | 270 |
| 172 | 242 | |
| 173 | | |
| 174 | | |
| 175 | 18 | 464 |

In Table 45 reference to an IC₅₀ of less than <30000 is based on the % inhibition at 1 μM (18-50% in each case).

### Activity of Prodrugs in Biological Assay B

Prodrugs 1-31 described herein displayed reduced potency in the order of 3- to >208-fold with respect to the corresponding parent compounds when tested in the FaDu cellular assay (Biological Assay B). For the majority of prodrugs tested, the fold-reduction in potency was between 10- and >208-fold compared to the parent compound.

### Activity in Biological Assay C

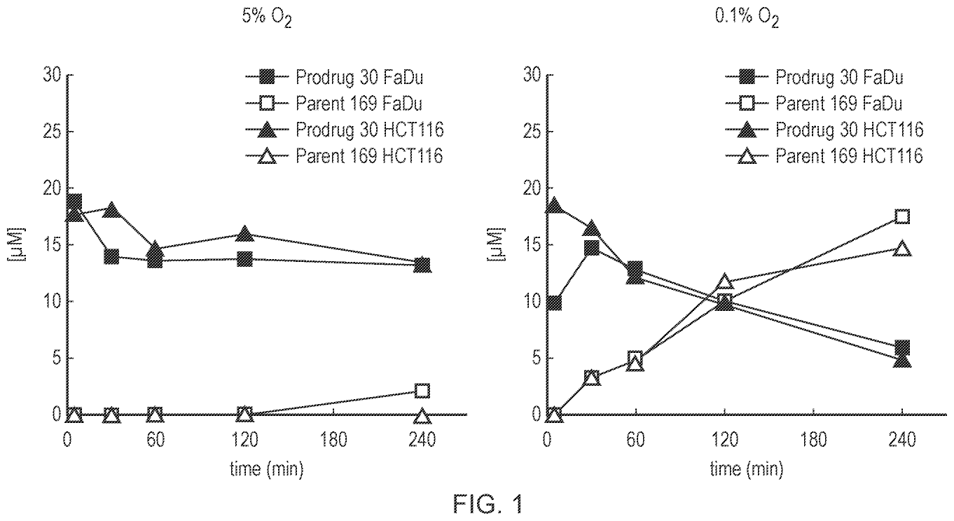

Prodrug 30 was tested in the Stirred cell assay (Biological Assay C) under conditions of 5% oxygen and 0.1% oxygen using FaDu and HCT116 cells. The results of these tests are shown in FIG. 1. This shows that under 5% oxygen the concentration of prodrug remained essentially constant and very little parent compound (Parent Compound 169) was released during the test. In contrast under low oxygen concentration (0.1% O₂), parent compound was released from the prodrug compound resulting in an increase in the concentration of the Parent Compound 169 and a decrease in the concentration of the prodrug 30, illustrating release of the parent compound from the prodrug under hypoxic conditions in the presence of cells.

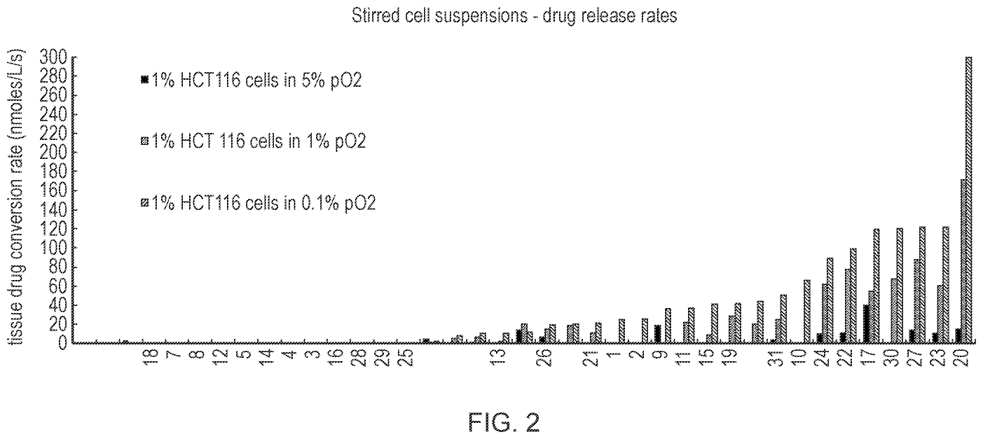

The conversion of prodrugs to the respective parent compounds in the stirred cell assay (Biological Assay C) under conditions of 5% oxygen, 1% oxygen and 0.1% oxygen using 1% HCT116 cells by volume in media with 10% FBS at 37° C. were further analysed as shown in FIG. 2 where conversion rates were calculated. The 1^{st}, 2^{nd} & 3^{rd} column for each compound represent data for 5%, 1% and 0.1% oxygen. The columns with no labels are compounds not described herein. Prodrugs 1, 2, 9, 10, 11, 13, 15, 17, 19, 20, 21, 22, 23, 24, 26, 27, 30 and 31 released the respective parent compound under hypoxic conditions in the presence of cells.

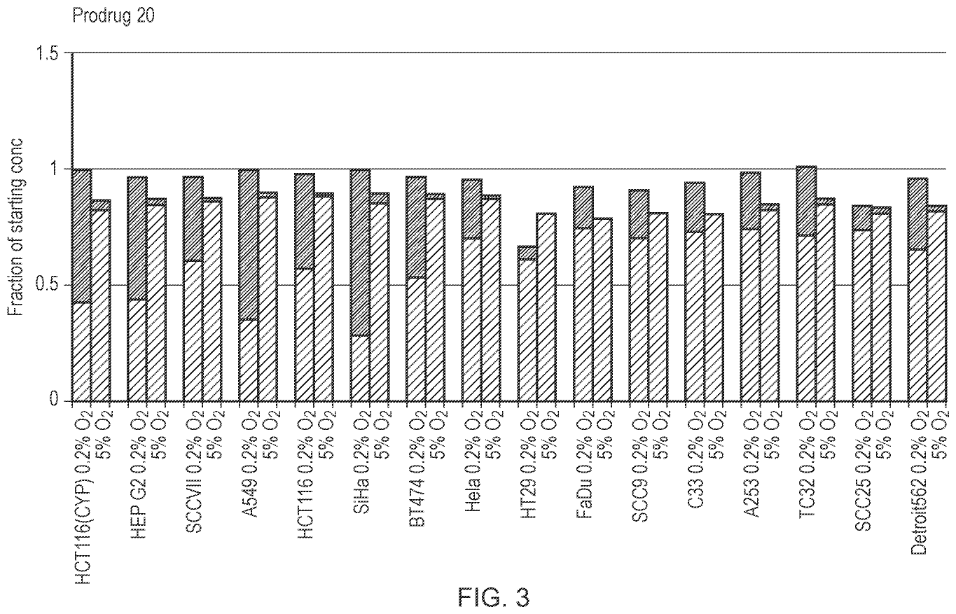

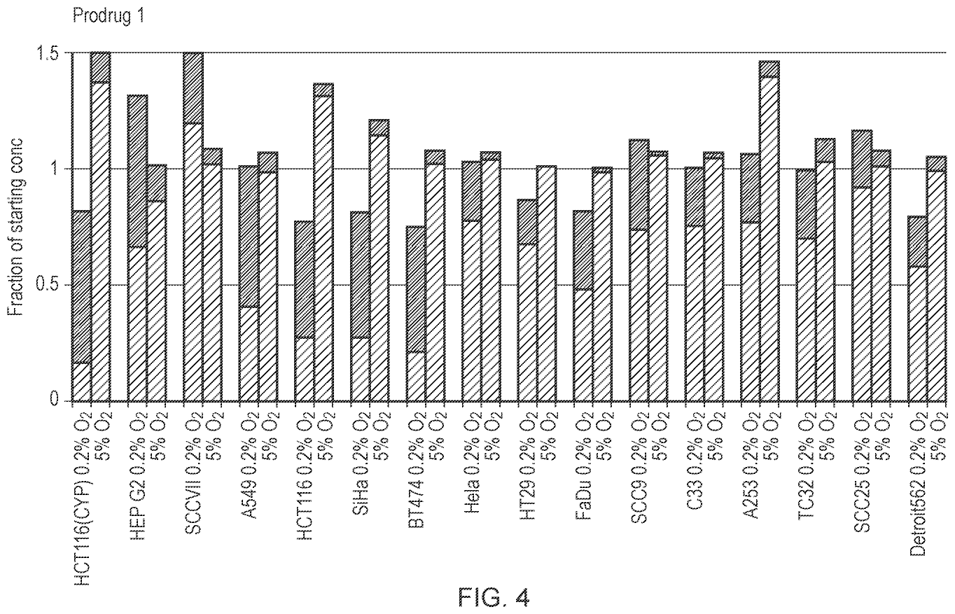

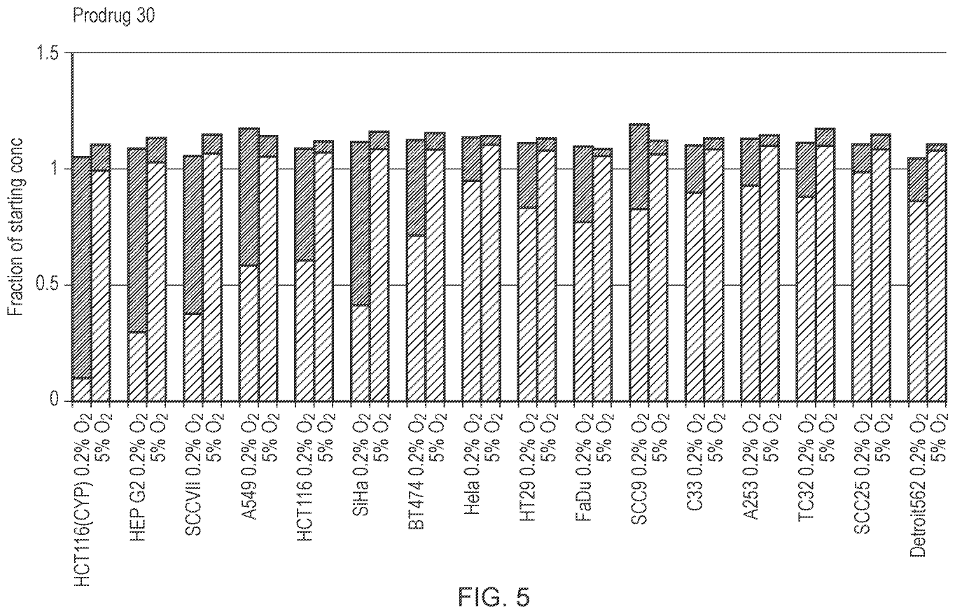

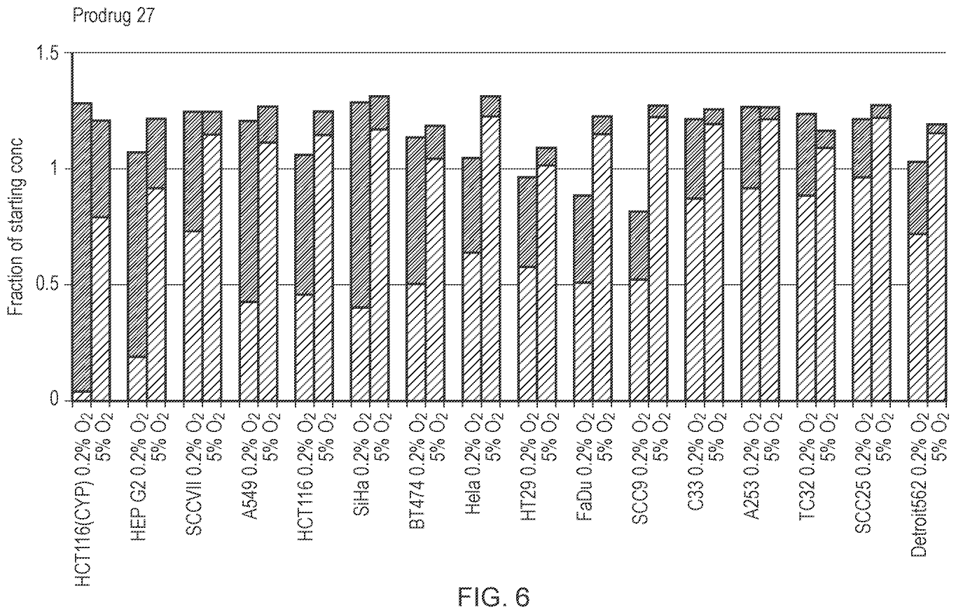

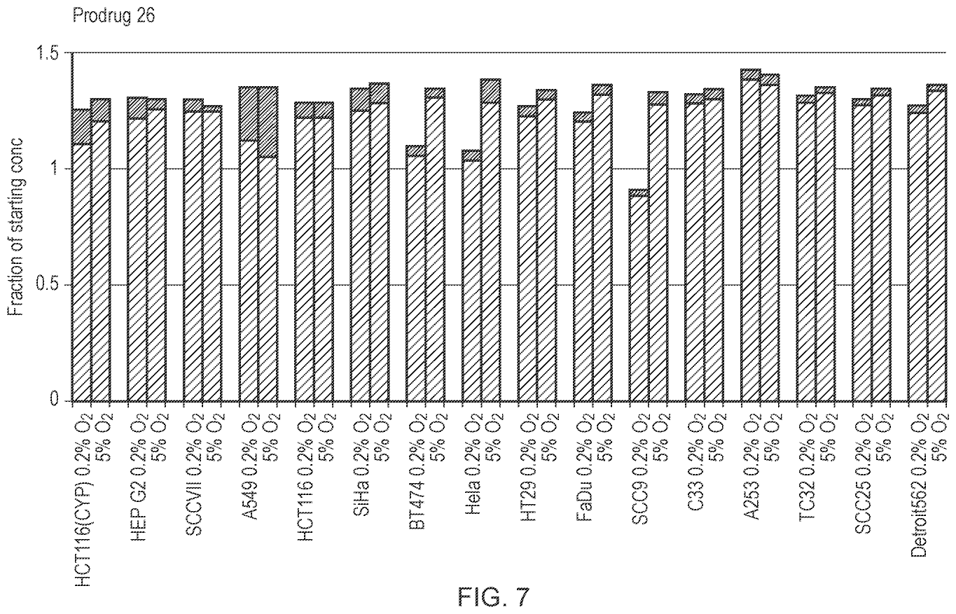

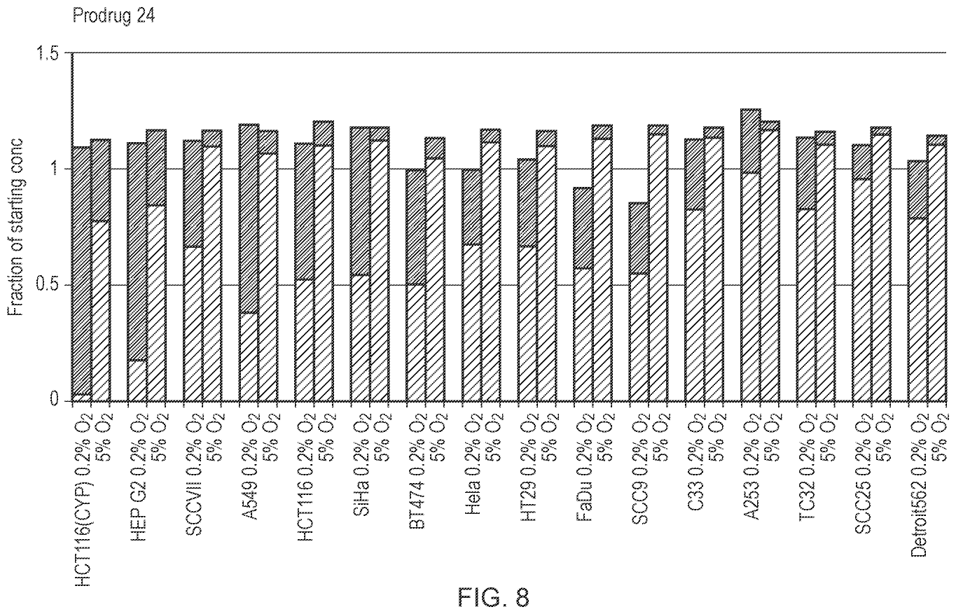

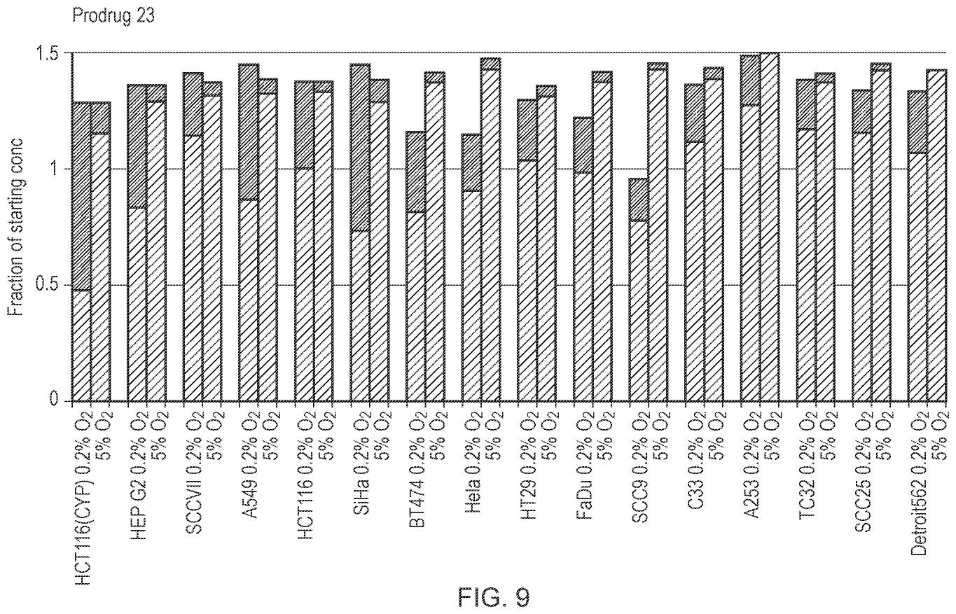

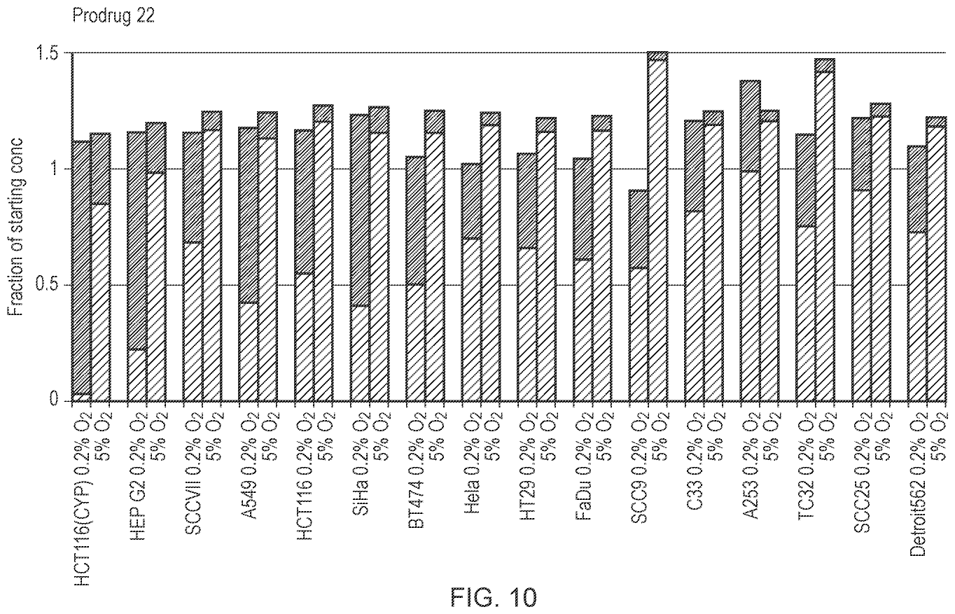

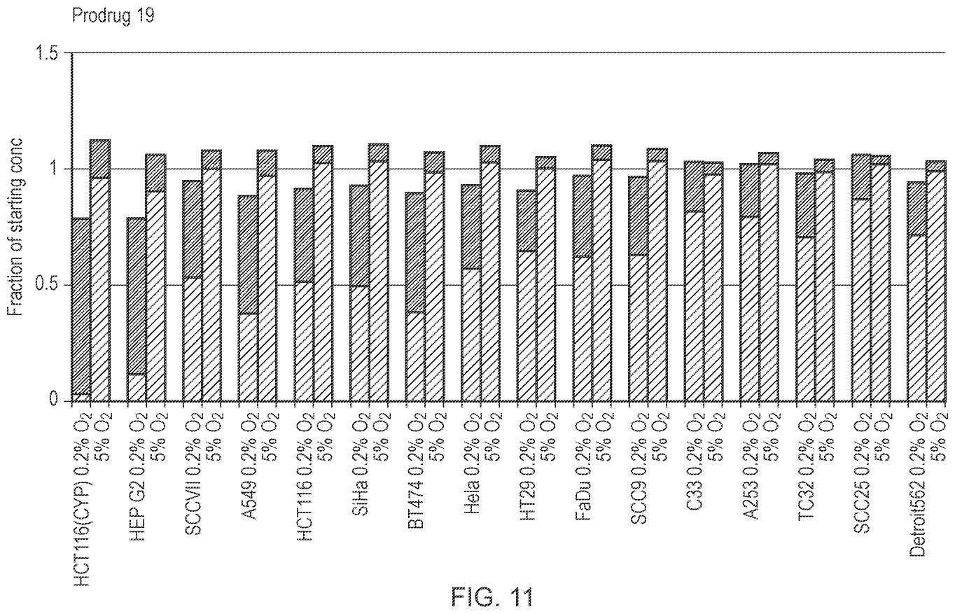

FIGS. 3-11 demonstrate activation in across a panel of cell lines under oxic and hypoxic conditions for prodrugs 20, 1, 30, 27, 26, 24, 23, 22 and 19, respectively. The conversion of indicated prodrug to active parent compound is shown in cell lines held at 0.2% and 5% oxygen in 1% (by volume of cells) stirred culture with starting concentrations of 25 μM of prodrug. Upper dark hashed segment shows the proportion of released parent compound. The lower light hashed segment indicates prodrug remaining after 4 hours. The left bar for each cell line indicates conversion at 0.2% oxygen and right bar for each cell line indicates conversion at 5% oxygen.

### Biological Assay D

Activation and activity in 3D HCT116 or FaDu cell spheroids

Multicellular HCT116 or FaDu cell spheroids, approximately 1 mm in diameter, were incubated with the compound tested for 1 h at 37° C., 5% CO₂ and either 5% or 20% oxygen whilst being stirred and then irradiated with 10Gy X-rays followed by a further 3 hours of incubation. The spheroids were then dissociated to a single cell suspension. Live cells were counted using Hoechst 33342 and propidium iodide and plated for clonogenic cell survival. Usually 12 concentrations of each compound were assessed from about 0.01 to 20 μM and a drug dose that caused 50% decrease in cell survival was determined (EC₅₀). Separation of EC₅₀ curves (5% and 20% oxygen) indicates prodrug release to parent inhibitor and subsequent sensitization to X-ray induce cell kill.

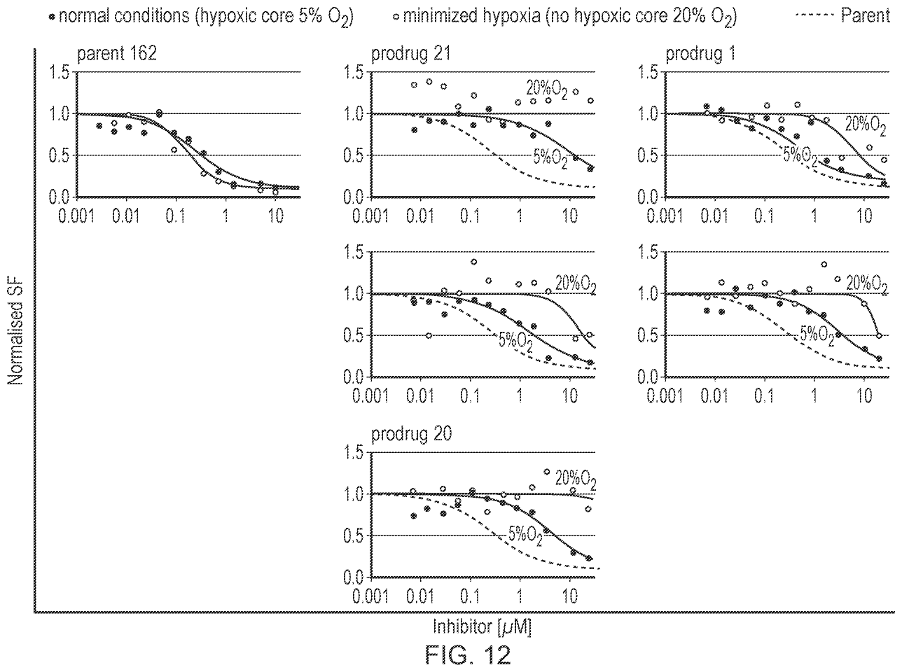

FIG. 12 shows the assessment of activation and activity of prodrugs 1, 20 and 21 in 3D spheroids. Data for parent compound 162 is also shown. The hatched line shown in the prodrug plots indicates the profile of the parent compound 162 for comparison.

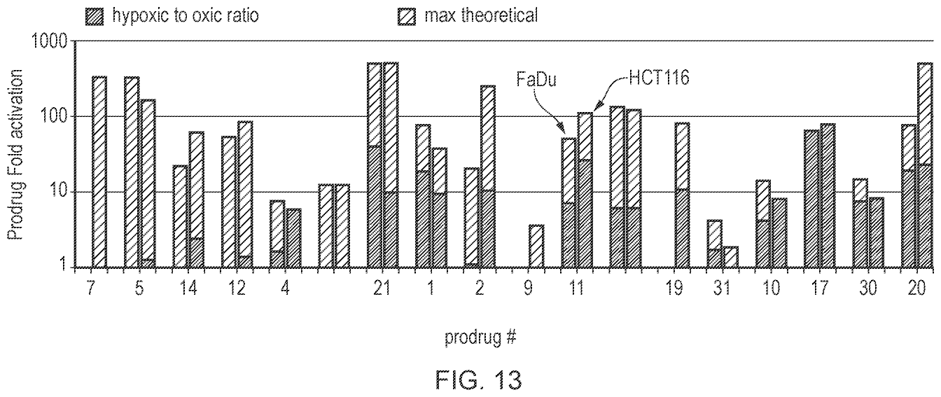

FIG. 13 shows the hypoxic to oxic ratio observed in the 3D spheroids assay. EC₅₀ values obtained from the 3D spheroid screen are shown for each prodrug indicated. Prodrugs 1, 2, 5, 7, 9, 10, 11, 12, 14, 17, 19, 20, 21, 30 and 31 were assessed. The bar graph shows how effective the prodrugs are in FaDu and HCT116 cell lines in comparison with the maximum theoretical activation of the parent compound. Prodrugs are ranked left to right with increasing hypoxic release rates.

### Biological Assay E

Clonogenic Cell Survival Following Treatment with 10Gy X-Rays

FaDu human tumours were implanted into Rag2M mice and grown to approximately 800 mg in size. Compounds as indicated were administered to mice IV 1 hour prior to 10Gy whole body X-irradiation. Tumours were excised 40 minutes after irradiation and single cell suspensions prepared, counted and plated for clonogenic survival.

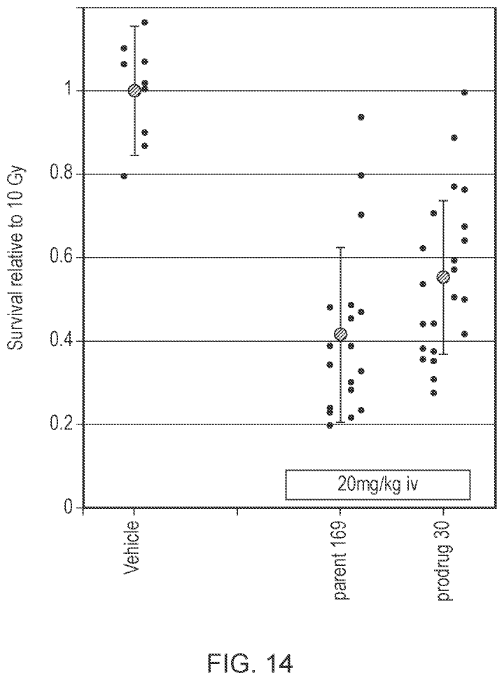

FIG. 14 demonstrates clonogenic cell survival after tumour excision following treatment with 10Gy X-rays after treatment with vehicle only, prodrug 30 and parent compound 169, respectively.

### Biological Assay F

### Pharmacokinetic Assessment

The tested compounds were administered to mice (Rag2M) intravenously (IV) at a dose of 10 mg/kg and orally (PO) at a dose of 40 mg/kg, respectively.

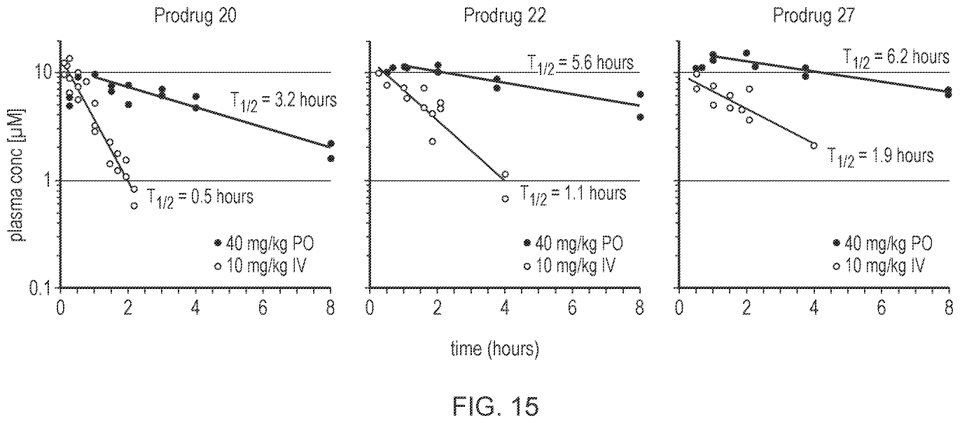

FIG. 15 shows the pharmacokinetics of prodrugs 20, 22 and 27 and compares mouse (Rag2M) plasma pharmacokinetics for prodrugs 20, 22 and 27 after IV (intravenous) and PO (per oral) administration as indicated.

### Biological Assay G

Western blots of tumour lysates following treatment with 10Gy X-rays FaDu human tumours were implanted into Rag2M mice. Compounds as indicated were administered to mice IV 1 hour prior to 10Gy whole body X-irradiation. Tumours were excised 40 minutes after irradiation and tumour lysates prepared. Gels were stained for pDNA-PK.

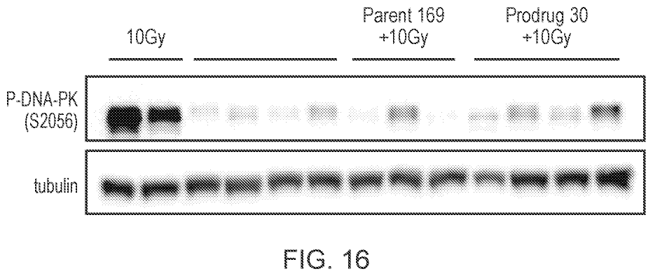

FIG. 16 shows Western blots of tumour lysates following treatment with 10Gy X-rays after treatment with prodrug 30 and parent compound 169, respectively.

### Biological Assay H

### Evaluation of Effect of Radiotherapy

SiHa human tumours were grown subcutaneously in Rag2M mice. The mice were treated with vehicle, radiotherapy alone (10Gy single dose to tumour and minimal surrounding tissue), and radiotherapy+prodrug administered at 40 mg/kg PO.

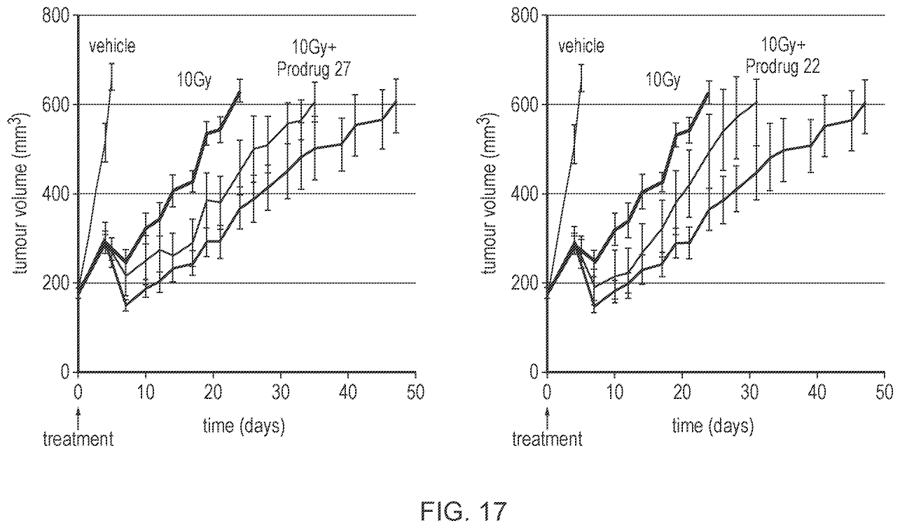

FIG. 17 shows tumour growth measurements after administration of vehicle alone, after 10Gy, after 10Gy+prodrug 27 and after 10Gy+Prodrug 22. Far right line in each case represent a positive control with a non-prodrug (parental) DNA-PK inhibitor.

1. A compound of formula (I), or prodrug thereof or a pharmaceutically acceptable salt or N-oxide of the compound of formula (I) or the prodrug thereof: 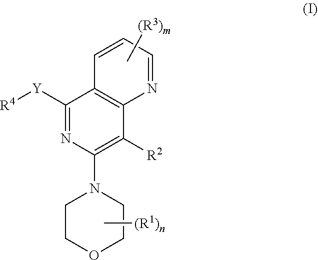
wherein
Y is independently selected from O and NR⁵;
R¹ is independently at each occurrence selected from C₁-C₆-alkyl and C₁-C₆-haloalkyl;
R² is independently selected from H, C₁-C₆-alkyl, C₁-C₆-haloalkyl, cyano and halo;
R³ is independently at each occurrence selected from C₁-C₆-alkyl, C₁-C₆-haloalkyl, cyano, halo, OR^{6a}, NR^{7a}R^{8a};
R⁴ is -L¹-L²-R^{9a};
R⁵ is independently selected from: H and C₁-C₆-alkyl;
or R⁴ and R⁵ together with the nitrogen to which they are attached form a 3- to 11-membered heterocycloalkyl group or a 5-membered heteroaryl group, said heterocycloalkyl group being optionally substituted with from 1 to 4 R^{10a} substituents and/or a single R¹¹ substituent and said heteroaryl group being optionally substituted with from 1 to 4 R^{12a} substituents and/or a single R¹¹ substituent; wherein said heterocycloalkyl group may be monocyclic, bicyclic or a spirocyclic bicycle;
-L¹- is independently either absent or is —C₁-C₆-alkylene, wherein said alkylene group is optionally substituted with from 1 to 4 R^{10b} substituents;
-L²- is independently either absent or is -L³-L⁴-;
-L³- is independently selected from: C₁-C₆-alkylene, C₃-C₈-cycloalkyl, 3- to 8-membered heterocycloalkyl, wherein said cycloalkyl or heterocycloalkyl group may be monocyclic, bicyclic or a spirocyclic bicycle and wherein said alkylene, cycloalkyl or heterocycloalkyl group may be optionally substituted with from 1 to 4 R^{10c} substituents;
-L⁴- is independently either absent or is selected from —NR^{13a}— and —O—;
R^{9a} and R^{9b} are each independently selected from: phenyl, naphthyl, 5, 6, 9 or 10 membered heteroaryl, 3- to 8-membered heterocycloalkyl, C₃-C₈-cycloalkyl and C₁-C₃-alkylene-R¹⁴; wherein R¹⁴ is independently selected from: phenyl, naphthyl, 5, 6, 9 or 10 membered heteroaryl, 3- to 8-membered heterocycloalkyl and C₃-C₈-cycloalkyl; wherein any phenyl, napthyl or heteroaryl group of which R^{9a} or R^{9b} is comprised is optionally substituted with from 1 to 4 R¹⁵ substituents and any alkylene, cycloalkyl or heterocycloalkyl group of which R^{9a} or R^{9b} is comprised is optionally substituted with from 1 to 4 R^{10d} substituents;
R¹¹ is -L⁵-L⁶-R^{9b};
-L⁵- is independently either absent or is selected from C₁-C₃-alkylene, C(O) and S(O)₂, wherein said alkylene group is optionally substituted with from 1 to 4 R^{10e} substituents;
-L⁶- is independently either absent or is independently selected from —NR^{13b}— and —O—;
R^{6a}, R^{6b}, R^{6c} and R^{6d} are each independently at each occurrence selected from: H, C₁-C₆-alkyl (which may be optionally substituted with from 1 to 3 O—C₁-C₄-alkyl groups) and C₁-C₆-haloalkyl;
R^{7a}, R^{7b}, R^{7c} and R^{7d}, are each independently at each occurrence selected from H and C₁-C₆-alkyl (which may be optionally substituted with from 1 to 3 O—C₁-C₄-alkyl groups);
R^{8a}, R^{8b} and R^{8c} are each independently at each occurrence selected from H, C₁-C₆-alkyl (which may be optionally substituted with from 1 to 3 O—C₁-C₄-alkyl groups), C(O)—C₁-C₆-alkyl, S(O)₂—C₁-C₆-alkyl, C(O)—O—C₁-C₆-alkyl, C(O)-phenyl and S(O)₂-phenyl; wherein said phenyl groups are optionally substituted with from 1 to 4 R^{12b} groups;
R^{10a}, R^{10b}, R^{10c}, R^{10d} and R^{10e} are each independently at each occurrence selected from: ═O, ═S, C₁-C₆-alkyl, C₂-C₆-alkenyl, C₂-C₆-alkynyl, C₁-C₆-haloalkyl, cyano, halo, nitro, (CR^{7b}R^{7b})ₓOR^{6b}, (CR^{7b}R^{7b})ₓNR^{7b}R^{8b}, C(O)R^{7b}, C(O)NR^{7b}R^{7b}, C(O)OR^{7b}, S(O)₂R^{7b}, S(O)R^{7b}, S(O)₂NR^{7b}R^{7b} and phenyl; wherein said phenyl group is optionally substituted with from 1 to 4 R^{12c} groups;
R^{13a} and R^{13b} are each independently at each occurrence selected from H and C₁-C₆-alkyl;
R¹⁵ is independently at each occurrence selected from C₁-C₆-alkyl, C₂-C₆-alkenyl, C₂-C₆-alkynyl, C₁-C₆-haloalkyl, cyano, halo, nitro, (CR^{7c}R^{7c})ₓOR^{6c}, (CR^{7c}R^{7c})ₓNR^{7c}R^{8c}, C(O)R^{7c}, C(O)NR^{7c}R^{7c}, C(O)OR^{7c}, S(O)₂R^{7c}, S(O)R^{7c}, S(O)₂NR^{7c}R^{7c}, and phenyl; wherein said phenyl group is optionally substituted with from 1 to 4 R^{12d} groups;
R^{12a}, R^{12b}, R^{12c} and R^{12d} are each independently at each occurrence selected from: C₁-C₆-alkyl, C₂-C₆-alkenyl, C₂-C₆-alkynyl, C₁-C₆-haloalkyl, cyano, halo, nitro, OR^{6d}, NR^{7d}R¹⁷, C(O)R^{7d}, C(O)NR^{7d}, C(O)OR^{7d}, S(O)₂R^{7d}, S(O)R^{7d} and S(O)₂NR^{7d}R^{7d};
R¹⁷ is independently at each occurrence selected from H, C₁-C₆-alkyl, C(O)—C₁-C₆-alkyl, S(O)₂—C₁-C₆-alkyl and C(O)—O—C₁-C₆-alkyl;
n is an integer selected from 0, 1, 2 and 3;
m is an integer selected from 0, 1, 2, 3 and 4;
x is independently at each occurrence an integer selected from 0, 1, 2 and 3;
where the compound is optionally a prodrug of a compound of formula (I) or a salt or N-oxide of a prodrug of formula (I), the prodrug comprises a trigger moiety that releases the compound of formula (I) under reductive conditions.

2. A compound of claim 1, wherein the compound is a prodrug of a compound of formula (I), or a salt or N-oxide of a prodrug of formula (I), and the prodrug comprises a trigger moiety that releases the compound of formula (I) under reductive conditions.

3. A compound of claim 2, wherein the trigger moiety has the structure: 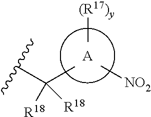
wherein ring A is a phenyl ring or a 5- or 6-membered heteroaryl ring;
R¹⁷ is independently at each occurrence selected from C₁-C₆-alkyl, C₁-C₆-haloalkyl, C₃-C₆-cycloalkyl, O—C₁-C₆-alkyl, cyano and halo;
R¹⁸ is independently at each occurrence selected from H, C₁-C₆-alkyl and C₁-C₆-haloalkyl; or the two R¹⁸ groups together form a C₃-C₆-cycloalkyl ring;
y is an integer from 0 to 3;
wherein the nitro group and the carbon attached to the two R¹⁸ groups are either attached to adjacent carbon atoms in Ring A or are attached to two carbon atoms in Ring A that are separated by two sp2 hybridised atoms selected from carbon and nitrogen.

4. A compound of claim 2, wherein the trigger moiety is attached to that portion of the prodrug that will be released as the compound of formula (I) via a functional group derived from an attachment point on the compound of formula (I), said attachment point being selected from OH, NH, NH₂ and a quaternisable nitrogen.

5. A compound of claim 1, wherein Y is O.

6. A compound of claim 1, wherein Y is NR⁵.

7. A compound of claim 5, wherein R⁴ is -L¹-L²-R^{9a}.

8. A compound of claim 7, wherein -L¹- is absent.

9. A compound of claim 7, wherein -L²- is -L³-L⁴-.

10. A compound of claim 9, wherein -L³- is C₃-C₆-cycloalkyl.

11. A compound of claim 9, wherein -L³- is a 3- to 8-membered heterocycloalkyl group wherein said heterocycloalkyl group may be monocyclic, bicyclic or a spirocyclic bicycle and wherein heterocycloalkyl group may be optionally substituted with from 1 to 4 R^{10c} substituents.

12. A compound of claim 9, wherein -L⁴- is absent.

13. A compound of claim 9, wherein -L⁴- is —NH—.

14. A compound of claim 7, wherein R^{9a} is a 5 or 6 membered heteroaryl.

15. A compound of claim 6, wherein R⁴ and R⁵ together with the nitrogen to which they are attached form a 3- to 11-membered heterocycloalkyl group; said heterocycloalkyl group being optionally substituted with from 1 to 4 R^{10a} substituents; wherein said heterocycloalkyl group may be monocyclic, bicyclic or a spirocyclic bicycle and wherein said heterocycloalkyl group is substituted with a single substituent.

16. A compound of claim 15, wherein -L⁵- is absent.

17. A compound of claim 15, wherein -L⁶- is absent.

18. A compound of claim 15, wherein -L⁶- is —NH—.

19. A compound of claim 15, wherein R^{9b} is a 5 or 6 membered heteroaryl.

20. A compound of claim 1, wherein n is 0.

21. A compound of claim 1, wherein R² is H.

22. A compound of claim 1, wherein m is 0.

23. A compound of claim 1, wherein m is 1, R³ is selected from OH and NHR^{7a} and the R³ group is positioned meta to the nitrogen in the pyridine ring to which (R³)ₘ is attached.

24. A compound of claim 1 wherein the compound of formula (I) is selected from: 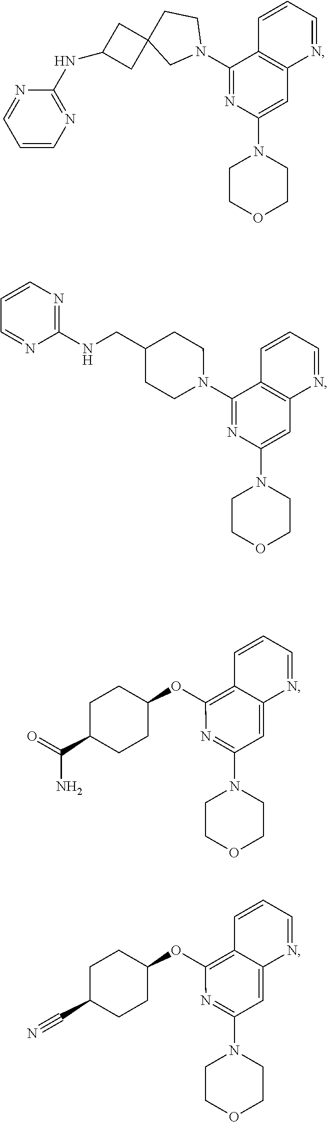 [SMILES: c1cnc(NC2CC3(CCN(c4nc(N5CCOCC5)cc5ncccc45)C3)C2)nc1 ; c1cnc(NCC2CCN(c3nc(N4CCOCC4)cc4ncccc34)CC2)nc1 ; NC(=O)[C@H]1CC[C@@H](Oc2nc(N3CCOCC3)cc3ncccc23)CC1 ; N#C[C@H]1CC[C@@H](Oc2nc(N3CCOCC3)cc3ncccc23)CC1] 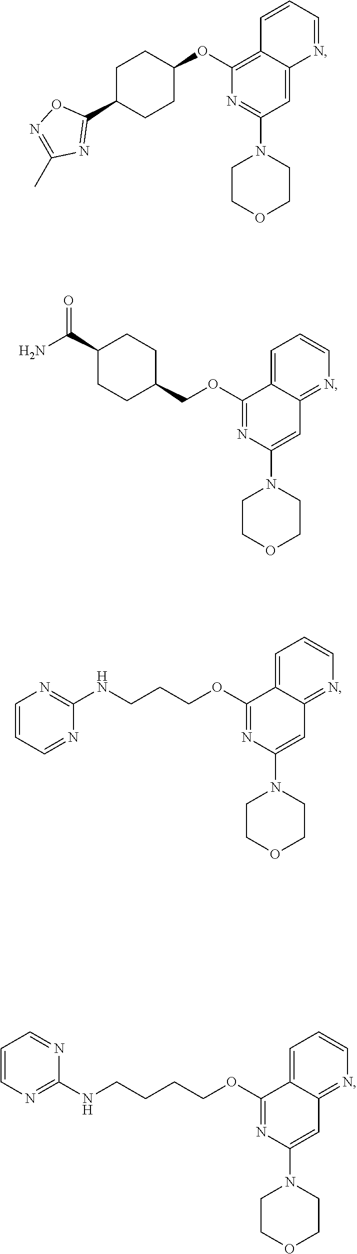 [SMILES: Cc1noc([C@H]2CC[C@@H](Oc3nc(N4CCOCC4)cc4ncccc34)CC2)n1 ; NC(=O)[C@H]1CC[C@@H](COc2nc(N3CCOCC3)cc3ncccc23)CC1 ; c1cnc(NCCCOc2nc(N3CCOCC3)cc3ncccc23)nc1 ; c1cnc(NCCCCOc2nc(N3CCOCC3)cc3ncccc23)nc1] 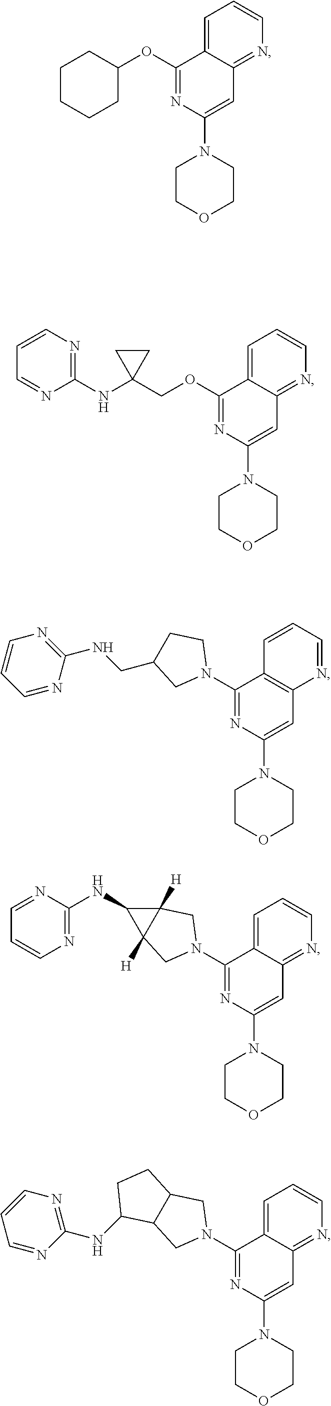 [SMILES: c1cnc2cc(N3CCOCC3)nc(OC3CCCCC3)c2c1 ; c1cnc(NC2(COc3nc(N4CCOCC4)cc4ncccc34)CC2)nc1 ; c1cnc(NCC2CCN(c3nc(N4CCOCC4)cc4ncccc34)C2)nc1 ; c1cnc(N[C@H]2[C@@H]3CN(c4nc(N5CCOCC5)cc5ncccc45)C[C@@H]32)nc1 ; c1cnc(NC2CCC3CN(c4nc(N5CCOCC5)cc5ncccc45)CC32)nc1] 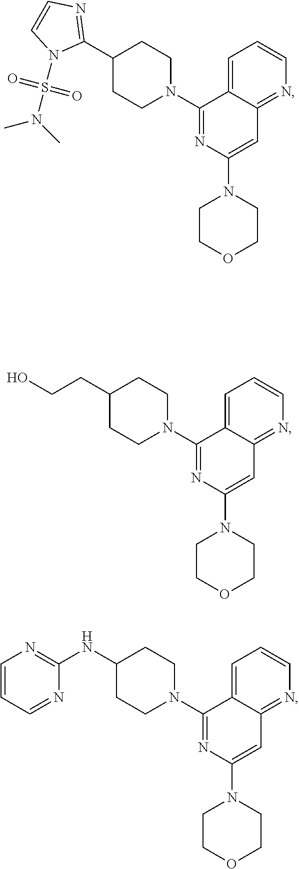 [SMILES: CN(C)S(=O)(=O)n1ccnc1C1CCN(c2nc(N3CCOCC3)cc3ncccc23)CC1 ; OCCC1CCN(c2nc(N3CCOCC3)cc3ncccc23)CC1 ; c1cnc(NC2CCN(c3nc(N4CCOCC4)cc4ncccc34)CC2)nc1] 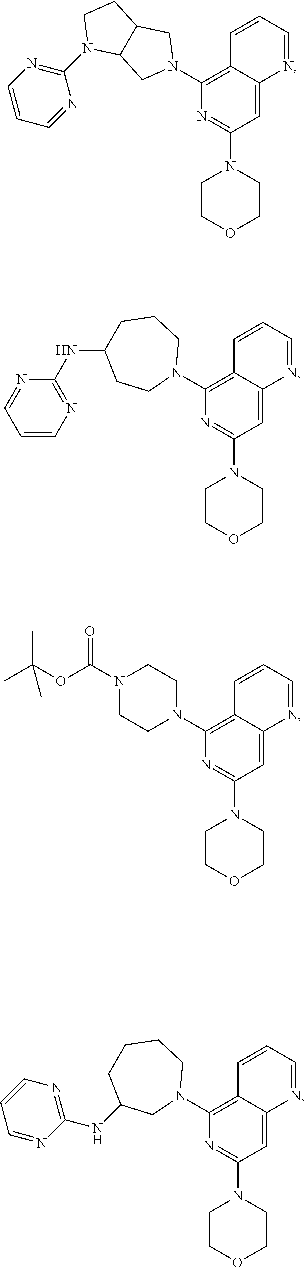 [SMILES: c1cnc(N2CCC3CN(c4nc(N5CCOCC5)cc5ncccc45)CC32)nc1 ; c1cnc(NC2CCCN(c3nc(N4CCOCC4)cc4ncccc34)CC2)nc1 ; CC(C)(C)OC(=O)N1CCN(c2nc(N3CCOCC3)cc3ncccc23)CC1 ; c1cnc(NC2CCCCN(c3nc(N4CCOCC4)cc4ncccc34)C2)nc1] 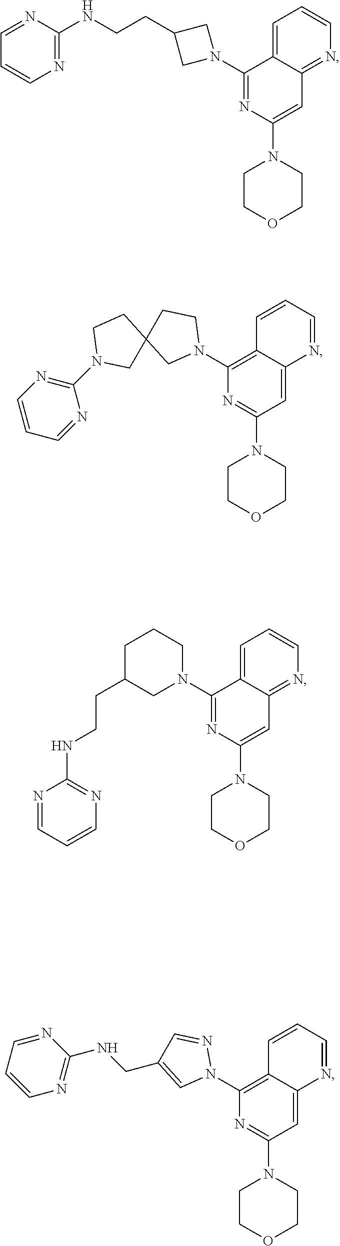 [SMILES: c1cnc(NCCC2CN(c3nc(N4CCOCC4)cc4ncccc34)C2)nc1 ; c1cnc(N2CCC3(CCN(c4nc(N5CCOCC5)cc5ncccc45)C3)C2)nc1 ; c1cnc(NCCC2CCCN(c3nc(N4CCOCC4)cc4ncccc34)C2)nc1 ; c1cnc(NCc2cnn(-c3nc(N4CCOCC4)cc4ncccc34)c2)nc1] 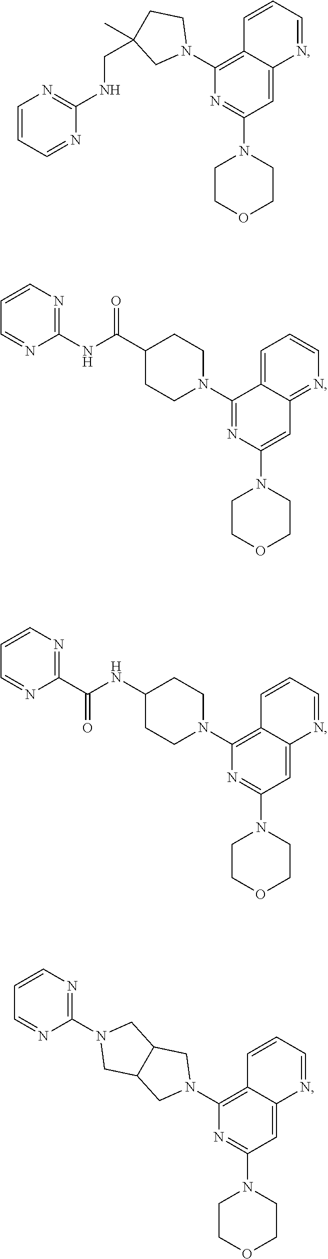 [SMILES: CC1(CNc2ncccn2)CCN(c2nc(N3CCOCC3)cc3ncccc23)C1 ; O=C(Nc1ncccn1)C1CCN(c2nc(N3CCOCC3)cc3ncccc23)CC1 ; O=C(NC1CCN(c2nc(N3CCOCC3)cc3ncccc23)CC1)c1ncccn1 ; c1cnc(N2CC3CN(c4nc(N5CCOCC5)cc5ncccc45)CC3C2)nc1] 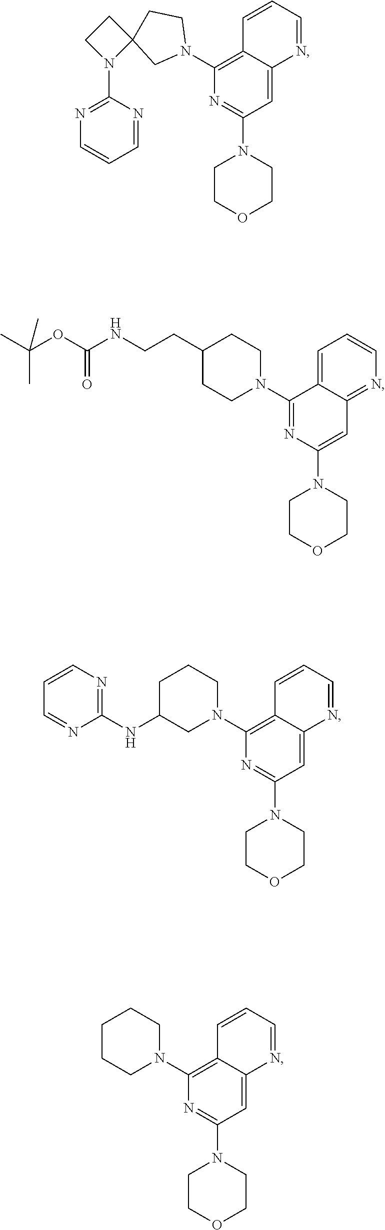 [SMILES: c1cnc(N2CCC23CCN(c2nc(N4CCOCC4)cc4ncccc24)C3)nc1 ; CC(C)(C)OC(=O)NCCC1CCN(c2nc(N3CCOCC3)cc3ncccc23)CC1 ; c1cnc(NC2CCCN(c3nc(N4CCOCC4)cc4ncccc34)C2)nc1 ; c1cnc2cc(N3CCOCC3)nc(N3CCCCC3)c2c1] 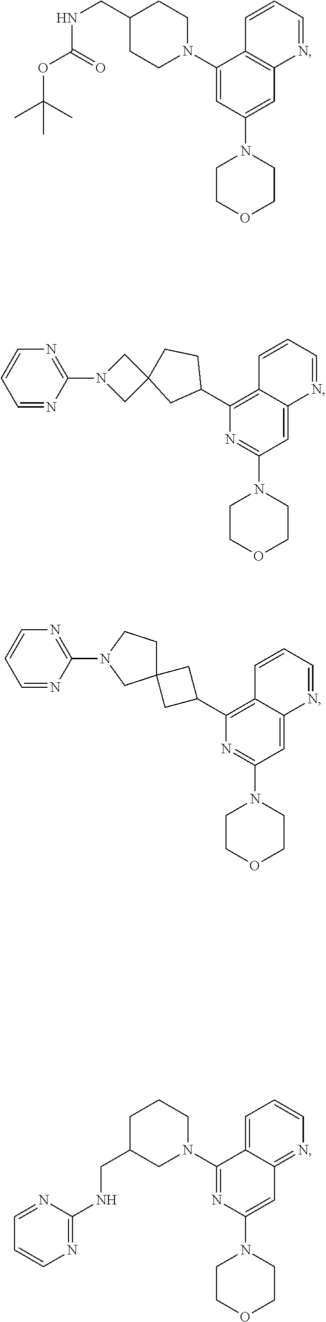 [SMILES: CC(C)(C)OC(=O)NCC1CCN(c2cc(N3CCOCC3)cc3ncccc23)CC1 ; c1cnc(N2CC3(CCC(c4nc(N5CCOCC5)cc5ncccc45)C3)C2)nc1 ; c1cnc(N2CCC3(CC(c4nc(N5CCOCC5)cc5ncccc45)C3)C2)nc1 ; c1cnc(NCC2CCCN(c3nc(N4CCOCC4)cc4ncccc34)C2)nc1] 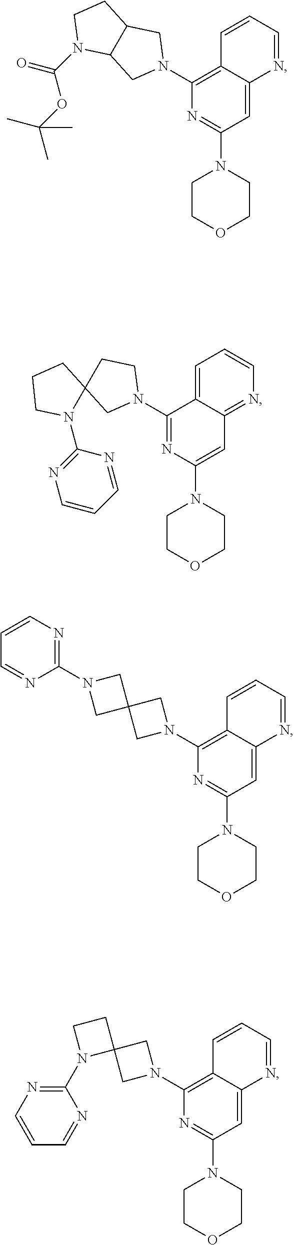 [SMILES: CC(C)(C)OC(=O)N1CCC2CN(c3nc(N4CCOCC4)cc4ncccc34)CC21 ; c1cnc(N2CCCC23CCN(c2nc(N4CCOCC4)cc4ncccc24)C3)nc1 ; c1cnc(N2CC3(C2)CN(c2nc(N4CCOCC4)cc4ncccc24)C3)nc1 ; c1cnc(N2CCC23CN(c2nc(N4CCOCC4)cc4ncccc24)C3)nc1] 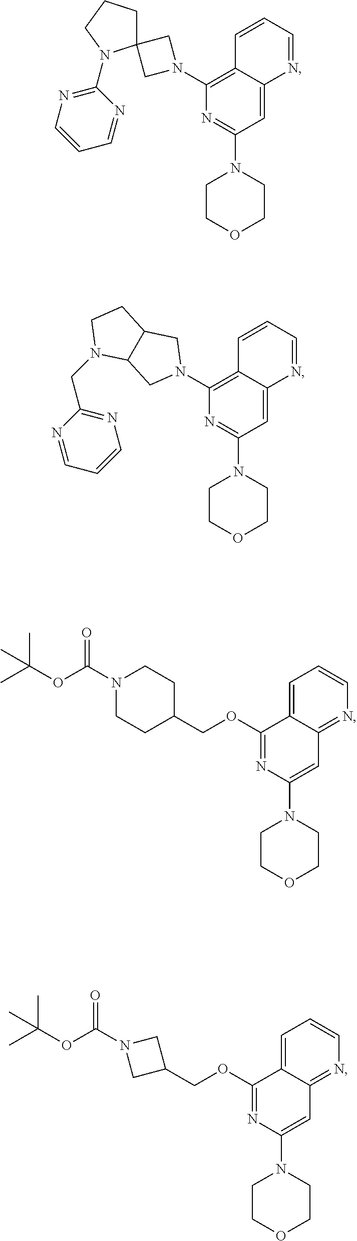 [SMILES: c1cnc(N2CCCC23CN(c2nc(N4CCOCC4)cc4ncccc24)C3)nc1 ; c1cnc(CN2CCC3CN(c4nc(N5CCOCC5)cc5ncccc45)CC32)nc1 ; CC(C)(C)OC(=O)N1CCC(COc2nc(N3CCOCC3)cc3ncccc23)CC1 ; CC(C)(C)OC(=O)N1CC(COc2nc(N3CCOCC3)cc3ncccc23)C1] 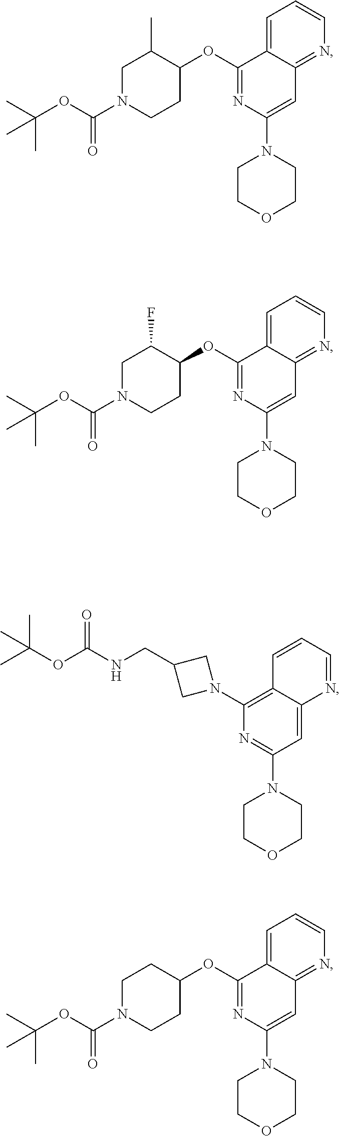 [SMILES: CC1CN(C(=O)OC(C)(C)C)CCC1Oc1nc(N2CCOCC2)cc2ncccc12 ; CC(C)(C)OC(=O)N1CC[C@H](Oc2nc(N3CCOCC3)cc3ncccc23)[C@@H](F)C1 ; CC(C)(C)OC(=O)NCC1CN(c2nc(N3CCOCC3)cc3ncccc23)C1 ; CC(C)(C)OC(=O)N1CCC(Oc2nc(N3CCOCC3)cc3ncccc23)CC1] 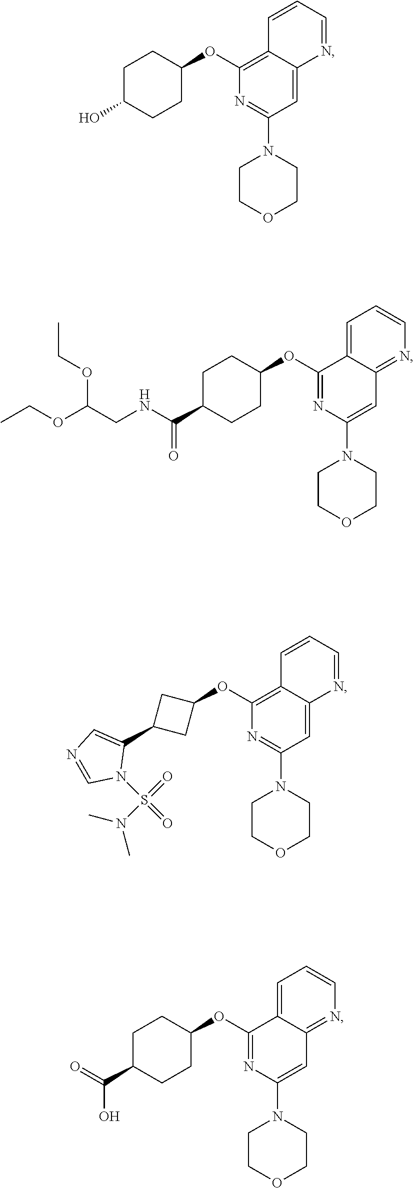 [SMILES: O[C@H]1CC[C@H](Oc2nc(N3CCOCC3)cc3ncccc23)CC1 ; CCOC(CNC(=O)[C@H]1CC[C@@H](Oc2nc(N3CCOCC3)cc3ncccc23)CC1)OCC ; CN(C)S(=O)(=O)n1cncc1[C@H]1C[C@@H](Oc2nc(N3CCOCC3)cc3ncccc23)C1 ; O=C(O)[C@H]1CC[C@@H](Oc2nc(N3CCOCC3)cc3ncccc23)CC1] 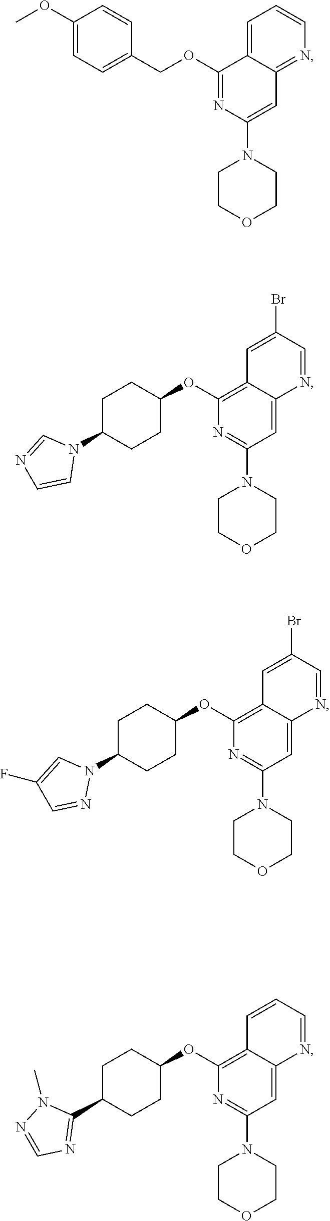 [SMILES: COc1ccc(COc2nc(N3CCOCC3)cc3ncccc23)cc1 ; Brc1cnc2cc(N3CCOCC3)nc(O[C@H]3CC[C@@H](n4ccnc4)CC3)c2c1 ; Fc1cnn([C@H]2CC[C@@H](Oc3nc(N4CCOCC4)cc4ncc(Br)cc34)CC2)c1 ; Cn1ncnc1[C@H]1CC[C@@H](Oc2nc(N3CCOCC3)cc3ncccc23)CC1] 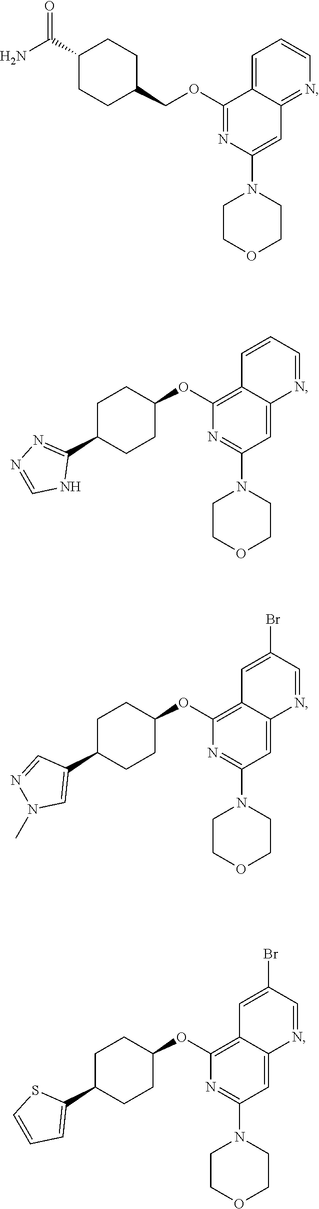 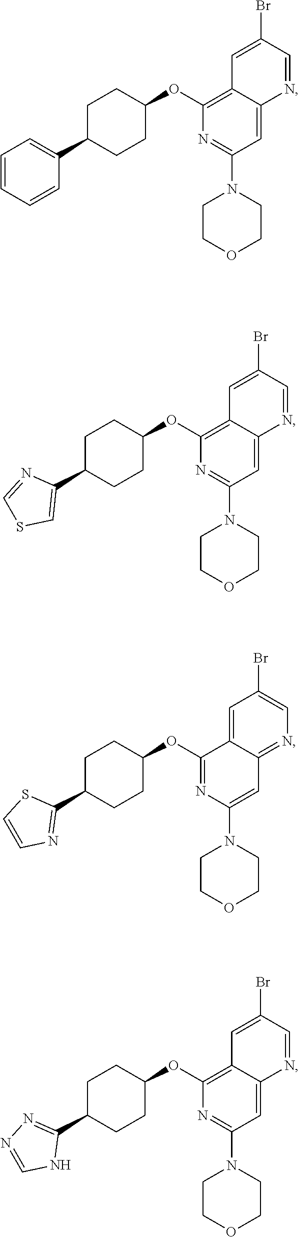 [SMILES: Brc1cnc2cc(N3CCOCC3)nc(O[C@H]3CC[C@@H](c4ccccc4)CC3)c2c1 ; Brc1cnc2cc(N3CCOCC3)nc(O[C@H]3CC[C@@H](c4cscn4)CC3)c2c1 ; Brc1cnc2cc(N3CCOCC3)nc(O[C@H]3CC[C@@H](c4nccs4)CC3)c2c1 ; Brc1cnc2cc(N3CCOCC3)nc(O[C@H]3CC[C@@H](c4nnc[nH]4)CC3)c2c1] 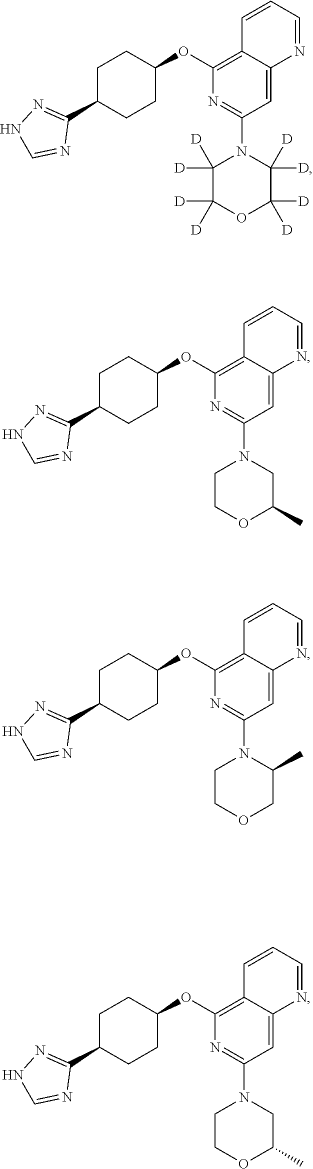 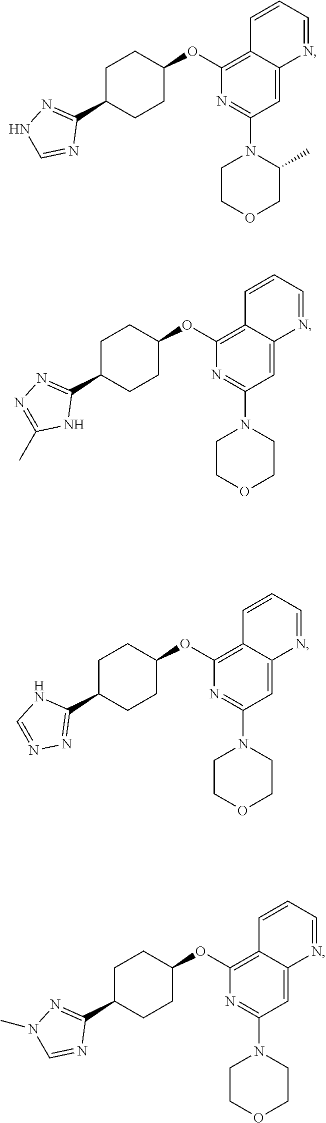 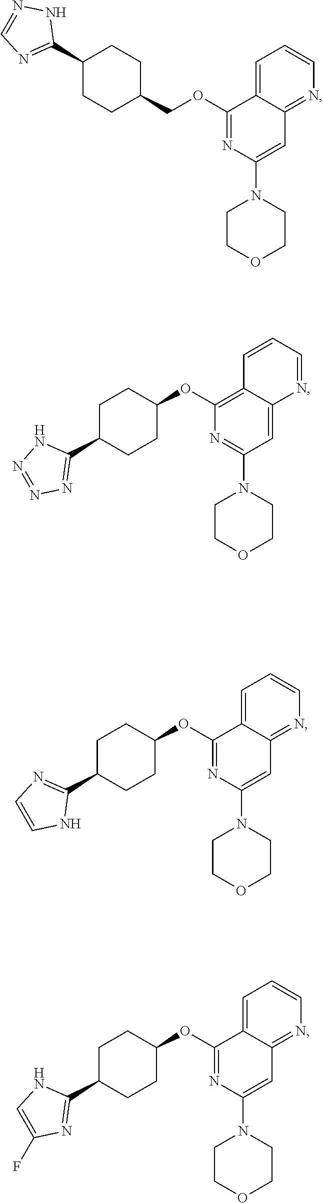 [SMILES: c1cnc2cc(N3CCOCC3)nc(OC[C@H]3CC[C@@H](c4ncn[nH]4)CC3)c2c1 ; c1cnc2cc(N3CCOCC3)nc(O[C@H]3CC[C@@H](c4nnn[nH]4)CC3)c2c1 ; c1cnc2cc(N3CCOCC3)nc(O[C@H]3CC[C@@H](c4ncc[nH]4)CC3)c2c1 ; Fc1c[nH]c([C@H]2CC[C@@H](Oc3nc(N4CCOCC4)cc4ncccc34)CC2)n1] 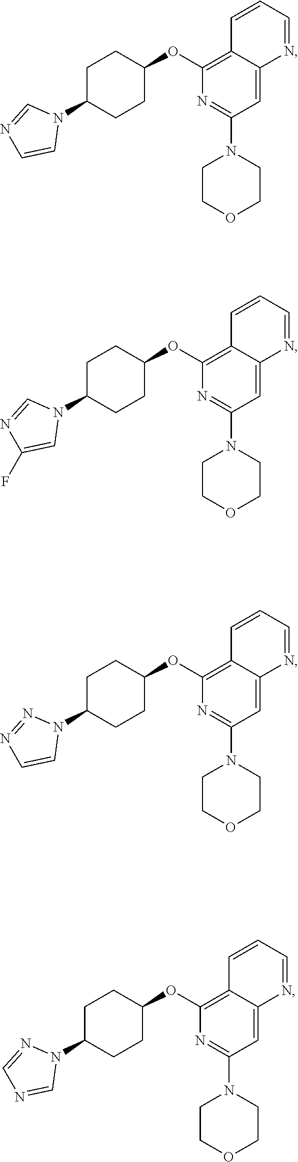 [SMILES: c1cnc2cc(N3CCOCC3)nc(O[C@H]3CC[C@@H](n4ccnc4)CC3)c2c1 ; Fc1cn([C@H]2CC[C@@H](Oc3nc(N4CCOCC4)cc4ncccc34)CC2)cn1 ; c1cnc2cc(N3CCOCC3)nc(O[C@H]3CC[C@@H](n4ccnn4)CC3)c2c1 ; c1cnc2cc(N3CCOCC3)nc(O[C@H]3CC[C@@H](n4cncn4)CC3)c2c1] 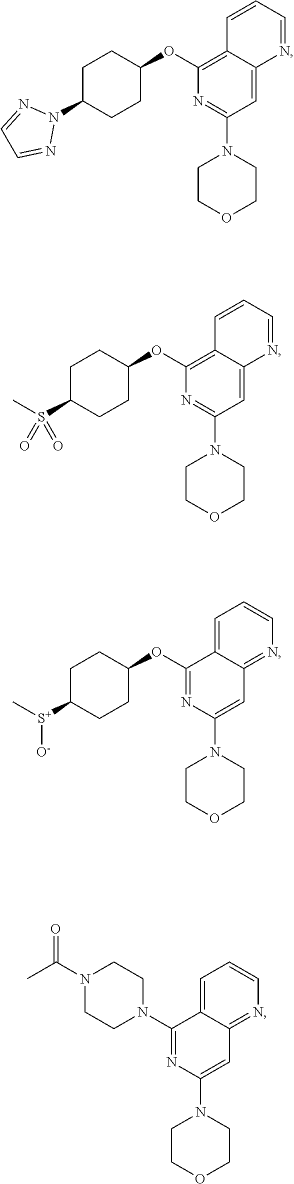 [SMILES: c1cnc2cc(N3CCOCC3)nc(O[C@H]3CC[C@@H](n4nccn4)CC3)c2c1 ; CS(=O)(=O)[C@H]1CC[C@@H](Oc2nc(N3CCOCC3)cc3ncccc23)CC1 ; C[S+]([O-])[C@H]1CC[C@@H](Oc2nc(N3CCOCC3)cc3ncccc23)CC1 ; CC(=O)N1CCN(c2nc(N3CCOCC3)cc3ncccc23)CC1] 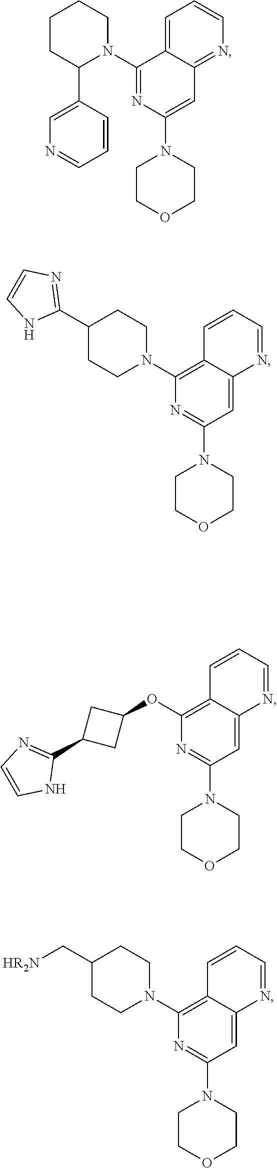 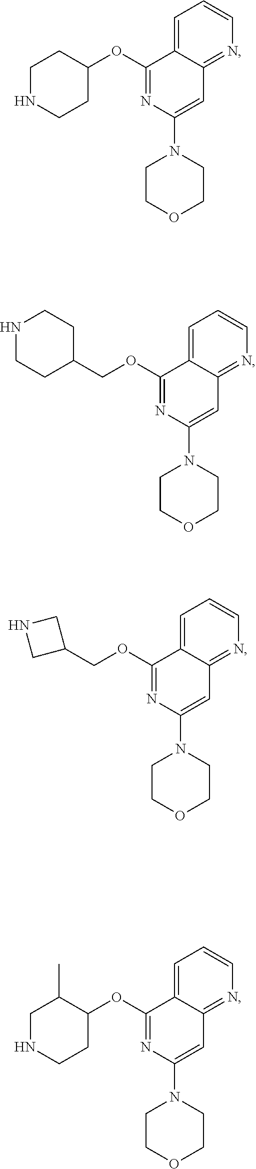 [SMILES: c1cnc2cc(N3CCOCC3)nc(OC3CCNCC3)c2c1 ; c1cnc2cc(N3CCOCC3)nc(OCC3CCNCC3)c2c1 ; c1cnc2cc(N3CCOCC3)nc(OCC3CNC3)c2c1 ; CC1CNCCC1Oc1nc(N2CCOCC2)cc2ncccc12] 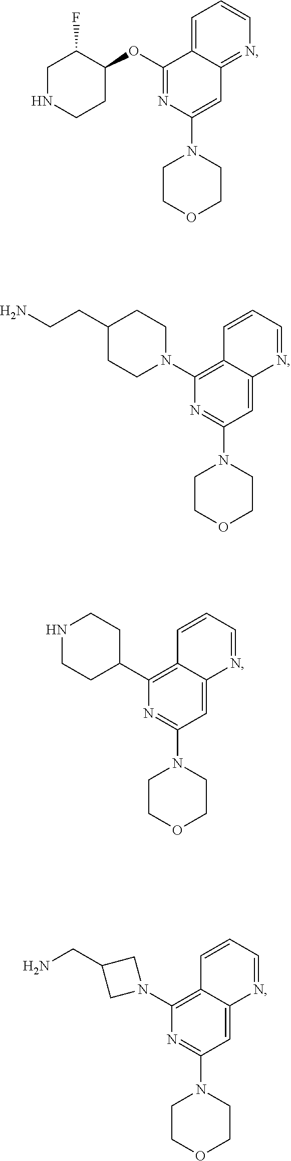 [SMILES: F[C@H]1CNCC[C@@H]1Oc1nc(N2CCOCC2)cc2ncccc12 ; NCCC1CCN(c2nc(N3CCOCC3)cc3ncccc23)CC1 ; c1cnc2cc(N3CCOCC3)nc(C3CCNCC3)c2c1 ; NCC1CN(c2nc(N3CCOCC3)cc3ncccc23)C1] 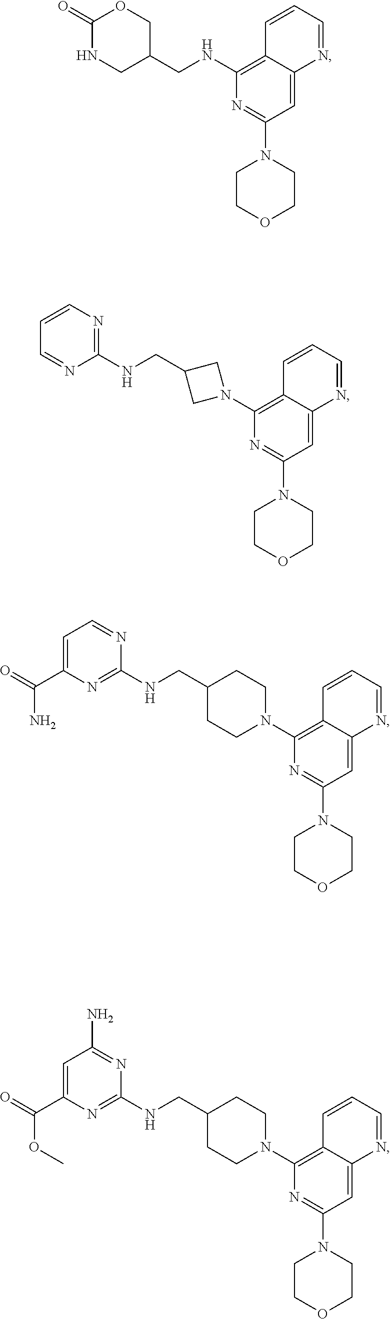 [SMILES: O=C1NCC(CNc2nc(N3CCOCC3)cc3ncccc23)CO1 ; c1cnc(NCC2CN(c3nc(N4CCOCC4)cc4ncccc34)C2)nc1 ; NC(=O)c1ccnc(NCC2CCN(c3nc(N4CCOCC4)cc4ncccc34)CC2)n1 ; COC(=O)c1cc(N)nc(NCC2CCN(c3nc(N4CCOCC4)cc4ncccc34)CC2)n1] 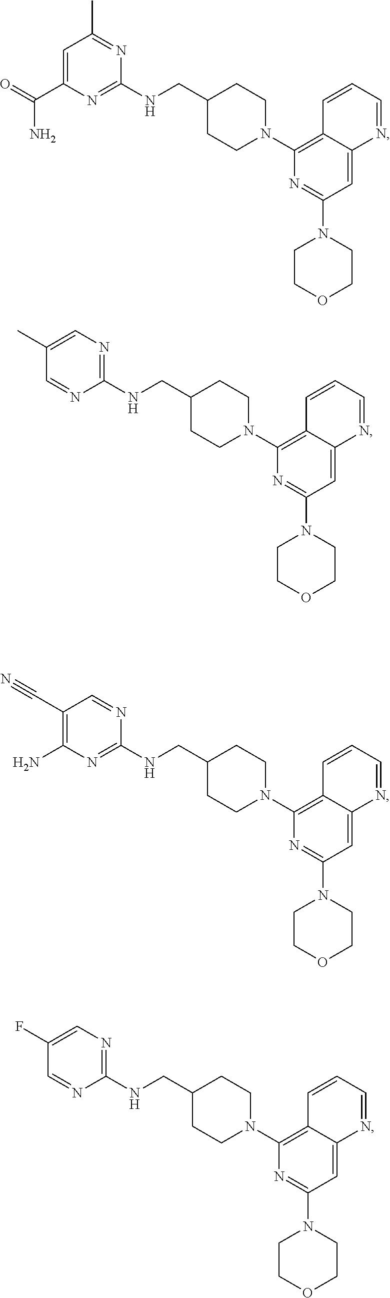 [SMILES: Cc1cc(C(N)=O)nc(NCC2CCN(c3nc(N4CCOCC4)cc4ncccc34)CC2)n1 ; Cc1cnc(NCC2CCN(c3nc(N4CCOCC4)cc4ncccc34)CC2)nc1 ; N#Cc1cnc(NCC2CCN(c3nc(N4CCOCC4)cc4ncccc34)CC2)nc1N ; Fc1cnc(NCC2CCN(c3nc(N4CCOCC4)cc4ncccc34)CC2)nc1] 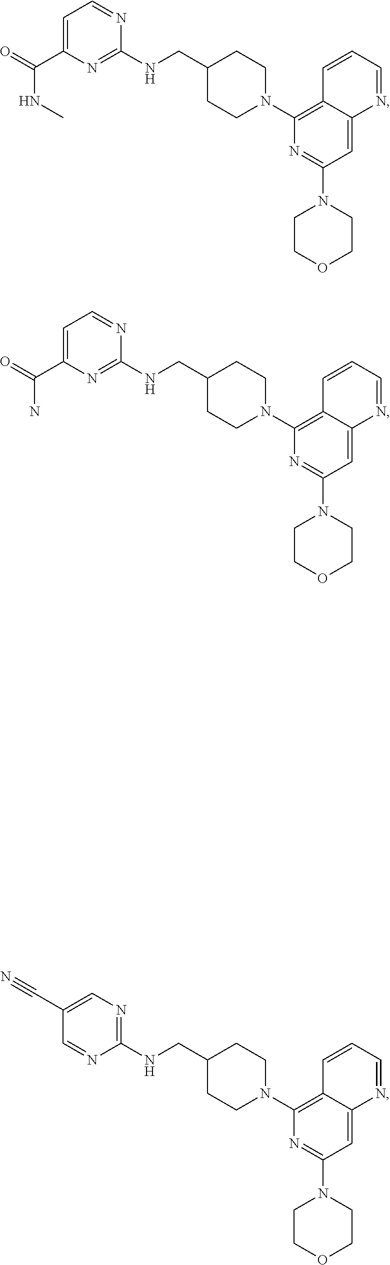 [SMILES: CNC(=O)c1ccnc(NCC2CCN(c3nc(N4CCOCC4)cc4ncccc34)CC2)n1 ; NC(=O)c1ccnc(NCC2CCN(c3nc(N4CCOCC4)cc4ncccc34)CC2)n1 ; N#Cc1cnc(NCC2CCN(c3nc(N4CCOCC4)cc4ncccc34)CC2)nc1] 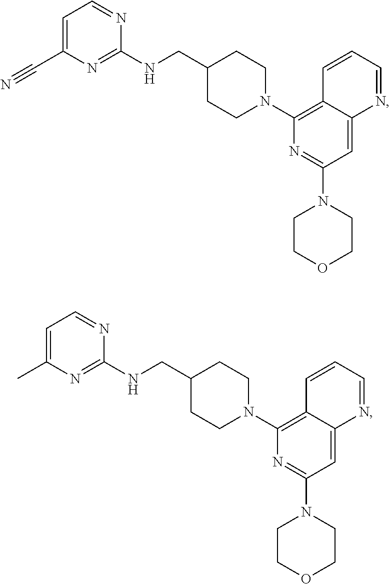 [SMILES: N#Cc1ccnc(NCC2CCN(c3nc(N4CCOCC4)cc4ncccc34)CC2)n1 ; Cc1ccnc(NCC2CCN(c3nc(N4CCOCC4)cc4ncccc34)CC2)n1] 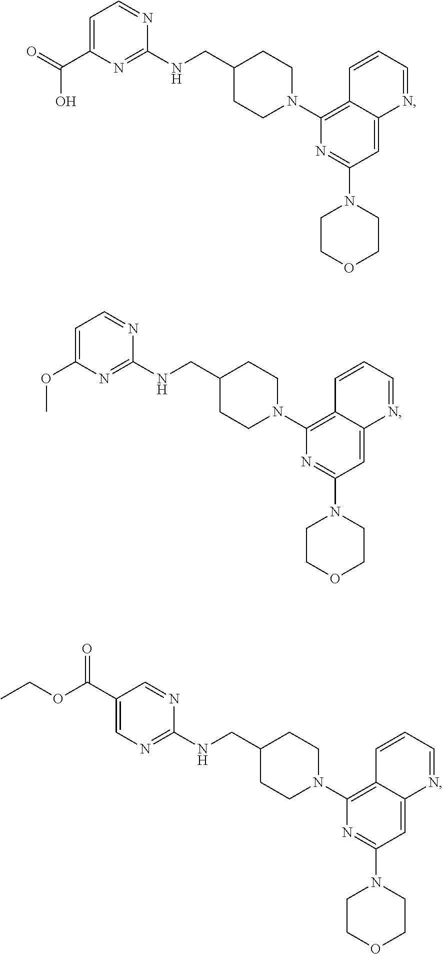 [SMILES: O=C(O)c1ccnc(NCC2CCN(c3nc(N4CCOCC4)cc4ncccc34)CC2)n1 ; COc1ccnc(NCC2CCN(c3nc(N4CCOCC4)cc4ncccc34)CC2)n1 ; CCOC(=O)c1cnc(NCC2CCN(c3nc(N4CCOCC4)cc4ncccc34)CC2)nc1] 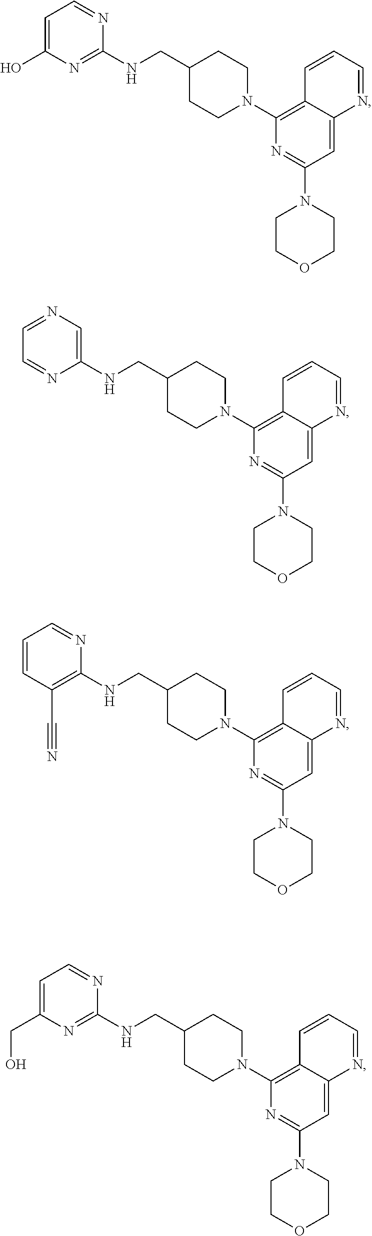 [SMILES: Oc1ccnc(NCC2CCN(c3nc(N4CCOCC4)cc4ncccc34)CC2)n1 ; c1cnc2cc(N3CCOCC3)nc(N3CCC(CNc4cnccn4)CC3)c2c1 ; N#Cc1cccnc1NCC1CCN(c2nc(N3CCOCC3)cc3ncccc23)CC1 ; OCc1ccnc(NCC2CCN(c3nc(N4CCOCC4)cc4ncccc34)CC2)n1] 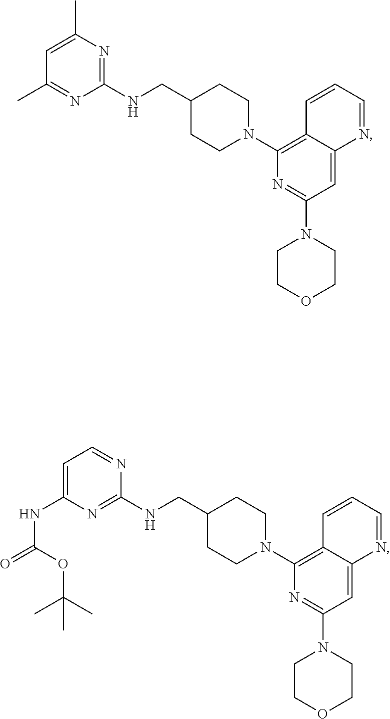 [SMILES: Cc1cc(C)nc(NCC2CCN(c3nc(N4CCOCC4)cc4ncccc34)CC2)n1 ; CC(C)(C)OC(=O)Nc1ccnc(NCC2CCN(c3nc(N4CCOCC4)cc4ncccc34)CC2)n1] 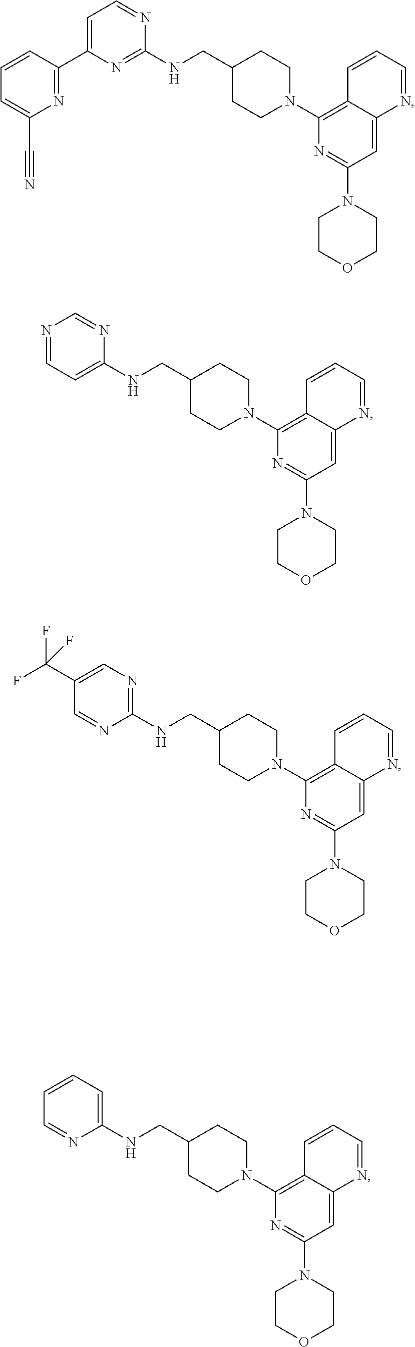 [SMILES: N#Cc1cccc(-c2ccnc(NCC3CCN(c4nc(N5CCOCC5)cc5ncccc45)CC3)n2)n1 ; c1cnc2cc(N3CCOCC3)nc(N3CCC(CNc4ccncn4)CC3)c2c1 ; FC(F)(F)c1cnc(NCC2CCN(c3nc(N4CCOCC4)cc4ncccc34)CC2)nc1 ; c1ccc(NCC2CCN(c3nc(N4CCOCC4)cc4ncccc34)CC2)nc1] 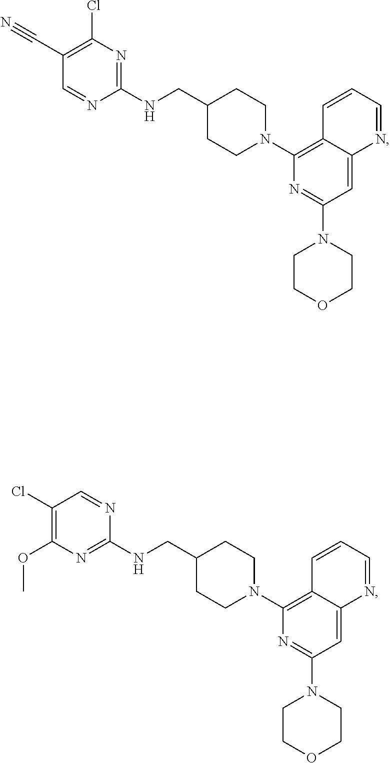 [SMILES: N#Cc1cnc(NCC2CCN(c3nc(N4CCOCC4)cc4ncccc34)CC2)nc1Cl ; COc1nc(NCC2CCN(c3nc(N4CCOCC4)cc4ncccc34)CC2)ncc1Cl] 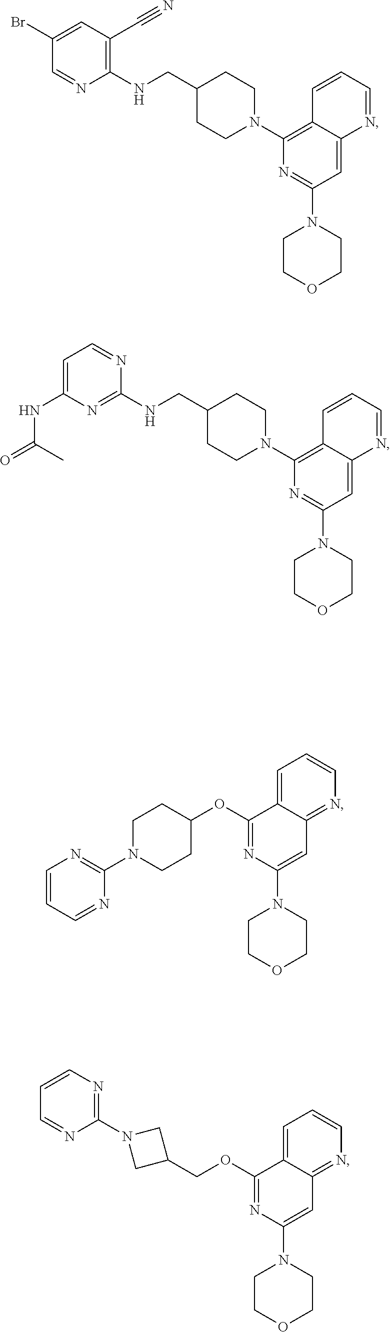 [SMILES: N#Cc1cc(Br)cnc1NCC1CCN(c2nc(N3CCOCC3)cc3ncccc23)CC1 ; CC(=O)Nc1ccnc(NCC2CCN(c3nc(N4CCOCC4)cc4ncccc34)CC2)n1 ; c1cnc(N2CCC(Oc3nc(N4CCOCC4)cc4ncccc34)CC2)nc1 ; c1cnc(N2CC(COc3nc(N4CCOCC4)cc4ncccc34)C2)nc1] 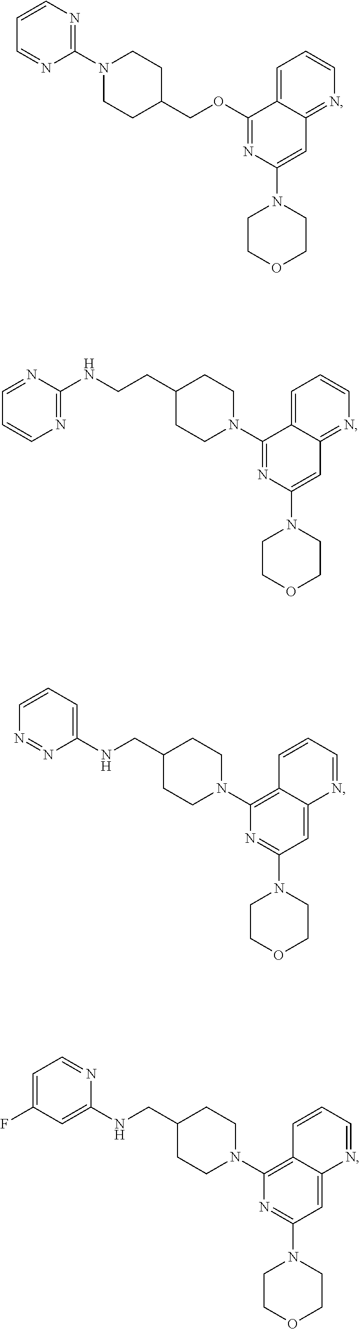 [SMILES: c1cnc(N2CCC(COc3nc(N4CCOCC4)cc4ncccc34)CC2)nc1 ; c1cnc(NCCC2CCN(c3nc(N4CCOCC4)cc4ncccc34)CC2)nc1 ; c1cnnc(NCC2CCN(c3nc(N4CCOCC4)cc4ncccc34)CC2)c1 ; Fc1ccnc(NCC2CCN(c3nc(N4CCOCC4)cc4ncccc34)CC2)c1] 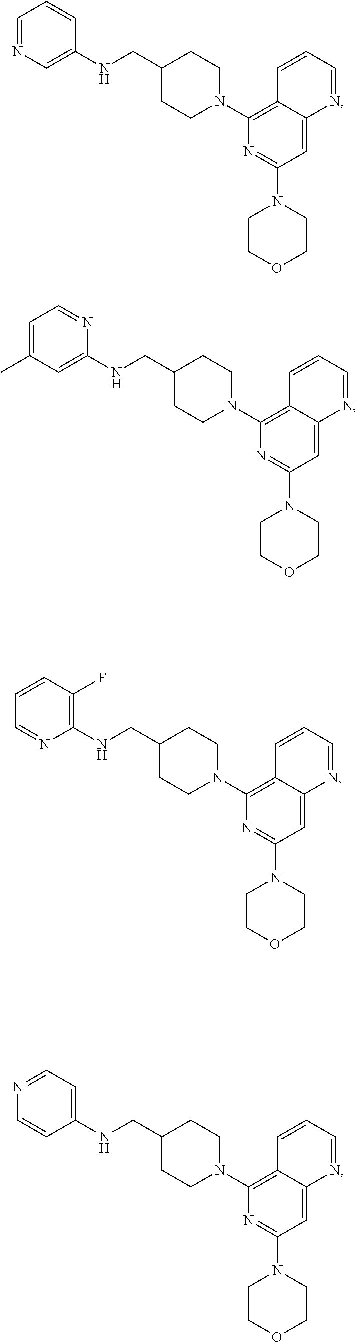 [SMILES: c1cncc(NCC2CCN(c3nc(N4CCOCC4)cc4ncccc34)CC2)c1 ; Cc1ccnc(NCC2CCN(c3nc(N4CCOCC4)cc4ncccc34)CC2)c1 ; Fc1cccnc1NCC1CCN(c2nc(N3CCOCC3)cc3ncccc23)CC1 ; c1cnc2cc(N3CCOCC3)nc(N3CCC(CNc4ccncc4)CC3)c2c1] 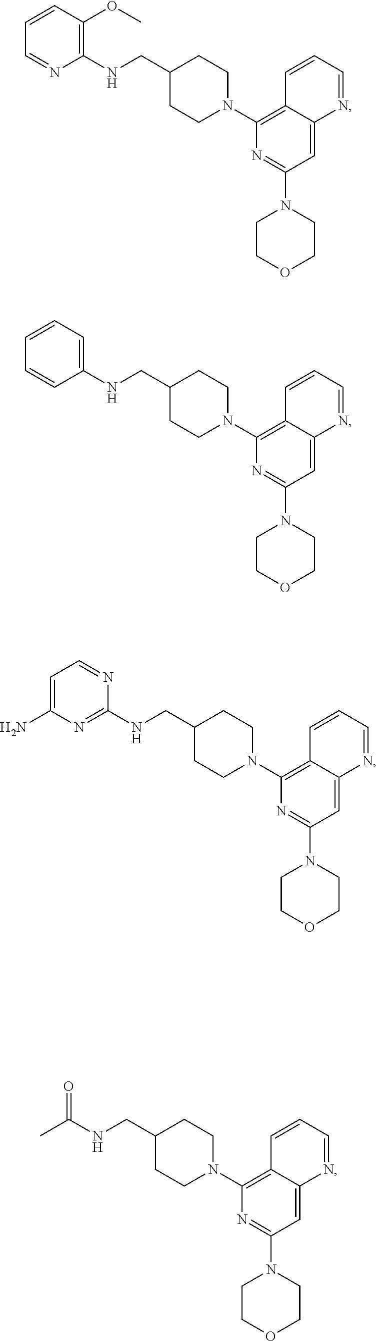 [SMILES: COc1cccnc1NCC1CCN(c2nc(N3CCOCC3)cc3ncccc23)CC1 ; c1ccc(NCC2CCN(c3nc(N4CCOCC4)cc4ncccc34)CC2)cc1 ; Nc1ccnc(NCC2CCN(c3nc(N4CCOCC4)cc4ncccc34)CC2)n1 ; CC(=O)NCC1CCN(c2nc(N3CCOCC3)cc3ncccc23)CC1] 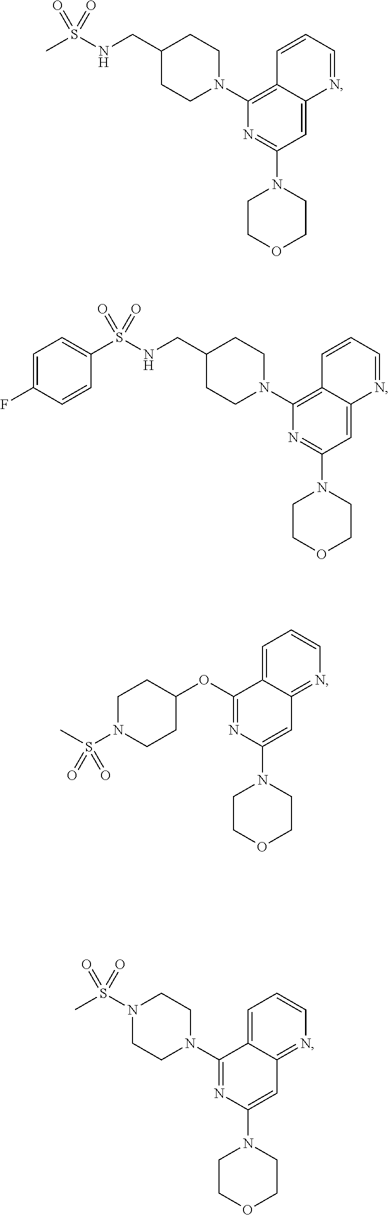 [SMILES: CS(=O)(=O)NCC1CCN(c2nc(N3CCOCC3)cc3ncccc23)CC1 ; O=S(=O)(NCC1CCN(c2nc(N3CCOCC3)cc3ncccc23)CC1)c1ccc(F)cc1 ; CS(=O)(=O)N1CCC(Oc2nc(N3CCOCC3)cc3ncccc23)CC1 ; CS(=O)(=O)N1CCN(c2nc(N3CCOCC3)cc3ncccc23)CC1] 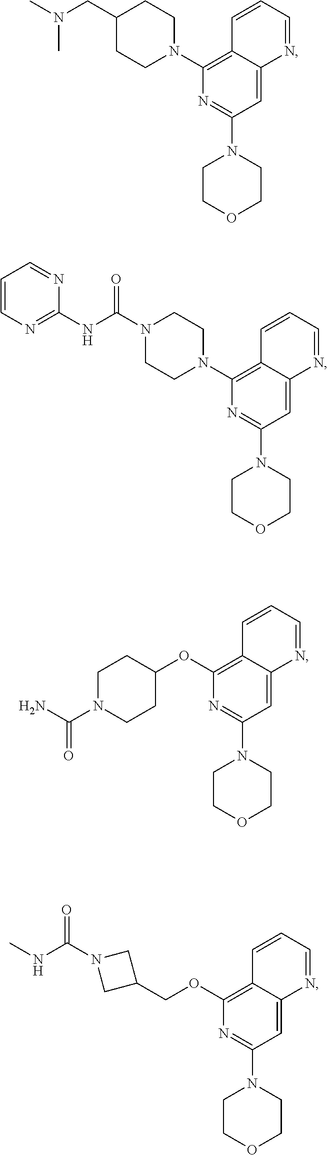 [SMILES: CN(C)CC1CCN(c2nc(N3CCOCC3)cc3ncccc23)CC1 ; O=C(Nc1ncccn1)N1CCN(c2nc(N3CCOCC3)cc3ncccc23)CC1 ; NC(=O)N1CCC(Oc2nc(N3CCOCC3)cc3ncccc23)CC1 ; CNC(=O)N1CC(COc2nc(N3CCOCC3)cc3ncccc23)C1] 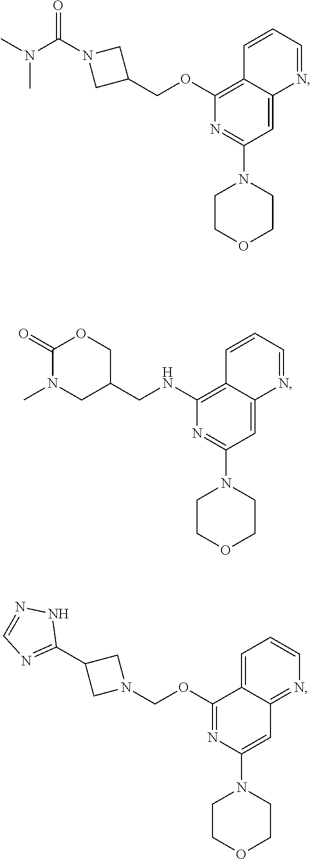 [SMILES: CN(C)C(=O)N1CC(COc2nc(N3CCOCC3)cc3ncccc23)C1 ; CN1CC(CNc2nc(N3CCOCC3)cc3ncccc23)COC1=O ; c1cnc2cc(N3CCOCC3)nc(OCN3CC(c4ncn[nH]4)C3)c2c1] 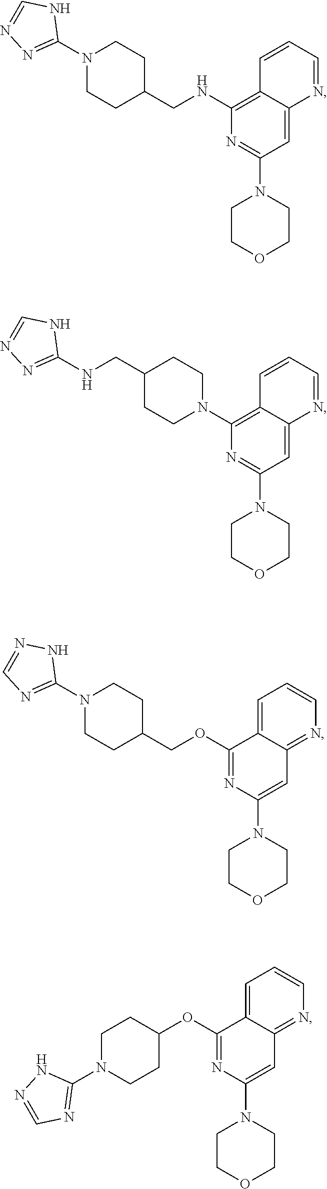 [SMILES: c1cnc2cc(N3CCOCC3)nc(NCC3CCN(c4nnc[nH]4)CC3)c2c1 ; c1cnc2cc(N3CCOCC3)nc(N3CCC(CNc4nnc[nH]4)CC3)c2c1 ; c1cnc2cc(N3CCOCC3)nc(OCC3CCN(c4ncn[nH]4)CC3)c2c1 ; c1cnc2cc(N3CCOCC3)nc(OC3CCN(c4ncn[nH]4)CC3)c2c1] 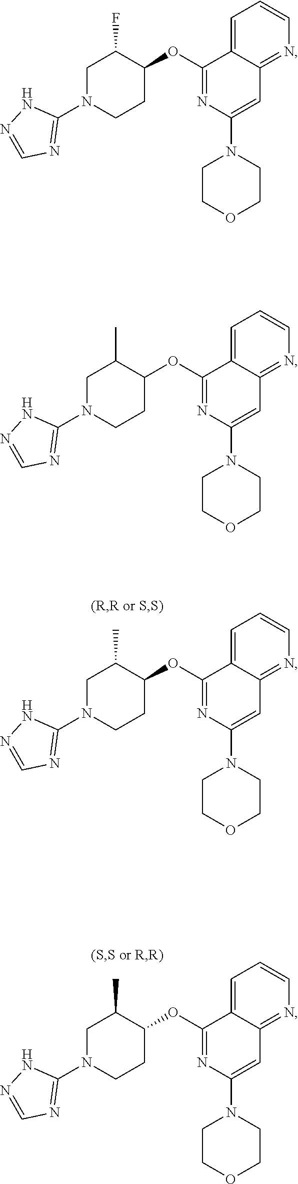 [SMILES: F[C@H]1CN(c2ncn[nH]2)CC[C@@H]1Oc1nc(N2CCOCC2)cc2ncccc12 ; CC1CN(c2ncn[nH]2)CCC1Oc1nc(N2CCOCC2)cc2ncccc12 ; C[C@H]1CN(c2ncn[nH]2)CC[C@@H]1Oc1nc(N2CCOCC2)cc2ncccc12 ; C[C@@H]1CN(c2ncn[nH]2)CC[C@H]1Oc1nc(N2CCOCC2)cc2ncccc12] 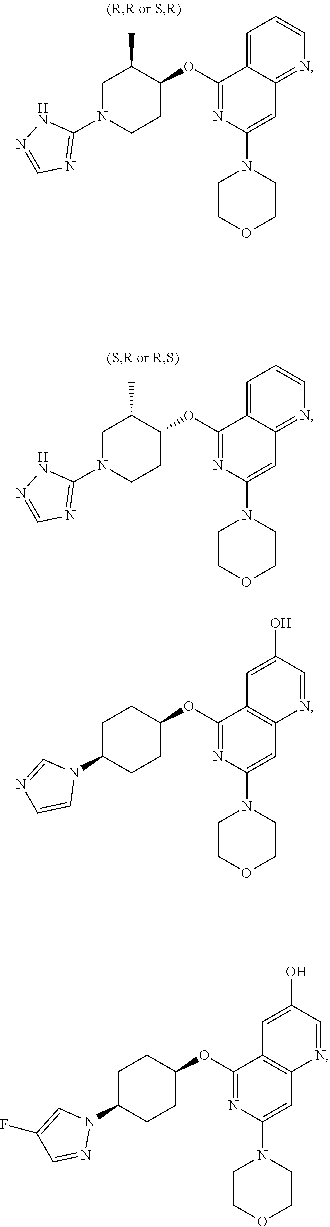 [SMILES: C[C@@H]1CN(c2ncn[nH]2)CC[C@@H]1Oc1nc(N2CCOCC2)cc2ncccc12 ; C[C@H]1CN(c2ncn[nH]2)CC[C@H]1Oc1nc(N2CCOCC2)cc2ncccc12 ; Oc1cnc2cc(N3CCOCC3)nc(O[C@H]3CC[C@@H](n4ccnc4)CC3)c2c1 ; Oc1cnc2cc(N3CCOCC3)nc(O[C@H]3CC[C@@H](n4cc(F)cn4)CC3)c2c1] 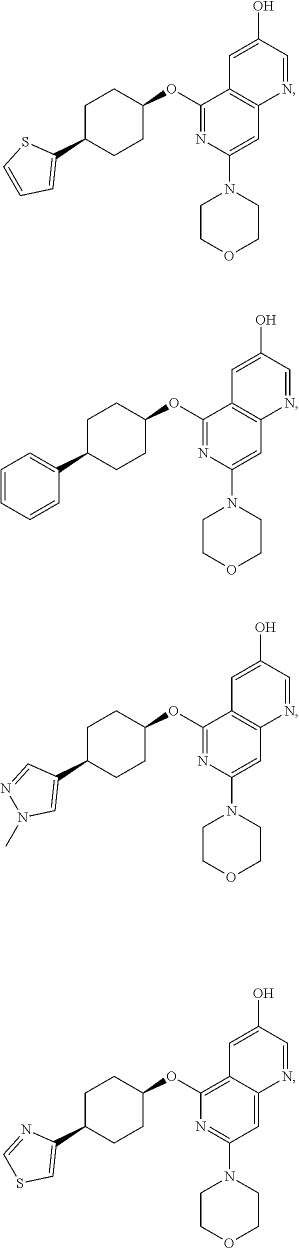 [SMILES: Oc1cnc2cc(N3CCOCC3)nc(O[C@H]3CC[C@@H](c4cccs4)CC3)c2c1 ; Oc1cnc2cc(N3CCOCC3)nc(O[C@H]3CC[C@@H](c4ccccc4)CC3)c2c1 ; Cn1cc([C@H]2CC[C@@H](Oc3nc(N4CCOCC4)cc4ncc(O)cc34)CC2)cn1 ; Oc1cnc2cc(N3CCOCC3)nc(O[C@H]3CC[C@@H](c4cscn4)CC3)c2c1] 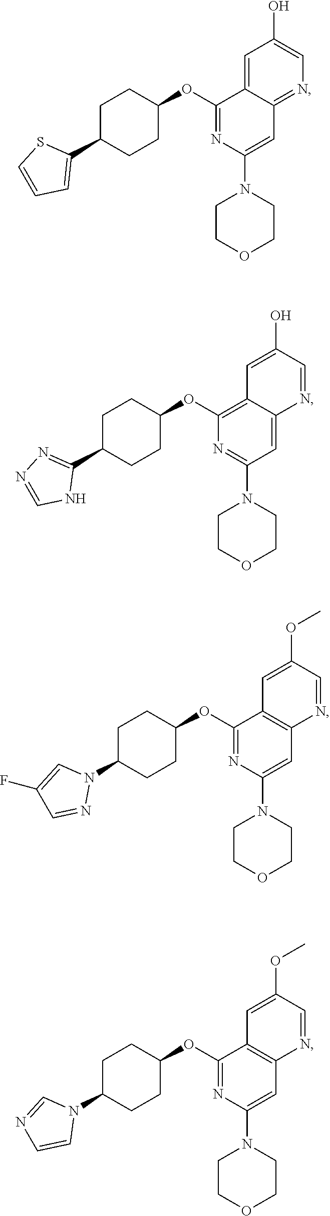 [SMILES: Oc1cnc2cc(N3CCOCC3)nc(O[C@H]3CC[C@@H](c4cccs4)CC3)c2c1 ; Oc1cnc2cc(N3CCOCC3)nc(O[C@H]3CC[C@@H](c4nnc[nH]4)CC3)c2c1 ; COc1cnc2cc(N3CCOCC3)nc(O[C@H]3CC[C@@H](n4cc(F)cn4)CC3)c2c1 ; COc1cnc2cc(N3CCOCC3)nc(O[C@H]3CC[C@@H](n4ccnc4)CC3)c2c1] 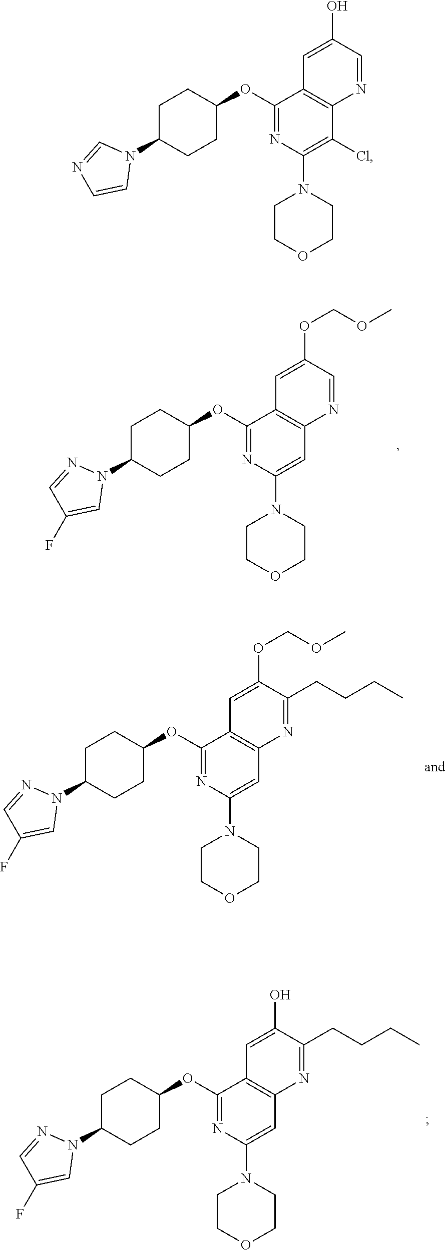 [SMILES: Oc1cnc2c(Cl)c(N3CCOCC3)nc(O[C@H]3CC[C@@H](n4ccnc4)CC3)c2c1 ; COCOc1cnc2cc(N3CCOCC3)nc(O[C@H]3CC[C@@H](n4cc(F)cn4)CC3)c2c1 ; CCCCc1nc2cc(N3CCOCC3)nc(O[C@H]3CC[C@@H](n4cc(F)cn4)CC3)c2cc1OCOC ; CCCCc1nc2cc(N3CCOCC3)nc(O[C@H]3CC[C@@H](n4cc(F)cn4)CC3)c2cc1O]
or a pharmaceutically acceptable salt or N-oxide thereof;
or a prodrug thereof or a pharmaceutically acceptable salt or N-oxide of the prodrug;
wherein the prodrug comprises a trigger moiety that releases the compound of formula (I) under reductive conditions; optionally wherein the trigger moiety is as defined in claim 3 or claim 4.

25. A compound of claim 24 or a pharmaceutically acceptable salt or N-oxide thereof.

26. A compound of claim 1 wherein the compound of formula (I) is selected from: 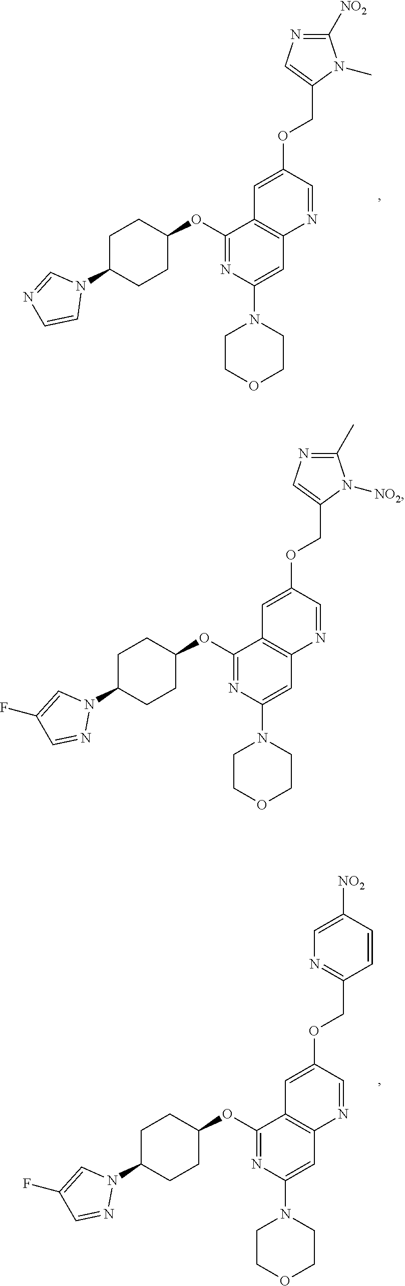 [SMILES: Cn1c(COc2cnc3cc(N4CCOCC4)nc(O[C@H]4CC[C@@H](n5ccnc5)CC4)c3c2)cnc1[N+](=O)[O-] ; Cc1ncc(COc2cnc3cc(N4CCOCC4)nc(O[C@H]4CC[C@@H](n5cc(F)cn5)CC4)c3c2)n1[N+](=O)[O-] ; O=[N+]([O-])c1ccc(COc2cnc3cc(N4CCOCC4)nc(O[C@H]4CC[C@@H](n5cc(F)cn5)CC4)c3c2)nc1] 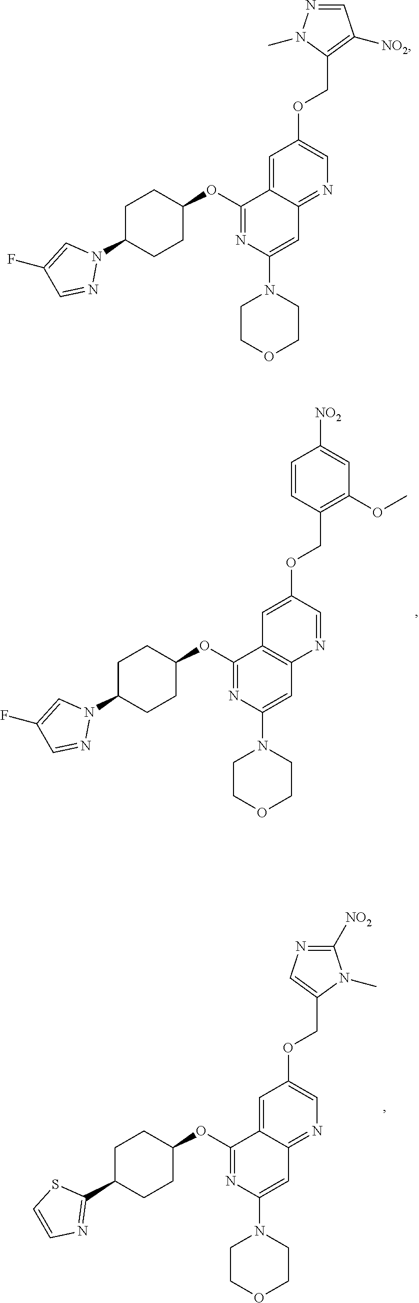 [SMILES: Cn1ncc([N+](=O)[O-])c1COc1cnc2cc(N3CCOCC3)nc(O[C@H]3CC[C@@H](n4cc(F)cn4)CC3)c2c1 ; COc1cc([N+](=O)[O-])ccc1COc1cnc2cc(N3CCOCC3)nc(O[C@H]3CC[C@@H](n4cc(F)cn4)CC3)c2c1 ; Cn1c(COc2cnc3cc(N4CCOCC4)nc(O[C@H]4CC[C@@H](c5nccs5)CC4)c3c2)cnc1[N+](=O)[O-]] 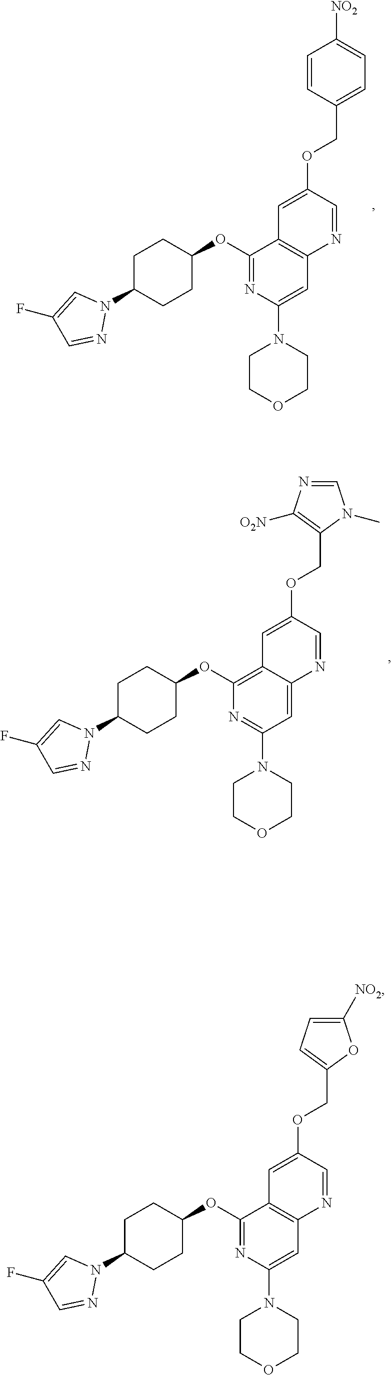 [SMILES: O=[N+]([O-])c1ccc(COc2cnc3cc(N4CCOCC4)nc(O[C@H]4CC[C@@H](n5cc(F)cn5)CC4)c3c2)cc1 ; Cn1cnc([N+](=O)[O-])c1COc1cnc2cc(N3CCOCC3)nc(O[C@H]3CC[C@@H](n4cc(F)cn4)CC3)c2c1 ; O=[N+]([O-])c1ccc(COc2cnc3cc(N4CCOCC4)nc(O[C@H]4CC[C@@H](n5cc(F)cn5)CC4)c3c2)o1] 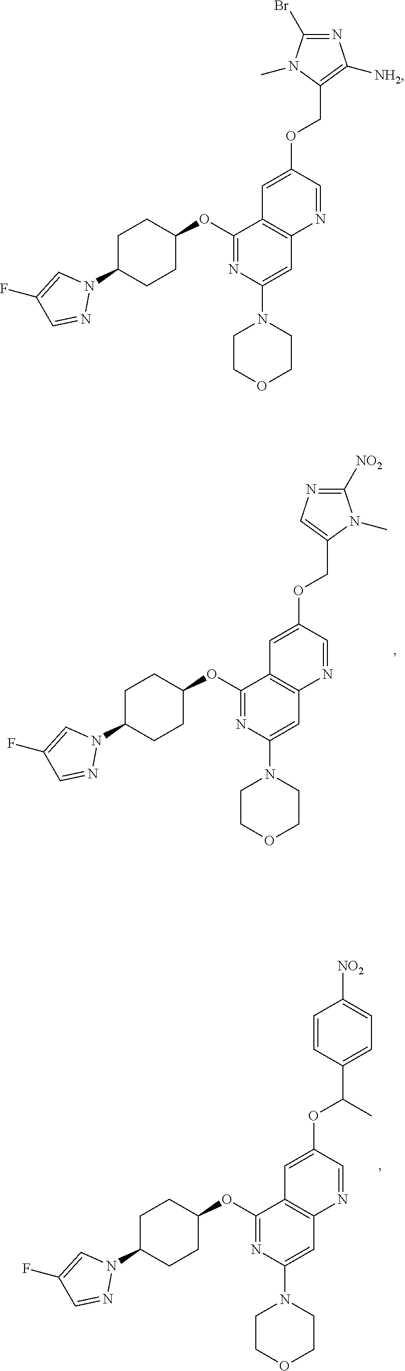 [SMILES: Cn1c(Br)nc(N)c1COc1cnc2cc(N3CCOCC3)nc(O[C@H]3CC[C@@H](n4cc(F)cn4)CC3)c2c1 ; Cn1c(COc2cnc3cc(N4CCOCC4)nc(O[C@H]4CC[C@@H](n5cc(F)cn5)CC4)c3c2)cnc1[N+](=O)[O-] ; CC(Oc1cnc2cc(N3CCOCC3)nc(O[C@H]3CC[C@@H](n4cc(F)cn4)CC3)c2c1)c1ccc([N+](=O)[O-])cc1] 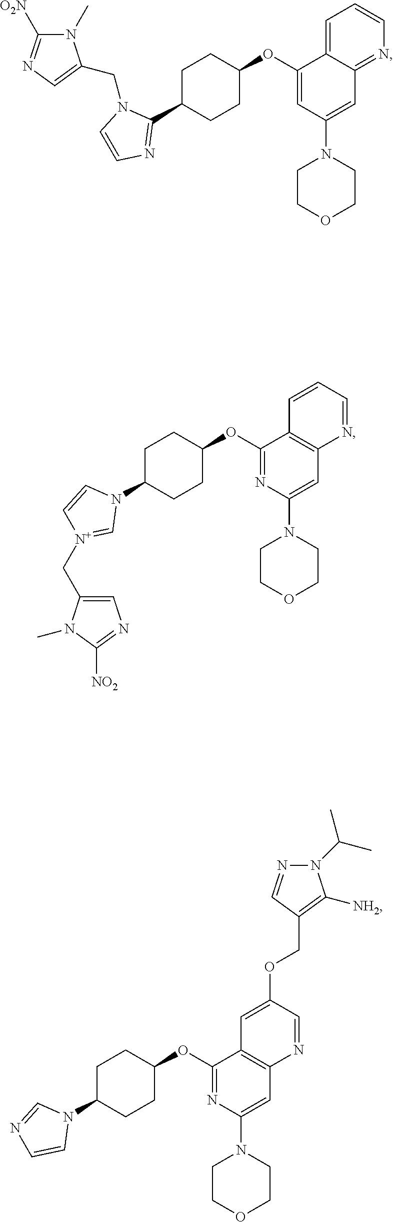 [SMILES: Cn1c(Cn2ccnc2[C@H]2CC[C@@H](Oc3cc(N4CCOCC4)cc4ncccc34)CC2)cnc1[N+](=O)[O-] ; Cn1c(C[n+]2ccn([C@H]3CC[C@@H](Oc4nc(N5CCOCC5)cc5ncccc45)CC3)c2)cnc1[N+](=O)[O-] ; CC(C)n1ncc(COc2cnc3cc(N4CCOCC4)nc(O[C@H]4CC[C@@H](n5ccnc5)CC4)c3c2)c1N] 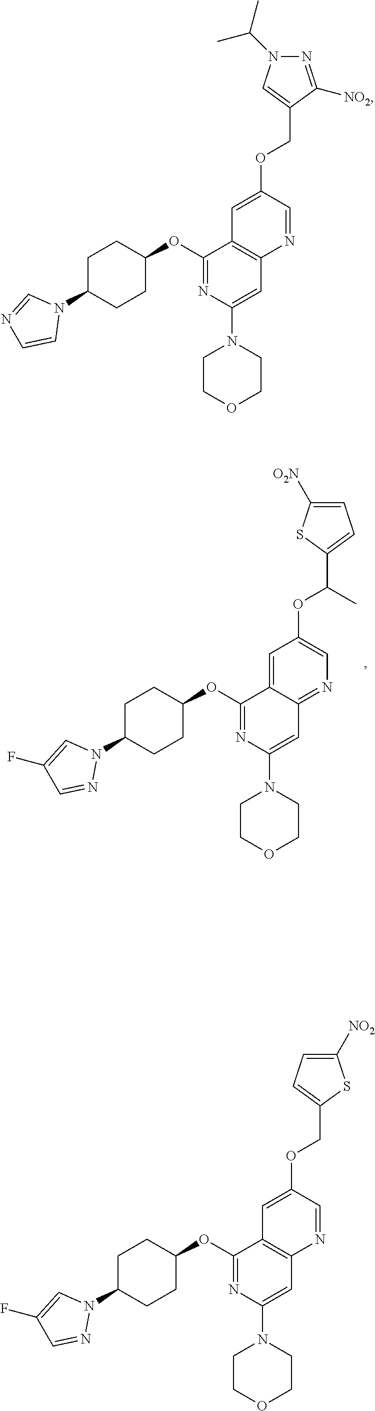 [SMILES: CC(C)n1cc(COc2cnc3cc(N4CCOCC4)nc(O[C@H]4CC[C@@H](n5ccnc5)CC4)c3c2)c([N+](=O)[O-])n1 ; CC(Oc1cnc2cc(N3CCOCC3)nc(O[C@H]3CC[C@@H](n4cc(F)cn4)CC3)c2c1)c1ccc([N+](=O)[O-])s1 ; O=[N+]([O-])c1ccc(COc2cnc3cc(N4CCOCC4)nc(O[C@H]4CC[C@@H](n5cc(F)cn5)CC4)c3c2)s1] 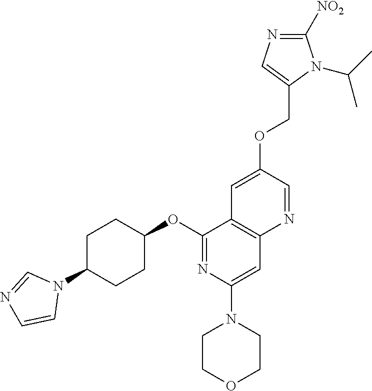 [SMILES: CC(C)n1c(COc2cnc3cc(N4CCOCC4)nc(O[C@H]4CC[C@@H](n5ccnc5)CC4)c3c2)cnc1[N+](=O)[O-]] 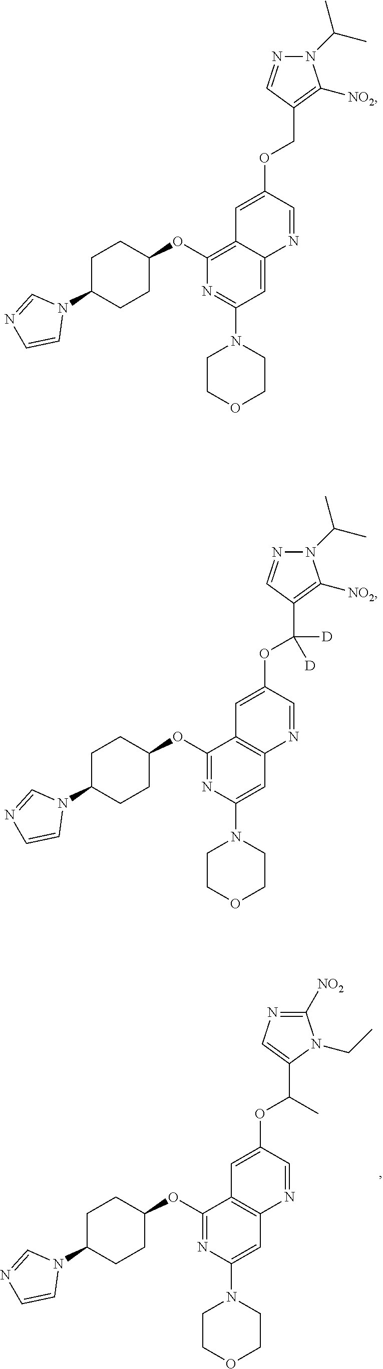 [SMILES: CC(C)n1ncc(COc2cnc3cc(N4CCOCC4)nc(O[C@H]4CC[C@@H](n5ccnc5)CC4)c3c2)c1[N+](=O)[O-] ; [2H]C([2H])(Oc1cnc2cc(N3CCOCC3)nc(O[C@H]3CC[C@@H](n4ccnc4)CC3)c2c1)c1cnn(C(C)C)c1[N+](=O)[O-] ; CCn1c(C(C)Oc2cnc3cc(N4CCOCC4)nc(O[C@H]4CC[C@@H](n5ccnc5)CC4)c3c2)cnc1[N+](=O)[O-]] 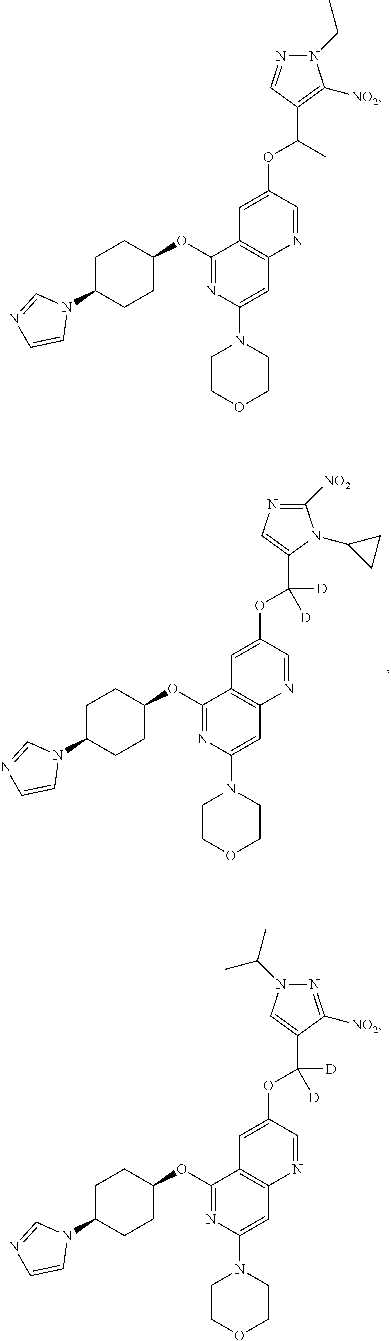 [SMILES: CCn1ncc(C(C)Oc2cnc3cc(N4CCOCC4)nc(O[C@H]4CC[C@@H](n5ccnc5)CC4)c3c2)c1[N+](=O)[O-] ; [2H]C([2H])(Oc1cnc2cc(N3CCOCC3)nc(O[C@H]3CC[C@@H](n4ccnc4)CC3)c2c1)c1cnc([N+](=O)[O-])n1C1CC1 ; [2H]C([2H])(Oc1cnc2cc(N3CCOCC3)nc(O[C@H]3CC[C@@H](n4ccnc4)CC3)c2c1)c1cn(C(C)C)nc1[N+](=O)[O-]] 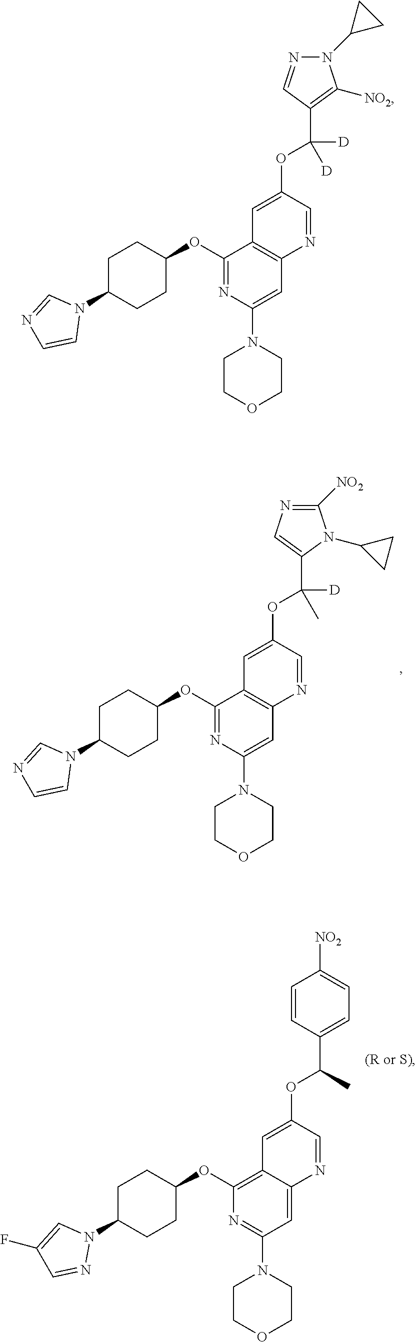 [SMILES: [2H]C([2H])(Oc1cnc2cc(N3CCOCC3)nc(O[C@H]3CC[C@@H](n4ccnc4)CC3)c2c1)c1cnn(C2CC2)c1[N+](=O)[O-] ; [2H]C(C)(Oc1cnc2cc(N3CCOCC3)nc(O[C@H]3CC[C@@H](n4ccnc4)CC3)c2c1)c1cnc([N+](=O)[O-])n1C1CC1 ; C[C@@H](Oc1cnc2cc(N3CCOCC3)nc(O[C@H]3CC[C@@H](n4cc(F)cn4)CC3)c2c1)c1ccc([N+](=O)[O-])cc1] 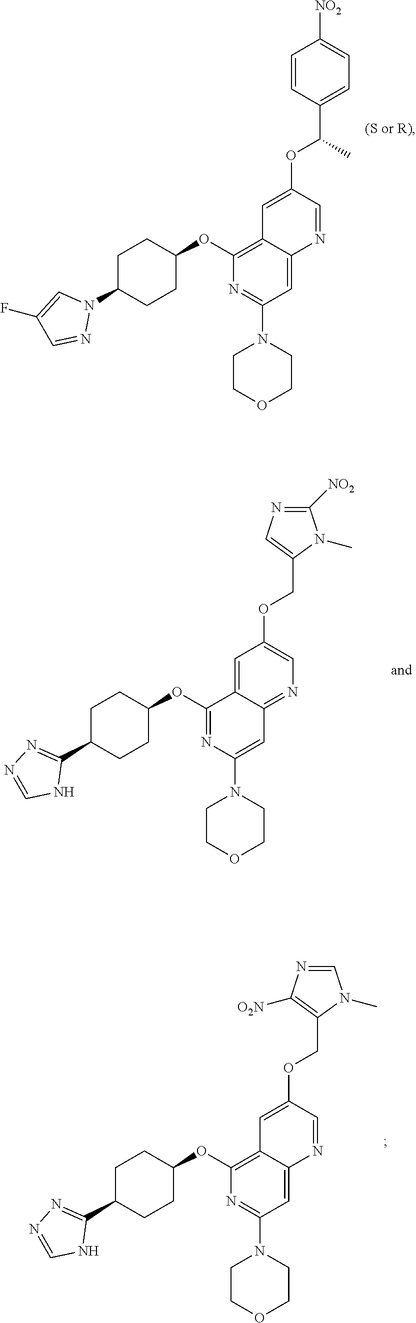 [SMILES: C[C@H](Oc1cnc2cc(N3CCOCC3)nc(O[C@H]3CC[C@@H](n4cc(F)cn4)CC3)c2c1)c1ccc([N+](=O)[O-])cc1 ; Cn1c(COc2cnc3cc(N4CCOCC4)nc(O[C@H]4CC[C@@H](c5nnc[nH]5)CC4)c3c2)cnc1[N+](=O)[O-] ; Cn1cnc([N+](=O)[O-])c1COc1cnc2cc(N3CCOCC3)nc(O[C@H]3CC[C@@H](c4nnc[nH]4)CC3)c2c1]
or a pharmaceutically acceptable salt or N-oxide thereof.

27. A pharmaceutical formulation comprising a compound of claim 1, or prodrug thereof or a pharmaceutically acceptable salt or N-oxide of the compound or the prodrug thereof and a pharmaceutically acceptable excipient.

28.-32. (canceled)

33. A method for the treatment of cancer in a subject, the method comprising administering to the subject a therapeutically effective amount of a compound of claim 1, or prodrug thereof or a pharmaceutically acceptable salt or N-oxide of the compound or the prodrug thereof, wherein the treatment further comprises radiotherapy, a DNA damaging chemotherapeutic agent, or both.

34. The method of claim 33, wherein the treatment further comprises radiotherapy.

35. The method of claim 33, wherein the cancer is a solid cancer.

36. The method of claim 33, wherein the cancer is head and neck cancer.
